SEQUENCE LISTING (41 sequences)

SEQ ID NO: 1 - protein, 28 aa, Homo sapiens
GSSFLSPEHQRVQQRKESKKPPAKLQPR

SEQ ID NO: 2 - protein, 23 aa, Artificial Sequence
  Description of Artificial Sequence: Synthetic peptide
GSSFLSPEHQRVQVRPPKAPHVV

SEQ ID NO: 3 - protein, 24 aa, Artificial Sequence
  Description of Artificial Sequence: Synthetic peptide
  MOD_RES (3)..(3): 2,3-diaminopropionic acid; optionally octanoylated
GSXFLSPEHQRVQVRPPHKAPHVV

SEQ ID NO: 4 - protein, 24 aa, Artificial Sequence
  Description of Artificial Sequence: Synthetic peptide
  MOD_RES (2)..(2): 2,3-diaminopropionic acid
  MOD_RES (3)..(3): 2,3-diaminopropionic acid; optionally octanoylated
GXXFLSPEHQRVQVRPPHKAPHVV

SEQ ID NO: 5 - protein, 28 aa, Artificial Sequence
  Description of Artificial Sequence: Synthetic peptide
GSSFLSPEHQRVQVRPPHKAPHVVPALP

SEQ ID NO: 6 - protein, 5 aa, Artificial Sequence
  Description of Artificial Sequence: Synthetic peptide
  MOD_RES (1)..(1): Inp
  MOD_RES (2)..(2): D-2Nal
  MOD_RES (3)..(3): D-Trp
  C-term NH2
XXWTK

SEQ ID NO: 7 - skipped

SEQ ID NO: 8 - DNA, 84 bp, Homo sapiens
ggctccagcttcctgagccctgaacaccagagagtccagcagagaaaggagtcgaagaagccaccagccaagctgcagccccga

SEQ ID NO: 9 - protein, 29 aa, Artificial Sequence
  Description of Artificial Sequence: Synthetic peptide
GSSFLSPEHQRVQVRPPHKAPHVVPALPL

SEQ ID NO: 10 - protein, 94 aa, Artificial Sequence
  Description of Artificial Sequence: Synthetic polypeptide
GSSFLSPEHQRVQVRPPHKAPHVVPALPLSNQLCDLEQQRHLWASVFSQSTKDSGSDLTVSGRTWGLRVLNQLFPPSSRERSRRSHQPSCSPEL

SEQ ID NO: 11 - protein, 4 aa, Homo sapiens
GSSF

SEQ ID NO: 12 - protein, 5 aa, Homo sapiens
GSSFL

SEQ ID NO: 13 - protein, 6 aa, Homo sapiens
GSSFLS

SEQ ID NO: 14 - protein, 7 aa, Homo sapiens
GSSFLSP

SEQ ID NO: 15 - protein, 8 aa, Homo sapiens
GSSFLSPE

SEQ ID NO: 16 - protein, 9 aa, Homo sapiens
GSSFLSPEH

SEQ ID NO: 17 - protein, 10 aa, Homo sapiens
GSSFLSPEHQ

SEQ ID NO: 18 - protein, 20 aa, Artificial Sequence
  Description of Artificial Sequence: Synthetic peptide
GSSFLSPSQKPQNKVKSSRI

SEQ ID NO: 19 - protein, 28 aa, Artificial Sequence
  Description of Artificial Sequence: Synthetic peptide
GSSFLSPEHQKAQQRKESKKPPAKLQPR

SEQ ID NO: 20 - protein, 28 aa, Artificial Sequence
  Description of Artificial Sequence: Synthetic peptide
GSSFLSPEHQKVQQRKESKKPAAKLKPR

SEQ ID NO: 21 - protein, 28 aa, Artificial Sequence
  Description of Artificial Sequence: Synthetic peptide
GSSFLSPEHQRAQQRKESKKPPAKLQPR

SEQ ID NO: 22 - protein, 26 aa, Artificial Sequence
  Description of Artificial Sequence: Synthetic peptide
GSSFLSPTYKNIQQQKDTRKPTARLH

SEQ ID NO: 23 - protein, 28 aa, Artificial Sequence
  Description of Artificial Sequence: Synthetic peptide
GSSFLSPEHQKLQQRKESKKPPAKLQPR

SEQ ID NO: 24 - protein, 28 aa, Artificial Sequence
  Description of Artificial Sequence: Synthetic peptide
  MOD_RES (3)..(3): Ser(O-n-octanoyl)
GSSFLSPEHQRVQQRKESKKPPAKLQPR

SEQ ID NO: 25 - protein, 29 aa, Artificial Sequence
  Description of Artificial Sequence: Synthetic peptide
  MOD_RES (1)..(1): Fluorescein-Ahx
  MOD_RES (4)..(4): Ser(O-n-octanoyl)
XGSSFLSPEHQRVQQRKESKKPPAKLQPR

SEQ ID NO: 26 - protein, 28 aa, Artificial Sequence
  Description of Artificial Sequence: Synthetic peptide
  MOD_RES (3)..(3): Ser(O-n-octanoyl)
GSSYLSPEHQRVQQRKESKKPPAKLQPR

SEQ ID NO: 27 - protein, 107 aa, Rattus sp.
MEEKVEDDFLDLDAVPETPVFDCVMDIKPETDPASLTVKSMGLQERRGSNVSLTLDMCTPGCNEEGFGYLVSPREESAHEYLLSASRVLRAEELHEKALDPFLLQAE

SEQ ID NO: 28 - protein, 107 aa, Homo sapiens
MEEKIEDDFLDLDPVPETPVFDCVMDIKPEADPTSLTVKSMGLQERRGSNVSLTLDMCTPGCNEEGFGYLMSPREESAREYLLSASRVLQAEELHEKALDPFLLQAE

SEQ ID NO: 29 - protein, 107 aa, Artificial Sequence
  Description of Artificial Sequence: Synthetic polypeptide
MEEKIEDDFLDLDPVPETPVFDCVMDIKPEADPTSLTVKSMGLQERRGANVSLTLDMCTPGCNEEGFGYLMSPREESAREYLLSASRVLQAEELHEKALDPFLLQAE

SEQ ID NO: 30 - protein, 107 aa, Artificial Sequence
  Description of Artificial Sequence: Synthetic polypeptide
MEEKIEDDFLDLDPVPETPVFDCVMDIKPEADPTSLTVKSMGLQERRGANVSLTLDMCEPGCNEEGFGYLMEPREESAREYLLSASRVLQAEELHEKALDPFLLQAE

SEQ ID NO: 31 - protein, 107 aa, Artificial Sequence
  Description of Artificial Sequence: Synthetic polypeptide
MEEKIEDDFLDLDPVPETPVFDAVMDIKPEADPTSLTVKSMGLQERRGSNVSLTLDMCTPGCNEEGFGYLMSPREESAREYLLSASRVLQAEELHEKALDPFLLQAE

SEQ ID NO: 32 - protein, 107 aa, Artificial Sequence
  Description of Artificial Sequence: Synthetic polypeptide
MEEKIEDDFLDLDPVPEEPVFDCVMDIKPEADPTSLTVKSMGLQERRGSNVSLTLDMCTPGCNEEGFGYLMSPREESAREYLLSASRVLQAEELHEKALDPFLLQAE

SEQ ID NO: 33 - protein, 107 aa, Artificial Sequence
  Description of Artificial Sequence: Synthetic polypeptide
MEEKIEDDFLDLDPVPEAPVFDCVMDIKPEADPTSLTVKSMGLQERRGSNVSLTLDMCTPGCNEEGFGYLMSPREESAREYLLSASRVLQAEELHEKALDPFLLQAE

SEQ ID NO: 34 - protein, 107 aa, Artificial Sequence
  Description of Artificial Sequence: Synthetic polypeptide
MEEKIEDDFLDLDPVPEEPVFDAVMDIKPEADPTSLTVKSMGLQERRGANVSLTLDMCEPGCNEEGFGYLMEPREESAREYLLSASRVLQAEELHEKALDPFLLQAE

SEQ ID NO: 35 - protein, 121 aa, Artificial Sequence
  Description of Artificial Sequence: Synthetic polypeptide
MALYGRKKRRQRRRGEEKIEDDFLDLDPVPETPVFDCVMDIKPEADPTSLTVKSMGLQERRGANVSLTLDMCEPGCNEEGFGYLMEPREESAREYLLSASRVLQAEELHEKALDPFLLQAE

SEQ ID NO: 36 - protein, 121 aa, Artificial Sequence
  Description of Artificial Sequence: Synthetic polypeptide
MALYGRKKRRQRRRGEEKVEDDFLDLDAVPETPVFDCVMDIKPETDPASLTVKSMGLQERRGANVSLTLDMCEPGCNEEGFGYLVEPREESAHEYLLSASRVLRAEELHEKALDPFLLQAE

SEQ ID NO: 37 - protein, 22 aa, Artificial Sequence
  Description of Artificial Sequence: Synthetic peptide
  C-term NH2
GIGKFLKKAKKFGKAFVKILLK

SEQ ID NO: 38 - protein, 28 aa, Artificial Sequence
  Description of Artificial Sequence: Synthetic peptide
  MOD_RES (3)..(3): Ser(O-(C=O)-(CH2)6-CH3)
GSSFLSPEHQKAQQRKESKKPPAKLQPR

SEQ ID NO: 39 - DNA, 27 bp, Artificial Sequence
  Description of Artificial Sequence: Synthetic primer
atgtggaacgcgacgcccagcgaagag

SEQ ID NO: 40 - DNA, 27 bp, Artificial Sequence
  Description of Artificial Sequence: Synthetic primer
tcatgtattaatactagattctgtcca

SEQ ID NO: 41 - protein, 4 aa, Artificial Sequence
  Description of Artificial Sequence: Synthetic peptide
  MOD_RES (3)..(3): Ser(n-octanoyl)
GSSF

(19) United States
(12) Patent Application Publication
(10) Pub. No.: US 2020/0261541 A1
(43) Pub. Date: Aug. 20, 2020

(54) METHODS OF TREATING MILD BRAIN INJURY

(71) Applicant: Oxeia Biopharmaceuticals, Inc., San Diego, CA (US)

(72) Inventors: Kartik Kiran Shah, San Diego, CA (US); Amit Dilip Munshi, San Diego, CA (US)

(73) Assignee: Oxeia Biopharmaceuticals, Inc., San Diego, CA (US)

(21) Appl. No.: 16/672,063

(22) Filed: Nov. 1, 2019

Related U.S. Application Data
(63) Continuation of application No. 15/505,067, filed on Feb. 17, 2017, now abandoned.
(60) Provisional application No. 62/046,748, filed on Sep. 5, 2014.
(60) Provisional application No. 62/050,690, filed on Sep. 15, 2014.
(60) Provisional application No. 62/057,184, filed on Sep. 29, 2014.
(60) Provisional application No. 62/057,181, filed on Sep. 29, 2014.
(60) Provisional application No. 62/063,897, filed on Oct. 14, 2014.
(60) Provisional application No. 62/108,948, filed on Jan. 28, 2015.
(60) Provisional application No. 62/118,423, filed on Feb. 19, 2015.
(60) Provisional application No. 62/192,448, filed on Jul. 14, 2015.
(60) Provisional application No. 62/201,530, filed on Aug. 5, 2015.
(60) Provisional application No. 62/039,358, filed on Aug. 19, 2014.

(51) Int. Cl. A61K 38/25; A61P 25/00; A61K 45/06; C07K 14/47; A61K 31/137; C07K 14/60

(52) U.S. Cl. CPC A61K 38/25; A61P 25/00; A61K 45/06; G01N 2800/28; A61K 31/137; C07K 14/60; C07K 14/4702

(57) ABSTRACT

The present disclosure provides methods for treating mild brain injury and other neurological disorders in a subject in need thereof, comprising administering to the subject an effective amount of a compound comprising a ghrelin or ghrelin variant.

### CROSS-REFERENCE TO RELATED APPLICATION

This application claims benefit of priority to U.S. patent application Ser. No. 15/505,067, filed Feb. 17, 2017, which is a U.S. National Stage Application filed under 35 U.S.C. § 371 and claims priority to International Application No. PCT/US2015/045777, filed Aug. 18, 2015, which claims benefit of priority to U.S. Provisional Application No. 62/039,358, filed Aug. 19, 2014, U.S. Provisional Application No. 62/046,748, filed Sep. 5, 2014, U.S. Provisional Application No. 62/050,690, filed Sep. 15, 2014, U.S. Provisional Application No. 62/057,181, filed Sep. 29, 2014, U.S. Provisional Application No. 62/057,184, filed Sep. 29, 2014, U.S. Provisional Application No. 62/063,897, filed Oct. 14, 2014, U.S. Provisional Application No. 62/108,948, filed Jan. 28, 2015, U.S. Provisional Application No. 62/118,423, filed Feb. 19, 2015, U.S. Provisional Application No. 62/192,448, filed Jul. 14, 2015, U.S. Provisional Application No. 62/201,530, filed Aug. 5, 2015, the disclosures of each of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Sep. 23, 2015, is named 107653-0210_SL.txt and is 21,418 bytes in size.

### FIELD OF THE INVENTION

The present disclosure provides methods for treating mild brain injuries, concussion, and other neurological disorders arising from such an injury in a subject in need thereof by administering to the subject an effective amount of a composition comprising a ghrelin variant.

### BACKGROUND

Mild brain injuries (mBI), typically including concussions, having “your bell rung”, and the like, describe an insult to the brain that, in turn, can cause long term injury to the brain. It most often occurs from direct contact to the head, but can also result from indirect injury (e.g., whiplash injury or violent shaking of the head). Individuals who have suffered one brain injury are more at risk for a second brain injury and more susceptible for subsequent injuries. The damage from successive mBIs is cumulative (Cantu, R. C, Second-impact syndrome, Clinics in Sports Medicine, 17(I):37-44, 1998).

The long term damage arising from mBI include cognitive and motor skill deterioration such as psychomotor slowing, poor concentration and attention retrieval resulting in increased variability of performance, and overall executive dysfunction (Stuns et al., Adult Clinical Neuropsychology: Lessons from Studies of the Frontal Lobes, Annual Review of Psychology, 53, 401-433 (2003)). Common examples of the long term effect of mBI are found in soldiers, boxers, football players, soccer players, and the like. Well documented examples are individuals who, long after the occurrence of the mBI(s), begin to manifest the cumulative damage to the brain by loss of one or more cognitive skills and/or motor skills.

The difference between mBI and other brain diseases is that mBI is caused by one or more injuries as opposed to a disease. In the former case, the injuries to the brain cannot be attributed to an underlying pathology but, rather, are the results of the injuries.

Ghrelin is a neuroendocrine hormone that acts as an endogenous ligand for growth hormone secretagogue receptor. It is a 28-amino acid and an endogenously produced peptide predominantly secreted by gastric mucosa. It has been referred to as the “hunger hormone,” due to its well-studied effects on appetite, but it also is believed to play a significant role in regulating the distribution and rate of use of energy.

Regardless, the majority of brain injuries is considered mild in nature and arise from non-disease related phenomena. To date, there has been little to no credible treatment of such mild brain injuries (mBI). Thus, there is a significant unmet need for a therapy for treating mBI that does not impose undesirable costs and delays and are effective. As provided below, this invention is predicated upon the discovery of a profound and surprising effect of ghrelin variants and analogs in the treatment of mBI. Such treatments can be easily administered without delays for individuals suffering from mBI to ensure that they are capable of performing certain tasks safely without risk to themselves or others.

### SUMMARY OF THE INVENTION

This invention provides for methods for treating a mild brain injury or concussion in a patient wherein said method comprises administering to the subject in need thereof suffering from said mild brain injury or concussion an effective amount of a ghrelin variant or a composition comprising a ghrelin variant so as to treat said mild brain injury or concussion. In one embodiment, the ghrelin variant is administered in a non-endogenous carrier.

In some embodiments, the ghrelin variants can be a sequence that includes any of a number of modifications to the wild type ghrelin sequence, which comprises a polypeptide having an amino acid sequence of Gly-Ser-Ser-Phe-Leu-Ser-Pro-Glu-His-Gln-Arg-Val-Gln-Gln-Arg-Lys-Glu-Ser-Lys-Lys-Pro-Pro-Ala-Lys-Leu-Gln-Pro-Arg (SEQ ID NO. 1). Non-limiting examples of potential modifications include modifying the length (shorter or longer) of the sequences, modifying the chemistry of the amino acids, substituting one or more of the amino acids with another amino acid, a synthetic amino acid or otherwise rare or non-naturally occurring amino acid, introducing protecting groups at the N and/or C termini, etc. In some embodiments, the polypeptide is modified with one or more fatty acids. In some embodiments, the fatty acid is an octanoic acid. In some embodiments, the polypeptide is modified at serine at amino acid position 2 and/or serine at amino acid position 3 of SEQ ID No. 1.

For example, in some embodiments, the ghrelin variants include C₁-C₂₀ acylation of the carboxyl group of one or both of the glutamic acid residues or of the C-terminus arginine group. In other embodiments, ghrelin variants include C₁-C₂₀ acylation of one or more of the hydroxyl groups of the serine residues. Yet, in other embodiments, ghrelin variants include replacing one or more of the L-amino acids with a D-amino acid. Every amino acid with the exception of glycine can occur in two isomeric forms, which are called L- and D-forms, analogous to left-handed and right-handed configurations. L-amino acids are the form commonly manufactured in cells and incorporated into proteins. As mentioned above, some ghrelin variants can have one or more of L-amino acids substituted with D-amino acids. In some embodiments, one or more of the ghrelin variants listed above, can be specifically excluded.

In some embodiments, the ghrelin variant comprises or consists of a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO. 1 provided that in some embodiments such variants retain at least 50% of the activity of native ghrelin.

In some embodiments, the ghrelin variant is a ghrelin mimetic such as a compound which is one or more of RM-131 (Rhythm Pharmaceuticals, Boston, Mass.) (or BIM-28131 (Ipsen Group), Dln-101 (DiaLean Ltd., Israel), Growth hormone (GH) releasing hexapeptide (GHRP)-6, EP 1572, Ape-Ser(Octyl)-Phe-Leu-aminoethylamide, isolated ghrelin splice variant-like compound, ghrelin splice variant, growth hormone secretagogue receptor GHS-R 1a ligand, and a combination thereof. In some embodiments, one or more of the ghrelin variants listed above, can be specifically excluded. In some embodiments, those ghrelin variants which are a polypeptide have at least 80%, 85%, 89%, 90%, 91%, 92%, 93%, 94%, 95%, 96%, 97%, 98%, or 99% sequence identity to the amino acid sequence of one or more of the compounds described in the present disclosure. In some embodiments, ghrelin variants, which comprise short amino acid sequences, such as the RM-131 pentapeptide molecule, can have a substitution of one of its amino acids, for example, a conservative or other type of substitution as described herein with a natural or non-natural amino acids, as well as isomers of the same. Other chemical modifications also are contemplated, such as those described elsewhere herein (e.g., protecting groups, octanoylation, acylation, etc.). In some embodiments, one or more of the substitution listed above, can be specifically excluded.

In some embodiments, the ghrelin variant is one or more of RM-131 (or BIM-28131), Dln-101, Growth hormone (GH) releasing hexapeptide (GHRP)-6, EP 1572, Ape-Ser(Octyl)-Phe-Leu-aminoethylamide, isolated ghrelin splice variant-like compound, ghrelin splice variant, growth hormone secretagogue receptor GHS-R 1a ligand, LY444711, LY426410, hexarelin/examorelin, growth hormone releasing hexapeptide-1 (GHRP-I), GHRP-2, GHRP-6 (SK&F-110679), ipamorelin, MK-0677, NN703, capromorelin, CP 464709, pralmorelin, macimorelin (acetate), anamorelin, relamorelin, ulimorelin, ipamorelin, tabimorelin, ibutamoren, G7039, G7134, G7203, G-7203, G7502, SM-130686, RC-1291, L-692429, L-692587, L-739943, L-163255, L-163540, L-163833, L-166446, CP-424391, EP-51389, NNC-26-0235, NNC-26-0323, NNC-26-0610, NNC 26-0703, NNC-26-0722, NNC-26-1089, NNC-26-1136, NNC-26-1137, NNC-26-1187, NNC-26-1291, MK-0677, L-692,429, EP 1572, L-252,564, NN703, S-37435, EX-1314, PF-5190457, AMX-213, and a combination thereof. In some embodiments, one or more of the ghrelin variants listed above, can be specifically excluded.

In some embodiments, the ghrelin variant comprises a polypeptide comprising the sequence of Gly Ser Ser Phe Leu Ser Pro Glu His Gln Arg Val Gln Val Arg Pro Pro Lys Ala Pro His Val Val (SEQ ID No. 2). In some embodiments, the ghrelin variant comprises a polypeptide comprising the sequence of Gly Ser Xaa Phe Leu Ser Pro Glu His Gln Arg Val Gln Val Arg Pro Pro His Lys Ala Pro His Val Val (SEQ ID No. 3), wherein the third position is a 2,3-diaminopropionic acid (Dpr), with the Dpr in the third position being optionally octanoylated. In some embodiments, the ghrelin variant comprises a polypeptide comprising the sequence of Gly Xaa Xaa Phe Leu Ser Pro Glu His Gln Arg Val Gln Val Arg Pro Pro His Lys Ala Pro His Val Val (SEQ ID No. 4), wherein the second and third position are 2,3-diaminopropionic acid (Dpr) residues, with the Dpr in the third position being optionally octanoylated. In some embodiments, the ghrelin variant comprises a polypeptide comprising the sequence of Gly Ser Ser Phe Leu Ser Pro Glu His Gln Arg Val Gln Val Arg Pro Pro His Lys Ala Pro His Val Val Pro Ala Leu Pro (SEQ ID No. 5). In some embodiments, the ghrelin variant comprises a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of one or more of the compounds described in the present paragraph. In some embodiments, the ghrelin variant comprises a polypeptide comprising the sequence of Inp-D-2Nal-D-Trp-Thr-Lys-NH₂ (SEQ ID No. 6). In some embodiments, one or more of the ghrelin variants listed above, can be specifically excluded.

In some embodiments, one or more of the amino acids of the sequence are substituted or replaced by another amino acid or a synthetic or otherwise rare amino acid (e.g., 4-fluoroproline, 4-hydroxyproline, 4-ketoproline, H₂NCD₂COOH, and the like). Some non-limiting examples of potential molecules that can be substituted for amino acids are provided below in Table 3. In some embodiments, one or more of the substitutions listed above, can be specifically excluded.

In some embodiments, the substitution is between 1 and 5 amino acids. Substitution of one amino acid for another can be based on accepted and art recognized substitution principles. In some embodiments, one or more amino acids can be substituted with an amino acid or synthetic amino acid that has a similar property or a different property at its side chain or otherwise, such as charge, polarity, hydrophobicity, antigenicity, propensity to form or break α-helical structures or β-sheet structures. Non-limiting examples of common substitutions for various residues can be found in the NCBI Amino Acid Explorer database, which includes listings of common substitutions for each amino acid, along other types of information on each amino acid as part of its BLOSUM62 matrix database (see Substitutes in BLOSUM62 on the worldwide web at: ncbi.nlm.nih.gov/Class/Structure/aa/aa_explorer.cgi. In some embodiments the substitutions can be conservative substitutions, which are well known in the art (see for example Creighton (1984) Proteins. W. H. Freeman and Company (Eds), which is incorporated herein by reference in its entirety). Table 2 below depicts non-limiting examples of conservative substitutions that can be made. In some embodiments, one or more of the substitutions listed above, can be specifically excluded.

Some embodiments include the deletion or substitution of one or more amino acids from the sequences described herein. A deletion refers to removal of one or more amino acids from a sequence. An insertion refers to one or more amino acid residues being introduced into a site in a sequence. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. In some non-limiting embodiments, N- or C-terminal fusion proteins or peptides can include linking another molecule of the same sequence or a completely different molecule, including any of those described herein to the N- or C-terminus of a ghrelin variant. In some cases, the linker can be via an ester bond or other bond or linker that can rapidly degrade in the body to liberate both of the linked molecules. In some embodiments, natural ghrelin or a fragment thereof can be linked to a ghrelin variant as described herein. In still further embodiments, the two or more molecules can be linked via a cyclized linker. For example, a diacid such as those represented by the formula R—CH(COOH)(CH₂)ₙCOOH where R is a saturated or unsaturated aliphatic group of from 1 to 20 carbon atoms and n is 0, 1, 2, 3, or 4, can be utilized. In some cases the cyclized linker can provide added benefit, for example, resistance against protease degradation. In some embodiments, one or more of the substitutions or modifications listed above, can be specifically excluded.

In some embodiments, the ghrelin variant is one or more of LY444711 (2(R)-(2-Amino-2-methylpropanamido)-N-[1-[1(R)-(4-methoxyphenyl)-1-methyl-2-oxo-2-(1-pyrrolidinyl)ethyl]-1H-imidazol-4-yl]-5-phenylpentanamide dihydrochloride, C₃₂H₄₄Cl₂N₆O₄; Ely Lilly), MK-0677 (2-Amino-N-[2-benzyloxy-(1R)-[1-(methanesulfonyl)spiro[indoline-3,4′-piperidin]-1′-ylcarbonyl]ethyl]isobutyramide methanesulfonate, C₂₈H₄₀N₄O₈S₂; Merck & Co., Inc.), L-692,429 (Merck Research Laboratories), Tabimorelin (NNC 26-0703, NN703; N-[5-Amino-5-methyl-2(E)-hexenoyl]-N-methyl-3-(2-naphthyl)-D-alanyl-N-methyl-D-phenylalanine methylamide, C₃₂H₄₀N₄O₃; Novo Nordisk), Capromorelin (CP-424,391-18; RQ-00000005; CP-424391; 2-Amino-N-[2-[3a(R)-benzyl-2-methyl-3-oxo-3,3a,4,5,6,7-hexahydro-2H-pyrazolo[4,3-c]pyridin-5-yl]-1(R)-(benzyloxymethyl)-2-oxoethyl]isobutyramide, C₂₈H₃₅N₅O₄; Pfizer and RaQualia, Japan), L-252,564 (Merck), G-7203, S-37435 (N-[1(R)-[N-(3-Amino-2-hydroxypropyl)carbamoyl]-2-naphthylethyl]-4-(4-oxo-2,3,4,5-tetrahydro-1,5-benzothiazepin-5-yl)butyramide hydrochloride, C₂₉H₃₅ClN₄O₄S; Kaken/Molecular Research Institute), SM-130868 ((+)-(3S)-3-(2-chlorophenyl)-1-[2-(diethylamino)ethyl]-3-hydroxo-2-oxo-4-(trifluoromethyl)indoline-6-carboxamide, C₂₂H₂₃ClF₃N₃O₃; Sumitomo), EX-1314 (Carbamic acid, (2-amino-2-oxoethyl)methyl-, (3-((1S)-1-((2-amino-2-methyl-1-oxopropyl)amino)-2-(phenylmethoxy)ethyl)-1,2,4-triazolo(4,3-a)pyridin-5-yl)methyl ester, monohydrochloride, C₂₄H₃₁N₇O₅ HCl; Elixir Pharmaceuticals), ulimorelin((2R,5S,8R,11R)-5-Cyclopropyl-11-(4-fluorobenzyl)-2,7,8-trimethyl-4,5,7,8,10,11,13,14,15,16-decahydro-2H-1,4,7,10,13-benzoxatetraazacyclooctadecine-6,9,12(3H)-trione, C₃₀H₃₉FN₄O₄; Tranzyme Pharma, Inc., Ocera Therapeutics, Lyric Pharmaceuticals, Inc.), macimorelin (acetate) (EP 1572; 2-Amino-N-[(2R)-1-[[(1R)-1-formamido-2-(1H-indol-3-yl)ethyl]amino]-3-1H-indol-3-yl)-1-oxopropan-2-yl]-2-methylpropanamide, C₂₆H₃₀N₆O₃; AEterna Zentaris Inc.), anamorelin (HCl) (2-amino-N—((R)-1-((R)-3-benzyl-3-(1,2,2-trimethylhydrazinecarbonyl)piperidin-1-yl)-3-(1H-indol-3-yl)-1-oxopropan-2-yl)-2-methylpropanamide hydrochloride, C₃₁H₄₃ClN₆O₃; Helsinn Group), ipamorelin (2-Methylalanyl-L-histidyl-3-(2-naphthyl)-D-alanyl-D-phenylalanyl-L-lysinamide, C₃₈H₄₉N₉O₅;), PF-5190457, GHRP-6 (L-histidyl-D-tryptophyl-L-alanyl-L-tryptophyl-D-phenylalanyl-L-Lysinamide), AMX-213 (Ammunix), and a combination thereof. In some embodiments, one or more of the ghrelin variants listed above, can be specifically excluded.

In some embodiments, the ghrelin variant is one or more of LY426410 (2-Methylalanyl-N-[1-[1(R)-(4-methoxyphenyl)-2-(4-methyl-1-piperidinyl)-2-oxoethyl]-1H-imidazol-4-yl]-O-benzyl-D-serinamide; 2-Amino-N-[1(R)-(benzyloxymethyl)-2-[1-[1(R)-(4-methoxyphenyl)-2-(4-methyl-1-piperidinyl)-2-oxoethyl]-1H-imidazol-4-ylamino]-2-oxoe), hexarelin/examorelin ((2S)-6-amino-2-[[(2R)-2-[[(2S)-2-[[(2S)-2-[[(2R)-2-[[(2S)-2-amino-3-(1H-imidazol-5-yl)propanoyl]amino]-3-(2-methyl-1H-indol-3-yl)propanoyl]amino]propanoyl]amino]-3-(1H-indol-3-yl)propanoyl]amino]-3-phenylpropanoyl]amino]hexanamide), growth hormone releasing hexapeptide-1 (GHRP-I), GHRP-2, GHRP-6 (SK&F-110679), CP 464709 (2-amino-N-[(2R)-1-[(3aR)-3-oxo-3a-(pyridin-2-ylmethyl)-2-(2,2,2-trifluoroethyl)-6,7-dihydro-4H-pyrazolo[4,3-c]pyridin-5-yl]-3-[(2,4-difluorophenyl)methoxy]-1-oxopropan-2-yl]-2-methylpropanamide), pralmorelin ((2S)-6-amino-2-[[(2S)-2-[[(2S)-2-[[(2S)-2-[[(2R)-2-[[(2R)-2-aminopropanoyl]amino]-3-naphthalen-2-ylpropanoyl]amino]propanoyl]amino]-3-(1H-indol-3-yl)propanoyl]amino]-3-phenylpropanoyl]amino]hexanamide), relamorelin (4-[[(2S)-2-[[(2R)-2-[[(2R)-3-(1-benzothiophen-3-yl)-2-(piperidine-3-carbonylamino)propanoyl]amino]-3-(1H-indol-3-yl)propanoyl]amino]-3-phenylpropanoyl]amino]piperidine-4-carboxamide), ulimorelin ((7R,10R,13S,16R)-13-cyclopropyl-7-[(4-fluorophenyl)methyl]-10,11,16-trimethyl-17-oxa-5,8,11,14-tetrazabicyclo[16.4.0]docosa-1(22),18,20-triene-6,9,12-trione), tabimorelin (N-[(2E)-5-amino-5-methylhex-enoyl]-N-methyl-3-(2-naphthyl)alanyl-N,Nα-dimethyl-D-phenylalaninamide; NN703; NNC 26-0703), ibutamoren (2-amino-2-methyl-N-[(2R)-1-(1-methylsulfonylspiro[2H-indole-3,4T-piperidine]-1T-yl)-1-oxo-3-phenylmethoxypropan-2-yl]propanamide), G7039 (Genentech), G7134 (Genentech), G7502 ([[5-(2-amino-6-oxo-3H-purin-9-yl)-3,4-dihydroxyoxolan-2-yl]methoxy-hydroxyphosphoryl] [3,4,5-trihydroxy-6-(hydroxymethyl)oxan-2-yl] hydrogen phosphate), Anamorelin/RC-1291 (2-amino-N-[(2R)-1-[(3R)-3-benzyl-3-[dimethylamino(methyl)carbamoyl]piperidin-1-yl]-3-(1H-indol-3-yl)-1-oxopropan-2-yl]-2-methylpropanamide), L-692429 (3-amino-3-methyl-N-[(3R)-2-oxo-1-[[4-[2-(2H-tetrazol-5-yl)phenyl]phenyl]methyl]-4,5-dihydro-3H-1-benzazepin-3-yl]butanamide), Pexiganan [INN]/L-692587 (Glycyl-L-isoleucylglycyl-L-lysyl-L-phenylalanyl-L-leucyl-L-lysyl-L-lysyl-L-alanyl-L-lysyl-L-lysyl-L-phenylalanylglycyl-L-lysyl-L-alanyl-L-phenylalanyl-L-valyl-L-lysyl-L-isoleucyl-L-leucyl-L- lysyl-L-lysinamide) (SEQ ID NO: 37), CHEMBL291200/L-739943 (3-amino-3-methyl-N-[(3R)-1-[[4-[2-[(methylcarbamoylamino)methyl]phenyl]phenyl]methyl]-2-oxo-4,5-dihydro-3H-1-benzazepin-3-yl]butanamide), L-163255 (2-amino-2-methyl-N-[(2R)-1-(1-methylsulfonylspiro[2H-indole-3,4′-piperidine]-1′-yl)-1-oxo-5-phenylpentan-2-yl]propanamide), L-163,540 (1-[2(R)-(2-amino-2-methylpropionylamino)-3-(1H-indol-3-yl)propionyl]-3-benzylpiperidine-3(S)-carboxylic acid ethyl ester), L-163833, L-166446, EP-51389, NNC-26-0235 (3-(aminomethyl)benzoyl-2-naphthylalanyl-N-methylphenylalanyl-lysinamide, 3-(aminomethyl)benzoyl-2Nal-N-Me-Phe-Lys-NH2, NNC-26-0235), NNC-26-0323 (3-(aminomethyl)benzoyl-D-2Nal-N-Me-D-Phe-Lys-NH2), NNC-26-0610 (Novo Nordisk), NNC-26-0722, NNC-26-1089, NNC-26-1136, NNC-26-1137, NNC-26-1187, NNC-26-1291, EP 1572 (Aib-DTrp-DgTrp-CHO), S-37435 (N-[(2R)-1-[(3-amino-2-hydroxypropyl)amino]-3-naphthalen-2-yl-1-oxopropan-2-yl]-4-(1,1,4-trioxo-2,3-dihydro-1{6},5-benzothiazepin-5-yl)butanamide), and a combination thereof. In some embodiments, one or more of the ghrelin variants listed above, can be specifically excluded.

In some embodiments, the ghrelin variant binds to the growth hormone secretagogue receptor GHS-R 1a (GHSR). In some embodiments, the ghrelin variant has an EC₅₀ potency on the GHSR of less than 500 nM. In some embodiments, the ghrelin variant has a dissociation constant from the GHSR of less than 500 nM.

In some embodiments, the ghrelin variant has at least about 50% of the functional activity of ghrelin. In some embodiments, the functional activity comprises one or more of feeding regulation, nutrient absorption, gastrointestinal motility, energy homeostasis, anti-inflammatory regulation, suppression of inflammatory cytokines, activation of Gq/G11, accumulation of inositol phosphate, mobilization of calcium from intracellular stores, activation or deactivation of MAP kinases, NFκB translocation, CRE driven gene transcription, binding of arrestin to ghrelin receptor, reduction in reactive oxygen species (ROS), NAMPT enzyme activation, or a combination thereof. In some embodiments, one or more of the functional activities listed above, can be specifically excluded.

In some embodiments, the ghrelin variant increases uncoupling protein-2 (UCP-2) expression. In some embodiments, the ghrelin variant increases UCP-2 expression in mitochondria.

In some embodiments, the ghrelin variant prevents or reduces the metabolic consequences of mBI and/or any associated sequelae, including any associated chronic conditions. In some embodiments, the ghrelin variant prevents or reduces one or more post concussive syndrome symptoms or delays the onset thereof.

In some embodiments, the ghrelin variant is coupled to a protein that extends the serum half-lives of the ghrelin variant. In some embodiments, the protein is a long, hydrophilic, and unstructured polymer that occupies a larger volume than a globular protein containing the same number of amino acids. In some embodiments, the molecule that extends the half-life can be a molecule set forth in WO 2013/130683 entitled “XTEN Conjugate Compositions and Methods of Making the Same,” and U.S. Pat. No. 8,673,860, entitled Extended Recombinant Polypeptides and Compositions Comprising the Same,” each of which is incorporated herein by reference in its entirety. One example of a molecule that incorporates such XTEN molecules is AMX-213 (Ammunix). In some embodiments, the protein comprises the sequence of XTEN (SEQ ID NO. 7). In some embodiments, the mild brain injury comprises a concussion. In some embodiments, the extender sequence can have the chemical structure:

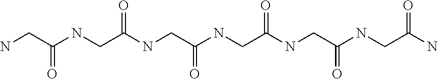 [SMILES: NCC(=O)NCC(=O)NCC(=O)NCC(=O)NCC(=O)NCC(N)=O]

In some embodiments, the subject that undergoes the method of treatment is a mammal. In some embodiments, the subject is a human. In some embodiments, the ghrelin variant is administered within not more than about 8, 24 or 72 hours of the mBI. In some embodiments, the ghrelin variant is administered within not more than about 24 hours after the mBI. In some embodiments, the ghrelin variant is administered within or at about 0.1, 0.3, 0.5, 0.7, 1, 2, 3, 6, 8, 12, 18, 24, 36, 48, or 72 hours after the mBI. Additionally, in some embodiments, 1, 2, 3 or more follow up dosages can be provided in the 1-14 days after the injury occurs or after the initial administration after the injury.

The present disclosure provides for methods of reducing the incidence or severity of mBI in a subject, comprising administering to the subject an effective amount of ghrelin, thereby reducing the incidence or severity of the mBI. In some embodiments, ghrelin is administered prior to an event or activity with a potential for occurrence of mBI.

This invention provides for methods of reducing the amount of time required for recovery from a mild brain injury or concussion, comprising administering to a patient suffering from a mild brain injury or concussion a therapeutically effective amount of ghrelin within 72 hours of the mild brain injury or concussion. In some embodiments, ghrelin is administered in a single dose. In some embodiments, ghrelin is administered at a dosage from 10 ng/kg per day to 10 mg/kg per day.

The present disclosure also provides for a method of reducing the incidence or severity of mBI or the symptoms or sequelae of a mild brain injury or concussion in a subject, comprising administering to the subject an effective amount of a ghrelin variant, a composition comprising a ghrelin variant or a therapeutic product as described herein, thereby reducing the incidence or severity of the mBI or concussion. Some embodiments relate to methods of preventing the development of, or reducing the risk of developing, short- and long-term sequelae associated with mild brain injury or concussion. Such methods can be used for immediate treatment of the injury, the symptoms thereof and the sequelae, and also can be used to prevent or reduce long term or chronic sequelae.

In some embodiments, the composition comprising ghrelin or a ghrelin variant is administered prior to an event or activity with a potential for occurrence of mBI or concussion. In some embodiments, the event or activity is participation in a sporting event, physical training, or combat. In some embodiments, the event or activity is baseball, basketball, rugby, football, hockey, lacrosse, soccer, cycling, boxing, a martial art, a mixed martial art, a military exercise, automobile racing, snow skiing, snowboarding, ice skating, skateboarding, motocross, mountain biking, motorcycle and ATV riding, and the like. In some embodiments, the subject has not suffered a mBI or concussion. In some embodiments, the subject has a history of mBI or is susceptible to mBI or concussion. When administered or used, a ghrelin variant can be administered up to 72 hour immediately preceding an event that may induce a mild brain injury or concussion.

In some embodiments, the composition comprising ghrelin or the ghrelin variant is administered via a powder or stable formulation, wherein the ghrelin variant is formulated in a dosage form selected from the group consisting of: liquid, beverage, medicated sports drink, powder, capsule, chewable tablet, swallowable tablet, buccal tablet, troche, lozenge, soft chew, solution, suspension, spray, suppository, tincture, decoction, infusion, and a combination thereof.

In some embodiments, the composition comprising ghrelin or the ghrelin variant is administered via inhalation, oral, intravenous, parenteral, buccal, subcutaneous (including “EpiPens”), transdermal, patch, sublingual, into the inner ear, intramuscular, or intranasal. In some embodiments, the ghrelin variant is administered in a single dose. In some embodiments, the ghrelin variant is administered at a dosage from 10 ng/kg per day to 10 mg/kg per day. In one preferred embodiment, a ghrelin variant is administered passively such as by sublingual, inner ear or pulmonary delivery.

In some embodiments, the composition comprising ghrelin or a ghrelin variant is administered in combination with a therapeutic agent. In some embodiments, the therapeutic agent is one or more of an anti-inflammatory agent, anti-pain medication, acetylsalicylic acid, an antiplatelet agent, a thrombolytic enzyme, an aggregation inhibitor, a glycoprotein IIb/IIIa inhibitor, a glycosaminoglycan, a thrombin inhibitor, an anticoagulant, heparin, coumarin, tPA, GCSF, streptokinase, urokinase, Ancrod, melatonin, a caspase inhibitor, NMDA receptor agonist or antagonist (e.g., amantadine or gacyclidine/OTO-311, 1-[(1R,2S)-2-methyl-1-thiophen-2-ylcyclohexyl]piperidine, C₁₆H₂₅NS), P7C3, P2Y receptor agonist, glucagon, GLP-1R agonists, GLP-1, GLP-1 analog, synthetic form of GLP-1, GLP-1 (7-36) amide, Exendin-4 (Ex-4), Ex-4 analog, synthetic form of Ex-4, Lixisenatide, Liraglutide, a molecule in a biological pathway involving GLP-1R signaling pathway, incretin, incretin mimetic, Gastric inhibitory polypeptide (GIP), sulfonamide compounds, Ebselen (SPI-1005 or 2-phenyl-1,2-benzisoselenazol-3(2H)-one; Sound Pharmaceuticals; Kil J, et al. Hear Res. 2007 April; 226 (1-2):44-51. Lynch E D, et al. Laryngoscope. 2004 February; 114(2):333-7), glutathione peroxidase, glutathione peroxidase mimics and inducers, aducanumab (BIIB037, Biogen ldec), or a combination thereof. In some embodiments, one or more of the therapeutic agents listed above, can be specifically excluded.

In some embodiments, the therapeutic agent is a biologically equivalent polynucleotide that has the specified percent homology (for example, 60%, 65%, 70%, 75%, 80%, 85%, 90%, 95%, 98% or 99%) and encoding a polypeptide having the same or similar biological activity as the therapeutic agent described above.

In some embodiments, the therapeutic agent is not a SARM compound. In some embodiments, one or more therapeutic agents disclosed herein are excluded from the combination therapy with ghrelin or the ghrelin variant.

Some embodiments relate to methods of reducing the amount of time needed to recover from a mild brain injury or concussion, which methods can include for example, administering to a patient or subject suffering from a mild brain injury or concussion a therapeutically effective amount of a ghrelin variant within 72 hours of the mild brain injury or concussion.

In some aspects, of the various methods and uses described herein, the patient or subject can be a human infant between the age of newly born and 1 year, a child between the age of 1 and 12, a child between the age of 12 and 18, an adult between the age of 18 and 65 or an elderly adult age 65 and older.

In some embodiments, the composition comprising ghrelin or the ghrelin variant can be administered via a powder or stable formulation, wherein the ghrelin variant is formulated in a dosage form such as a liquid, beverage, medicated sports drink, powder, capsule, chewable tablet, hydrogel, swallowable tablet, buccal tablet, troche, lozenge, soft chew, solution, suspension, spray, suppository, tincture, decoction, infusion, and a combination thereof. In some embodiments, the ghrelin variant can be administered via inhalation, oral, intravenous, parenteral, buccal, subcutaneous (including “EpiPens”), transdermal, patch, sublingual, intramuscular, intratympanic injection or placement, or intranasal. Without limitation, the ghrelin variant can be administered in a single dose, in two doses, in three doses, in four doses, in five doses or in multiple doses. In some embodiments, the ghrelin variant can be administered at a dosage from 10 ng/kg per day to 10 mg/kg per day.

In some embodiments, the composition comprising ghrelin or the ghrelin variant can be administered in combination with a therapeutic agent. Without being limited thereto, the therapeutic agent can be one or more of an anti-inflammatory agent, anti-pain medication, acetylsalicylic acid, an antiplatelet agent, a thrombolytic enzyme, an aggregation inhibitor, a glycoprotein IIb/IIIa inhibitor, a glycosaminoglycan, a thrombin inhibitor, an anticoagulant, heparin, coumarin, tPA, GCSF, streptokinase, urokinase, Ancrod, melatonin, a caspase inhibitor, an NMDA receptor agonist or antagonist (e.g. OTO-311), an anti-TNF-α compound, an antibody, erythropoietin/EPO, angiotensin II lowering agent, selective androgen receptor modulator, leptin or leptin mimetics and variants, an agonists of the renin-angiotensin system, an opioid receptor agonist, progesterone or progesterone mimetics and variants, a peroxisome proliferator-activated receptor gamma agonist, amantadine (e.g. ADS-5102), P7C3, P2Y receptor agonist, glucagon, GLP-1R agonists, GLP-1, GLP-1 analog, synthetic form of GLP-1, GLP-1 (7-36) amide, Exendin-4 (Ex-4), Ex-4 analog, synthetic form of Ex-4, Lixisenatide, Liraglutide, a molecule in a biological pathway involving GLP-1R signaling pathway, incretin, incretin mimetic, Gastric inhibitory polypeptide (GIP), sulfonamide compounds, Ebselen (2-phenyl-1,2-benzisoselenazol-3(2H)-one), SPI-1005 (Sound Pharmaceuticals), glutathione peroxidase, glutathione peroxidase mimics and inducers, aducanumab (BIIB037, Biogen Idec), or a combination thereof. In some embodiments, one or more therapeutic agents disclosed herein are excluded from the combination therapy with ghrelin or the ghrelin variant.

Some embodiments relate to therapeutic products that include, for example, at least two agents selected from ghrelin, a ghrelin variant, an anti-inflammatory agent, anti-pain medication, acetylsalicylic acid, an antiplatelet agent, a thrombolytic enzyme, an aggregation inhibitor, a glycoprotein IIb/IIIa inhibitor, a glycosaminoglycan, a thrombin inhibitor, an anticoagulant, heparin, coumarin, tPA, GCSF, streptokinase, urokinase, Ancrod, melatonin, a caspase inhibitor, an NMDA receptor agonist or antagonist, an anti-TNF-α compound, an antibody, erythropoietin/EPO, angiotensin II lowering agent, selective androgen receptor modulator, leptin or leptin mimetics and variants, an agonists of the renin-angiotensin system, an opioid receptor agonist, progesterone or progesterone mimetics and variants, a peroxisome proliferator-activated receptor gamma agonist, an NMDA receptor agonist or antagonist (e.g. OTO-311), P7C3, P2Y receptor agonist, and amantadine (e.g. ADS-5102). In some embodiments, the at least two agents can be bound together. For example, they can be bound together to form a dimer, a trimer, a tetramer or a pentamer. They can be conjugated, fused, or otherwise bound together in such a manner that upon administration in vivo, the agents separate or disassociate. For example, the two agents can be ghrelin molecules bound together. In some embodiments at least one of the two agents is ghrelin or at least one is a ghrelin variant. For example, the ghrelin variant can be a peptide of between 15 amino acids and 40 amino acids, a peptide of between 4 amino acids and 14 amino acids, a small molecule pharmaceutical or a combination of the same. In some embodiments, the products further may include a pharmaceutically acceptable excipient, such as saline. In some embodiments, one or more the agents listed above can be specifically excluded.

Still some embodiments relate to methods of treating a mild brain injury or concussion or reducing the onset of or severity of a mild brain injury or concussion, for example, by administering a therapeutically effect amount of the therapeutic product as described herein. Some embodiments relate to methods method of reducing the onset of or severity of a one or more symptoms or sequelae of a mild brain injury or concussion, comprising administering a therapeutically effect amount of the therapeutic product as described herein.

Still further, ghrelin can be used in an assay to assess the ability of candidate compounds to effect increased uncoupling protein-2 (UCP-2) expression including increased UCP-2 expression in mitochondria. In such assays, ghrelin is used as a control to determine the relative efficacy of the candidate compound or compounds. Suitable assays include by way of example only competitive assays for binding of a candidate compound or compounds to growth hormone secretagogue receptor 1a (i.e., GHSR) in the presence of ghrelin as well as frontal affinity chromatography.

In yet another embodiment, a patient suffering loss of cognitive or motor skills due to mBI or concussion and, in particular, repetitive mBI or repetitive concussion, can be monitored for therapy or progression of such skills by correlating the ghrelin level in the patient's brain over time. As the ghrelin levels decrease, there will be an increased need for intervention. This invention also provides for methods of measuring ghrelin levels before an activity, for example before the start of football, soccer, rugby or any other sport or activity season, and monitoring during the season to ascertain if the player is at a level not qualified to play or participate by utilizing a test or an assay for measuring ghrelin levels, such as a test or assay for determining levels in the blood.

In some embodiments, the present invention provides for a method for treating a subject suffering from metabolic derangements associated with mBI or concussion, wherein such method comprises administration of an effective amount of ghrelin or a ghrelin variant to the subject, thereby treating the subject suffering from metabolic derangements associated with mBI or concussion.

In some embodiments, the present invention provides for a method for treating a subject suffering from increased levels of reactive oxygen species (ROS) in neurons associated with mBI or concussion, wherein such method comprises administration of an effective amount of a ghrelin or ghrelin variant to the subject so as to decrease levels of ROS in neurons associated with mBI or concussion, thereby treating the subject suffering from metabolic derangements associated with mBI or concussion.

In some embodiments, the present invention provides for a method for preventing chronic traumatic encephalopathy (CTE) associated with repeated mBI or concussions in a subject, wherein such method comprises administration of one or more doses of a ghrelin or ghrelin variant to the subject, thereby preventing chronic traumatic encephalopathy (CTE) associated with repeated mBI or concussions in a subject.

In some embodiments, the present invention provides for a method for preventing damage to neurons associated with oxidative stress and overproduction of reactive oxygen species (ROS) in a subject with one or more incidence of mBI or concussion, wherein such method comprises administration of one or more doses of a ghrelin variant to the subject, thereby preventing damage to neurons associated with oxidative stress and overproduction of reactive oxygen species (ROS) in a subject with one or more incidence of mBI or concussion.

In some embodiments, the present invention provides for a method for preventing memory loss or headaches in a subject with mBI or concussion, wherein such method comprises administration of an effective amount of a ghrelin variant to the subject in one or more doses, thereby preventing memory loss and/or headaches in a subject with mBI or concussion.

Some embodiments relate to formulations for administration to a subject, which formulations can include a pharmaceutically acceptable carrier and ghrelin or a ghrelin variant having a carbon 14 (C14) content of less than 1 part per trillion (ppt), wherein said formulation is suitable for delivery of an effective amount of ghrelin or the ghrelin variant to the brain of said patient so as to treat mild brain injuries or concussion. For example, any of the methods described above and elsewhere herein can utilize a ghrelin or ghrelin variant molecule having a C14 content of less than 1 ppt.

Some embodiments relate to methods of monitoring a mild brain injury or concussion, the severity of an injury and/or the recovery from such an injury. The methods can include, for example, administering a purified ghrelin variant compound, including for example, ghrelin with a C14 content of less than 1 ppt, in a pharmaceutically acceptable composition to a subject that has suffered a mild brain injury or concussion.

Some embodiments relate to methods of treating mild brain injury or concussion, reducing the incidence or severity of mBI or concussion in a subject, and/or reducing the amount of time needed to recover from a mild brain injury or concussion. The methods include providing or administering to a subject in need (e.g., a subject that has suffered, is at risk of suffering, is prone to suffer, and/or is about to participate in an activity with a high risk for suffering, a mBI) an amount of a ghrelin or ghrelin variant (including a ghrelin variant with a C14 content of less than 1 ppt) sufficient to provide a therapeutically effective in vivo level of ghrelin to treat or reduce according to the method, wherein the level is greater than the endogenous level of ghrelin in the subject. For example, the amount of administered ghrelin or ghrelin variant can be an amount sufficient to provide a blood level of ghrelin that is greater than the usual or average endogenous blood level of ghrelin, such as 1.5, 2, 3, 5, 10, 20, 50, 100, 1,000 or up to 2,000 times the normal endogenous blood level (or any sub value or sub range there between). In some case, the amount administered can result in a blood or plasma concentration of at least 55 picograms per milliliter. In some embodiments the greater ghrelin levels can be achieved within hours of the injury (e.g., less than 8 hours after the injury). They also can be maintained above endogenous levels for some period of time that is sufficient to provide the desired therapeutic benefit, for example, for at least 30 minutes to 24 hours (or any sub value or sub range there between). Endogenous ghrelin levels are not sufficient for treating mBI or concussion or reducing the incidence, severity or the time needed to recover as readily evidenced by the long term damage done to the brain by repetitive concussive injuries (mBI) or concussion. The instant embodiments provide a benefit and result that do not occur naturally in the body with endogenous levels. Such a benefit was unknown prior to the instant disclosure. In some embodiments, the methods can further include selecting or identifying a subject that has suffered, is at risk of suffering, is prone to suffer, and/or is about to participate in an activity with a high risk for suffering, a mBI or concussion, prior to administration of the ghrelin or ghrelin variant.

Some embodiments relate to methods for determining the efficacy of a compound treating a patient suffering mild brain injury (mBI) or concussion. The methods can include, for example, i) determining the expression level of uncoupling protein-2 (UCP-2) in a biological sample obtained from the patient treated with the compound; ii) comparing the expression level of UCP-2 to a biological sample obtained from a subject treated with a ghrelin variant (as described herein including with the modifications described herein); and iii) determining the efficacy of the compound, wherein the compound is efficacious when the expression level of UCP-2 induced by the compound is equal to (within at least 10%) or greater than the expression level of UCP-2 induced by the ghrelin variant.

Some embodiments relate to methods for treating a patient suffering loss of cognitive or motor skills due to mild brain injury (mBI) or concussion. The methods can include, for example, i) determining the expression level of ghrelin in the patient's brain over a period of time; ii) administering a ghrelin or ghrelin variant (including the modified and C14 versions described herein) to the patient; and iii) periodically repeating step ii) during treatment when the expression level of ghrelin falls below a normal range as a basis to determine the efficacy of the treatment, wherein an increase in the expression level of ghrelin in the brain demonstrates an improvement in the patient's cognitive or motor skill condition.

Some embodiments relate to methods of treatment, prevention, inhibition and/or reduction of a condition, symptom or sequelae as described herein according the one or more of the methodologies described herein, further comprising monitoring injury severity and/or recovery after having administered ghrelin or a ghrelin variant, or monitoring after an initial administration and administering a purified ghrelin compound or a ghrelin variant in a pharmaceutically acceptable composition subsequent to monitoring.

The present disclosure provides for a method of treating mBI or concussion in a subject, comprising administering to the subject an effective amount of a compound comprising the ghrelin variant that is encoded by or administered as a nucleic acid. In some embodiments, the nucleic acid is any that encodes the sequence of SEQ ID NO. 1. In some embodiments, the nucleic acid sequence comprises 5′-ggctccagct tcctgagccc tgaacaccag agagtccagc agagaaagga gtcgaagaag ccaccagcca agctgcagcc ccga-3′ (SEQ ID NO. 8). In some embodiments, the ghrelin variant encodes a nucleic acid sequence comprises SEQ ID NO. 8 with one or more mutations. In some embodiments, the mutation is selected from the group consisting of nucleic acid insertion, deletion, substitution and translocation. In some embodiments, the mutation occurs at one or more positions.

The present disclosure provides for a method for detecting and treating mild brain injury or concussion in a subject in need thereof, comprising measuring the amount of biomarkers in a sample of the subject after the occurrence of a mild brain injury or concussion; comparing the amount of the biomarkers in the sample with a sample from an uninjured subject; and administering to the subject a therapeutically effective amount of a composition comprising ghrelin and/or ghrelin variant.

In some embodiments, the biomarker is one or more of, but not limited to, SBDP150, S100, GFAP, UCH-L1, Axonal Proteins: α II spectrin (and SPDB)-1, NF-68 (NF-L)-2, Tau-3, α II, III spectrin, NF-200 (NF—H), NF-160 (NF-M), spectrin, βII-spectrin and βII-spectrin breakdown products (βII-SBDPs), βII-SBDP-80, βII-SBDP-85, β-SBDP-108, βII-SBDP-110, microtubule-associated proteins (MAPS), MAP-2 (e.g., MAP-2A, MAP-2B, MAP-2C, MAP-2D), MAP breakdown products (MAP-BDP), Amyloid precursor protein, α internexin; Dendritic Proteins: beta III-tubulin-1, p24 microtubule-associated protein-2, alpha-Tubulin (P02551), beta-Tubulin (P04691), MAP-2A/B-3, MAP-2C-3, Stathmin-4, Dynamin-1 (P21575), Phocein, Dynactin (Q13561), Vimentin (P31000), Dynamin, Profilin, Cofilin 1,2; Somal Proteins: UCH-L1 (Q00981)-1, Glycogen phosphorylase-BB-2, PEBP (P31044), NSE (P07323), CK-BB (P07335), Thy 1.1, Prion protein, Huntingtin, 14-3-3 proteins (e.g. 14-3-3-epsolon (P42655)), SM22-α, Calgranulin AB, alpha-Synuclein (P37377), beta-Synuclein (Q63754), HNP 22; Neural nuclear proteins: NeuN-1, S/G(2) nuclear autoantigen (SG2NA), Huntingtin; Presynaptic Proteins: Synaptophysin-1, Synaptotagmin (P21707), Synaptojanin-1 (Q62910), Synaptojanin-2, Synapsin1 (Synapsin-Ia), Synapsin2 (Q63537), Synapsin3, GAP43, Bassoon(NP-003449), Piccolo (aczonin) (NP-149015), Syntaxin, CRMP1, 2, Amphiphysin-1 (NP-001626), Amphiphysin-2 (NP-647477); Post-Synaptic Proteins: PSD95-1, NMDA-receptor (and all subtypes)-2, PSD93, AMPA-kainate receptor (all subtypes), mGluR (all subtypes), Calmodulin dependent protein kinase II (CAMPK)-alpha, beta, gamma, CaMPK-IV, SNAP-25, a-/b-SNAP; Myelin-Oligodendrocyte: Myelin basic protein (MBP) and fragments, Myelin proteolipid protein (PLP), Myelin Oligodendrocyte specific protein (MOSP), Myelin Oligodendrocyte glycoprotein (MOG), myelin associated protein (MAG), Oligodendrocyte NS-1 protein; Glial Protein Biomarkers: GFAP (P47819), Protein disulfide isomerase (PDI)-P04785, Neurocalcin delta, S100beta; Microglia protein Biomarkers: lba1, OX-42, OX-8, OX-6, ED-1, PTPase (CD45), CD40, CD68, CD11b, Fractalkine (CX3CL1) and Fractalkine receptor (CX3CR1), 5-d-4 antigen; Schwann cell markers: Schwann cell myelin protein; Glia Scar: Tenascin; Hippocampus: Stathmin, Hippocalcin, SCG10; Cerebellum: Purkinje cell protein-2 (Pcp2), Calbindin D9K, Calbindin D28K (NP-114190), Cerebellar CaBP, spot 35; Cerebrocortex: Cortexin-1 (P60606), H-2Z1 gene product; Thalamus: CD15 (3-fucosyl-N-acetyl-lactosamine) epitope; Hypothalamus: Orexin receptors (OX-1R and OX-2R)-appetite, Orexins (hypothalamus-specific peptides); Corpus callosum: MBP, MOG, PLP, MAG; Spinal Cord: Schwann cell myelin protein; Striatum: Striatin, Rhes (Ras homolog enriched in striatum); Peripheral ganglia: Gadd45a; Peripheral nerve fiber (sensory+motor): Peripherin, Peripheral myelin protein 22 (AAH91499); Other Neuron-specific proteins: PH8 (S Serotonergic Dopaminergic, PEP-19, Neurocalcin (NC), a neuron-specific EF-hand Ca2+-binding protein, Encephalopsin, Striatin, SG2NA, Zinedin, Recoverin, Visinin; Neurotransmitter Receptors: NMDA receptor subunits (e.g. NR1A2B), Glutamate receptor subunits (AMPA, Kainate receptors (e.g. GluR1, GluR4), beta-adrenoceptor subtypes (e.g. beta(2)), Alpha-adrenoceptors subtypes (e.g. alpha(2c)), GABA receptors (e.g. GABA(B)), Metabotropic glutamate receptor (e.g. mGluR3), 5-HT serotonin receptors (e.g. 5-HT(3)), Dopamine receptors (e.g. D4), Muscarinic Ach receptors (e.g. M1), Nicotinic Acetylcholine Receptor (e.g. alpha-7); Neurotransmitter Transporters: Norepinephrine Transporter (NET), Dopamine transporter (DAT), Serotonin transporter (SERT), Vesicular transporter proteins (VMAT1 and VMAT2), GABA transporter vesicular inhibitory amino acid transporter (VIAAT/VGAT), Glutamate Transporter (e.g. GLT1), Vesicular acetylcholine transporter, Vesicular Glutamate Transporter 1, [VGLUT1; BNPI] and VGLUT2, Choline transporter, (e.g. CHT1); Cholinergic Biomarkers: Acetylcholine Esterase, Choline acetyltransferase (ChAT); Dopaminergic Biomarkers: Tyrosine Hydroxylase (TH), Phospho-TH, DARPP32; Noradrenergic Biomarkers: Dopamine beta-hydroxylase (DbH); Adrenergic Biomarkers: Phenylethanolamine N-methyltransferase (PNMT); Serotonergic Biomarkers: Tryptophan Hydroxylase (TrH); Glutamatergic Biomarkers: Glutaminase, Glutamine synthetase; GABAergic Biomarkers: GABA transaminase (GABAT)), GABA-B-R2, or a combination thereof. In some embodiments one or more of the biomarkers listed above, can be specifically excluded.

### DETAILED DESCRIPTION

It is to be understood that the present disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

The detailed description of the present disclosure is divided into various sections only for the reader's convenience and disclosure found in any section may be combined with that in another section. Unless defined otherwise, all technical and scientific terms used herein have the same meaning as commonly understood by one of ordinary skill in the art to which the present disclosure belongs.

### Definitions

It must be noted that as used herein and in the appended claims, the singular forms “a”, “an”, and “the” include plural referents unless the context clearly dictates otherwise. Thus, for example, reference to “a compound” includes a plurality of compounds.

Unless defined otherwise, all technical and scientific terms used herein have the same meaning as commonly understood by one of ordinary skill in the art to which the present disclosure belongs. As used herein the following terms have the following meanings.

As used herein, the term “about” when used before a numerical designation, e.g., temperature, time, amount, concentration, and such other, including a range, indicates approximations which may vary by (+) or (−) 10%, 5% or 1%.

As used herein, the term “administration” can be effected in one dose, continuously or intermittently or by several subdoses which in the aggregate provide for a single dose. Dosing can be conducted throughout the course of treatment. Methods of determining the most effective means and dosage of administration are known to those of skill in the art and will vary with the composition used for therapy, the purpose of the therapy, the target cell being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include oral administration, vaginal, nasal administration, injection, topical application, sublingual, pulmonary, and by suppository.

As used herein, the term “affinity” refers to the strength of binding between receptors and their ligands, for example, between an antibody and its antigen.

As used herein, the term “amino acid residue” refers to an amino acid formed upon chemical digestion (hydrolysis) of a polypeptide at its peptide linkages. Unless otherwise specified, the amino acid encompasses L-amino acid, including both natural amino acid and synthetic amino acid or the like as long as the desired functional property is retained by the polypeptide. NH₂ refers to the free amino group present at the amino terminus of a polypeptide. COOH refers to the free carboxy group present at the carboxy terminus of a polypeptide. Standard polypeptide abbreviations for amino acid residues are as follows: A (Ala or Alanine); C (Cys or Cysteine); D (Asp or Aspartic Acid); E (Glu or Glutamic Acid); F (Phe or Phenylalanine); G (Gly or Glycine); H (His or Histidine); I (Ile or Isoleucine); K (Lys or Lysine); L (Leu or Leucine); M (Met or Methionine); N (Asn or Asparagine); P (Pro or Proline); Q (Gln or Glutamine); R (Arg or Arginine); S (Ser or Serine); T (Thr or Threonine); V (Val or Valine); W (Trp or Tryptophan); X (Xaa or Unknown or Other); Y (Tyr or Tyrosine); and Z (Glx/Gln/Glu or Glutamic Acid/Glutamine); and Dpr (2,3-diaminopropionic acid). All amino acid residue sequences represented herein by formula have a left-to-right orientation in the conventional direction of amino terminus to carboxy terminus. The phrase “amino acid residue” is broadly defined to include the naturally occurring and modified and non-naturally occurring amino acids. A dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

As used herein, the term “amino acid derivatives” include, for example, alkyl-substituted tryptophan, β-naphthylalanine, naphthylalanine, 3,4-dihydrophenylalanine, and methylvaline. The amino acids and amino acid derivatives include both of L forms and D-forms.

The term “amino acid side chain” refers to any one of the twenty groups attached to the α-carbon in naturally occurring amino acids. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc.

As used herein, the term “acylated ghrelin variant” is a ghrelin variant, which contains an acyl group attached to any of its constituent amino acids. Acylation of a hydroxyl containing amino acid group can be conducted on the native peptide by conventional blocking of all reactive amino groups using conventional blocking agents such as T-Boc, CBZ and benzyl groups. Subsequently, reaction of the OH functionality of serine, threonine, tyrosine, and the like is accomplished by reaction with an C₁-C₂₀ aliphatic carboxylic acid or its halide, anhydride or activated form thereof to form the acyl group —OC(O)C₁-C₅alkyl. Suitable aliphatic acids include, by way of example, formic acid, acetic acid, propanoic acid, butyric acid, and the like. Acylation of a carboxyl containing amino acid group can be conducted to the native peptide by conventional blocking of all reactive amino groups using conventional blocking agents such as T-Boc, CBZ and benzyl groups. Subsequently, reaction of the —COOH functionality of glutamic acid or aspartic acid is accomplished by converting the carboxylic acid group to its corresponding halide, or activated form followed by reaction with an C₁-C₂₀ aliphatic alcohol such as methanol, ethanol, propanol and the like to form an acyl group of the formula —C(O)O—C₁-C₂₀ alkyl. Such acylated groups are converted in vivo to the corresponding amino acids by enzymatic processes using endogenous esterases. Alternatively, synthetic ghrelin analogs can be made by standardized amino acid coupling well known in the art. In such cases, the C-terminus amino acid is attached to a solid support and each successive amino acid (directionally from the C to the N terminus) is added with the appropriate blocking groups. An alternative amino acid as described above can be introduced into the polypeptide at any point or points.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the present disclosure. “Consisting of” shall mean excluding more than trace elements of other ingredients and substantial method steps. Embodiments defined by each of these transition terms are within the scope of the present disclosure.

As used herein, the term “concussion” is a form of head injury that refers to an immediate or transient loss or perturbation of consciousness accompanied by a brief period of amnesia after a blow to the head.

As used herein, the term “dissociation constant” or “Kd” is a measure describing the strength of binding (or affinity or avidity) between receptors and their ligands, for example an antibody and its antigen. The smaller the Kd, the stronger the binding.

As used herein, the term “fusion polypeptide” is a polypeptide comprised of at least two polypeptides and a linking sequence to operatively link the two polypeptides into one continuous polypeptide. Without limitation, fusion peptides can refer to dimer compounds as well as to conjugates. The fusion polypeptide can include, for example, two linked polypeptides not normally found linked in nature. The two polypeptides linked in a fusion polypeptide can be derived from two independent sources, or can be two of the same molecule. One example of such a fusion polypeptide is ghrelin-L-ghrelin where L is a biologically acceptable linker. Such linkers can be synthetic or naturally occurring. Synthetic linkers can range from 1 to 20 carbon atoms in length with up to 5 carbon atoms being replaced by heteroatoms such as —O—, —S— —S(O), —S(O)2-, —NH and the like. Other non-limiting examples of fusions include linking a ghrelin variant to another ghrelin variant, linking a ghrelin variant to ghrelin or a ghrelin fragment (e.g., less than all of the 28 amino acids of ghrelin), and so forth.

As used herein, the term “ghrelin” is a polypeptide having 28 amino acid sequence as set forth in SEQ ID No. 1, and can include the octanoyl acylation as described above. Human ghrelin is a polypeptide having the amino acid sequence as set forth in GenBank® Accession No. NP_057446 or Swiss-Prot Identifier GHRL_HUMAN. Human ghrelin preprotein has 117 amino acids. This preprotein undergoes the following post-translational processing. The signal peptide (amino acids 1-23) is removed and the remaining 94 amino acids are cleaved by a protease to provide a mature 28 amino acid ghrelin (amino acids 24-51) or a mature 27 amino acid ghrelin (amino acids 24-50) and a mature 23 amino acid obestatin (amino acids 76-98). The 28 amino acid mature ghrelin peptide can be further modified at the serine at position 26 in the preprotein by either an O-octanoyl group or an O-decanoyl group. The obestatin mature peptide can be further modified at the lysine at position 98 of the preprotein by an amide group. An additional ghrelin preprotein is known, which lacks the glutamine at position 37 of the preprotein.

As used herein, the term “ghrelin variant” refers to any compound (e.g., peptides, small molecule drugs) has at least about 50% of the functional activity of ghrelin. The functional activity includes, without limitation, feeding regulation, nutrient absorption, gastrointestinal motility, energy homeostasis, anti-inflammatory regulation, suppression of inflammatory cytokines, activation of Gq/G11, accumulation of inositol phosphate, mobilization of calcium from intracellular stores, activation or deactivation of MAP kinases, NFκB translocation, CRE driven gene transcription, reduction in reactive oxygen species (ROS), NAMPT enzyme activation, and binding of arrestin to ghrelin receptor. Examples of ghrelin variants are provided above.

As used herein, the term “ghrelin receptor” refers to any naturally occurring molecule to which ghrelin binds and induces a biological activity. Ghrelin is known to bind to growth hormone secretagogue receptor 1a (i.e., GHSR), however, the present disclosure is not limited to a specific type of receptor.

As used herein, the term “growth hormone secretion promoting substance receptors,” are a family of receptors including receptors called Type 1a and Type 1b discovered in experiments on binding to MK-0677 (Hormon Res., 1999, 51(suppl 3), 1, Science, 1996, 273, 974), receptors called FM1, FM2 and FM3 (Hormon Res., 1999, 51(suppl 3), 1, Endocrine Reviews, 1997, 18(5), 621), and receptors called a hexarelin binding site (Hormon Res., 1999, 51(suppl 3), 1, Endocrinology 1998, 139, 432, J. Clin. Endocrinol. Metab., 2000, 85, 3803). However, these publicly known receptors are not restrictive.

As used herein, the term “growth hormone releasing peptides (GHRP)” refer to peptides having the pharmacological activity that promotes growth hormone release. Various derivatives (for example, derivatives formed by substitution of amino acids constituting the peptides, ester derivatives) are also included, as long as these derivatives have functions equivalent to such function. There are no restrictions on the numbers and origins of the amino acid residues or amino acid derivative residues in the peptides (for example, the peptides or derivatives isolated or purified from human cells, synthetic products, semi-synthetic products, and those obtained by genetic engineering).

As used herein, the term “GH secretagogues (GHS)” refer to non-peptide substances having the pharmacological activity that promotes growth hormone secretion. Various derivatives (for example, ester derivatives) are also included, as long as these derivatives have functions equivalent to such function.

Details of the “growth hormone secretion promoting substances” are disclosed, for example, in the following patent specifications. Furthermore, all of those classified in the following documents as growth hormone releasing peptides (GHRP), growth hormone releasing peptide(GHRP)-like compounds, growth hormone releasing peptide-mimetics (GHRP-mimetics), and growth hormone secretagogues (GH secretagogoes, GHS) are included in the growth hormone secretion promoting substances. However, such classifications are not strict, and are not to be interpreted as restrictive. WO00/48623, WO99/09991, WO99/08699, WO98/58950, WO98/58949, WO98/58948, WO98/58947, WO98/51687, WO98/50036, WO98/46569, WO98/46220, WO98/25897, WO98/25622, WO98/16527, WO98/10653, WO98/03473, WO97/42223, WO97/40071, WO97/40023, WO97/39768, WO97/34604, WO97/27298, WO97/25057, WO97/24369, WO97/23508, WO97/22622, WO97/22620, WO97/22367, WO97/21730, WO97/18233, WO97/15574, WO97/15573, WO97/15191, WO97/11697, WO97/00894, WO96/38471, WO96/35713, WO96/33189, WO96/32943, WO96/32126, WO96/24587, WO96/24580, WO96/22997, WO96/22782, WO96/15148, WO96/13265, WO96/10040, WO96/05195, WO96/02530, WO95/34311, WO95/17423, WO95/17422, WO95/16707, WO95/16692, WO95/16675, WO95/14666, WO95/13069, WO95/12598, WO95/09633, WO95/03290, WO95/03289, WO94/19367, WO94/18169, WO94/13696, WO94/11397, WO94/11012, WO94/08583, WO94/07519, WO94/07486, WO94/07483, WO94/05634, WO93/04081, WO92/16524, WO92/01711, WO89/10933, WO89/07111, WO89/07110, WO83/02272, U.S. Pat. Nos. 5,936,089, 5,877,182, 5,872,100, 5,854,211, 5,830,433, 5,817,654, 5,807,985, 5,804,578, 5,798,337, 5,783,582, 5,777,112, 5,776,901, 5,773,448, 5,773,441, 5,767,124, 5,767,118, 5,767,085, 5,731,317, 5,726,319, 5,726,307, 5,721,251, 5,721,250, 5,691,377, 5,672,596, 5,668,254, 5,663,171, 5,663,146, 5,656,606, 5,652,235, 5,646,301, 5,635,379, 5,583,130, 5,578,593, 5,576,301, 5,559,128, 5,545,735, 5,536,716, 5,534,494, 5,506,107, 5,494,919, 5,492,920, 5,492,916, 5,486,505, 5,434,261, 5,430,144, 5,416,073, 5,374,721, 5,317,017, 5,310,737, 5,284,841, 5,283,241, 5,206,235, 5,030,630, 4,880,777, 4,851,408, 4,650,787, 4,485,101, 4,411,890, 4,410,513, 4,410,512, 4,228,158, 4,228,157, 4,228,156, 4,228,155, 4,226,857, 4,224,316, 4,223,021, 4,223,020, and 4,223,019. The contents of these documents are incorporated by reference.

As used herein, the term “individual” is an animal or human susceptible to a condition, in particular mBI or concussion. In some embodiments, the individual is a mammal, including human, and non-human mammals such as dogs, cats, pigs, cows, sheep, goats, horses, rats, and mice.

As used herein, the term “mild brain injury” (mBI), sometimes referred as a “mild traumatic brain injury (mTBI), refers to a non-disease event commonly caused by an injury resulting in an insult to the brain. mBI may be caused, for example, by impact forces, in which the head strikes or is struck by something, or impulsive forces, in which the head moves without itself being subject to blunt trauma (for example, when the chest hits something and the head snaps forward; or as a result of rapid acceleration or deceleration of the head). mBI commonly results, for example, from a sports-related injury, a motor vehicle accident, an accidental fall, or an assault. Although the vast majority of such injuries improve through natural recovery the damage caused by such an injury or repetitive injuries can cause long term deficits in cognitive, and/or motor skill functions. mBI is different from and has a distinct pathology as compared to diseases such as acute traumatic brain insults such as strokes (ischemic or hemorrhagic), AVM's, brain tumors, and the like.

As used herein, the term “non-acylated ghrelin variant” or “unacylated ghrelin variant” is a ghrelin variant, which does not contain an acyl group attached to any of its constituent amino acids. It should be understood that in some embodiments, the ghrelin variant can be partially non- or unacylated at one or more of it residues.

As used herein, the term “polypeptide” or “peptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, acylation, acylation by fatty acid, fatty acid-modification, derivatization by known protecting/blocking groups, proteolytic cleavage, or modification by non-naturally occurring amino acids. Modification may be fatty acid modification or triglyceride modification. Fatty acid modification may be a short to medium-chain fatty acid. The short fatty acid may be a two-carbon fatty acid or acetic acid. Medium chain fatty acid may be 14-carbon fatty acid or tetradecanoic acid. Modification with a fatty acid may be acylation of SEQ ID NO. 1 at serine amino acid position 2 and/or serine amino acid position 3. Modification may be catalyzed by ghrelin O-acyl transferase (GOAT) of fatty acid thioester and ghrelin as substrates. In one embodiment, post-translationally modified ghrelin may be bound and/or recognized by growth hormone secretagogue receptor type 1a (GHSR-1a) or ghrelin receptor. In one embodiment, post-translationally modified ghrelin may be fatty acid-acylated ghrelin at serine amino acid position 2 and/or serine amino acid position 3 bound and/or recognized by GHSR-1a or ghrelin receptor. A polypeptide may be derived from a natural biological source or produced by recombinant technology, but is not necessarily translated from a designated nucleic acid sequence. It may be generated in any manner, including by chemical synthesis. The term “Polypeptide” or “Peptide” also refers to a compound comprising a plurality of amino acids linked therein via peptide linkages. Here, the amino acid (also called an amino acid residue) includes naturally occurring amino acids represented by formula: NH2-CH(R′)—COOH, wherein R′ is a naturally occurring substituent group, as well as its D, L-optical isomers etc. There is also a peptide, wherein a certain naturally occurring amino acid is replaced by a modified amino acid. The modified amino acid includes the amino acids of the above formula wherein the substituent group R′ is further modified, its D, L-optical isomers thereof, and non-natural amino acids wherein e.g. various substituent groups are bound to the substituent group R′ of the above formula via or not via an ester, ether, thioester, thioether, amide, carbamide or thiocarbamide linkage. The modified amino acid also includes non-natural amino acids whose amino groups are replaced by lower alkyl groups. Antibodies can be covered by the above definition of peptide and polypeptide, include antibody ghrelin variants.

As used herein, the term “peptide analogue” refers to a compound wherein at least one amino acid in a peptide is replaced by a non-amino acid compound, and thus at least one linkage of said substituent compound to the peptide analogue is not a peptide linkage.

As used herein, the term “homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Biologically equivalent polynucleotides are those having the above-noted specified percent homology and encoding a polypeptide having the same or similar biological activity.

As used herein, the term “secretagogue” is a substance stimulating growth hormone release, such as ghrelin or a ghrelin variant. A secretagogue according to the present disclosure may for example be selected from L-692-429 and L-692-585 (benzoelactam compounds; available from Merck & Co, Inc., Whitehouse Station, N.J.), MK677 (spiroindaner; available from Merck), G-7203, G-7039, G-7502 (isonipecotic acid peptidomimetic; available from Genentech, Inc., South San Francisco, Calif.), NN703 (Novo Nordisk Inc., Princeton, N.J.), or ipamorelin. The growth hormone secretagogue may in one embodiment be non-acylated, for instance a non-acylated form of ghrelin variant. In some embodiments, the ghrelin variant binds to the growth hormone secretagogue receptor GHS-R 1a (GHSR). The ghrelin variant compounds described herein are active at the receptor for growth hormone secretagogue (GHS), e.g., the receptor GHS-R 1a. The compounds can bind to GHS-R 1a, and stimulate receptor activity. In some embodiments, the compounds can bind other receptors and, optionally, stimulate their activity. In some embodiments, the ghrelin variant increases uncoupling protein-2 (UCP-2) expression. In some embodiments, the ghrelin variant increases UCP-2 expression in mitochondria. In some embodiments, the ghrelin variant prevents the metabolic consequence of mBI and any associated chronic conditions.

As used herein, the term “purinergic receptor” generally refers to a family of cell surface receptors which are activated by purine-containing compounds such as adenosine and the nucleotides ATP and UTP. The members of the family are broadly classified as follows: P2X receptors are ligand-gated ion channels; P1 receptors are adenosine-activated G protein-coupled receptors; and P2Y receptors, which form the basis of this application, are nucleotide-activated G protein-coupled receptors.

As used herein, the term “P2Y receptor” or “P2Y-R” generally refers to a class of G protein-coupled purinergic receptors that are stimulated by nucleotides such as ATP (P2Y2, P2Y11), ADP, UTP (P2Y2, P2Y4), UDP (P2Y6) and UDP-glucose. To date 8 P2Y receptors have been cloned in humans: P2Y1, P2Y2, P2Y4, P2Y6, P2Y11, P2Y12, P2Y13 and P2Y14. P2Y receptors are present in almost all human tissues where they exert various biological functions based on their G-protein coupling. The biological effects of P2Y receptor activation depend on how they couple to downstream signaling pathways, either via Gi, Gq or Gs G proteins. Human P2Y receptors have the following G protein coupling: Gq/11 coupled: P2Y1, P2Y2, P2Y6, P2Y14; Gi and Gq/11 coupled: P2Y4 Gs and Gq/11 coupled: P2Y11; Gi coupled: P2Y12, P2Y13.

As used herein, the term “receptor agonist” is generally used to refer to a synthetic or naturally occurring molecule that mimics the action of an endogenous biochemical molecule (such as hormone or neurotransmitter) when bound to the cognate receptor of that hormone or neurotransmitter. An agonist is the opposite of an antagonist in the sense that while an antagonist also binds to the receptor, the antagonist does not activate the receptor and actually blocks it from activation by agonists. A partial agonist activates a receptor, but only produces a partial physiological response compared to a full agonist. A co-agonist works with other co-agonists to produce the desired effect together. Receptors can be activated or inactivated by endogenous (such as hormones and neurotransmitters) or exogenous (such as drugs) agonists and antagonists, resulting in stimulating or inhibiting the cell. The term “P2Y receptor agonist” or “P2Y purinergic receptor agonist” generally refers to any molecule that binds to P2Y receptors and elicits at least a portion of the cellular responses typically associated with P2Y receptor activation in that cell type.

As used herein, the term “alkyl” generally refers to C₁₋₁₀ inclusive, linear, branched, or cyclic, saturated or unsaturated (i.e., alkenyl and alkynyl)hydrocarbon chains, for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, allenyl and optionally substituted arylalkenyl and arylalkyny groups. As used herein, the term “acyl” refers to an organic acid group wherein the —OH of the carboxyl group has been replaced with another substituent (i.e., as represented by RCO—, wherein R is an alkyl or an aryl group). As such, the term “acyl” specifically includes arylacyl groups. Specific examples of acyl groups include acetyl and benzoyl. As used herein, the term “aryl” refers to 5 and 6-membered hydrocarbon and heterocyclic aromatic rings. Examples of aryl groups include cyclopentadienyl, phenyl, furan, thiophene, pyrrole, pyran, pyridine, imidazole, isothiazole, isoxazole, pyrazole, pyrazine, pyrimidine, and the like. The term “alkoxyl” as used herein refers to C₁₋₁₀ inclusive, linear, branched, or cyclic, saturated or unsaturated oxo-hydrocarbon chains, including for example methoxy, ethoxy, propoxy, isopropoxy, butoxy, t-butoxy, and pentoxy. The term “aryloxyl” as used herein refers to aryloxy such as phenyloxyl, and alkyl, halo, or alkoxyl substituted aryloxyl. As used herein, the terms “substituted alkyl” and “substituted aryl” include alkyl and aryl groups as defined herein in which one or more atoms or functional groups of the aryl or alkyl group are replaced with another atom or functional group, for example, halogen, aryl, alkyl, alkoxy, hydroxy, nitro, amino, alkylamino, dialkylamino, sulfate, and mercapto. The terms “halo,” “halide,” or “halogen” as used herein refer to fluoro, chloro, bromo, and iodo groups.

The term “alkenyl” refers to a hydrocarbon chain that may be a straight chain or branched chain having one or more carbon-carbon double bonds. The alkenyl moiety contains the indicated number of carbon atoms. For example, C2-C10 indicates that the group may have from 2 to 10 (inclusive) carbon atoms in it. The term “lower alkenyl” refers to a C2-C8 alkenyl chain. In the absence of any numerical designation, “alkenyl” is a chain (straight or branched) having 2 to 10 (inclusive) carbon atoms in it.

The term “alkynyl” refers to a hydrocarbon chain that may be a straight chain or branched chain having one or more carbon-carbon triple bonds. The alkynyl moiety contains the indicated number of carbon atoms. For example, C2-C10 indicates that the group may have from 2 to 10 (inclusive) carbon atoms in it. The term “lower alkynyl” refers to a C2-C8 alkynyl chain. In the absence of any numerical designation, “alkynyl” is a chain (straight or branched) having 2 to 10 (inclusive) carbon atoms in it.

The terms “cycloalkyl” or “cyclyl” as employed herein includes saturated and partially unsaturated cyclic hydrocarbon groups having 3 to 12 carbons, preferably 3 to 8 carbons, and more preferably 3 to 6 carbons, wherein the cycloalkyl group may be optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms if N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkenyl” refers to an alkenyl substituted with a heteroaryl. The term “heteroarylalkynyl” refers to an alkynyl substituted with a heteroaryl. The term “heteroarylalkoxy” refers to an alkoxy substituted with heteroaryl.

The term “heterocyclyl” or “heterocyclylalkyl” refers to a nonaromatic 5-8 membered monocyclic, 5-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morphonlinyl, tetrahydrofuranyl, and include both bridged and fused ring systems. The term “heterocyclylalkyl” refers to an alkyl substituted with a heterocyclyl.

The term “sulfinyl” refers to a sulfur attached to two oxygen atoms through double bonds. An “alkylsulfonyl” refers to an alkyl substituted with a sulfonyl. The term “amino acid” refers to a molecule containing both an amino group and a carboxyl group. Suitable amino acids include, without limitation, both the D- and L-isomers of the 20 naturally occurring amino acids found in peptides (e.g., A, R, N, C, D, Q, E, G, H, I, L, K, M, F, P, S, T, W, Y, V (as known by the one letter abbreviations)) as well as unnaturally occurring amino acids prepared by organic synthesis or other metabolic routes.

The term “substituents” refers to a group “substituted” on an alkyl, cycloalkyl, aryl, heterocyclyl, or heteroaryl group at any atom of that group. Any moiety described herein can be further substituted with a substituent. Suitable substituents include, without limitation, halo, hydroxy, mercapto, oxo, nitro, haloalkyl, alkyl, aryl, aralkyl, alkoxy, thioalkoxy, aryloxy, amino, alkoxycarbonyl, amido, carboxy, alkanesulfonyl, alkylcarbonyl, and cyano groups.

### Ghrelin Variants

As noted herein, embodiments generally related to methods of using ghrelin variants for treating, reducing the severity of and in some cases preventing mild brain injury, related symptoms and sequelae. Some embodiments relate to methods of reducing the risk of, or preventing the development of symptoms or sequelae. Non-limiting examples of various ghrelin variants are described. For example, in some embodiments, the ghrelin variant comprises a polypeptide having an amino acid sequence of Gly-Ser-Ser-Phe-Leu-Ser-Pro-Glu-His-Gln-Arg-Val-Gln-Gln-Arg-Lys-Glu-Ser-Lys-Lys-Pro-Pro-Ala-Lys-Leu-Gln-Pro-Arg (SEQ ID NO. 1), which has at least one change or modification to the sequence selected from the various changes described herein such that the variant is different from the natural ghrelin molecule. In some embodiments, the ghrelin variant comprises a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO. 1. In some embodiments, one or more of the modifications or substitutions listed above, can be specifically excluded.

In some embodiments, in the amino acid sequence set forth in SEQ ID NO: 1, an amino acid sequence of amino acids 1 to 4 refers to Gly Ser Ser Phe (SEQ ID NO. 11), an amino acid sequence of amino acids 1 to 5 refers to Gly Ser Ser Phe Leu (SEQ ID NO. 12), an amino acid sequence of amino acids 1 to 6 refers to Gly Ser Ser Phe Leu Ser (SEQ ID NO. 13), an amino acid sequence of amino acids 1 to 7 refers to Gly Ser Ser Phe Leu Ser Pro (SEQ ID NO. 14), an amino acid sequence of amino acids 1 to 8 refers to Gly Ser Ser Phe Leu Ser Pro Glu (SEQ ID NO. 15), an amino acid sequence of amino acids 1 to 9 refers to Gly Ser Ser Phe Leu Ser Pro Glu His (SEQ ID NO. 16), and, an amino acid sequence of amino acids 1 to 10 refers to Gly Ser Ser Phe Leu Ser Pro Glu His Gln (SEQ ID NO. 17).

In some embodiments, the polypeptide includes both acylated and non-acylated forms. One non-limiting example of a ghrelin variant is ASP-531 (Alize Pharma), which is a clinical stage, unacylated ghrelin molecule. In some embodiments, maximum ghrelin variant activity requires acylation of the third residue of ghrelin. Naturally-occurring ghrelin is acylated with octanoic acid, however, any bulky hydrophobic group attached to the side chain of the third residue is sufficient for ghrelin variant function (Matsumoto et al., 2001, Biochem. Biophys. Res. Commun. 287: 142-146). Non-limiting examples of such hydrophobic groups include n-lauroyl, palmitoyl, 3-octenoyl and 4-methylpentanoyl. Furthermore, ghrelin variants wherein the ester bond between octanoic acid and Ser3 is more chemically stable, such as a thioether (Cys3(octyl)) or ether (Ser3(octyl) bond), are also useful. Ghrelin variants also include truncation mutants of ghrelin. There is a wealth of information regarding the structure and function of ghrelin to guide a skilled artisan in preparing ghrelin variants useful in the present disclosure. See, for example, Kojima et al., 2005, Physiol. Rev. 85: 495-522 and references cited therein. Structurally, ghrelin is a random coil in aqueous solution (Silva Elipe et al., 2001, Biopolymers 59:489-501). Various truncated ghrelin peptides also demonstrate random coil structure. The minimum active ghrelin core is the first four amino acids with Ser3 acylated (Bednarek et al., 2000, J. Med. Chem. 43:4370-4376; Matsumoto et al., 2001, Biochem. Biophys. Res. Commun. 284:655-659). Thus a ghrelin variant comprising only the first four amino acids, e.g., Gly-Ser-Ser(n-octanoyl)-Phe (SEQ ID No. 41), is also useful in the present disclosure. In some embodiments, ghrelin variant is 5-aminopentanoyl-Ser(Octyl)-Phe-Leu-aminoethylamide (Ser-Phe-Leu are residues 3-5 of SEQ ID NO. 13), which was found to have potent ghrelin activity (Matsumoto et al., 2001, Biochem. Biophys. Res. Commun. 284:655-659). Each reference described above, is incorporated herein by reference in its entirety for all of its disclosure, including all methods, materials, etc. In some embodiments, one or more of the modifications or substitutions listed above, can be specifically excluded.

In some embodiments, the side-chain hydroxyl group of third serine from the N-terminus of the ghrelin or ghrelin variants has been acylated with fatty acid. In some embodiments, the third serine from the N-terminus of the ghrelin or ghrelin variants has been replaced by threonine.

In some embodiments, at least one amino acid deleted, replaced and/or added in a part outside the amino acid sequences of ghrelin or ghrelin variants. In some embodiments, ghrelin or ghrelin variants is a peptide or compound having the activity of increasing the intracellular calcium ion concentration and the activity of inducing secretion of growth hormone, and (a) constitutional amino acids are modified or not modified and (b) at least one amino acid is replaced or not replaced by a non-amino acid compound.

In some embodiments, ghrelin variants comprise a modified amino acid or modified amino acids in which (a) a saturated or unsaturated alkyl chain containing one or more carbon atoms was introduced at the a carbon atom of the amino acid via or not via an alkylene group containing one or more carbon atoms and via an ester, ether, thioether, amide or disulfide linkage, or (b) a saturated or unsaturated alkyl chain containing one or more carbon atoms was introduced at the a carbon atom of the amino acid, and the symbol D is an amino acid having a hydrophobic residue.

In some embodiments, the ghrelin variants comprise a modified amino acid at the second position from the N-terminal residue of ghrelin. In some embodiments, ghrelin variants comprise a modified amino acid at the third position from the N-terminal residue of ghrelin. In some embodiments, ghrelin variants comprise modified amino acids at the second and the third position from the N-terminal residue of ghrelin. In some embodiments, the amino acid in the modified amino acid is serine or cysteine.

In some embodiments, ghrelin variants comprise a modified amino acid modified by conversion of a functional group in a side chain of said amino acid into an ester linkage. In some embodiments, ghrelin variants comprise an amino acid having a fatty acid bound via an ester linkage to a side-chain hydroxyl group of said amino acid. For example, the fatty acid can be bound in such a manner to a residue of ghrelin.

In some embodiments, ghrelin variants comprise an amino acid having a fatty acid bound via an ester linkage to a side-chain hydroxyl group of said amino acid or via a thioester linkage to a side-chain mercapto group of said amino acid. In some embodiments, ghrelin variants comprise an amino acid to which a fatty acid containing 2 to 35 carbon atoms was bound. In some embodiments, ghrelin variants comprise an amino acid to which a fatty acid selected from the group consisting of fatty acids containing 2, 4, 6, 8, 10, 12, 14, 16 and 18 carbon atoms was bound. For example, the variant can be the natural ghrelin sequence that is modified as suggested. In some embodiments, ghrelin variants comprise a fatty acid, which is octanoic acid, decanoic acid, a monoene fatty acid thereof or a polyene fatty acid thereof. In other embodiments, acylation can be accomplished by use of a diacid result in acylation of one or both of the hydroxyl groups of Ser(2) and Ser(3). Exemplary diacids can be represented by the formula R—CH(COOH)(CH₂)ₙCOOH where R is a saturated or unsaturated aliphatic group of from 1 to 20 carbon atoms. Diacylation will lead to a ring structure which is contemplated to be more resistant to protease degradation.

In some embodiments, ghrelin variants comprise a basic amino acid bound to the carboxyl-terminal, wherein the amino-terminal is modified with a saturated or unsaturated alkyl or acyl group containing one or more carbon atoms, and/or a hydroxyl group of the carboxyl-terminal carboxyl group is OZ or NR2R3 wherein Z is a pharmaceutically acceptable cation or a lower branched or linear alkyl group, and R2 and R3 are the same or different and represent H or a lower branched or linear alkyl group.

In some embodiments, ghrelin variants can also be modified using ordinary molecular biological techniques so as to improve their resistance to proteolytic degradation or to optimize solubility properties or to render them more suitable as a therapeutic agent. Even natural ghrelin can be so modified in order to produce a ghrelin variant. Analogs of such ghrelin variants include those containing residues other than naturally-occurring L-amino acids, e.g., D-amino acids or non-naturally occurring synthetic amino acids. The ghrelin variants are not limited to products of any of the specific exemplary processes listed herein. The ghrelin variants useful may further be conjugated to non-amino acid moieties that are useful in their therapeutic application. In particular, moieties that improve the stability, biological half-life, water solubility, and immunologic characteristics of the peptide are useful. A non-limiting example of such a moiety is polyethylene glycol (PEG). In some embodiments, ghrelin variants include peptide analogs.

In some embodiments, covalent attachment of biologically active compounds to water-soluble polymers is one method for alteration and control of biodistribution, pharmacokinetics and toxicity for ghrelin variant compounds (Duncan et al., 1984, Adv. Polym. Sci. 57:53-101). For example, natural ghrelin can be so modified to produce a ghrelin variant. Many water-soluble polymers have been used to achieve these effects, such as poly(sialic acid), dextran, poly(N-(2-hydroxypropyl)methacrylamide) (PHPMA), polyvinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, 1980, Polyethylene glycol. In R. L. Davidson (Ed.), Handbook of Water Soluble Gums and Resins, McGraw-Hill, New York, chapter 18). PEG possesses an ideal set of properties: very low toxicity (Pang, 1993, J. Am. Coll. Toxicol. 12: 429-456) excellent solubility in aqueous solution (Powell, supra), low immunogenicity and antigenicity (Dreborg et al., 1990, Crit. Rev. Ther. Drug Carrier Syst. 6: 315-365). PEG-conjugated or “PEGylated” protein therapeutics, containing single or multiple chains of polyethylene glycol on the protein, have been described in the scientific literature (Clark et al., 1996, J. Biol. Chem. 271: 21969-21977). Each reference in this paragraph is incorporated in its entirety herein.

In some embodiments, ghrelin variants may incorporate amino acid residues which are modified without affecting activity. For example, the termini may be derivatized to include blocking groups, i.e. chemical substituents suitable to protect and/or stabilize the N- and C-termini from “undesirable degradation,” a term meant to encompass any type of enzymatic, chemical or biochemical breakdown of the compound at its termini which is likely to affect the function of the compound, i.e. sequential degradation of the compound at a terminal end thereof. Blocking groups include protecting groups conventionally used in the art of peptide chemistry which will not adversely affect the in vivo activities of the variants. For example, suitable N-terminal blocking groups can be introduced by alkylation or acylation of the N-terminus.

Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH2), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the ghrelin variant's C-terminal residue or used in place of it. Further, the free amino and carboxyl groups at the termini can be removed altogether from the ghrelin variant to yield desamino and descarboxylated forms thereof without effect on the ghrelin variant activity.

In some embodiments, the ghrelin variant compound is one or more Dln-101, Growth hormone (GH) releasing hexapeptide (GHRP)-6, EP 1572, Ape-Ser(Octyl)-Phe-Leu-aminoethylamide, isolated ghrelin splice variant-like compound, ghrelin splice variant, growth hormone secretagogue receptor GHS-R 1a ligand, and a combination thereof. In some embodiments, one or more of the variants listed above, can be specifically excluded. In some embodiments, the ghrelin variant comprises a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of one or more of the compounds described in the present disclosure. In some embodiments the ghrelin agonist can be a short peptide, for example a pentapeptide such as of RM-131 (or BIM-28131). In some embodiments, at least 1 amino acid of such a pentapeptide can be substituted with a natural or a non-natural amino acid such as those described herein, removed, and/or chemically modified (e.g., octanoylated, acylated, etc.). In some embodiments, one or more of the modifications or substitutions listed above, can be specifically excluded.

In some embodiments, the ghrelin variant is Dln-101, which is a small peptide ghrelin agonist. In some embodiments, the ghrelin variant comprises a polypeptide comprising the sequence of Gly Ser Ser Phe Leu Ser Pro Glu His Gln Arg Val Gln Val Arg Pro Pro Lys Ala Pro His Val Val (SEQ ID No. 2). In some embodiments, the ghrelin variant comprises a polypeptide comprising the sequence of Gly Ser Xaa Phe Leu Ser Pro Glu His Gln Arg Val Gln Val Arg Pro Pro His Lys Ala Pro His Val Val (SEQ ID No. 3), wherein the third position is a 2,3-diaminopropionic acid (Dpr), with the Dpr in the third position being optionally octanoylated. In some embodiments, the ghrelin variant comprises a polypeptide comprising the sequence of Gly Xaa Xaa Phe Leu Ser Pro Glu His Gln Arg Val Gln Val Arg Pro Pro His Lys Ala Pro His Val Val (SEQ ID No. 4), wherein the second and third position are 2,3-diaminopropionic acid (Dpr) residues, with the Dpr in the third position being optionally octanoylated. In some embodiments, the ghrelin variant comprises a polypeptide comprising the sequence of Gly Ser Ser Phe Leu Ser Pro Glu His Gln Arg Val Gln Val Arg Pro Pro His Lys Ala Pro His Val Val Pro Ala Leu Pro (SEQ ID No. 5). In some embodiments, the ghrelin variant is a ghrelin splice variant, comprising a polypeptide comprising the sequence of Gly Ser Ser Phe Leu Ser Pro Glu His Gln Arg Val Gln Val Arg Pro Pro His Lys Ala Pro His Val Val Pro Ala Leu Pro Leu (SEQ ID No. 9). In some embodiments, one or more of the modifications or substitutions listed above, can be specifically excluded.

In some embodiments, the ghrelin variant is RM-131 (also known as BIM-28131), which is a small peptide ghrelin agonist, and BIM-28163, which is a full-length ghrelin analog antagonist. In some embodiments, the ghrelin variant is a polypeptide comprises the sequence of Inp-D-2Nal-D-Trp-Thr-Lys-NH₂ (SEQ ID NO. 6). One or more of the amino acids of SEQ ID NO. 6 can be substituted with a natural or non-natural amino acid. Additional small peptides are disclosed in U.S. Pat. Nos. 8,377,865 and 7,456,253, each of which is incorporated herein by reference in its entirety. Additionally, one or more of the amino acids can be chemically modified, for example, with an octanoyl or like group, with an acyl group, a protecting group, and the like.

In some embodiments, the ghrelin variant is an isolated ghrelin splice variant-like compound with the formula Z1-(X1)m-(X2)-(X3)n-Z2, wherein Z1 is an optionally present protecting group; each X1 is independently selected from a naturally occurring amino acid and a synthetic amino acid; X2 is selected from a naturally occurring amino acid and a synthetic amino acid, said amino acid being modified with a bulky hydrophobic group; each X3 is independently selected from a naturally occurring amino acid and a synthetic amino acid, wherein one or more of X1 and X3 optionally may be modified with a bulky hydrophobic group; Z2 is an optionally present protecting group; m is an integer in the range of from 1-10; n is an integer in the range of from 4-92; provided that the compound according to formula Z1-(X1)m-(X2)-(X3)n-Z2 is 15-94 amino acids in length, and has at least 80% homology to the sequence of Gly Ser Ser Phe Leu Ser Pro Glu His Gln Arg Val Gln Val Arg Pro Pro His Lys Ala Pro His Val Val Pro Ala Leu Pro Leu Ser Asn Gln Leu Cys Asp Leu Glu Gln Gln Arg His Leu Trp Ala Ser Val Phe Ser Gln Ser Thr Lys Asp Ser Gly Ser Asp Leu Thr Val Ser Gly Arg Thr Trp Gly Leu Arg Val Leu Asn Gln Leu Phe Pro Pro Ser Ser Arg Glu Arg Ser Arg Arg Ser His Gln Pro Ser Cys Ser Pro Glu Leu (SEQ ID NO. 10).

In some embodiments, the ghrelin variant is EP1572 or UMV1843 (Aib-DTrp-DgTrp-CHO), which is a peptido-mimetic GH secretagogue with selective GH-releasing activity. In some embodiments, the ghrelin variant is a growth hormone secretagogue receptor GHS-R 1a ligand, which binds to growth hormone secretagogue receptor GHS-R 1a.

In some embodiments, the ghrelin variant is Growth hormone (GH) releasing hexapeptide (GHRP)-6, which is a compound of the chemical nomenclature: L-histidyl-D-tryptophyl-L-alanyl-L-tryptophyl-D-phenylalanyl-L-Lysinamide.

In some embodiments, the ghrelin variant is a growth hormone releasing peptide (GHRP) including, but not limited to, Pralmorelin (GHRP 2, GPA 748, growth hormone-releasing peptide 2, KP-102 D, KP-102 LN, KP-102D, KP-102LN; Kaken Pharma and Stella Pharma), Examorelin/Hexarelin, GHRP-1, GHRP-6 (SK&F-110679), Ipamorelin (NNC-260161), NNC-260194, NNC-260235, and salts and esters thereof. In some embodiments, the ghrelin variant is Pralmorelin as described in U.S. Pat. No. 6,468,974, which is incorporated by reference in its entirety. In some embodiments, one or more of the variants listed above, can be specifically excluded.

In some embodiments, the ghrelin variants of the present disclosure include, but not limited to, molecules and compounds described in U.S. Pat. Nos. 6,849,597, 7,452,862, 7,476,653, 7,521,420, 7,550,431, 7,491,695, 7,589,058, and 8,088,733; US Patent Application Publication Nos. US 2010/0216706 and US 2012/0232001; and WO 2004/009616, WO 2008/143835, and WO 2013/113916, which are incorporated by reference in their entireties. In some embodiments, one or more of the variants listed above, can be specifically excluded.

Examples of these salts are described below, and hydrochlorides are named as preferred examples (e.g., pralmorelin dihydrochloride, hexarelin hydrochloride).

No restrictions are imposed on the origins of the amino acid residues or amino acid derivative residues in the peptides (for example, the peptides or derivatives may have been isolated or purified from human or rat cells, or may be synthetic products or semi-synthetic products, or may have been obtained by genetic engineering).

The ghrelin variants of the present disclosure include, but not limited to, one of the amino acids that has been deficient are typified by des-G1n14-ghrelin, i.e., ghrelin with the 14th Gln residue deleted, and the compounds described in J. Med. 5 Chem. 2000, 43, 4370-4376.

Examples of the ghrelin variants include, but not limited to, peptides and their derivatives which have the third and fourth amino acids from the N-terminal among the 28 amino acids of ghrelin (preferably, the four amino acids at the N-terminal) and in which the side chain of the third amino acid (Ser) from the N-terminal has been substituted, the peptides and derivatives having a growth hormone secretion promoting action. In some embodiments, one or more of the modifications or substitutions listed above, can be specifically excluded.

Examples of the side chain of the third amino acid from the N-terminal include, but not limited to, an acyl group and an alkyl group (the number of their carbon atoms is preferably 6 to 18) other than octanoyl which is the side chain of ghrelin.

Concrete examples of the side chain are as follows: —CH₂(CH₂)₉CH₃, —CO—(CH₂)₆CH₃, —CO—CH═CH—CH═CH—CH═CH—CH₃, —CO—CH (CH₂CH₂CH₃)₂, —CO—(CH₂)₉CH₃, —CO—(CH₂)₁₄CH₃, —CO—(CH₂)₆CH₂Br, —CO—CH(CH₂)₂CONH(CH₂)₂ CH₃, —COPh, and a group of the following formula

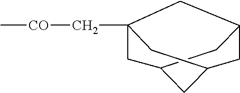

A concrete example of the ghrelin variant, which has the third and fourth amino acids from the N-terminal and in which the side chain of the third amino acid (Ser) from the N-terminal has been substituted, is the compound reported at the 37th Peptide Forum (Oct. 18 to 20, 2000), i.e., NH₂—(CH₂)₄—CO-Ser(octyl)-Phe-Leu-NH—(CH₂)₂—NH₂

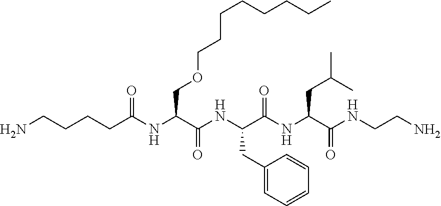 [SMILES: CCCCCCCCOC[C@H](NC(=O)CCCCN)C(=O)N[C@@H](Cc1ccccc1)C(=O)N[C@@H](CC(C)C)C(=O)NCCN]

The GH secretagogues (GHS) include compounds expressed by the following formulae:

### (1) Compounds of the Following General Formula

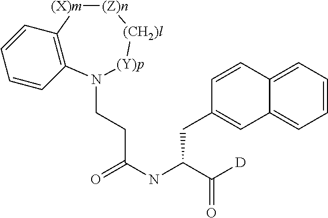

where
1 denotes 0, 1 or 2,
X represents —CH₂—, —O—, —S(O)r-(r=0, 1 or 2), —C(O)—, —C(S)—, —CH═CH—, —CH(OH)— or —NR—,
R represents a hydrogen atom, a (C₁-C₅ alkyl group, a (C₃-C₈)cycloalkyl group, an acyl group or an alkoxycarbonyl group,
m denotes 0, 1 or 2,
Y represents —C(O)—, —C(S)—, or a (C₁-C₅)alkylene group which may be substituted by (C₁-C₅)alkyl group(s),
p denotes 0, 1 or 2,
Z represents a substituted or unsubstituted (C₁-C₅) alkylene group, —NR— (R is a hydrogen atom, a (C₁-C₅) alkyl group, a (C₃-C₈) cycloalkyl group, an acyl group or an alkoxycarbonyl group), or a group of the formula

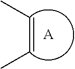

where A is a 5- or 6-membered aromatic ring optionally containing at least one hetero atom, and
A may further be substituted by a group selected from a halogen atom, a hydroxyl group, a (C₁-C₅) alkyl group, a (C₁-C₅) alkoxy group, a (C₁-C₅) perfluoroalkyl group, a (C₁-C₅) perfluoroalkoxy group, a nitro group, a cyano group, an amino group, a substituted amino group, a phenyl group and/or a substituted phenyl group,
n denotes 0 or 1,
D represents

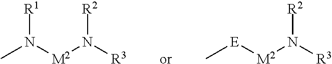

where R¹ represents a hydrogen atom, an alkyl group, a substituted alkyl group, a cycloalkyl group or a substituted cycloalkyl group,
R² and R³ each represent, independently of each other, a hydrogen atom, an alkyl group, a substituted alkyl group, an acyl group, an amidino group or an alkoxycarbonyl group, or one of R² and R³, taken together with R¹, may constitute an alkylene group,
further, R² and R³ may together constitute an alkylene group or a hetero ring,
M² is represented by the formula

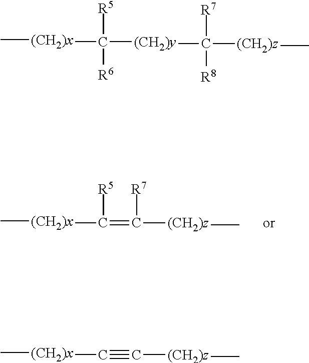

where x, y and z each represent, independently of each other, an integer of 0 to 4,
R⁵, R⁶, R⁷ and R⁸ each represent, independently of each other, a hydrogen atom, a halogen atom, an alkyl group, a substituted alkyl group, —OR⁹, —SR⁹, —NR⁹R¹⁰, —NHC(O)R⁹, —C(O)OR⁹, —OCOR⁹, —OC(O)OR⁹ or —CONR⁹R¹⁰, or may constitute an alkylene group or a hetero ring taken together with R¹ or R²,
R⁹ and R¹⁰ each represent, independently of each other, a hydrogen atom, an alkyl group or a substituted alkyl group,
R⁹ may constitute an alkylene group taken together with R¹ or R²,
R⁵ and R⁷, or R⁶ and R⁸ may together constitute an alkylene group or a hetero ring, or
R⁵ and R⁶, or R⁷ and R⁸ may constitute a carbonyl group, a thiocarbonyl group or an imino group taken together with the carbon atom to which R⁵ and R⁶, or R⁷ and R⁸ have been bound, and
E represents an oxygen atom or a sulfur atom.

Of the compounds (1), preferred compounds are those of the above-mentioned formula: where 1 denotes 0, 1 or 2, preferably 0,
X represents —CH₂—, —O—, —S(O)r-, —C(O)—, —C(S)—, —CH═CH—, —CH(OH)— or —NR,
R represents a hydrogen atom, a (C₁-C₅) alkyl group, a (C₃-C₈) cycloalkyl group, an acyl group, or an alkoxycarbonyl group,
r denotes 0, 1 or 2,
m denotes 0, 1 or 2, preferred being —CH₂— as X and m as 2,
Y represents —C(O)—, —C(S)—, or a (C₁-C₅) alkylene group which may be substituted by (C₁-C₅)alkyl group(s),
p denotes 0, 1 or 2, preferred being —C(O)— as Y, and 1 as p,
Z represents a substituted or unsubstituted (C₁-C₅) alkylene group, —NR—(R is a hydrogen atom, a (C₁-C₅) alkyl group, a (C₃-C₈) cycloalkyl group, an acyl group or
an alkoxycarbonyl group), or a 6-membered aromatic ring represented by the formula

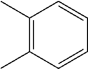

N denotes 0 or 1, and
D represents a group of the formula

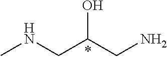

In the above formula, the asterisk (*) represents an asymmetric center, so that isolated pure optical isomers, partially purified optical isomers or racemic mixtures are included.

### (2) Compounds of the Following General Formula

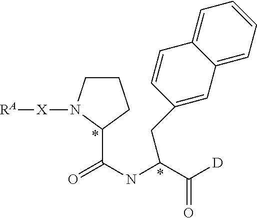 [0164] where
R^{A} represents a substituted or unsubstituted alkyl, a substituted or unsubstituted cycloalkyl, a substituted or unsubstituted alkoxy, a substituted or unsubstituted aryl, or a substituted or unsubstituted amino,
X represents a single bond, —CO— or —SO₂—,
D represents

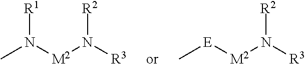

where R1 represents a hydrogen atom, an alkyl group, a substituted alkyl group, a cycloalkyl group, or a substituted cycloalkyl group,
R² and R³ each represent, independently of each other, a hydrogen atom, an alkyl group, a substituted alkyl group, an acyl group, an amidino group or an alkoxy-carbonyl group, or either R² or R³ and R¹ may together constitute an alkylene group,
further, R² and R³ may together constitute an alkylene group or a hetero ring,
M² is represented by the formula

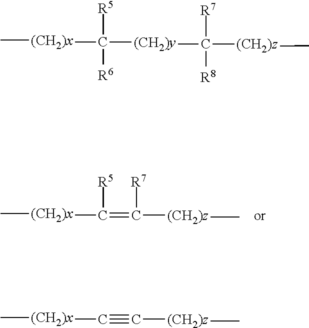

where x, y and z each represent, independently of each other, an integer of 0 to 4,
R⁵, R⁶, R⁷ and R⁸ each represent, independently of each other, a hydrogen atom, a halogen atom, an alkyl group, a substituted alkyl group, —OR⁹, —SR⁹, —NR⁹R¹⁰, —NHC(O)R⁹, —C(O)OR⁹, —OCOR⁹, —OC(O)OR⁹ or —CONR⁹R¹⁰, or may constitute an alkylene group or a hetero ring taken together with R1 or R²,
R⁹ and R¹⁰ each represent, independently of each other, a hydrogen atom, an alkyl group or a substituted alkyl group,
R⁹ may constitute an alkylene group taken together with R¹ or R²,
R⁵ and R⁷, or R⁶ and R⁸ may together constitute an alkylene group or a hetero ring, or
R⁵ and R⁶, or R⁷ and R⁸ may constitute a carbonyl group, a thiocarbonyl group or an imino group taken together with the carbon atom to which R⁵ and R⁶, or R⁷ and R⁸ have been bound, and
E represents an oxygen atom or a sulfur atom, and
the asterisk (*) represents an asymmetric center, so that the compounds (2) include isolated pure optical isomers, partially purified optical isomers, racemic mixtures or diastereomer mixtures (all such optical isomers are included in the scope of the present disclosure).
Of the compounds (2), preferred aspects are as follows:
X is preferably —CO—.
R^{A} is preferably a C₁-C₁₁ alkyl which may be substituted by a substituted or unsubstituted cycloalkyl, a substituted or unsubstituted alkoxy, a substituted or unsubstituted aryl and/or a hydroxy; a C₃-C₆ cycloalkyl which may be substituted by a substituted or unsubstituted alkyl, a substituted or unsubstituted alkoxy, a substituted or unsubstituted aryl and/or a hydroxy; a C₁-C₁₁ alkoxy which may be substituted by a substituted or unsubstituted cycloalkyl, a substituted or unsubstituted alkoxy, a substituted or unsubstituted aryl and/or a hydroxy; an aryl which may be substituted by a substituted or unsubstituted cycloalkyl, a substituted or unsubstituted alkoxy, a substituted or unsubstituted aryl and/or a hydroxy; or an amino which may be substituted by a substituted or unsubstituted alkyl or a substituted or unsubstituted aryl.

More preferably, R^{A} is represented by any of the following formulas:

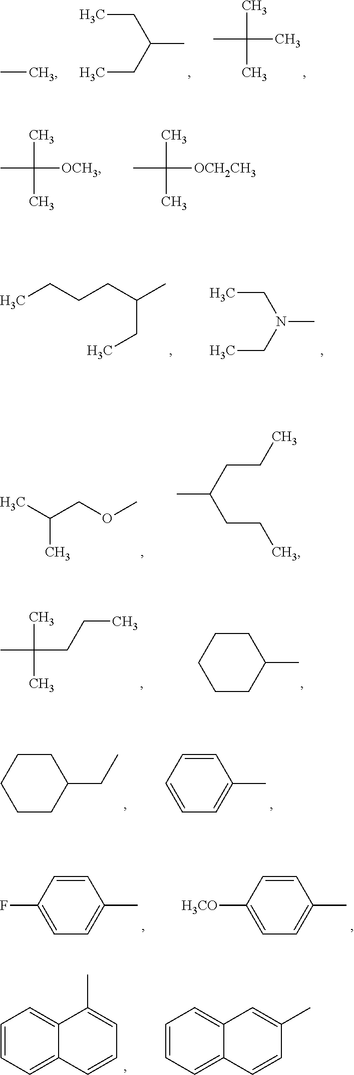

D is preferably represented by the following formula:

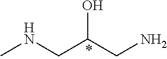

In some embodiments, the ghrelin variant is a GH secretagogues (GHS). Concrete compounds as the GH secretagogues (GHS) are exemplified by, but not limited to, S-38855, S-37555, S-39100, ibutamorelin [e.g., ibutamorelin mesylate (MK-0677)], capromorelin (CP-424391), NNC-260722, NNC-260323, L-163661, L-163540, L-168721, LY-426410, LY-444711, L-692,429, L-692,585, L-700,653, L-252,564, L-162,752, L-164,080, G-7203, G-7039, G-7052, G-7220, tabimorelin (NN-703), or salts and esters thereof. In some embodiments, one or more of the variants listed above, can be specifically excluded.

Typical of the above-described compounds are described in detail in Table 1:

Chemical Names and Structural Formulas of Compounds as Concrete Examples

| General Name | Chemical Name | Chemical Structure |
|---|---|---|
| Pralmorelin | D-alanyl-3-(2-naphthalenyl)-D-alanyl- | H-D-Ala-D-2-NaI-Ala-Trp-D-Phe-Lys-NH₂ |
| (GHRP-2) | L-alanyl-L-tryptophyl-D-phenylalanyl- | |
| | L-lysineamide | |
| Hexarelin | L-histidyl-2-methyl-D-tryptophyl-L- | H-His-D-2-Me-Trp-Ala-Trp-D-Phe-Lys-NH₂ |
| (examorelin) | alanyl-L-tryptophyl-D-phenylalanyl-L- | |
| | lysineamide | |
| GHPR-6 (SK&F- | -histidyl-D-tryptophyl-L-alanyl-L- | H-His-D-Trp-Ala-Trp-D-Phe-Lys-NH₂ |
| 110679) | tryptophyl-D-phenylalanyl-L- | |
| | lysineamide | |
| GHRP-1 | | Ala-His-D-(2′)-NaI-Ala-Trp-D-Phe-Lys-NH₂ |
| Ghrelin | | 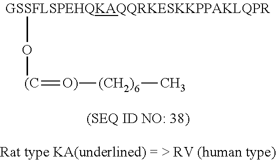 |
| S-38855 | N-(3-amino-2-hydroxypropy1)-2(R)41- (2,2-dimethylpropionyl)pyrrolidine- 2(S)-carbonylamino]-3-naphthalen-2- yl-propionamid | 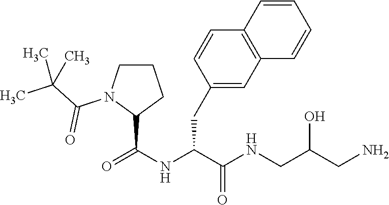 |
| S-37555 | N-[1(R)-(3-amino-2-hydroxy- propylcarbamoy1)-2-yl-ethyl]-3-(6- oxo-11,12-dihydro-6H- dibenzo[b,f]azocinn-5-y1)- propionamide | 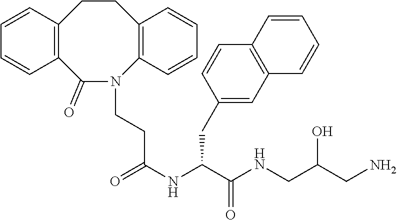 [SMILES: NCC(O)CNC(=O)[C@@H](Cc1ccc2ccccc2c1)NC(=O)CCN1C(=O)c2ccccc2CCc2ccccc21] |
| S-39100 | N-(3-amino-2-hydroxypropyl)-2(S)-[1- (2-ethylbutyryl)pyrrolidine-2(R)- carbonylamino]-3-naphthalen-2-yl- propionamide | 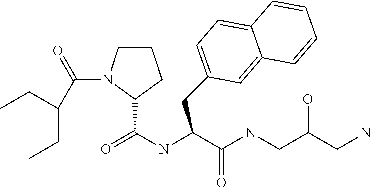 |
| MK-0677 (ibutamorelin mesylate) | 2-Amino-N-[(R)-2-(benzyloxy)-1-[[1- (methylsulfonyOspiro[indoline-3,4′- piperidin]-1′-yl]carbonyl]ethyl]-2- methylpropionamidomonomethane- sulfonate | 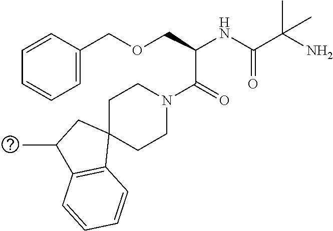 [SMILES: CC1CC2(CCN(C(=O)[C@@H](COCc3ccccc3)NC(=O)C(C)(C)N)CC2)c2ccccc21] |
| CP-424391 (Capromorelin) | 2-Amino-N-[(1R)-1-[[(3aR)-3a-benzyl- 2,3,3a,4,6,7-hexahydro-2-methy1-3- oxo-5H-pyrazolo[4,3,c]pyridin-5- yl]carbony1]-2-(benzyloxy)ethyl]-2- methylpropionamide | 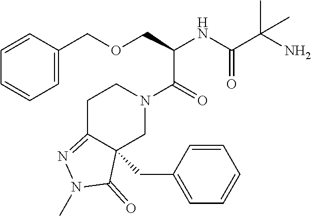 [SMILES: CN1N=C2CCN(C(=O)[C@@H](COCc3ccccc3)NC(=O)C(C)(C)N)C[C@@]2(Cc2ccccc2)C1=O] |
| NNC-260703 (Tabimorelin) NN-703 | 5-Amino-5-methylhexa-(2E)-enoic acid N-methyl-N-[(1R)-1-[N-methyl-N- [(1R)-1-(methylcarbamoy1)-2- phenylethyl]carbamoy1]-2-(2- naphthyl)ethyl]amide | 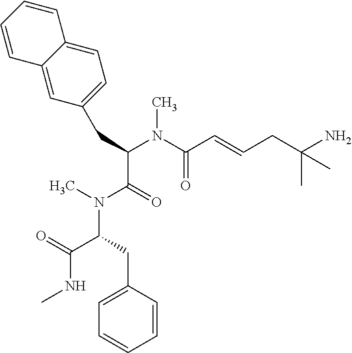 [SMILES: CNC(=O)[C@@H](Cc1ccccc1)N(C)C(=O)[C@@H](Cc1ccc2ccccc2c1)N(C)C(=O)/C=C/CC(C)(C)N] |
| NNC-260722 | 5-Amino-5-methylhexa-(2E)-enoic acid N-methyl-N-[(1R)-1-[N-methyl-N- [(1R)-1-(2-hydroxypropylcarbamoyl)- 2-phenylethyl]carbamoyl]-2-(2- naphthyl)ethyl]amide | 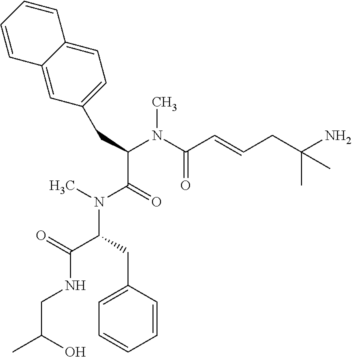 [SMILES: CC(O)CNC(=O)[C@@H](Cc1ccccc1)N(C)C(=O)[C@@H](Cc1ccc2ccccc2c1)N(C)C(=O)/C=C/CC(C)(C)N] |
| NNC-260194 | 3-(4-Imidazolyl)propionyl-D-Phe-Ala- Trp-D-Phe(CH₂NH)-Lys-NH-ol | |
| NNC-260235 | 3-Aminomethyl-benzoyl-D-2-NaI-N- Me-D-Phe-Lys-NH₂ | 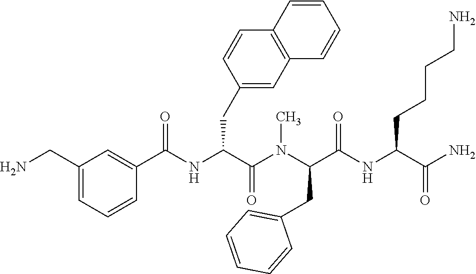 [SMILES: CN(C(=O)[C@@H](Cc1ccc2ccccc2c1)NC(=O)c1cccc(CN)c1)[C@H](Cc1ccccc1)C(=O)N[C@@H](CCCCN)C(N)=O] |
| NNC-260323 | (2R)-2-[N-(3-aminomethylbenzoy1)-N- methyl-D-2-NaI]-N-methyl-3-phenyl-1- propanol | 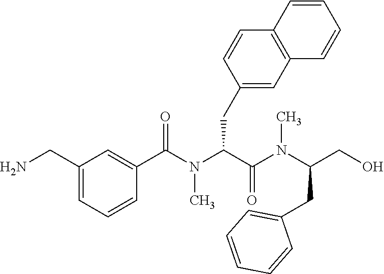 [SMILES: CN(C(=O)[C@@H](Cc1ccc2ccccc2c1)N(C)C(=O)c1cccc(CN)c1)[C@@H](CO)Cc1ccccc1] |
| NNC-260161 (ipamorelin) | 2-Methylalanyl-L-histidyl 3 (2 naphthaleny1)-D-alanyl-D- phenylalanyl-L-lysineamide | 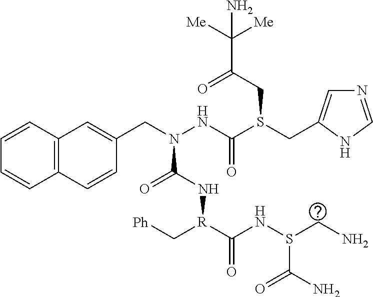 |
| L-163540 | 1-[2(R)-(2-amino-2- methylpropionylamino)-3-(1H-indo1- 3-yl)propionyl]3-benzylpiperidine-3(S)- carboxylic acid ethyl ester | 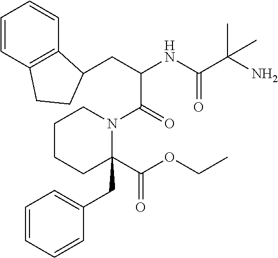 [SMILES: CCOC(=O)[C@]1(Cc2ccccc2)CCCCN1C(=O)C(CC1CCc2ccccc21)NC(=O)C(C)(C)N] |
| L-168721 | N-(6-aminohexyl)-2-(4-oxo-2- phenethy1-6-pheny1-4H-quinazolin-3- yl)acetamide | 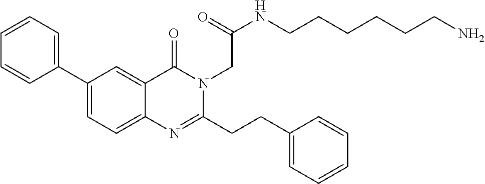 [SMILES: NCCCCCCNC(=O)Cn1c(CCc2ccccc2)nc2ccc(-c3ccccc3)cc2c1=O] |
| LY-426410 | 2-Amino-N-[2-benzyloxy-(1R)-[1-[(1R)- (4-methoxyphenyl)-2-(4- methylpiperidin 1 yl)-2-oxo-ethyl]-1H- imidazol-4-ylcarbamoyl]ethyl]-2- methylpropionamide | 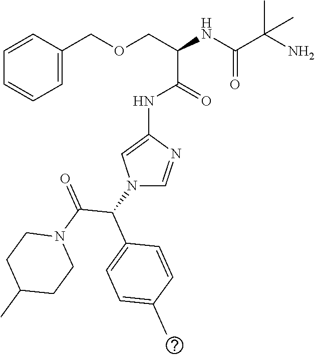 [SMILES: Cc1ccc([C@H](C(=O)N2CCC(C)CC2)n2cnc(NC(=O)[C@@H](COCc3ccccc3)NC(=O)C(C)(C)N)c2)cc1] |
| LY-444711 | 2-Methylalanyl-N-[1-[(112)-1-(4- methoxypheny1)-1-methyl-2-oxo-2-(1- pyrrolidinypethy1]-1H-imidazol-4-yl]- 5-phenyl-D-norvalineamide | 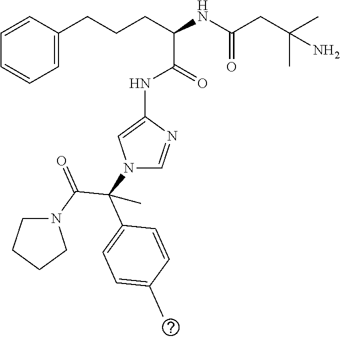 [SMILES: Cc1ccc([C@@](C)(C(=O)N2CCCC2)n2cnc(NC(=O)[C@@H](CCCc3ccccc3)NC(=O)CC(C)(C)N)c2)cc1] |

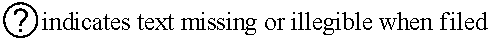

Salts of the above-described compounds include but are not limited to, for example, salts with mineral acids such as hydrochloric acid, sulfuric acid or phosphoric acid, salts with organic acids such as methanesulfonic acid, benzenesulfonic acid, malic acid, citric acid or succinic acid, salts with alkali metals such as sodium or potassium, salts with alkaline earth metals such as calcium or magnesium, and salts with basic amino acids such as arginine.

In some embodiments, the growth hormone secretion promoting substances and their salts may be used in combination of two or more.

The growth hormone secretion promoting substance used in the present disclosure can be formed into ordinary oral preparations and parenteral preparations, for example, liquids and solutions (injections, nasal drops, syrups, dry syrups), tablets, troches, capsules (hard capsules, soft capsules, microcapsules), powder, subtle granules, granules, ointments and suppositories, by publicly known pharmaceutical manufacturing techniques, when used alone or combined with pharmaceutically acceptable carriers, additives, etc. The growth hormone secretion promoting substance of the present disclosure can also be made into dosage forms, such as drug delivery systems (for example, slow-release preparations).

The carriers and additives usable in the preventive/therapeutic agents of the present disclosure include, for example, those which are ordinarily used in preparing pharmaceuticals: aqueous vehicles such as physiological saline, water (tap water, distilled water, purified water, water for injection) and Ringer solution, nonaqueous vehicles such as oily solvents (vegetable oils) and water-soluble solvents (propylene glycol, macrogol, ethanol, glycerin), bases such as cacao butter, polyethylene glycol, microcrystalline wax, white beeswax, liquid petrolatum and white petrolatum, excipients such as sucrose, starch, mannitol, sorbitol, lactose, glucose, cellulose, talc, calcium phosphate and calcium carbonate, binders such as cellulose, methylcellulose, hydroxypropylcellulose, polypropylpyrrolidone, gelatin, acacia, polyethylene glycol, sucrose and starch, disintegrators such as starch, carboxymethylcellulose, hydroxypropyl starch, sodium bicarbonate, calcium phosphate, calcium carboxymethylcellulose and calcium citrate, lubricants such as magnesium stearate, talc and sodium lauryl sulfate, taste correctives such as citric acid, menthol, glycine, sorbitol and orange powder, preservatives and antiseptics such as parahydroxybenzoate esters, benzyl alcohol, chlorobutanol and quaternary ammonium salts (benzalkonium chloride, benzethonium chloride), stabilizers such as albumin, gelatin, sorbitol and mannitol, suspending agents such as methylcellulose, polyvinylpyrrolidone and aluminum stearate, plasticizers such as glycerin and sorbitol, dispersing agents such as hydroxypropyl methylcellulose, solution adjuvants such as hydrochloric acid and cyclodextrin, emulsifying agents such as sodium monostearate, electrolytes such as sodium chloride, and nonelectrolyte tonicity regulating agents and flavors, such as sugar alcohols, sugars and alcohols.

In the oral preparation, water-swellable cellulose (carboxymethylcellulose, calcium carboxymethylcellulose, sodium croscarboxymethylcellulose, low substitution degree hydroxypropylcellulose), such as microcrystalline cellulose (“Avicel” [trade name, a product of Asahi Chemical Industry]) as described in Japanese Patent Publication No. 1998-456194 can be incorporated for increasing absorbability.

Normally, the preparation of the present disclosure is administered to mammals (e.g., mouse, rat, hamster, rabbit, cat, dog, cattle, horse, sheep, monkey), including humans, by the oral route or by such means as subcutaneous injection, nasal dropping, intra-arterial injection (including drip infusion), intravenous injection, intra-spinal injection or local cerebral administration.

In some embodiments, the ghrelin variant comprises a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of one or more of the compounds described in the present disclosure. The term “sequence identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, Madison, Wis.). This software matches similar sequences by assigning degrees of homology to various substitutions, deletions, and other modifications.

A ghrelin variant homologue of one or more of the sequences specified herein may vary in one or more amino acids as compared to the sequences defined, but is capable of performing the same function, i.e. a homologue may be envisaged as a functional equivalent of a predetermined sequence.

A homologue of any of the predetermined sequences herein may be defined as i) homologues comprising an amino acid sequence capable of being recognized by an antibody, said antibody also recognizing the ghrelin variant, including the acylated ghrelin variant (also un-acylated ghrelin variants in some embodiments), and/or ii) homologues comprising an amino acid sequence capable of binding selectively to GHS-R 1a, and/or iii) homologues having a substantially similar or higher binding affinity to GHS-R 1a than the ghrelin variant described herein. The antibodies used herein may be antibodies binding the N-terminal region of ghrelin variant or the C-terminal region of ghrelin variant, the N-terminal region. The antibodies may be antibodies as described in Ariyasu H. et al, Endocrinology 143:3341-50 (2002), which is incorporated herein by reference in its entirety.

In some embodiments, one or more of the amino acids of the sequence are substituted or replaced by another amino acid or a synthetic amino acid. In some embodiments, the substitution is between 1 and 5 substitutions.

Exemplary homologues comprise one or more conservative amino acid substitutions including one or more conservative amino acid substitutions within the same group of predetermined amino acids, or a plurality of conservative amino acid substitutions, wherein each conservative substitution is generated by substitution within a different group of predetermined amino acids. Homologues may thus comprise conservative substitutions independent of one another, wherein at least one glycine (Gly) of said homologue is substituted with an amino acid selected from the group of amino acids consisting of Ala, Val, Leu, and Ile, and independently thereof, homologues, wherein at least one of said alanines (Ala) of said homologue thereof is substituted with an amino acid selected from the group of amino acids consisting of Gly, Val, Leu, and Ile; and independently thereof, homologues wherein at least one valine (Val) of said homologue thereof is substituted with an amino acid selected from the group of amino acids consisting of Gly, Ala, Leu, and Ile; and, independently thereof, homologues wherein at least one of said leucines (Leu) of said homologue thereof is substituted with an amino acid selected from the group of amino acids consisting of Gly, Ala, Val, and Ile; and independently thereof, homologues wherein at least one isoleucine (Ile) of said homologues thereof is substituted with an amino acid selected from the group of amino acids consisting of Gly, Ala, Val and Leu; and, independently thereof homologues wherein at least one of said aspartic acids (Asp) of said homologue thereof is substituted with an amino acid selected from the group of amino acids consisting of Glu, Asn, and Gln; and independently thereof, homologues wherein at least one of said phenylalanines (Phe) of said homologues thereof is substituted with an amino acid selected from the group of amino acids consisting of Tyr, Trp, His, and Pro, and selected from the group of amino acids consisting of Tyr and Trp; and independently thereof, homologues wherein at least one of said tyrosines (Tyr) of said homologues thereof is substituted with an amino acid selected from the group of amino acids consisting of Phe, Trp, His, and Pro, or an amino acid selected from the group of amino acids consisting of Phe and Trp; and, independently thereof, homologues wherein at least one of said arginines (Arg) of said fragment is substituted with an amino acid selected from the group of amino acids consisting of Lys and His; and, independently thereof, homologues wherein at least one lysine (Lys) of said homologues thereof is substituted with an amino acid selected from the group of amino acids consisting of Arg and His; and, independently thereof homologues wherein at least one of said asparagines (Asn) of said homologues thereof is substituted with an amino acid selected from the group of amino acids consisting of Asp, Glu, and Gln; and, independently thereof, homologues wherein at least one glutamine (Gin) of said homologues thereof is substituted with an amino acid selected from the group of amino acids consisting of Asp, Glu, and Asn; and, independently thereof homologues wherein at least one proline (Pro) of said homologues thereof is substituted with an amino acid selected from the group of amino acids consisting of Phe, Tyr, Trp, and His; and, independently thereof, homologues wherein at least one of said cysteines (Cys) of said homologues thereof is substituted with an amino acid selected from the group of amino acids consisting of Asp, Glu, Lys, Arg, His, Asn, Gln, Ser, Thr, and Tyr. Non-limiting examples of common substitutions for various residues can be found in the NCBI Amino Acid Explorer database, which includes listings of common substitutions for each amino acid, along other types of information on each amino acid as part of its BLOSUM62 matrix database (see Substitutes in BLOSUM62 on the worldwide web at: ncbi.nlm.nih.gov/Class/Structure/aa/aa_explorer.cgi.

Conservative substitutions may be introduced in any position of a predetermined sequence. It may however also be desirable to introduce non-conservative substitutions, particularly, but not limited to, a non-conservative substitution in any one or more positions. In some embodiments the substitutions can be conservative substitutions, which are well known in the art (see for example Creighton (1984) Proteins. W. H. Freeman and Company (Eds). Table 2 below depicts non-limiting examples of conservative substitutions that can be made:

TABLE 2

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

In some embodiments, one or more amino acids can be substituted with an amino acid or synthetic amino acid that has a similar property or a different property at its side chain or otherwise, such as charge, polarity, hydrophobicity, antigenicity, propensity to form or break α-helical structures or β-sheet structures.

A non-conservative substitution leading to the formation of a functionally equivalent homologue of the sequences herein would, for example, i) differ substantially in polarity, for example a residue with a non-polar side chain (Ala, Leu, Pro, Trp, Val, Ile, Gly, Leu, Phe or Met) substituted for a residue with a polar side chain such as Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids may in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include, for example, arginine and lysine; glutamate and aspartate; serine and threonine; glutamine and asparagine; and valine, leucine, and isoleucine.

Some non-limiting examples of potential molecules that can be substituted for amino acids are provided below in Table 3.

TABLE 3

| Symbol | Meaning |
|---|---|
| A3c | 1-amino-1-cyclopropanecarboxylic acid |
| A4c | 1-amino-1-cyclobutanecarboxylic acid |
| A5c | 1-amino-1-cyclopentanecarboxylic acid |
| A6c | 1-amino-1-cyclohexanecarboxylic acid |
| Aad | 2-Aminoadipic acid |
| bAad | 3-aminoadipic acid |
| bAla | beta-Alanine, beta-Aminopropionic acid |
| Abu | 2-Aminobutyric acid |
| 4Abu | 4-Aminobutyric acid, piperidinic acid |
| Acc | 1-amino-1-cyclo(C₃-C₉)alkyl carboxylic acid |
| Acp | 6-Aminocaproic acid |
| Act | 4-amino-4-carboxytetrahydropyran |
| Ahe | 2-Aminoheptanoic acid |
| Aib | 2-Aminoisobutyric acid |
| bAib | 3-Aminoisobutyric acid |
| Apc | amino piperidinylcarboxylic acid |
| Apm | 2-Aminopimelic acid |
| hArg | homoarginine |
| Bal | 3-Benzothienylalanine |
| Bip | 4,4′-Biphenylalanine |
| Bpa | 4-Benzoylphenylalanine |
| Cha | β-cyclohexylalanine |
| Dbu | 2,4-Diaminobutyric acid |
| Des | Desmosine |
| Dip β | β-Diphenylalanine |
| Dmt | 5,5-dimethylthiazolidine-4-carboxylic acid |
| Dpm | 2,2-Diaminopimelic acid |
| Dpr | 2,3-Diaminopropionic acid |
| EtGly | N-Ethylglycine |
| EtAsn | N-Ethylasparagine |
| 2Fua | β-(2-furyl)-alanine |
| Hyl | Hydroxylysine |
| aHyl | allo-Hydroxylysine |
| 3Hyp | 3-Hydroxyproline |
| 4Hyp | 4-Hydroxyproline |
| Ide | Isodesmosine |
| aIle | allo-Isoleucine |
| Inc | indoline-2-carboxylic acid |
| Inp | isonipecotic acid |
| Ktp | 4-ketoproline |
| hLeu | homoleucine |
| MeGly | N-Methylglycine, sarcosine |
| MeIle | N-Methylisoleucine |
| MeLys | 6-N-Methyllysine |
| MeVal | N-Methylvaline |
| 1Nal | β-(1-Naphthyl)alanine |
| 2Nal | β-(2-Naphthyl)alanine |
| Nva | Norvaline |
| Nle | Norleucine |
| Oic | octahydroindole-2-carboxylic acid |
| Orn | Ornithine |
| 2Pal | β-2-Pyridyl)-alanine |
| 3Pal | β-(3-Pyridyl)-alanine |
| 4Pal | β-(4-Pyridyl)-alanine |
| Pff | pentafluorophenylalanine |
| hPhe | homophenylalanine |
| Pim | 2′-(4-Phenyl)imidazolyl |
| Pip | pipecolic acid |
| Taz | β-(4-thiazolyl)alanine |
| 2Thi | β-(2-thienyl)alanine |
| 3Thi | β-(3-thienyl)alanine |
| Thz | thiazolidine-4-carboxylic acid |
| Tic | 1,2,3,4-tetrahydroisoquinoline-3-carboxylic acid |
| Tle | tert-leucine |

In some embodiments, a ghrelin variant is des-acyl-ghrelin of the primary amino acid sequence as provided in SEQ ID NO. 1. In some embodiments, a ghrelin variant binds to a receptor other than GHSR-1a or ghrelin receptor, and wherein binding to a receptor other than GHSR-1a or ghrelin receptor provides a therapeutic benefit following mBI, for example, neuroprotection following mBI, repeated mBI or CTE (Chronic Traumatic Encephalopathy). The therapeutic benefit such as neuroprotection following mBI or repeated mBI or CTE may include reduced oxidative stress or reduced apoptosis.

In some embodiments, a ghrelin variant binds to CA36 receptor. In some embodiments, a ghrelin variant binds to CD36. CD36 (i.e., Cluster of Differentiation 36) is also known as FAT (fatty acid translocase), FAT/CD36, (FAT)/CD36, SCARB3, GP88, glycoprotein IV (gpIV), and glycoprotein IIIb (gpIIIb). CD36 is an integral membrane protein and is a member of the class B scavenger receptor family of cell surface proteins. CD36 participates in internalization of apoptotic cells, bacterial and fungal pathogens, contributes to inflammatory responses, and facilitates long-chain fatty acids transport into cells. CD36 is involved in, but not limited to, muscle lipid utilization, adipose energy storage, gut fat absorption and the pathogenesis of metabolic disorders, such as diabetes and obesity. Hexarelin, a growth hormone-releasing peptide, has been shown to bind CD36 and ghrelin receptor, GH secretagogue-receptor 1a to up-regulates sterol transporters and cholesterol efflux in macrophages through a peroxisome proliferator-activated receptor gamma-dependent pathway.

In some embodiments, a ghrelin variant comprises a polypeptide comprising at least one modification to the natural form of an amino acid sequence of Gly-Ser-Ser-Phe-Leu-Ser-Pro-Ser-Gln-Lys-Pro-Gln-Asn-Lys-Val-Lys-Ser-Ser-Arg-Ile (SEQ ID NO. 18). In another embodiment, a ghrelin variant comprises a polypeptide comprising at least one modification to the natural form of an amino acid sequence of Gly-Ser-Ser-Phe-Leu-Ser-Pro-Glu-His-Gln-Lys-Ala-Gln-Gln-Arg-Lys-Glu-Ser-Lys-Lys-Pro-Pro-Ala-Lys-Leu-Gln-Pro-Arg (SEQ ID NO. 19). In another embodiment, a ghrelin variant comprises a polypeptide comprising at least one modification to the natural form of an amino acid sequence of Gly-Ser-Ser-Phe-Leu-Ser-Pro-Glu-His-Gln-Lys-Val-Gln-Gln-Arg-Lys-Glu-Ser-Lys-Lys-Pro-Ala-Ala-Lys-Leu-Lys-Pro-Arg (SEQ ID NO. 20). In another embodiment, a ghrelin variant comprises a polypeptide comprising at least one modification to the natural form of an amino acid sequence of Gly-Ser-Ser-Phe-Leu-Ser-Pro-Glu-His-Gln-Arg-Ala-Gln-Gln-Arg-Lys-Glu-Ser-Lys-Lys-Pro-Pro-Ala-Lys-Leu-Gln-Pro-Arg (SEQ ID NO. 21). In another embodiment, a ghrelin variant comprises a polypeptide comprising at least one modification to the natural form of an amino acid sequence of Gly-Ser-Ser-Phe-Leu-Ser-Pro-Thr-Tyr-Lys-Asn-Ile-Gln-Gln-Gln-Lys-Asp-Thr-Arg-Lys-Pro-Thr-Ala-Arg-Leu-His (SEQ ID NO. 22). In yet another embodiment, a ghrelin variant comprises a polypeptide comprising at least one modification to the natural form of an amino acid sequence of Gly-Ser-Ser-Phe-Leu-Ser-Pro-Glu-His-Gln-Lys-Leu-Gln-Gln-Arg-Lys-Glu-Ser-Lys-Lys-Pro-Pro-Ala-Lys-Leu-Gln-Pro-Arg (SEQ ID NO. 23). In another embodiment, a ghrelin variant comprises a polypeptide comprising an amino acid sequence of Gly-Ser-Ser(O-n-octanoyl)-Phe-Leu-Ser-Pro-Glu-His-Gln-Arg-Val-Gln-Gln-Arg-Lys-Glu-Ser-Lys-Lys-Pro-Pro-Ala-Lys-Leu-Gln-Pro-Arg (SEQ ID NO. 24). In another embodiment, a ghrelin variant comprises a polypeptide comprising an amino acid sequence of Fluorescein-Ahx-Gly-Ser-Ser(O-n-octanoyl)-Phe-Leu-Ser-Pro-Glu-His-Gln-Arg-Val-Gln-Gln-Arg-Lys-Glu-Ser-Lys-Lys-Pro-Pro-Ala-Lys-Leu-Gln-Pro-Arg (Ahx=Aminohexanoic acid) (SEQ ID NO. 25). In another embodiment, a ghrelin variant comprises a polypeptide comprising an amino acid sequence of Gly-Ser-Ser(O-n-octanoyl)-Tyr-Leu-Ser-Pro-Glu-His-Gln-Arg-Val-Gln-Gln-Arg-Lys-Glu-Ser-Lys-Lys-Pro-Pro-Ala-Lys-Leu-Gln-Pro-Arg (SEQ ID NO. 26).

Some embodiments relate to and can utilize ghrelin or ghrelin variant molecules that have a carbon 14 (C14) content less than found in endogenously produced ghrelin or ghrelin variant molecules or in ghrelin or ghrelin variant that has a C14 content about the same as atmospheric C14 levels. For example, ghrelin or ghrelin variant molecules can have at least one carbon atom or carbon containing moiety that is from fossil derived reagents that have a C14 content less than found in endogenous molecules or less than atmospheric levels. In some embodiments, the ghrelin or ghrelin variant molecules can have all, substantially all or at least a some carbon having a C14 content less than found endogenously or less than atmospheric levels. For example, one or more of the amino acids of a sequence can include carbon and have a C14 content less than found in endogenous amino acids or less than atmospheric levels. In other cases an entire sequence can include carbon and have a C14 content less than found endogenously or less than atmospheric levels. Still, in other embodiments, a ghrelin or ghrelin variant molecule can be modified, for example to have an octanoyl or other like group, and that octanoyl group can have a C14 content less than endogenous ghrelin C14 levels or less than atmospheric levels. Further examples and embodiments are described below and elsewhere herein.

In some embodiments, ghrelin or ghrelin variant can have a C14 content of less than 0.9 ppt, 0.95 ppt, 1.05 ppt, 1.10 ppt, 1.15 ppt, 1.2 ppt or atmospheric content of C14. In some embodiments, ghrelin molecule can have a C14 content that is from about 1% to 50% (or any value or sub range therein) less than the content of C14 in endogenous ghrelin or the content of atmospheric C14. For example, a molecule according to some embodiments can have about 5% to about 11% less C14 content. Ghrelin with C14 content of less than 0.9 ppt, 0.95 ppt, 1.0 ppt, 1.05 ppt, 1.10 ppt, 1.15 ppt, 1.2 ppt or atmospheric content of C14, or with a lesser percentage of C14 as discussed herein, may be obtained by peptide or chemical synthesis using reactants with carbons free of C14, less than 1 ppt C14 or deficient in C14 relative to the atmospheric content of C14. Alternatively, ghrelin or ghrelin variant with C14 content of less than 0.9 ppt, 0.95 ppt, 1.0 ppt, 1.05 ppt, 1.10 ppt, 1.15 ppt, 1.2 ppt or atmospheric content of C14 may be produced in vitro by enzymatic methods using starting materials with a carbon content free of C14, substantially free of C14, less than 1 ppt C14 or deficient in C14 relative to the atmospheric content of C14. Such enzymatic methods may include cell-free protein synthesis system or coupled in vitro transcription-translation system based on cellular extracts prepared from bacteria, yeast, wheat germ, insect and/or mammalian cells using aminoacyl-tRNAs charged with amino acids with a carbon content free of C14, substantially free of C14, less than 1 ppt C14 or deficient in C14 relative to the atmospheric content of C14. In an alternative method, ghrelin or ghrelin variant with C14 content of less than 0.9 ppt, 0.95 ppt, 1.0 ppt, 1.05 ppt, 1.10 ppt, 1.15 ppt, 1.2 ppt or atmospheric content of C14 may be produced by recombinant methods in bacterial, yeast, insect and/or mammalian cells following introduction of an expression system with a cDNA comprising ghrelin-encoded sequences and culturing the cells in a medium with a carbon content free of C14, substantially free of C14, less than 1 ppt C14 or deficient in C14 relative to the atmospheric content of C14. Alternatively, the medium may include glucose, galactose, sugars, glycerol, pyruvate, acetates, metabolites, fatty acids, and/or amino acids with a carbon content free of C14, substantially free of C14, less than 1 ppt C14 or deficient in C14 relative to the atmospheric content of C14. Methods for changing stable isotopic content of proteins may be found in Becker et al., 2008 (G. W. Becker (2008) Stable isotopic labeling of proteins for quantitative proteomic applications. Briefings in Functional Genomics Proteins 7 (5): 371-382, which is incorporated herein by reference in its entirety) Ghrelin may be co-expressed with or exposed to ghrelin O-acyl transferase (GOAT) to permit fatty acid modification of the primary sequence of ghrelin or ghrelin variant at serine at amino acid position 3 so as to produce a biologically active ghrelin or ghrelin variant capable of being bound and activating the ghrelin receptor (GHSR-1a or growth hormone secretagogue receptor type 1a). The modification may be an octanoic acid modification of ghrelin or ghrelin variant so as to produce octanoyl-ghrelin with a carbon content free of C14, substantially free of C14, less than 1 ppt C14 or deficient in C14 relative to the atmospheric content of C14.

The biosynthesis of acyl-ghrelin involves a post-translational octanoylation of the serine at the 3 position of the ghrelin peptide. This octanoylation is necessary for its bioactivity, which occurs via interaction with the growth hormone secretagogue receptor (GHSR). GOAT is responsible for this esterification. GOAT is a member of the membrane-bound O-acyltransferase (mBOAT) family of membrane proteins. GOAT is a polytopic integral membrane protein that octanoylates Ser3 of proghrelin in the endoplasmic reticulum (ER)lumen after signal peptide cleavage. In some embodiment, GOAT is up-regulated to increase the endogenous acylated ghrelin after mild brain injury or concussion. In some embodiments, the up-regulation of GOAT is at the protein expression level. In some embodiments, the up-regulation of GOAT is at the mRNA expression level.

In some embodiments, ghrelin or ghrelin variant with C14 content of less than 0.9 ppt, 0.95 ppt, 1.0 ppt, 1.05 ppt, 1.10 ppt, 1.15 ppt, 1.2 ppt or atmospheric content of C14 (or having a percentage as discussed herein) may be obtained following modification of the primary sequence of ghrelin (SEQ ID NO. 1) with a fatty acid with a carbon content free of C14, substantially free of C14, less than 1 ppt C14 or deficient in C14 relative to the atmospheric content of C14. Such fatty acids may be chemically synthesized with a carbon content free of C14, substantially free of C14, less than 1 ppt C14 or deficient in C14 relative to the atmospheric content of C14 or produced in a cell cultured in a medium wherein carbon source used to synthesize the fatty acid or fatty acids is free of C14, substantially free of C14, less than 1 ppt C14 or deficient in C14 relative to the atmospheric content of C14. In some embodiments, the fatty acid or fatty acids are conjugated to coenzyme A (CoA) and the fatty acid in the resulting fatty acid-CoA thioesters is transferred to serine at amino acid position 3 of ghrelin by ghrelin O-acyl transferase (GOAT), so as to produce a fatty acid-modified ghrelin with a carbon content free of C14, substantially free of C14, less than 1 ppt C14 or deficient in C14 relative to the atmospheric content of C14. In some embodiments, fatty acids are straight chain fatty acids with a carbon content of C2, C3, C4, C5, C6, C7, C8, C9, C10, C11, C12, C13, C14, C15, C16, C17, C18, C19 or C20 and having a general chemical formula of (CH)3-(CH2)n-2-COOH, wherein “n” is the number of carbons in the fatty acid. In a preferred embodiment, the fatty acid is a C8 octanoic acid or C14 tetradecanoic acid. In a more preferred embodiment, the fatty acid is octanoic acid and the fatty acid-modified ghrelin is octanoyl-modified ghrelin or ghrelin variant at serine amino acid position 3.

In some embodiments, fatty acid or fatty acids may be conjugated to ghrelin or ghrelin variant at serine amino acid position 3. In some embodiments, fatty acid or fatty acids may be conjugated to ghrelin or ghrelin variant at a position other than serine amino acid position 3. In some embodiments, fatty acid or fatty acids may be conjugated to ghrelin or ghrelin variant at serine amino acid position 2. In some embodiments, fatty acid or fatty acids may be conjugated to ghrelin or ghrelin variant at serine amino acid position 2 and serine amino acid position 3. In some embodiments, fatty acid or fatty acids may be conjugated to ghrelin or ghrelin variant at one or more amino acids.

In some embodiments, fatty acid or fatty acids may be conjugated to immature ghrelin (such as preproghrelin or proghrelin) and then fatty acid- or fatty acids-modified ghrelin is processed to a mature ghrelin that can activate the ghrelin receptor (GHSR-1a). Processing of immature ghrelin may be in vitro or in vivo and may be carried out by proteolytic enzymes. In some embodiments, fatty acid or fatty acids may be conjugated to a mature ghrelin having the amino acid sequence as provided in SEQ ID NO. 1.

In some embodiments, ghrelin with one or more modifications is an isolated ghrelin with one or more modifications. In some embodiments, ghrelin with one or more modifications is an isolated ghrelin with one or more fatty acid modifications. In some embodiments, ghrelin with one or more modifications is an isolated ghrelin acylated at serine 3 with octanoic acid, such as an isolated octanoyl-ghrelin.

In some embodiments, C14-deficient starting material used in the synthesis of ghrelin or ghrelin variant with a carbon content free of C14, substantially free of C14, less than 1 ppt C14 or deficient in C14 relative to the atmospheric content of C14 may be obtained from carbon sources not participating in atmospheric carbon cycle or by fractionating naturally occurring carbon isotope to obtain carbons free of C14, substantially free of C14, less than 1 ppt C14 or deficient in C14 relative to the atmospheric content of C14. Such carbons will be enriched in carbon-12 (C12) and/or carbon-13 (C13) and depleted of C14. Methods for isotope fractionation, enrichment or depletion are known in the art and may be based on diffusion, centrifugation, electromagnetism, laser excitation, kinetic isotope effect, chemical methods, gravity, evaporation, and cryogenic distillation among many other methods of isotope fractionation.

In some embodiments, other types of ghrelin variants are mimetics, which include: peptidomimetics, small molecule mimetics and GHS-R agonists. A substantial number of ghrelin mimetics are known in the art. Non-limiting examples of ghrelin mimetics include LY444711 and LY426410 (Eli Lilly), hexarelin/examorelin (Diverse Academic), growth hormone releasing hexapeptide-1 (GHRP-I), GHRP-2, GHRP-6 (SK&F-110679), ipamorelin (Helsinn), MK-0677, NN703, capromorelin(Pfizer), CP 464709 (Pfizer), pralmorelin (GHRP 2, GPA 748, growth hormone-releasing peptide 2, KP-102 D, KP-102 LN, KP-102D, KP-102LN, Kaken Pharma, Sella Pharma), macimorelin (Aeterna Zentaris), anamorelin (Helsinn), relamorelin (Rhythm), ulimorelin (Tranzyme), ipamorelin ((NNC-260161, Helsinn), Tabimorelin (Novo Nordisk), ibutamoren (Merck), G7039, G7134, G7203, G7502, SM-130686 (Sumitomo), RC-1291, L-692429, L-692587, L-739943, L-163255, L-163540, L-163833, L-166446, CP-424391, EP-51389, NNC-26-0235, NNC-26-0323, NNC-26-0610, NNC 26-0703, NNC-26-0722, NNC-26-1089, NNC-26-1136, NNC-26-1137, NNC-26-1187, NNC-26-1291, MK-0677, L-692,429, EP 1572, L-252,564, NN703, S-37435, EX-1314, PF-5190457, AMX-213, and macrocyclic compounds (U.S. Publication No. 20060025566, which is incorporated herein by reference in its entirety). See also Smith, 2005, Endo. Rev. 26:346-360 (incorporated herein by reference in its entirety) for information on developing ghrelin mimetics. In some embodiments, one or more of the variants listed above, can be specifically excluded.

In some embodiments, the ghrelin variant is LY444711 (Eli Lilly), which is a compound of the chemical nomenclature: 2-(2-Amino-2-methyl-propionylamino)-5-phenyl-pentanoic acid [1-[1-(4-methoxy-phenyl)-1-methyl-2-oxo-2-pyrrolidin-1-yl-ethyl]-1H-imidazol-4-yl]-amide.

In some embodiments, the ghrelin variant is MK-0677 (or L-163,191), which is a compound of the formula:

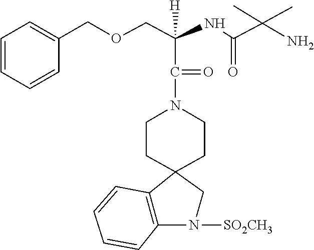 [SMILES: CC(C)(N)C(=O)N[C@H](COCc1ccccc1)C(=O)N1CCC2(CC1)CN(S(C)(=O)=O)c1ccccc12]

In some embodiments, the ghrelin variant is L-692,429, which is a compound of the formula:

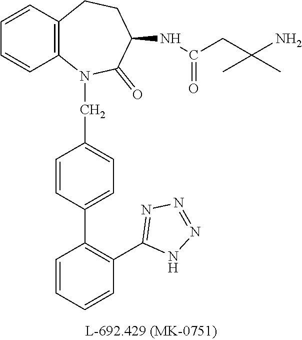 [SMILES: CC(C)(N)CC(=O)N[C@@H]1CCc2ccccc2N(Cc2ccc(-c3ccccc3-c3nnn[nH]3)cc2)C1=O]

In some embodiments, the ghrelin variant is NNC-26-0703 (or Tabimorelin, NN-703), which is a compound of the formula:

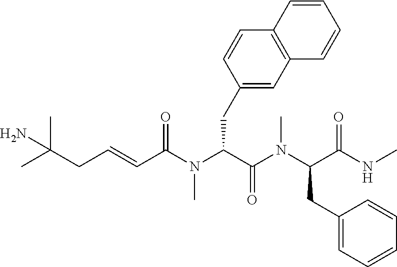 [SMILES: CNC(=O)[C@@H](Cc1ccccc1)N(C)C(=O)[C@@H](Cc1ccc2ccccc2c1)N(C)C(=O)/C=C/CC(C)(C)N]

In some embodiments, the ghrelin variant is Ape-Ser(Octyl)-Phe-Leu-aminoethylamide. In some embodiments, the ghrelin variant is Capromorelin (CP-424,391), which is a compound of the chemical nomenclature: (3aR)-3a-benzyl-2-methyl-5-(2-methylalanyl-O-benzyl-D-seryl)-3-oxo-3,3a,4,5,6,7-hexahydro-2H-pyrazolo[4,3-c]pyridine.

In some embodiments, the ghrelin variant is L-252,564, which is a compound of the chemical nomenclature: 2-({4-[3-(4,5-Dichloro-2-methylphenyl)-4,5-dihydro-1H-pyrazol-1-yl]phenyl}sulfonyl)ethyl acetate, and the formula:

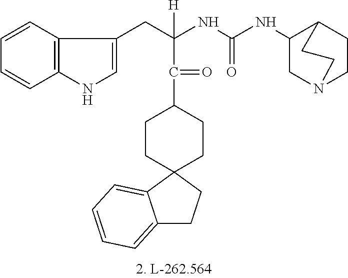 [SMILES: O=C(NC(Cc1c[nH]c2ccccc12)C(=O)C1CCC2(CCc3ccccc32)CC1)NC1CN2CCC1CC2]

In some embodiments, the ghrelin variant is S-37435 (Laken), which is a compound of the chemical nomenclature: N-[1(R)-[N-(3-Amino-2-hydroxypropyl)carbamoyl]-2-naphthylethyl]-4-(4-oxo-2,3,4,5-tetrahydro-1,5-benzothiazepin-5-yl)butyramide hydrochloride. In some embodiments, the ghrelin variant is G-7203 (Genentech). In some embodiments, the ghrelin variant is SM-130868 (Sumitomo). In some embodiments, the ghrelin variant is EX-1314.

In some embodiments, the ghrelin variant is ulimorelin which has the molecular formula, C₃₀H₃₉FN₄O₄, and the following structure:

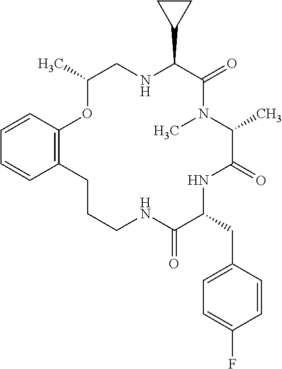 [SMILES: C[C@@H]1CN[C@@H](C2CC2)C(=O)N(C)[C@H](C)C(=O)N[C@H](Cc2ccc(F)cc2)C(=O)NCCCc2ccccc2O1]

U.S. Pat. No. 7,491,695, which is incorporated herein by reference in its entirety for all of its materials, compositions of matter, methods of use and methods of making, discloses various macrocyclic compounds, including ulimorelin.

In some embodiments, the ghrelin variant is macimorelin, which has the molecular formula, C₂₆H₃₀N₆O₃, and the following structure:

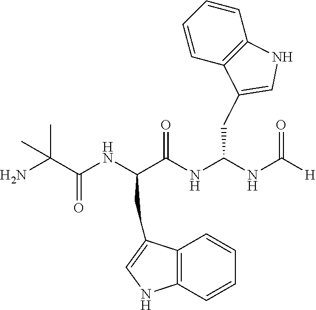 [SMILES: CC(C)(N)C(=O)N[C@H](Cc1c[nH]c2ccccc12)C(=O)N[C@H](Cc1c[nH]c2ccccc12)NC=O]

U.S. Pat. No. 8,192,719, which is incorporated herein by reference in its entirety for all of its materials, compositions of matter, methods of use and methods of making, discloses various compounds, including macimorelin, which can be utilized in the embodiments herein.

In some embodiments, the ghrelin variant is anamorelin, which has the molecular formula, C₃₁H₄₃ClN₆O₃, and the following structure:

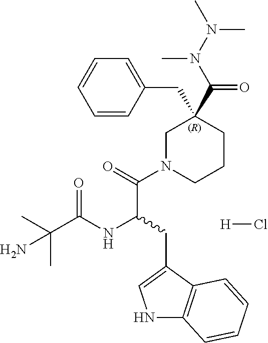

In some embodiments, the ghrelin variant is ipamorelin, which has the molecular formula, C₃₈H₄₉N₉O₅, and the following structure:

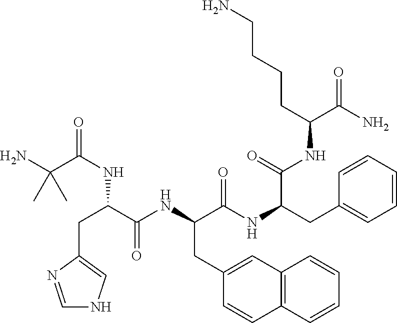 [SMILES: CC(C)(N)C(=O)N[C@@H](Cc1c[nH]cn1)C(=O)N[C@H](Cc1ccc2ccccc2c1)C(=O)N[C@H](Cc1ccccc1)C(=O)N[C@@H](CCCCN)C(N)=O]

In some embodiments, the ghrelin variant is PF-5190457, which has the following structure:

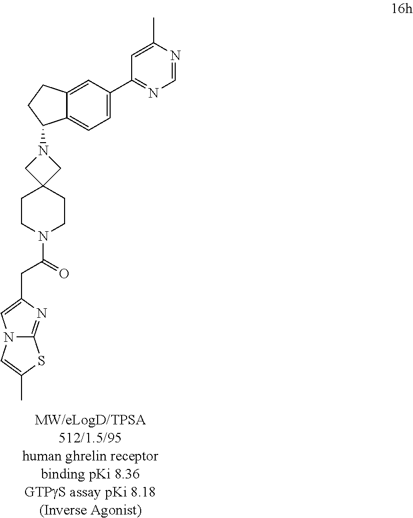 [SMILES: Cc1cc(-c2ccc3c(c2)CC[C@H]3N2CC3(CCN(C(=O)Cc4cn5cc(C)sc5n4)CC3)C2)ncn1]

In some embodiments, the ghrelin variant binds to the growth hormone secretagogue receptor GHS-R 1a (GHSR). The ghrelin variant compounds described herein are active at the receptor for growth hormone secretagogue (GHS), e.g., the receptor GHS-R 1a. The compounds can bind to GHS-R 1a, and stimulate receptor activity. In some embodiments, the compounds can bind other receptors and, optionally, stimulate their activity.

Ghrelin variants, in some embodiments, can activate the GHS receptors and additional yet to be identified receptors. These receptors are found on GH producing cells, in the hypothalamic centers and in a number of additional places in the organism. In the CNS, these receptors are tuned to receiving signals from neurons containing local molecules (e.g., ghrelin variants). Peripherally-secreted or artificially-administered ghrelin variants and combination products (including fusion therapeutic products) as described herein can reach such sites and pass the blood brain barrier specifically activating the appropriate receptors and triggering a specific pathway. GH secretagogues, which are small organic compounds such as MK-0677 (Merck), generally target to bind the GHS receptor will pass the blood brain barrier and also reach these sites, activating various GHS receptor related pathways and consequently having the danger of causing unwanted side effects such as dizziness, nausea, falling, elevated fasting serum glucose and insulin, and blurred vision. Such compounds which do have the advantage of being, for example, orally active. Other ghrelin variants, or homologues thereof, can be administered peripherally to ensure that only the relevant, appetite-regulating ghrelin splice variant receptors and pathways are reached and stimulated.

In some embodiments, the ghrelin variant increases uncoupling protein-2 (UCP-2) expression. In some embodiments, the ghrelin variant increases UCP-2 expression in mitochondria. In some embodiments, the ghrelin variant prevents the metabolic consequence of mBI and any associated chronic conditions.

In some embodiments, the ghrelin variant has at least about 50% of the functional activity of ghrelin. In some embodiments, the functional activity comprises one or more of feeding regulation, nutrient absorption, gastrointestinal motility, energy homeostasis, anti-inflammatory regulation, suppression of inflammatory cytokines, activation of Gq/G11, accumulation of inositol phosphate, mobilization of calcium from intracellular stores, activation or deactivation of MAP kinases, NFκB translocation, CRE driven gene transcription, binding of arrestin to ghrelin receptor, reduction in ROS, NAMPT enzyme activation, or a combination thereof.

Receptor activity can be measured using different techniques such as detecting a change in the intracellular conformation of the receptor, in the G-protein coupled activities, and/or in the intracellular messengers. One simple measure of the ability of a ghrelin variant-like compound to activate the ghrelin variant receptor is to measure its EC₅₀, i.e. the dose at which the compound is able to activate the signaling of the receptor to half of the maximal effect of the compound. When measuring, e.g., EC₅₀, the receptor can either be expressed endogenously on primary cell cultures, for example pituitary cells, or heterologously expressed on cells transfected with the ghrelin receptor. Whole cell assays or assays using membranes prepared from either of these cell types can be used depending on the type of assay. In some embodiments, the ghrelin variant has an EC₅₀ potency on the GHSR of less than 500 nM. In some embodiments, the ghrelin variant has a dissociation constant from the GHSR of less than 500 nM.

A ghrelin variant compound has at least about 50%, at least about 55%, at least about 60%, at least about 65%, at least about 70%, at least about 75%, at least about 80%, at least about 85%, at least about 90%, or at least about 95%, functional activity relative to the 28 amino acid human wild-type ghrelin as determined using an assay described herein, and/or an EC50 greater than about 1,000, greater than about 100, or greater than about 50, or greater than about 10. Greater refers to potency and thus indicates a lesser amount is needed to achieve binding inhibition.

In some embodiments, the ghrelin variant has potency (EC50) on the GHS-R 1a of less than 500 nM. In some embodiments, the ghrelin variant has a potency (EC50) on the GHS-R 1a of less than 100 nM, such as less than 80 nM, such as less than 60 nM, such as less than 40 nM, such as less than 20 nM, such as less than 10 nM, such as less than 5 nM, such as less than 1 nM, such as less than 0.5 nM, such as less than 0.1 nM, such as less than 0.05 nM, such as less than 0.01 nM.

In some embodiments, the dissociation constant (Kd) of the ghrelin variant is less than 500 nM. In some embodiments, the dissociation constant (Kd) of the ghrelin variant is less than 100 nM, such as less than 80 nM, such as less than 60 nM, such as less than 40 nM, such as less than 20 nM, such as less than 10 nM, such as less than 5 nM, such as less than 1 nM, such as less than 0.5 nM, such as less than 0.1 nM, such as less than 0.05 nM, such as less than 0.01 nM.

Binding assays can be performed using recombinantly-produced receptor polypeptides present in different environments. Such environments include, for example, cell extracts and purified cell extracts containing the receptor polypeptide expressed from recombinant nucleic acid or naturally occurring nucleic acid, and also include, for example, the use of a purified GHS receptor polypeptide produced by recombinant means or from naturally occurring nucleic acid which is introduced into a different environment.

Using a recombinant GHS receptor offers several advantages, such as the ability to express the receptor in a defined cell system, so that a response to a compound at the receptor can more readily be differentiated from responses at other receptors. For example, the receptor can be expressed in a cell line such as HEK 293, COS 7, and CHO not normally expressing the receptor by an expression vector, wherein the same cell line without the expression vector can act as a control.

In some embodiments, the ghrelin variant is coupled to a protein that extends the serum half-lives of the ghrelin variant. In some embodiments, the protein is a long, hydrophilic, and unstructured polymer that occupies a larger volume than a globular protein containing the same number of amino acids.

In some embodiments, the protein comprising the sequence of XTEN™ (SEQ ID NO. 7). XTEN™, is a long, hydrophilic, and unstructured polymer that occupies a much greater volume than any globular protein containing the same number of amino acids. When attached to molecules of interest, XTEN™ greatly increases their effective size, thereby prolonging their presence in serum by slowing kidney clearance in a manner analogous to that of polyethylene glycol (PEG). In addition to slowing kidney clearance, attachment to XTEN™ can also inhibit receptor-mediated clearance by reducing the ligand's affinity for its receptor. Such an effect is not accomplished by fusion to other half-life extension technologies like HSA or Fc. Thus, XTEN™ acts through multiple mechanisms to affect drug concentration, resulting in long half-lives and monthly dosing. Proteins and peptides can be produced as recombinant fusions with XTEN™, the length of which can be modified to reach the desired pharmacokinetic properties. XTEN™ also enhances the solubility of attached molecules typically permitting liquid formulation of drugs that otherwise would be lyophilized.

Treatment of Mild Brain Injury (mBI) and Other Neurological Disorders

The present disclosure is directed to the identification of a novel use for a ghrelin variant in mBI. mBI, including concussion, has a significantly different pathology than other traumas associated with brain disease and illness and severe traumatic brain injuries such as those cause ischemia. mBI does not cause the massive tissue and cellular damage as observed in severe traumatic brain disease. Rather mBI causes subtle metabolic changes within the brain, specifically oxidative stress and overproduction of reactive oxygen species (ROS) which, in turn, can damage neuroconnectivity and lead to neuron damage and encephalopathic and psychological changes with recurrent injury.

The present disclosure utilizes ghrelin variants in treating mBI. Unlike severe traumatic injury to the brain, mBI does not show acute neuronal histological changes, severe neuronal inflammation or significant cellular or vasogenic edema (i.e., blood brain barrier breakdown). Rather, mBI is an amorphous clinical definition generally associated with a list of symptoms. It sometimes is associated with loss of consciousness but does not necessarily with significant radiological changes. Symptoms that often are reported include, without limitation, headache, “pressure in head,” neck pain, nausea or vomiting, dizziness, blurred vision, balance problems, sensitivity to light sensitivity to noise, feeling slowed down, feeling like “in a fog,” “don't feel right,” difficulty concentrating, difficulty remembering, fatigue or low energy, confusion, drowsiness, trouble falling asleep, more emotional, irritability, sadness and being nervous or anxious. In some non-limiting embodiments the methods herein can include or relate to treating or reducing one or more of the above listed symptoms. Some embodiments also relate to treating, preventing or reducing post-concussion syndrome (PCS). PCS is a set of symptoms that may occur and/or continue weeks, months or a year after the mild brain injury. PCS is characterized by recurrent concussion symptoms that occur after being cleared from a concussion but not due to a secondary injury. PCS manifests weeks to months after clearance and can persist chronically. Most importantly, repeated concussions, as observed in the military and in sports such as football and hockey, cause a series of metabolic disturbances and the formation of ROS. These series of events lead to chronic traumatic encephalopathy (CTE) as witnessed in the autopsies of several deceased professional football players.

In some embodiments, ghrelin variant treatment, by increasing uncoupling protein-2 (UCP-2) expression in the brain, has the ability to decrease ROS and the subsequent damage caused to neurons following mBI. This is especially important for chronic concussions where preventing the inflammatory, excitatory milieu of ROS would have significant clinical impact. ghrelin variant treatments decrease ROS, and therefore prevent the metabolic consequence of concussions and the chronic conditions that are associated thereof.

In some embodiments, the therapeutic effect of ghrelin in mBI is compared to its therapeutic effect in severe TBI. The etiology, imaging, and assessment of mBI lack a cohesive explanation for the observed cognitive deficits of chronic headaches, memory loss and sleep impairment. Only the most advanced and cumbersome technology can even detect subtle changes associated with mBI. Therefore, unlike severe TBI, where a definitive mass lesion is identified with anatomic and cellular changes, mBI is undetectable radiologically, nevertheless, specific metabolic derangements occur. The present disclosure describes that ghrelin variants can be potent neuro-conservative agents in mBI.

Without being limited by any theory, the present disclosure also describes the biological function of ghrelin variants following mBI. After mBI, the metabolic needs of the cell, including glucose requirements, increase. Mild injured cells have significant metabolic derangements causing reactive oxygen species on neurons. This metabolic stress and increased metabolic needs are the fundamental concept underlying acute concussion management. Decreasing ROS and improving glucose uptake the cells and axons can restore intracellular function and remain viable. After a ghrelin variant treatment (including for example with levels far above endogenous levels), the ghrelin variant increases UCP-2 and therefore, increases hydrogen intake, decreases ROS and subsequent neuron damages caused to neurons following mBI. The ghrelin variant treatment prevents cellular apoptosis by increasing mitochondrial UCP-2 thus stabilizing mitochondrial post-mBI breakdown and the development of reactive oxygen free radical species. This prevents or reduces concussion-induced dysfunction and overall improves neurocognitive outcome, as well as prevents memory loss, and chronic BI states of headaches and development of chronic traumatic encephalopathy.

The present disclosure provides for a method of treating mild brain injury (mBI) or concussion in a subject, comprising administering to the subject (e.g., a subject that has a mBI) an effective amount of a compound comprising a ghrelin variant, thereby treating the mBI or concussion. The ghrelin variant can be administered for the purpose of treating the mBI or concussion in a therapeutically effective amount for the mBI or concussion. In some embodiments, the methods can further include selecting or identifying a subject that has suffered, is at risk of suffering, is prone to suffer, and/or is about to participate in an activity with a high risk for suffering, a mBI or concussion, prior to administration of the ghrelin variant.

In some embodiments, the mild brain injury comprises a concussion. In some embodiments, the subject that undergoes the method of treatment is a mammal. In some embodiments, the subject is a human. In some embodiments, the subject is a monkey, cow, goat, sheep, mouse, rat, cat, dog, horse, hamster, pig, fish and chicken. In some embodiments, the ghrelin variant is administered within about 72 hours of the mBI. In some embodiments, the ghrelin variant is administered within about 24 hours of the mBI. In some embodiments, the ghrelin variant is administered at about 0.1, 0.3, 0.5, 0.7, 1, 2, 3, 6, 12, 18, 24, 36, 48, or 72 hours after the mBI.

In some embodiments, an intravenous injection of ghrelin variant is employed. The administration route must ensure that the non-degraded, bioactive form of the peptide will be the dominating form in the circulation, which will reach and stimulate the ghrelin receptors in order to obtain the maximum effect of ghrelin/ghrelin variant treatment on mBI. In some embodiments, the ghrelin variant is administered within about 30 minutes of the incident that results in mBI. In some embodiments, the ghrelin variant is administered within about 30 minutes to about 2 hours of the incident that results in mBI. In some embodiments, the ghrelin variant is administered within about 30 minutes to about 6 hours of the incident that results in mBI. In some embodiments, the ghrelin variant is administered within about 30 minutes to about 12 hours of the incident that results in mBI. In some embodiments, the ghrelin variant is administered within about 30 minutes to about 24 hours of the incident that results in mBI.

A typical dosage is in a concentration equivalent to 10 ng to 10 mg ghrelin variant per kg bodyweight. The concentrations and amounts herein are given in equivalents of amount ghrelin variant, wherein the ghrelin variant is a 28 amino acid human ghrelin (SEQ ID NO:1) and/or a 24 amino acid human ghrelin splice variant having a Dpr residue at the third position (SEQ ID NO:3) and/or a 24 amino acid human ghrelin splice variant having Dpr residues at the second and third positions (SEQ ID NO:4) and being optionally octanoylated on the Dpr residue in the third position.

In some embodiments, ghrelin variants are administered in a concentration equivalent to from about 0.1 μg to about 1 mg ghrelin variant per kg bodyweight, such as from about 0.5 μg to about 0.5 mg ghrelin variant per kg bodyweight, such as from about 1.0 μg to about 0.1 mg ghrelin variant per kg bodyweight, such as from about 1.0 μg to about 50 μg ghrelin variant per kg bodyweight, such as from about 1.0 μg to about 10 μg ghrelin variant per kg bodyweight. In some embodiments, about 10 μg ghrelin powder is reconstituted in about 100 μL of a sterile saline solution before administration. In some embodiments, the sterile saline solution is contained in an IV bag for ease of delivery.

In some embodiments, a ghrelin or ghrelin variant is used in an assay to assess the ability of candidate compounds to effect increased uncoupling protein-2 (UCP-2) expression including increased UCP-2 expression in mitochondria. In such assays, ghrelin is used as a control to determine the relative efficacy of the candidate compound or compounds. Suitable assays include by way of example only competitive assays for binding of a candidate compound or compounds to growth hormone secretagogue receptor 1a (i.e., GHSR) in the presence of ghrelin as well as frontal affinity chromatography.

Any competitive binding assay known in the art is applicable for binding of a candidate compound or compounds to growth hormone secretagogue receptor in the presence of ghrelin, using either heterogeneous or homogeneous methods, with one or more reagents, and with labels and detection methods. By way of non-limiting example, detection methods may include radioactive methods; enzyme techniques using intact enzymes of many types including, for example, β-galactosidase, glucose 6-phosphate dehydrogenase, alkaline phosphatase, horseradish peroxidase, or glucose oxidase; techniques using enzyme fragments, such as β-galactosidase complementation assays; detection systems including chromogenic substrates; fluorescent methods detected by direct fluorescence, time-resolved fluorescence, fluorescence polarization, or fluorescence energy transfer; and chemical or bioluminescence detection systems.

In some embodiments, frontal affinity chromatography (FAC) can be used for screening of compound libraries. The basic premise of FAC is that continuous infusion of a compound will allow for equilibration of the ligand between the free and bound states, where the precise concentration of free ligand is known. The detection of compounds eluting from the column can be accomplished using methods such as fluorescence, radioactivity, or electrospray mass spectrometry. The former two methods usually make use of either a labeled library, or use a labeled indicator compound, which competes against known unlabeled compounds, getting displaced earlier if a stronger binding ligand is present.

In some embodiments, a patient suffering loss of cognitive or motor skills due to mBI and, in particular, repetitive mBI, can be monitored for therapy or progression of such skills by correlating the ghrelin level in the patient's brain over time. As the ghrelin levels decrease, there will be an increased need for intervention.

The present disclosure provides for a method of treating mBI in a subject, comprising administering to the subject an effective amount of a compound comprising the ghrelin variant that is encoded by or administered as a nucleic acid. In some embodiments, the nucleic acid is any that encodes the sequence of SEQ ID NO.1. In some embodiments, the nucleic acid sequence comprises 5′-ggctccagct tcctgagccc tgaacaccag agagtccagc agagaaagga gtcgaagaag ccaccagcca agctgcagcc ccga-3′ (SEQ ID NO. 8). In some embodiments, the ghrelin variant encodes a nucleic acid sequence comprises SEQ ID NO. 8 with one or more mutations. In some embodiments, the mutation is selected from the group consisting of nucleic acid insertion, deletion, substitution and translocation. In some embodiments, the mutation occurs at one or more positions.

Some embodiments relate to methods of treating mild brain injury or concussion or reducing the severity or duration of one or more symptoms or characteristics of the injury by utilizing the methods and compounds described herein in combination with one or more diagnostic devices or protocols, or with one or more recovery protocols. For example, a potential brain injury can be diagnosed and/or monitored utilizing the BTrackS™ System (http://balancetrackingsystems.com/; Balance Tracking Systems Inc.), utilizing the NFL Concussion Tool, “sports concussion assessment tool” (“SCAT-2;” http://static.nfl.com/static/content/public/photo/2014/02/20/0ap2000000327062.pdf) or other similar tools utilized by the NHL, the NBA, FIFA, Rugby leagues and unions, boxing organizations, etc. Examples include, SCAT-3, ImPACT, ICD-10, nPITEST, acute concussion evaluation (“ACE”), King-Devick, and the like. Other diagnostics or assessments can utilize serum biomarkers (Glial Fibrilliary Acid Protein (GFAP); see for example, Mannix et al., “Serum Biomarkers Predict Acute Symptom Burden in Children after Concussion: A Preliminary Study,” JOURNAL OF NEUROTRAUMA 31:1072-1075 (Jun. 1, 2014)), radiology imaging, self-reporting, accelerometers (for example, in helmets). Following a diagnosis of mild brain injury, such as a concussion, ghrelin or a ghrelin variant can be administered to the patient, preferably in not more than 72 hours initially.

This invention also provides for methods for measuring ghrelin levels before starting a sport or activity, for example prior to the beginning of football season (or any other sport or activity, including those listed elsewhere herein), and monitoring ghrelin levels during the season to ascertain if the player or participant is at a level not qualified to play or participate. The methods can include the use of any suitable measurement or assay technique for measuring ghrelin levels, such as from blood to determine if blood levels correlate to brain levels.

With the benefit of the instant embodiments, the skilled artisan can select any suitable technique for measuring ghrelin levels. A number of assays known in the art for measuring a protein or hormone level are applicable for measuring ghrelin levels. By way of non-limiting example, assays such as a blood sugar test by extracting a drop of blood and putting it into a device, can quantitatively assess the amount of ghrelin, or an assay involving measuring a range of substances whereby a specific reaction chemistry is followed photo-metrically with time, for example by utilizing an antibody specific to ghrelin that is coated onto latex particles and measuring the increased turbidity that is produced when ghrelin being measured promotes aggregation of the latex particles as the reaction between ghrelin and anti-ghrelin antibody proceeds. This measurement of increasing turbidity can be achieved using a conventional photometer and using the associated scientific principles of photometric measurements. Such concentration dependent turbidity is then compared to that produced by standards which are established in the art.

Further compatible, but non-limiting, methodologies include carrying out a series of enzyme-linked reactions in solution, where ghrelin in the plasma fraction of a whole blood sample is altered by an enzyme-promoted reaction to ultimately derive a colored dye from colorless reaction constituents. The color is developed in a time dependent way and monitored photo-metrically. This measurement of color change can also be achieved using a conventional photometer using the associated scientific principles of photometric measurements. Such concentration dependent change in transmission is then compared to that produced by standards.

In whole blood samples, hematocrit or percentage of red blood cells by volume in the whole blood sample is a variable can be taken into account when analyzing ghrelin levels that are present in the plasma component. As the hematocrit of a patient's blood rises, so the volume of plasma in a fixed volume sample, which is introduced into the test device decreases and vice versa. Since it is the plasma component which exclusively carries the ghrelin levels being measured, then the lower the volume of plasma component added to the reaction mix, the lower the resulting concentration of the substance being measured in that reaction mix and the resulting assayed value and vice versa.

Any analysis that produces a concentration of a plasma substance in whole blood may be corrected for variations in hematocrit to give a true plasma concentration. It can be most useful in these situations to measure two substances, one of which is ghrelin under investigation and the other which is considered to be a marker by which to estimate or normalize the sample hematocrit. The hemoglobin concentration of whole blood, after red blood cells are lysed, is directly proportional to the red blood cell volume in the whole blood sample.

Pharmacodynarnic measurements of Protein Biomarkers and micro-RNA screening are performed. A blood sample is obtained to measure the levels of protein biomarkers (e.g., SBDP150, 5100, GFAP, and UCH-L1) that are derived from the cytosol of cells, such as but not limited to neurons, astrocytes, and axons. Micro-RNA (mi-RNA) levels are also measured. The blood samples are obtained on Days 0, 1, 2, 3, 4, 5, 6, 7, 14, 21, and 28 days.

The present technology provides biomarkers that are indicative of mild brain injury or concussion, neuronal damage, neural disorders, brain damage, neural damage, and diseases associated with the brain or nervous system, such as the central nervous system. In some embodiments, the biomarkers are proteins, fragments or derivatives thereof, and are associated with neuronal cells, brain cells or any cell that is present in the brain and central nervous system. In some embodiments, the biomarkers are neural proteins, peptides, fragments or derivatives thereof. Examples of neural proteins, include, but are not limited to axonal proteins, amyloid precursor protein, dendritic proteins, somal proteins, presynaptic proteins, post-synaptic proteins and neural nuclear proteins.

In some embodiments, the biomarker is one or more of, but not limited to, Axonal Proteins: α II spectrin (and SPDB)-1, NF-68 (NF-L)-2, Tau-3, α II, III spectrin, NF-200 (NF-H), NF-160 (NF-M), Amyloid precursor protein, α internexin; Dendritic Proteins: beta Ill-tubulin-1, p24 microtubule-associated protein-2, alpha-Tubulin (P02551), beta-Tubulin (P04691), MAP-2A/B-3, MAP-2C-3, Stathmin-4, Dynamin-1 (P21575), Phocein, Dynactin (013561), Vimentin (P31000), Dynamin, Profilin, Cofilin 1,2; Somal Proteins: UCH-L1 (Q00981)-1, Glycogen phosphorylase-BB-2, PEBP (P31044), NSE (P07323), CK-BB (P07335), Thy 1.1, Prion protein, Huntingtin, 14-3-3 proteins (e.g. 14-3-3-epsolon (P42655)), SM22-α, Calgranulin AB, alpha-Synuclein (P37377), beta-Synuclein (063754), HNP 22; Neural nuclear proteins: NeuN-1, S/G(2) nuclear autoantigen (SG2NA), Huntingtin; Presynaptic Proteins: Synaptophysin-1, Synaptotagmin (P21707), Synaptojanin-1 (062910), Synaptojanin-2, Synapsin1 (Synapsin-Ia), Synapsin2 (063537), Synapsin3, GAP43, Bassoon(NP_003449), Piccolo (aczonin) (NP_149015), Syntaxin, CRMP1, 2, Amphiphysin-1 (NP_001626) Amphiphysin-2 (NP_647477); Post-Synaptic Proteins: PSD95-1, NMDA-receptor (and all subtypes)-2, PSD93, AMPA-kainate receptor (all subtypes), mGluR (all subtypes), Calmodulin dependent protein kinase II (CAMPK)-alpha, beta, gamma, CaMPK-IV, SNAP-25, a-/b-SNAP; Myelin-Oligodendrocyte: Myelin basic protein (MBP) and fragments, Myelin proteolipid protein (PLP), Myelin Oligodendrocyte specific protein (MOSP), Myelin Oligodendrocyte glycoprotein (MOG), myelin associated protein (MAG), Oligodendrocyte NS-1 protein; Glial Protein Biomarkers: GFAP (P47819), Protein disulfide isomerase (PDI)-P04785, Neurocalcin delta, S100beta; Microglia protein Biomarkers: Iba1, OX-42, OX-8, OX-6, ED-1, PTPase (CD45), CD40, CD68, CD11b, Fractalkine. (CX3CL1) and Fractalkine receptor (CX3CR1), 5-d-4 antigen; Schwann cell markers: Schwann cell myelin protein; Glia Scar: Tenascin; Hippocampus: Stathmin, Hippocalcin, SCG10; Cerebellum: Purkinje cell protein-2 (Pcp2), Calbindin D9K, Calbindin D28K (NP_114190), Cerebellar CaBP, spot 35; Cerebrocortex: Cortexin-1 (P60606), H-2Z1 gene product; Thalamus: CD15 (3-fucosyl-N-acetyl-lactosarnine) epitope; Hypothalamus: Orexin receptors (OX-1R and OX-2R)-appetite, Orexins (hypothalamus-specific peptides); Corpus callosum: MBP, MOG, PLP, MAG; Spinal Cord: Schwann cell myelin protein; Striatum: Striatin, Rhes (Ras homolog enriched in striatum); Peripheral ganglia: Gadd45a; Peripheral nerve fiber (sensory+motor): Peripherin, Peripheral myelin protein 22 (AAH91499); Other Neuron-specific proteins: PH8 (S Serotonergic Dopaminergic PEP-19, Neurocalcin (NC), a neuron-specific EF-hand Ca²⁺-binding protein, Encephalopsin, Striatin, SG2NA, Zinedin, Recoverin, Visinin; Neurotransmitter Receptors: NMDA receptor subunits (e.g. NR1A2B), Glutamate receptor subunits (AMPA, Kainate receptors (e.g. GluR1, GluR4), beta-adrenoceptor subtypes (e.g. beta(2)), Alpha-adrenoceptors subtypes (e.g. alpha(2c)), GABA receptors (e.g. GABA(B)), Metabotropic glutamate receptor (e.g. mGluR3), 5-HT serotonin receptors (e.g. 5-HT(3)), Dopamine receptors (e.g. D4), Muscarinic Ach receptors (e.g. M1), Nicotinic Acetylcholine Receptor (e.g. alpha-7); Neurotransmitter Transporters: Norepinephrine Transporter (NET), Dopamine transporter (DAT), Serotonin transporter (SERT), Vesicular transporter proteins (VMAT1 and VMAT2), GABA transporter vesicular inhibitory amino acid transporter (VIAAT/VGAT), Glutamate Transporter (e.g. GLT1), Vesicular acetylcholine transporter, Vesicular Glutamate Transporter 1, [VGLUT1; BNPI] and VGLUT2, Choline transporter, (e.g. CHT1); Cholinergic Biomarkers: Acetylcholine Esterase, Choline acetyltransferase (ChAT); Dopaminergic Biomarkers: Tyrosine Hydroxylase (TH), Phospho-TH, DARPP32; Noradrenergic Biomarkers: Dopamine beta-hydroxylase (DbH); Adrenergic Biomarkers: Phenylethanolamine N-methyltransferase (PNMT); Serotonergic Biomarkers: Tryptophan Hydroxylase (TrH); Glutamatergic Biomarkers: Glutaminase, Glutamine synthetase; GABAergic Biomarkers: GABA transaminase (GABAT)), GABA-B-R2, or a combination thereof.

In some embodiments, the biomarkers comprise at least one biomarker from each neural cell type including, but not limited to, δ 11 spectrin, SPDB-1, NF-68, NF-L-2, Tau-3, βIII-tubulin-1, p24 microtubule-associated protein-2, UCH-L1 (000981)-1, Glycogen phosphorylase-BB-2, NeuN-1, Synaptophysin-1, synaptotagmin (P21707), Synaptojanin-1 (Q62910), Synaptojanin-2, PSD95-1, NMDA-receptor-2 and subtypes, myelin basic protein (MBP) and fragments, GFAP (P47819), Iba1, OX-42, OX-8, OX-6, ED-1, Schwann cell myelin protein, tenascin, stathmin, Purkinje cell protein-2 (Pcp2), Cortexin-1 (P60606), Orexin receptors (OX-1R, OX-2R), Striatin, Gadd45a, Peripherin, peripheral myelin protein 22 (AAH91499), and Neurocalcin (NC).

In some embodiments, the biomarkers are spectrin, βII-spectrin and βII-spectrin breakdown products (βII-SBDPs) generated by calpain-2 and/or caspase-3 proteolysis.

In some embodiments, at least one biomarker, such as a protein, peptide, variant or fragment thereof, is used to detect a neural injury, neuronal disorder or neurotoxicity in a subject, wherein said at least one biomarker is βII-spectrin, βII-SBDP-80, βII-SBDP-85, β-SBDP-108, or βII-SBDP-110.

In some embodiments, a plurality of biomarkers, such as proteins, peptides, variant or fragment thereof, is used to detect a neural injury, neuronal disorder or neurotoxicity in a subject, where said plurality biomarker is βII-spectrin, βII-SBDP-80, βII-SBDP-85, βII-SBDP-108, βII-SBDP-110 or combinations thereof. In some embodiments, the biomarks are as disclosed in US 2014/0024053, which disclosure is hereby incorporated by reference in its entirety.

In some embodiments, at least one biomarker or a plurality of biomarkers, such as a protein, peptide, variant or fragment thereof, is used to detect a neural injury, neuronal disorder or neurotoxicity in a subject wherein said at least one biomarker is microtubule-associated proteins (MAPs), MAP-2 (e.g., MAP-2A, MAP-2B, MAP-2C, MAP-2D), MAP breakdown products (MAP-BDP) or combinations thereof. In some embodiments, the biomarks are as disclosed in US 2014/0018299, which disclosure is hereby incorporated by reference in its entirety.

In some embodiments, an expanded panel of biomarkers is used to provide highly enriched information of mechanism of injury, modes of cell death (necrosis versus apoptosis), sites of injury, sites and status of different cell types in the nervous system and enhanced diagnosis (better selectivity and specificity). In some embodiments, the biomarkers are selected to distinguish between different host anatomical regions. For example, at least one biomarker can be selected from neural subcellular protein biomarkers, nervous system anatomical markers such as hippocampus protein biomarkers and cerebellum protein biomarkers. Examples of neural subcellular protein biomarkers are NF-200, NF-160, and NF-68. Examples of hippocampus protein biomarkers are SCG10 and stathmin. An example of a cerebellum protein biomarker is Purkinje cell protein-2 (Pcp2).

In some embodiments, the biomarkers are selected to distinguish between mild brain injury or concussion at the cellular level, thereby detecting which cell type has been injured. For example at least one biomarker protein is selected from a representative panel of protein biomarkers specific for that cell type. Examples for biomarkers specific for cell types include myelin-oligodendrocyte biomarkers such as myelin basic protein (MBP), myelin proteolipid protein (PLP), myelin oligodendrocyte specific protein (MOSP), oligodendrocyte NS-1 protein, myelin oligodendrocyte glycoprotein (MOG). Examples of biomarkers specific for Schwann cells include, but not limited to Schwann cell myelin protein. Examples of Glial cell protein biomarkers include, but not limited to GFAP (protein accession number P47819), protein disulfide isomerase (PDI)-P04785. Thus, by detecting one or more specific biomarkers the specific cell types that have been injured can be determined.

In some embodiments, biomarkers specific for different subcellular structures of a cell can be used to determine the subcellular level of injury. Examples include but not limited to neural subcellular protein biomarkers such as, NF-200, NF-160, NF-68; dendritic biomarkers such as for example, alpha-tubulin (P02551), beta-tubulin (P04691), MAP-2A/B, MAP-2C, Tau, Dynamin-1 (P212575), Phoecin Dynactin (Q13561), p24 microtubule-associated protein, vimentin (P131000); somal proteins such as for example, UCH-L1 (Q00981), S100, SBDP150, GFAP, PEBP (P31044), NSE (P07323), CK-BB (P07335), Thy 1.1, prion protein, 14-3-3 proteins; neural nuclear proteins, such as for example S/G(2) nuclear autoantigen (SG2NA), NeuN. Thus, detection of specific biomarkers will determine the extent and subcellular location of injury.

In some embodiments, biomarkers specific for different anatomical regions, different cell types, and/or different subcellular structures of a cell are selected to provide information as to the location of anatomical injury, the location of the injured cell type, and the location of injury at a subcellular level. A number of biomarkers from each set can be used to provide highly enriched and detailed information of mechanism, mode and subcellular sites of injury, anatomical locations of injury and status of different cell types in the nervous system (neuronal subtypes, neural stem cells, astro-glia, oligodendrocyte and microglia cell).

In some embodiments, subcellular neuronal biomarkers for diagnosis and detection of mild brain injury or concussion are at least one or more of axonal proteins, dendritic proteins, somal proteins, neural nuclear proteins, presynaptic proteins, post-synaptic proteins, or a combination thereof.

In some embodiments, axonal proteins identified as biomarkers for diagnosis and detection of mild brain injury or concussion include, but not limited to, α II spectrin (and SPDB)-1, NF-68 (NF-L)-2, Tau-3, α II, Ill spectrin, NF-200 (NE-H), NF-160 (NF-M), Amyloid precursor protein, α internexin, peptides, fragments or derivatives thereof.

In some embodiments, dendritic proteins identified as biomarkers for diagnosis and detection of mild brain injury or concussion include, but not limited to, beta III-tubulin-1, p24 microtubule-associated protein-2, alpha-Tubulin (P02551), beta-Tubulin (P04691), MAP-2A/B-3, MAP-2C-3, Stathmin-4, Dynamin-1 (P21575), Phocein, Dynactin (013561), Vimentin (P31000), Dynamin, Profilin, Cofilin 1,2, peptides, fragments or derivatives thereof. In some embodiments, neural nuclear proteins identified as biomarkers for diagnosis and detection of mild brain injury or concussion include, but not limited to, NeuN-1, S/G(2) nuclear autoantigen (SG2NA), Huntingtin, peptides or fragments thereof.

In some embodiments, somal proteins identified as biomarkers for diagnosis and detection of mild brain injury or concussion include, but not limited to, UCH-L1 (000981)-1, Glycogen phosphorylase-BB-2, PEBP (P31044), NSE (P07323), CK-BB (P07335), Thy 1.1, Prion protein, Huntingtin, 14-3-3 proteins (e.g. 14-3-3-epsolon (P42655)), SM22-α, Calgranulin AB, alpha-Synuclein (P37377), beta-Synuclein (063754), HNP 22, peptides, fragments or derivatives thereof.

In some embodiments, presynaptic proteins identified as biomarkers for diagnosis and detection of mild brain injury or concussion include, but not limited to, Synaptophysin-1, Synaptotagmin (P21707), Synaptojanin-1 (062910), Synaptojanin-2, Synapsin1 (Synapsin-Ia), Synapsin2 (063537), Synapsin3, GAP43, Bassoon(NP_003449), Piccolo (aczonin) (NP_149015), Syntaxin CRMP1, 2, Amphiphysin-1 (NP_001626), Amphiphysin-2 (NP_647477), peptides, fragments or derivatives thereof.

In some embodiments, post-synaptic proteins identified as biomarkers for diagnosis and detection of mild brain injury or concussion include, but not limited to, PSD95-1, NMDA-receptor (and all subtypes)-2, PSD93, AMPA-kainate receptor (all subtypes), mGluR (all subtypes), Calmodulin dependent protein kinase II (CAMPK)-alpha, beta, gamma, CaMPK-IV, SNAP-25, a-/b-SNAP, peptides, fragments or derivatives thereof.

In some embodiments, identified biomarkers distinguish the damaged neural cell subtype such as, for example, myelin-oligodendrocytes glial, microglial, Schwann cells, glial scar. In some embodiments, Myelin-Oligodendrocyte biomarkers are but not limited to: Myelin basic protein (MBP) and fragments, Myelin proteolipid protein (PLP), Myelin Oligodendrocyte specific protein (MOSP), Myelin Oligodendrocyte glycoprotein (MOG), myelin associated protein (MAG), Oligodendrocyte NS-1 protein; Glial Protein Biomarkers: GFAP (P47819), Protein disulfide isomerase (PDI)-P04785, Neurocalcin delta, S100beta; Microglia protein Biomarkers: Iba1, OX-42, OX-8, OX-6, ED-1, PTPase (CD45), CD40, CD68, CD11b, Fractalkine (CX3CL1) and Fractalkine receptor (CX3CR1), 5-d-4 antigen; Schwann cell markers: Schwann cell myelin protein; Glia Scar: Tenascin.

In some embodiments, biomarkers identifying the anatomical location of neural injury or damage include, but not limited to: Hippocampus: Stathmin, Hippocalcin, SCG10; Cerebellum: Purkinje cell protein-2 (Pcp2), Calbindin D9K, Calbindin D28K (NP_114190), Cerebellar CaBP, spot 35; Cerebrocortex: Cortexin-1 (p60606), H-2Z1 gene product; Thalamus: CD15 (3-fucosyl-N-acetyl-lactosamine) epitope; Hypothalamus: Orexin receptors (OX-1R and OX-2R)-appetite Orexins (hypothalamus-specific peptides); Corpus callosum: MBP, MOG, PLP, MAG; Spinal Cord: Schwann cell myelin protein; Striatum: Striatin, Rhes (Ras homolog enriched in striatum); Peripheral ganglia: Gadd45a; Peripheral nerve fiber(sensory+motor): Peripherin, Peripheral myelin protein 22 (AAH91499); PH8 (S Serotonergic Dopaminergic), PEP-19, Neurocalcin (NC), a neuron-specific EF-hand Ca²⁺-binding protein, Encephalopsin, Striatin, SG2NA, Zinedin, Recoverin, Visinin, or a combination thereof.

In some embodiments, biomarkers identifying damaged neural subtypes include, but not limited to: Neurotransmitter Receptors: NMDA receptor subunits (e.g. NR1A2B), Glutamate receptor subunits (AMPA, Kainate receptors (e.g. GluR1, GluR4), beta-adrenoceptor subtypes (e.g. beta(2)), Alpha-adrenoceptors subtypes (e.g. alpha(2c)), GABA receptors (e.g. GABA(B)), Metabotropic glutamate receptor (e.g. mGluR3), 5-HT serotonin receptors (e.g. 5-HT(3)), Dopamine receptors (e.g. D4), Muscarinic Ach receptors (e.g. M1), Nicotinic Acetylcholine Receptor (e.g. alpha-7); Neurotransmitter Transporters: Norepinephrine Transporter (NET), Dopamine transporter (DAT), Serotonin transporter (SERT), Vesicular transporter proteins (VMAT1 and VMAT2), GABA transporter vesicular inhibitory amino acid transporter (VIAAT/VGAT), Glutamate Transporter (e.g. GLT1), Vesicular acetylcholine transporter, Vesicular Glutamate Transporter 1, [VGLUT1; BNPI] and VGLUT2, Choline transporter, (e.g. CHT1); Cholinergic Biomarkers: Acetylcholine Esterase, Choline acetyltransferase [ChAT]; Dopaminergic Biomarkers: Tyrosine Hydroxylase (TH), Phospho-TH, DARPP32; Noradrenergic Biomarkers: Dopamine beta-hydroxylase (DbH); Adrenergic Biomarkers: Phenylethanolamine N-methyltransferase (PNMT); Serotonergic Biomarkers: Tryptophan Hydroxylase (TrH); Glutamatergic Biomarkers: Glutaminase, Glutamine synthetase; GABAergic Biomarkers: GABA transaminase (GABAT), GABA-B-R2, or a combination thereof.

Demyelination proteins identified as biomarkers for diagnosis and detection of mild brain injury or concussion are, but not limited to: myelin basic protein (MBP), myelin proteolipid protein, peptides, fragments or derivatives thereof. In some embodiments, glial proteins identified as biomarkers for diagnosis and detection of mild brain injury or concussion are, but not limited to: GFAP (P47819), protein disulfide isomerase (PDI-P04785), peptides, fragments and derivatives thereof.

In some embodiments, cholinergic proteins identified as biomarkers for diagnosis and detection of mild brain injury or concussion are, but not limited to: acetylcholine esterase, choline acetyltransferase, peptides, fragments or derivatives thereof. In some embodiments, dopaminergic proteins identified as biomarkers for diagnosis and detection of mild brain injury or concussion are, but not limited to: tyrosine hydroxylase (TH), phospho-TH, DARPP32, peptides, fragments or derivatives thereof.

In some embodiments, noradrenergic proteins identified as biomarkers for diagnosis and detection of mild brain injury or concussion are, but not limited to: dopamine beta-hydroxylase (DbH), peptides, fragments or derivatives thereof. In some embodiments, serotonergic proteins identified as biomarkers for diagnosis and detection of mild brain injury or concussion are, but not limited to: tryptophan hydroxylase (TrH), peptides, fragments or derivatives thereof. In some embodiments, glutamatergic proteins identified as biomarkers for diagnosis and detection of mild brain injury or concussion are, but not limited to: glutaminase, glutamine synthetase, peptides, fragments or derivatives thereof. In some embodiments, GABAergic proteins identified as biomarkers for diagnosis and detection of mild brain injury or concussion are, but not limited to: GABA transaminase (4-aminobutyrate-2-ketoglutarate transaminase (GABAT)), glutamic acid decarboxylase (GAD25, 44, 65, 67), peptides, fragments and derivatives thereof.

In some embodiments, neurotransmitter receptors identified as biomarkers for diagnosis and detection of mild brain injury or concussion are, but not limited to: beta-adrenoreceptor subtypes, (e.g. beta (2)), alpha-adrenoreceptor subtypes, (e.g. (alpha (2c)), GABA receptors (e.g. GABA(B)), metabotropic glutamate receptor. (e.g. mGluR3), NMDA receptor subunits (e.g. NR1A2B), Glutamate receptor subunits (e.g. GluR4), 5-HT serotonin receptors (e.g. 5-HT(3)), dopamine receptors (e.g. D4), muscarinic Ach receptors (e.g. M1), nicotinic acetylcholine receptor (e.g. alpha-7), peptides, fragments or derivatives thereof. In some embodiments, neurotransmitter transporters identified as biomarkers for diagnosis and detection of mild brain injury or concussion are, but not limited to: norepinephrine transporter (NET), dopamine transporter (DAT), serotonin transporter (SERT), vesicular transporter proteins (VMAT1 and VMAT2), GABA transporter vesicular inhibitory amino acid transporter (VIAAT/VGAT), glutamate transporter (e.g. GLT1), vesicular acetylcholine transporter, choline transporter (e.g. CHT1), peptides, fragments, or derivatives thereof. In some embodiments, other proteins identified as biomarkers for diagnosis and detection of mild brain injury or concussion are, but not limited to, vimentin (P31000), CK-BB (P07335), 14-3-3-epsilon (P42655), MMP2, MMP9, peptides, fragments or derivatives thereof.

The markers are characterized by molecular weight, enzyme digested fingerprints and by their known protein identities. The markers can be resolved from other proteins in a sample by using a variety of fractionation techniques, e.g., chromatographic separation coupled with mass spectrometry, or by traditional immunoassays. In some embodiments, the method of resolution involves Surface-Enhanced Laser Desorption/Ionization (“SELDI”) mass spectrometry, in which the surface of the mass spectrometry probe comprises adsorbents that bind the markers. In some embodiments, a plurality of the biomarkers are detected, at least two, or three, or four of the biomarkers are detected.

In some embodiments, the amount of each biomarker is measured in the subject sample and the ratio of the amounts between the markers is determined. The amount of each biomarker in the subject sample and the ratio of the amounts between the biomarkers and compared to normal healthy individuals. The increase in ratio of amounts of biomarkers between healthy individuals and individuals suffering from injury is indicative of the injury magnitude, disorder progression as compared to clinically relevant data.

In some embodiments, biomarkers that are detected at different stages of injury and clinical disease are correlated to assess anatomical injury, type of cellular injury, subcellular localization of injury. Monitoring of which biomarkers are detected at which stage, degree of injury in disease or physical injury will provide panels of biomarkers that provide specific information on mechanisms of injury, identify multiple subcellular sites of injury, identify multiple cell types involved in disease related injury and identify the anatomical location of injury. In some embodiments, one or more of the biomarkers disclosed herein, can be specifically excluded.

In some embodiments, a single biomarker is used in combination with one or more biomarkers from normal, healthy individuals for diagnosing injury, location of injury and progression of disease and/or neural injury, or a plurality of the markers are used in combination with one or more biomarkers from normal, healthy individuals for diagnosing injury location of injury and progression of disease and/or neural injury. In some embodiments, one or more protein biomarkers are used in comparing protein profiles from patients susceptible to, or suffering from disease and/or neural injury, with normal subjects.

In some embodiments, detection methods include use of a biochip array. Biochip arrays useful include protein and nucleic acid arrays. One or more markers are immobilized on the biochip array and subjected to laser ionization to detect the molecular weight of the markers. Analysis of the markers is, for example, by molecular weight of the one or more markers against a threshold intensity that is normalized against total ion current. In some embodiments, logarithmic transformation is used for reducing peak intensity ranges to limit the number of markers detected. In some embodiments, data is generated on immobilized subject samples on a biochip array, by subjecting said biochip array to laser ionization and detecting intensity of signal for mass/charge ratio; and transforming the data into computer readable form; and executing an algorithm that classifies the data according to user input parameters for detecting signals that represent markers present in injured and/or diseased patients and are lacking in non-injured and/or diseased subject controls.

A follow up or recovery protocol, optionally can then be used to assess the recovery of the patient, including whether the patient can return to participate in certain activities such as a sport. Once such recovery protocol used for sports is the Zurich graduated return to play protocol, which is the five stage or phase return process commonly referenced in cases of NFL players rehabilitating from a recent concussion. It is based on a guideline recommended by the 2012 Zurich Consensus Statement on Concussion in Sport. The guideline was written by a group of authors that included Dr. Robert Cantu, a senior adviser to the NFL's Head, Neck and Spine committee, and Dr. Margot Putakian, who also serves on the committee and was involved in the development of the sideline assessment protocols. (http://subscribers.footballguys.com/apps/article.php?article=13bramel_inside_concussions_2) Thus, the ghrelin variant can be administered to a patient with a mild brain injury or concussion that needs a recovery protocol. After administration of at least an initial dose, a recovery protocol can be administered and followed to determine when the patient can undertake or resume certain activity.

### Pharmaceutical Compositions

Ghrelin, ghrelin variants and the combinations described herein can be formulated as a pharmaceutical composition, e.g., flash frozen or lyophilized for storage and/or transport. In some embodiments, the compound can be in a composition with sterile saline, for example. In some embodiments, a ghrelin, ghrelin variant, or combination material can be reconstituted in such saline or other acceptable diluent. In some embodiments, about 10 μg ghrelin powder is reconstituted in about 100 μL saline solution before administration. In addition, the composition can be administered alone or in combination with a carrier, such as a pharmaceutically acceptable carrier or a biocompatible scaffold. Compositions of the invention may be conventionally administered parenterally, by injection, for example, intravenously, subcutaneously, or intramuscularly. Additional formulations which are suitable for other modes of administration include oral formulations. Oral formulations include such normally employed excipients such as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, about 25% to about 70%.

Typically, compositions are administered in a manner compatible with the dosage formulation, and in such amount as will be therapeutically effective for the disease or condition by treated. The quantity to be administered depends on the subject to be treated. Precise amounts of the composition to be administered depend on the judgment of the practitioner. Suitable regimes for initial administration and boosters are also variable, but are typified by an initial administration followed by subsequent administrations.

In some embodiments, additional pharmaceutical compositions are administered to a subject to support or augment the compositions as described herein. Different aspects of the present invention involve administering an effective amount of the composition to a subject. Additionally, such compositions can be administered in combination with other agents. Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

Suitable pharmaceutical carriers include inert solid diluents or fillers, sterile aqueous solution and various organic solvents. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Nasal aerosol or inhalation formulations may be prepared, for example, as solutions in saline, employing benzyl alcohol or other suitable preservatives, absorption promoters to enhance bioavailability, employing fluorocarbons, and/or employing other solubilizing or dispersing agents.

The carrier may be a solvent or dispersion medium containing, for example, water (e.g., hydrogels), ethanol, polyol (for example, glycerol, propylene glycol, and liquid poly(ethylene glycol), and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of undesirable microorganisms can be brought about by various antibacterial and antifungal agents, for example, parabens, chlorobutanol, phenol, sorbic acid, thimerosal, and the like. In many cases, isotonic agents are included, for example, sugars or sodium chloride. Prolonged absorption of the injectable compositions can be brought about by the use in the compositions of agents delaying absorption, for example, aluminum monostearate and gelatin.

An effective amount of therapeutic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

A ghrelin variant can be administered subcutaneously in an amount allowing sufficient levels of the bioactive form of ghrelin variant, i.e., the acylated form, to reach the receptors.

The present disclosure also provides a procedure for an optimal administration of ghrelin variants to patients in order to obtain a maximal response and to avoid, for example, desensitization mechanisms.

The ghrelin receptor normally is exposed to short-lived surges in ghrelin concentration. The GHS-R 1a receptor (growth hormone secretagogue receptor 1a) belongs to the class of G protein coupled receptors or 7TM receptors, which upon continued exposure to an agonist will be desensitized, internalized, and down-regulated. These mechanisms, which are inherent to the overall signal transduction system, involve processes such as receptor phosphorylation (which, in itself, decreases the affinity of the receptor for the agonist) and binding of inhibitory proteins such as arrestin (which sterically block the binding of signal transduction molecules such as G proteins).

Another part of the agonist-mediated desensitization process is receptor internalization (physical removal of the receptor from the cell surface where it could bind the agonist) as well as receptor down regulation (decreased production/expression of the receptor). Receptor internalization could, after short-lived exposure of the receptor to agonist, be followed by a re-sensitization process, where the receptor is dephosphorylated and recycled to the cell surface to be used again. Without being bound by theory, upon prolonged stimulation which would occur for example during a long-lasting continuous infusion of the agonist, the receptor down-regulation process ensures that the target cell is adjusted in its signal transduction system to this situation.

Ghrelin variant compositions can be produced using techniques well known in the art. For example, a polypeptide region of a ghrelin variant can be chemically or biochemical synthesized and modified. Techniques for chemical synthesis of polypeptides are well known in the art (Lee V. H. L. in “Peptide and Protein Drug Delivery”, New York, N.Y., M. Dekker, 1990). Examples of techniques for biochemical synthesis involving the introduction of a nucleic acid into a cell and expression of nucleic acids are provided in Ausubel F. M. et al., “Current Protocols in Molecular Biology”, John Wiley, 1987-1998, and Sambrook J. et al., “Molecular Cloning, A Laboratory Manual”, 2d Edition, Cold Spring Harbor Laboratory Press, 1989, each of which is incorporated herein by reference. Another exemplary technique, described in U.S. Pat. No. 5,304,489, incorporated herein by reference, is the use of a transgenic mammal having mammary gland-targeted mutations which result in the production and secretion of synthesized ghrelin variant in the milk of the transgenic mammal.

The ghrelin variants can also be produced recombinantly using routine expression methods known in the art. The polynucleotide encoding the desired ghrelin variant is operably linked to a promoter into an expression vector suitable for any convenient host. Both eukaryotic and prokaryotic host systems are used in forming recombinant ghrelin variants. The ghrelin variant is then isolated from lysed cells or from the culture medium and purified to the extent needed for its intended use. Isolated ghrelin or ghrelin variant may be modified further at serine amino acid position 2 and/or serine amino acid position 3 by fatty acid acylation in vivo or in vitro, with the latter in vitro acylation reaction condition comprising fatty acid thioester, ghrelin, and microsomes comprising ghrelin O-acyl transferase (GOAT). In some embodiments, acyl ghrelin or ghrelin variant modified with fatty acid at serine amino acid position 2 and/or serine amino acid position 3 is isolated from cellular or reaction components.

Ghrelin compositions can include pharmaceutically acceptable salts of the compounds therein. These salts will be ones which are acceptable in their application to a pharmaceutical use, meaning that the salt will retain the biological activity of the parent compound and the salt will not have untoward or deleterious effects in its application and use in treating diseases. Pharmaceutically acceptable salts are prepared in a standard manner.

In some embodiments, a DNA coding an amino acid sequence of ghrelin variants described in the present disclosure, which comprises a nucleotide sequence coding a peptide containing an amino acid sequence recognizing at least one modifiable amino acid in the amino acid sequence encoded by said DNA. In some embodiments, a vector comprises a DNA described above. In some embodiments, cells comprise the vector described above.

In some embodiments, a method for producing a ghrelin variant compound by genetic recombination technology comprises transforming a vector containing a DNA described above into host cells capable of modifying a side chain of at least one amino acid in said peptide, then culturing the resulting transformed cells and recovering the desired ghrelin variant compound from the culture. In some embodiments, a method for producing a ghrelin variant compound by genetic recombination technology comprises using cells having the activity of binding a fatty acid via an ester linkage to a side-chain hydroxyl group of an amino acid or via a thioester linkage to a side-chain mercapto group of an amino acid in the ghrelin variant compound.

In some embodiments, a method for producing a ghrelin variant compound by genetic recombination technology comprises using cells having the serine acylation activity of binding a fatty acid via an ester linkage to a side-chain hydroxyl group of serine. In some embodiments, a method for producing a ghrelin variant compound by genetic recombination technology comprises using cells having the threonine acylation activity of binding a fatty acid via an ester linkage to a side-chain hydroxyl group of threonine.

In some embodiments, the present disclosure provides for a method of producing a ghrelin variant, said method comprising the steps of: (a) providing a cDNA comprising a polynucleotide sequence encoding a ghrelin variant; (b) inserting said cDNA in an expression vector such that the cDNA is operably linked to a promoter; and (c) introducing said expression vector into a host cell whereby said host cell produces said ghrelin variant.

In some embodiments, the method further comprises the step of recovering the ghrelin variant produced in step (c). The expression vector is any of the mammalian, yeast, insect, or bacterial expression systems known in the art. Commercially available vectors and expression systems are available from a variety of suppliers including Genetics Institute (Cambridge, Mass.), Stratagene (La Jolla, Calif.), Promega (Madison, Wis.), and Invitrogen (San Diego, Calif.). If desired, to enhance expression and facilitate proper protein folding, the codon context and codon pairing of the sequence is optimized for the particular expression organism in which the expression vector is introduced, as explained in U.S. Pat. No. 5,082,767, which disclosure is hereby incorporated by reference in its entirety.

In some embodiments, additional nucleotide sequencers, which codes for secretory or leader sequences, pro-sequences, sequences which aid in purification, such as multiple histidine residues, or an additional sequence for stability during recombinant production, are added to ghrelin variants or to ghrelin itself to produce a ghrelin variant.

In some embodiments, introduction of a polynucleotide encoding a ghrelin variant into a host cell can be affected by calcium phosphate transfection, DEAE-dextran mediated transfection, cationic lipid-mediated transfection, electroporation, transduction, infection, or other methods. Such methods are described in many standard laboratory manuals, such as Davis et al., (1986) Basic Methods in Molecular Biology, ed., Elsevier Press, NY, which disclosure is hereby incorporated by reference in its entirety.

ghrelin variants can be recovered and purified from recombinant cell cultures by well-known methods including differential extraction, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography (“Methods in Enzymology: Aqueous Two-Phase Systems”, Walter H et al. (eds.), Academic Press (1993), incorporated herein by reference, for a variety of methods for purifying proteins). In some embodiments, high performance liquid chromatography (“HPLC”) is employed for purification.

A recombinantly produced version of ghrelin variants can be substantially purified using techniques described herein or otherwise known in the art, such as, for example, by the one-step method described in Smith & Johnson, Gene 67:31 40 (1988), which disclosure is hereby incorporated by reference in its entirety. Ghrelin variants also can be purified from recombinant sources using antibodies directed against ghrelin variants, which are well known in the art of protein purification.

In some embodiments, depending upon the host employed in a recombinant production procedure, the ghrelin variants may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

The present disclosure provides for a pharmaceutical composition comprising a mixture of at least two different ghrelin variants, such as a mixture of a ghrelin variant acylated with a C₈ acyl and a ghrelin variant acylated with a C₁₀ acyl. Without being bound by theory, it is believed that such a mixture will have a longer half-life in plasma. In some embodiments, the pharmaceutical composition comprises acylated ghrelin variants, optionally compounds having different acyl chain lengths selected from the group consisting of C7 acyl group, C9 acyl group, and C_{H} acyl group, optionally in combination with a non- or un-acylated ghrelin variant.

In some embodiments, the pharmaceutical composition comprising any secretagogue, such as any ghrelin variant or a pharmaceutically acceptable salt thereof and pharmaceutical acceptable carriers, vehicles and/or excipients; said composition further comprising transport molecules. The transport molecules are primarily added in order to increase the half-life of the acylated compound, preventing premature des-acylation, since the des-acylated ghrelin variant might not be active at the GHS-R 1a.

Transport molecules act by having incorporated into or anchored to it a compound disclosed herein. Any suitable transport molecule known to the skilled person may be used. Examples of transport molecules are those described in the conjugate section, supra. Other examples are liposomes, micelles, and/or microspheres.

In some embodiments, the active ingredient can be mixed with excipients and non-endogenous carriers, which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

In some embodiments, the formulation has a pH within the range of 3.5-8, such as in the range 4.5-7.5, such as in the range 5.5-7, such as in the range 6-7.5, such as about 7.3. However, as is understood by one skilled in the art, the pH range may be adjusted according to the individual treated and the administration procedure. For example, certain ghrelin variants or ghrelin homologs, may be stabilized at a lower pH; thus in some embodiments, the formulation has a pH within the range 3.5-7, such as 4-6, such as 5-6, such as 5.3-5.7, such as about 5.5.

Ghrelin variant compositions can include pharmaceutically acceptable salts of the compounds therein. These salts will be ones which are acceptable in their application to a pharmaceutical use, meaning that the salt will retain the biological activity of the parent compound and the salt will not have untoward or deleterious effects in its application and use in treating diseases. Pharmaceutically acceptable salts are prepared in a standard manner. If the parent compound is a base, it is treated with an excess of an organic or inorganic acid in a suitable solvent. If the parent compound is an acid, it is treated with an inorganic or organic base in a suitable solvent.

Ghrelin variant compositions may be administered in the form of an alkali metal or earth alkali metal salt thereof, concurrently, simultaneously, or together with a pharmaceutically acceptable carrier or diluent, especially and in the form of a pharmaceutical composition thereof, whether by various routes (e.g., oral, rectal, parenteral, subcutaneous) in an effective amount.

Other suitable pharmaceutically acceptable salts include the acid addition salts (formed with the free amino groups of the polypeptide). Other examples of salts include pharmaceutically acceptable acid addition salts, pharmaceutically acceptable metal salts, ammonium salts and alkylated ammonium salts. Acid addition salts include salts of inorganic acids as well as organic acids.

In some embodiments, compounds or pharmaceutical acceptable acid addition salts are any hydrates (hydrated forms) thereof. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

For parenteral administration, solutions of the present compounds in sterile aqueous solution, aqueous propylene glycol or sesame or peanut oil may be employed. Such aqueous solutions should be suitably buffered if necessary, and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Nasal aerosol or inhalation formulations may be prepared, for example, as solutions in saline, employing benzyl alcohol or other suitable preservatives, absorption promoters to enhance bioavailability, employing fluorocarbons, and/or employing other solubilizing or dispersing agents.

The present disclosure provides a pharmaceutical composition stably containing ghrelin or ghrelin variants and a method for preventing degradation of modifying hydrophobic group of ghrelin or ghrelin variants in an aqueous solution. The modifying hydrophobic group of the ghrelin or ghrelin variants, is not limited to octanoyl (C₈) group, and is a residue of fatty acid having 2 to 20, preferably 4 to 12 carbon atoms, such as hexanoyl (C₅) group, decanoyl (C₁₀) group or dodecanoyl (C₁₂) group. The hydrophobic group can also be a residue of branched, saturated or unsaturated fatty acid, a residue of fatty acid having an aromatic group such as phenylpropionyl group, and an adamantane skeleton.

In some embodiments, the ghrelin variants of the present disclosure include the ghrelin or ghrelin variant peptides, in which the amino acid sequence is modified by the insertion addition and deletion of one or more amino acid, and/or the substitution by other amino acid to said amino acid sequence, and is modified chemically if necessary. In some embodiments, the ghrelin variants include the peptides in which modifying hydrophobic group is bonded to amino acid chain by ester bond and having same or similar physiologically activity and function as ghrelin.

In some embodiments, the ghrelin or ghrelin variant is to be used in the pharmaceutical composition of the present disclosure includes free form peptides and salts thereof. The free form peptide and salt thereof can be reciprocally converted. The free form peptide can be converted to a pharmaceutically acceptable salt by reacting with an inorganic or an organic acid. The examples of the inorganic acid include, but are not limited to, carbonate, bicarbonate, hydrochloride, sulfate, nitrate, borate or a combination thereof; and the examples of the organic acid include, but are not limited to, succinate, acetate, propionate, trifluoroacetate, or a combination thereof. Other examples of the salt include, but are not limited to, the salt with alkali metal such as sodium salt or potassium salt; the salt with alkali earth metal such as calcium salt or magnesium salt; the salt with organic amine such as triethylamine salt; and the salt with basic amino acid such alginic acid salt, or a combination thereof. The ghrelin or ghrelin variant peptides of the present disclosure can exist as metal complex such as copper complex or zinc complex. The form of the salt as mentioned above has a role is the stability of the ghrelin or ghrelin variants. That is, pH values of the aqueous solution of the salts above are different from each other, and therefore, these salts play the role as pH adjuster for the aqueous solution of the ghrelin or ghrelin variants.

In some embodiments, The ghrelin or ghrelin variants to be used as raw materials for medicines are commonly supplied as lyophilized powder after purified by reverse liquid chromatography and so on. The aqueous solution is the solution used water as the solvent; however, other solvent such as ethanol, 2-propanol and the like can be used within a pharmaceutically acceptable range.

The concentration of the ghrelin or ghrelin variants in the pharmaceutical composition is not limited to, and is preferably within a pharmaceutically acceptable range. The lower limit of concentration is the concentration wherein the ghrelin or ghrelin variants exhibit the pharmacologically activities, and the upper limit of concentration is the concentration wherein the ghrelin or ghrelin variants can be dissolve in the aqueous solutions. In some embodiments, the concentration ghrelin or ghrelin variants used in the pharmaceutical composition is about 0.01 nmol/mL to about 10 μmol/mL, or about 0.03 nmol/mL to about 3 μmol/mL.

In some embodiments, in the physiological composition of the present disclosure, containing ghrelin or ghrelin variants, the pH value of the solution is in the range of 2 to 7, more preferably 3 to 6. In some embodiments, the pH value of the solution containing the ghrelin or ghrelin variants that are stable is in the range of 2 to 7. The adjustment of pH of the solution containing the ghrelin or ghrelin variants is conducted with pH adjuster or buffer agent.

Examples of pH adjuster include, but not limited to, hydrochloric acid sulfuric acid, nitric acid, boric acid, carbonic acid, bicarbonic acid, gluconic acid, sodium hydroxide, potassium hydroxide, aqueous ammonia, citric acid, monoethanolamine, lactic acid, acetic acid, succinic acid, fumaric acid, maleic acid, phosphoric acid, methanesulfonic acid, malic acid, propionic acid, trifluoroacetic acid, and salt thereof.

Examples of buffer agent include, but not limited to, glycine, acetic acid, citric acid, boric acid, phthalic acid, phosphoric acid, succinic acid, lactic acid, tartaric acid, carbonic acid, hydrochloric acid, sodium hydroxide, and the salt thereof. In some embodiments, glycine, acetic acid or succinic acid are used as buffer agent.

Considering the stability of the ghrelin or ghrelin variants in the aqueous solution, it is desired that the fluctuation of pH values of the solution have to be reduced. Therefore, the pharmaceutical composition of the present disclosure is the solution having buffer capacity, that is, the buffer solution.

In some embodiments, the buffer solution, having the pH range wherein the degradation of the ghrelin or ghrelin variants is inhibited, and the solution having the pH range of 2 to 7, more preferably 3 to 6 is used. The suitable buffer solution include, but not limited to, glycine hydrochloride buffer, acetate buffer, citrate buffer, lactate buffer, phosphate buffer, citric acid-phosphate buffer (including Mcllvaine buffer), phosphate-acetate-borate buffer (including Britton-Robinson buffer), and phthalate buffer. The examples of the components of each buffers include the buffer agents mentioned above.

In some embodiments, the concentration of pH adjuster is not limited and can be the concentration commonly used to adjust the solution with the desired pH range, and in general, the concentration of 0.01 to 100 mM is used. In some embodiments, the concentration of buffer agent is also not limited and can be the concentration maintaining the buffer capacity. Generally, the concentration is about 0.01 to about 100 mM, or about 0.1 to about 100 mM, or about 1 to about 100 mM.

In some embodiments, the pharmaceutical composition stably containing the ghrelin or ghrelin variants in the aqueous solution is provided. The composition contains other additives in consideration of osmolality, solubility, low irritation of the solution, as well as antisepsis effect and prevention of absorption of the ingredient in the solution.

In some embodiments, anti-adsorbents are used to prevent ghrelin or ghrelin variants peptide from absorbing to glass vessels or polypropylene vessels. Examples of anti-adsorbent include, but not limited to, surfactants, saccharides, amino acids and proteins.

Examples of the surfactant include, but not limited to, quaternary ammonium salts, polyoxyethylene sorbitan fatty acid esters, sorbitan fatty acid esters, parabens, polyethylene glycols phospholipids, bile acids polyoxyethylene castor oils, polyoxyethylenes, polyoxyethylene polyoxypropylenes, polyalcohols, anionic surfactant, synthetic or semi-synthetic polymers.

The suitable quaternary ammonium salts include, but not limited to, benzalkonium chloride, benzethonium chloride and cetylpyridinium chloride.

The suitable polyoxyethylene sorbitan fatty acid esters include, but not limited to, polyoxyethylene sorbitan monolaurate (Polysorbate® 20 or Tween® 20), polyoxyethylene sorbitan monopalmitate (Polysorbate® 40 or Tween® 40), polyoxyethylene sorbitan monostearate (Polysorbate® 60 or Tween® 60), polyoxyethylene sorbitan tristearate (Polysorbate® 65 or Tween® 65), polyoxyethylene sorbitan monooleate (Polysorbate® 80 or Tween® 80), and polyoxyethylene sorbitan trioleate (Polysorbate® 85 or Tween® 85).

The suitable sorbitan fatty acid esters include, but not limited to, sorbitan monolaurate (Span®20), sorbitan monopalmitate (Span®40), sorbitan monostearate (Span® 60), sorbitan monooleate (Span® 80), sorbitan trioleate (Span® 85), and sorbitan sesquioleate.

The suitable parabens include, but not limited to, methyl paraoxybenzoate, ethyl paraoxybenzoate, propyl paraoxybenzoate, butyl paraoxybenzoate, and isobutyl paraoxybenzoate.

The suitable polyethylene glycols include, but not limited to, glycofuro (glycofurol 75), Mcrogol® 400 (polyethylene glycol 400), Mcrogol® 600 (polyethylene glycol 600), and Mcrogol® 9000 (polyethylene glycol 4000); the suitable phospholipids include refined soybean lecithin and refined yolk lecithin; and suitable bile acids include sodium desoxycholic acid.

The suitable polyoxyethylene castor oils include but not limited to, polyoxyethylene castor oil, polyoxyethylene hydrogenated castor oil, polyoxyethylene hydrogenated castor oil 50, and polyoxyethylene hydrogenated castor oil 60. Examples of other polyoxyethylenes include polyoxyethylene oleyl ether, polyoxyethylene stearyl ether, polyoxyethylene cetyl ether, and polyoxyethylene lauryl sulfate salt.

The suitable polyoxyethylene polyoxypropylenes include, but not limited to, polyoxyethylene polyoxypropylene glycol (Pluronic®) and polyoxyethylene polyoxypropylene cetyl ether.

The suitable polyalcohols include, but not limited to, glycerin (glycerol), propylene glycol, and monoglyceryl stearate; and the suitable anionic surfactants include, but not limited to, alkyl ether sulfate such as sodium cetyl sulfate, sodium lauryl sulfate and sodium oleyl sulfate; alkyl sulfosuccinate such as sodium lauryl sulfosuccinate. The suitable synthetic or semi-synthetic polymers include, but not limited to, polyvinyl alcohol, carboxyvinyl polymer, polyvinyl pyrrolidone and sodium polyacrylate.

Examples of saccharides include, but not limited to, monosaccharide such as mannitol glucose, fructose, inositol; sorbitol, and xylitol; disaccharide such as lactose, sucrose maltose, and trehalose; polysaccharide such as starch, dextran, pullulan, alginic acid, hyaluronic acid, pectinic acid, phytic acid, phytin, chitin, and chitosan. Examples of dextrin include, but not limited to, α-cyclodextrin, β-cyclodextrin, γ-cyclodextrin, dextrin, hydroxypropyl starch, and hydroxyl starch. Examples of celluloses include, but not limited to, methylcellulose, ethylcellulose, hydroxyethyl cellulose, hydroxypropyl cellulose, and hydroxypropyl methylcellulose, sodium carboxymethyl cellulose.

The suitable amino acids include, but not limited to, glycine and taurine; and polyamino acid such as polyglutamic acid, polyaspartic acid, polyglycine and polyleucine. The Examples of proteins include but not limited to, albumin and gelatin.

Non-human serum albumin can be used as anti-adsorbent for the pharmaceutical composition of the present invention when the composition is used as a reagent for examination or as veterinary medicines; however, it is preferable to use human serum albumin when the composition is used for a medicine for treating human being. These anti-adsorbents can be used in combination. The concentration of the anti-adsorbent is in the range wherein the amount of the anti-adsorbent is pharmaceutically acceptable one and the adsorption of the ghrelin or ghrelin variants to the vessel is inhibited and the aggregation of the components does not occur during the manufacturing process or the long-term storage. For example, the concentration of the anti-adsorbent is in the range of about 0.001 to about 5%, or from about 0.01 to about 1%.

The pharmaceutical composition of the present disclosure can contain further additives for any purpose, and examples of the additives is selected from the “Handbook of PHARMACEUTICAL. EXCIPIENTS 2000” (Japan Pharmaceutical Excipients Council: Yakuji Nippoh Sha). These include isotonizing agent such as, but not limited to, sodium chloride and mannitol; antiseptic agent such as, but not limited to, sodium benzoate; antioxidant such as, but not limited to, sodium bisulfite, sodium pyrosulfite and ascorbic acid; soothing agent such as, but not limited to, lidocaine hydrochloride and mepivacaine hydrochloride, as explained in U.S. Pat. No. 8,518,893, which disclosure is hereby incorporated by reference in its entirety.

The manufacture of the pharmaceutical composition of the present disclosure is conducted by mean of the common procedure applied in the pharmaceutical field. For example, first, freeze dried ghrelin is dissolved in the purified water, and then, buffer agent, anti-adsorbent and other additives are also dissolved in another purified water. Then the resulting water solutions are combined and sterilize by filtration if necessary, and the obtained solution is filled in ampoules or vials to obtain the pharmaceutical composition containing the ghrelin or ghrelin variants of the present disclosure.

### Administration of Compositions

The present disclosure provides for a method of reducing the incidence or severity of mBI or concussion and/or associated symptoms in a subject, comprising administering to the subject an effective amount of a compound comprising a ghrelin variant, thereby reducing the incidence or severity of the mBI or concussion and/or associated symptoms. The present disclosure also provides for methods of reducing the incidence or severity of mBI or concussion in a subject, comprising administering to the subject an effective amount of ghrelin, thereby reducing the incidence or severity of the mBI or concussion. This invention further provides for methods of reducing the amount of time needed to recover from a mild brain injury or concussion, comprising administering to a patient suffering from a mild brain injury or concussion a therapeutically effective amount of ghrelin within a certain period (e.g., 72 hours) of the mild brain injury or concussion.

In some embodiments, the ghrelin variant, is administered prior to an event or activity with a potential for occurrence of mBI or concussion. In some embodiments, the event or activity is participation in a sporting event, physical training, or combat. In some embodiments, the event or activity is baseball, basketball, rugby, football, hockey, lacrosse, soccer, cycling, boxing, gymnastics, a martial art, a mixed martial art, a military exercise, automobile racing, snow skiing, snowboarding, ice skating, skateboarding, motorcross, mountain biking, motorcycle and ATV riding, and the like. In some embodiments, the subject has not suffered a mBI or concussion. In some embodiments, the subject has a history of mBI or concussion or is susceptible to mBI or concussion.

In some embodiments, an administration route for a ghrelin variant is selected from: buccal delivery, sublingual delivery, transdermal delivery, inhalation and needle-free injection, such as using the methods developed by PowderJet. For inhalation, the a ghrelin variant can be formulated using methods known to those skilled in the art, for example an aerosol, dry powder or solubilized such as in microdroplets, in a device intended for such delivery (such as commercially available devices and formulation technologies from Aradigm Corp. (Hayward, Calif.), Alkermes, Inc. (Cambridge, Mass.), Nektar Therapeutics (San Carlos, Calif.), or MannKind Corporation (Valencia, Calif.; e.g., Technosphere®, Dreamboat®, and Cricket™ technologies)).

In some embodiments, the DANA mobile medical application (AnthroTronix, www.atinc.com) is utilized to determine the therapeutic effectiveness of ghrelin variant compositions in treating mild brain injury or concussion. DANA provides clinicians with objective measurements of reaction time (speed and accuracy) to aid in the assessment of an individual's medical or psychological state. DANA is a phone or tablet-based app on Android or iOS operating systems and is indicated for use as part of any clinical assessment where concerns for changes in cognitive or psychological status are present. DANA's battery of cognitive and psychological tests are administered and the results are evaluated by a qualified health professional who can assess factors that may affect measurement of reaction time such as concussion, dementia, post-traumatic stress, depression, stress, fatigue, prescription and non-prescription medications, and some nutritional supplements, among others.

In some embodiments, the ghrelin variant is administered via a powder or stable formulation, wherein the ghrelin variant is formulated in a dosage form selected from the group consisting of: liquid, beverage, medicated sports drink, powder, capsule, chewable tablet, caplet, swallowable tablet, buccal tablet, troche, lozenge, soft chew, solution, suspension, spray, suppository, tincture, decoction, infusion, and a combination thereof.

In some embodiments, the composition comprising ghrelin or the ghrelin variant is administered via inhalation, oral, intravenous, parenteral, buccal, subcutaneous (including “EpiPens”), transdermal, patch, sublingual, intramuscular, or intranasal. In some embodiments, EpiPens is either EpiPen 0.3 mg or EpiPen Jr® (epinephrine) 0.15 mg Auto-Injectors for people who have a history of life-threatening allergic reactions (anaphylaxis) to things like bee stings, peanuts or seafood, or are at increased risk for a severe allergic reaction. EpiPen and EpiPen Jr are self-injectable devices (auto-injectors) that contain epinephrine.

In some embodiments, the composition comprising ghrelin or the ghrelin variant is administered via the automatic mixing device and delivery system by Windgap Medical Devices as described in U.S. Patent Application No. 2013/0178823, which is incorporated by reference in its entirety. In some embodiments, the automatic mixing device and delivery system by Windgap Medical Devices is a wet/dry auto-mixing injector having a mixing device containing at least one microfluidic channel for mixing or dissolving a dry component with a wet component stored in the injector device. In some embodiments, the automatic mixing device and delivery system by Windgap Medical Devices is a mixing and/or automatic injection device having an interior chamber containing a wet component that may be pH optimized to be mixed with a dry component contained in a mixing assembly. The wet component being confined or sealed in the interior chamber by a seal or valve, where upon activation of the seal or valve the wet interior chamber becomes in fluid communication with the mixing assembly and dissolution of the dry component into the wet component occurs. The mixing assembly can contain at least one fluidic conduit, for example at least one fluidic channel. In some embodiments, the mixing assembly contains at least one microfluidic channel. The mixing assembly is also configured to transfer the dissolved or reconstituted wet and dry components into a needle assembly or other delivery assembly configured to inject or deliver said components into a subject, person or animal.

In some embodiments, the composition comprising ghrelin or the ghrelin variant is administered via inhalation, oral, intravenous, parenteral, buccal, subcutaneous, transdermal, patch, sublingual, intramuscular, or intranasal. In some embodiments, ghrelin is administered in a single dose. In some embodiments, ghrelin is administered in multi-doses. In some embodiments, ghrelin is administered at a dosage from 10 ng/kg per day to 10 mg/kg per day (or any sub value or sub range there between, e.g., 0.1 μg/kg per day to 5 mg/kg per day). In some embodiments, a dosing regimen (2 μg/kg per day, for example delivered intravenously) is administered within 8 hours following injury. The dosing is a one-time dose with possible recurrent dosing based on patient symptoms.

Nasal delivery is a non-invasive route for therapeutics targeting the central nervous system because of relatively high permeability of nasal epithelium membrane, avoidance of hepatic first pass elimination. Nasal delivery is easy to administer and allows for self-medication by an individual. Nasal mucociliary clearance is an important limiting factor to nasal drug delivery. Nasal mucociliary clearance severely limits the time allowed for drug absorption to occur and may effectively prevent sustained drug administration. However, it has been documented that nasal administration of certain hormones has resulted in a more complete administration. In some embodiments, the present disclosure utilizes nasal delivery of ghrelin.

In some embodiments, a composition comprising ghrelin or the ghrelin variant that is suitable for nasal administration may include one or more bioadhesive polymers. Some polymers such as carbopol, can adhere onto the nasal mucosa for reasonably prolonged periods, preventing rapid nasal clearance. In some embodiments, a composition suitable for nasal administration, the percentage of bioadhesive polymer in a suitable solution of ghrelin is about 0.1%. In some embodiments, a composition suitable for nasal administration, the percentage of bioadhesive polymer in a suitable solution of ghrelin is about 0.5%. In some embodiments, a composition suitable for nasal administration, the percentage of bioadhesive polymer in a suitable solution of ghrelin is about 1%. In some embodiments, a composition suitable for nasal administration, the percentage of bioadhesive polymer in a suitable solution of ghrelin is about 5%.

In some embodiments, a composition comprising ghrelin or the ghrelin variant that is suitable for nasal administration may include one or more surfactants. Surfactants that may be used in the compositions of the present invention include different polyethylene glycols (PEGS) or polyethylene glycol-derivatives. In some embodiments, a composition suitable for nasal administration, the percentage of surfactant in a suitable solution of ghrelin is about 1%. In some embodiments, a composition suitable for nasal administration, the percentage of surfactant in a suitable solution of ghrelin is about 2%. In some embodiments, a composition suitable for nasal administration, the percentage of surfactant in a suitable solution of ghrelin is about 5%. In some embodiments, a composition suitable for nasal administration, the percentage of surfactant in a suitable solution of ghrelin is about 10%.

In some embodiments, a composition comprising ghrelin or the ghrelin variant that is suitable for nasal administration may include one or more buffering agents for controlling the pH of the composition. Buffering agents that may be used in the compositions of the present invention include citric acid and sodium citrate dihydrate. In some embodiments, a composition suitable for nasal administration, the percentage of buffering agent in a suitable solution of ghrelin is about 0.001%. In some embodiments, a composition suitable for nasal administration, the percentage of buffering agent in a suitable solution of ghrelin is about 0.005%. In some embodiments, a composition suitable for nasal administration, the percentage of buffering agent in a suitable solution of ghrelin is about 0.01%. In some embodiments, a composition suitable for nasal administration, the percentage of buffering agent in a suitable solution of ghrelin is about 0.1%.

In some embodiments, the osmolarity of the composition comprising ghrelin or the ghrelin variant may be controlled by propylene glycol. When a composition comprising ghrelin or the ghrelin variant is a gel, the composition may include a gelling agent such as hydroxylpropyl cellulose, carbopols, carboxymethylcellulose, and ethylcellulose. In some embodiments, the composition comprising ghrelin or the ghrelin variant may include a preservative such as ethylenediaminetetraacetic acid (EDTA) and benzalkonium chloride. Non-limiting examples of suitable solvents for compositions of the present invention include water, vegetable oil and ethanol. In some embodiments, the use of a nasal inhalant reduces the concentration required to treat mBI and prevent unwanted side effects.

In some embodiments, nasal administration is a more practical means of delivery in a military or sport setting. In some embodiments, the present invention provides a method for improving the standard of care for preventing or treating mBI in military personnel or athletes through a prophylactic and post-acute intranasal therapeutic. In some embodiments, the active ingredient of the therapeutic is ghrelin. In some embodiments, ghrelin may be part of a formulation that is delivered intranasally to facilitate ease of access and use in the field and to minimize the dose required further limiting side effects.

In some embodiments, the composition comprising ghrelin or the ghrelin variant is administered via the iSPERSE (inhaled small particles easily respirable and emitted) technology, which is a dry powder technology developed by Pulmatrix. iSPERSE particles are engineered to be small, dense and easily dispersible. In some embodiments, the iSPERSE technology allows flexible drug loading for delivery of microgram to tens of milligrams per dose: iSPERSE particles do not require lactose or other carriers and can be engineered to include <1% to greater than 80% API to allow for dosing of low potency and high drug load therapeutics. In some embodiments, the iSPERSE technology allows reproducible and one-step manufacture: iSPERSE powders are manufactured by a scalable and reproducible one-step spray drying process with high and consistent yields. Formulations are created independent of API physical chemistry in either crystalline or amorphous excipient matrices. In some embodiments, the iSPERSE technology allows superior flow rate independent pulmonary administration: iSPERSE formulations are dispersible across a range of flow rates with consistent emitted dose and particle size. Performance across flow rates provides reliable dose delivery across patient populations and reduces patient-to-patient variability. In some embodiments, the iSPERSE technology allows delivery of macromolecules and biologics: iSPERSE enables delivery of antibodies, peptides and nucleic acids across a range of drug loads and with robust product performance. In some embodiments, the iSPERSE technology allows homogenous combinations of multiple drugs: iSPERSE creates homogenous particles including excipients and API. Dual and triple iSPERSE combinations have been manufactured to date. In some embodiments, the iSPERSE technology allows flexibility of patient interface. iSPERSE dry powders are compatible with a range of inhalers, allowing for a product's configuration to be tailored to the specific needs of a patient population. The iSPERSE technology is disclosed in U.S. Patent Application No. 2015/0136130, the disclosure of which is incorporated herein by reference in its entirety.

In some embodiments, the respirable dry powder comprises respirable dry particles that contain at least one therapeutic agent and at least one metal cation salt, such as a sodium salt, a potassium salt, a magnesium salt, or a calcium salt, and that have a volume median geometric diameter (VMGD) about 10 micrometers or less. These dry particles can be further characterized by a tap density at least about 0.45 g/cm3 to about 1.2 g cm3, at least about 0.55 g/cm3 to about 1.1 g cm3, or at least about 0.65 g cm3 to about 1.0 g cmJ; and a total content of therapeutic agent or agents of at least 25%, at least 35%, at least 50%, at least 65%, or at least 80% by weight (i.e., dry weight relative to the total dry weight of dry powder). The powders can be further characterized by an angle of repose of 50° or less, 40° or less, or 30° or less. The particles can be further characterized by a dispersibility ratio (1 bar/4 bar) of less than about 2 as measured by laser diffraction (RODOS/HELOS system), less than about 1.7, less than about 1.4, or less than about 1.2. The particles can be further characterized by a fine particle fraction (e.g., FPF<5.6, <5.0, <4.4 or <3.4) of 30% or greater, 40% or greater, 50% or greater, or 60% or greater.

In some embodiments, the respirable dry powders comprising respirable dry particles, are “processable.” For example, the dry powders can be deposited or filled into a sealable receptacle that has a volume of about 12 cubic millimeters (mm3) or less, a volume of about 9 mm3 or less, a volume of about 6 mm3 or less, a volume of about 3 mm3 or less, a volume of about 1 mm3 or less, or a volume of about 0.5 mm3 or less, preferably to substantially fill the volume of the receptacle. Alternatively or in addition, the powders can be deposited or filled into a sealable receptacle to provide a mass of about 1 mg or less, about 0.75 mg or less, about 0.5 mg or less, about 0.3 mg or less, about 0.1 mg or less, or about 0.05 mg or less of powder in the receptacle.

The respirable dry powders consisting of respirable dry particles can be deposited into receptacles to provide a total dry powder mass of between about 5 mg to about 15 mg, between about 5 mg and less than 10 mg, between about 5 mg and about 9 mg, between about 5 mg and about 8 mg, or between about 5 mg and about 8 mg. The receptacles that contain the dry powder mass can be sealed if desired.

The dry powders comprising respirable dry particles can be deposited into receptacles to provide a total dry powder mass of about 5 mg or less, about 4 mg or less, about 3 mg or less or about 2 mg or less, and provide about 1 mg or more, wherein the total dry powder mass contains 1.5 mg or more, or about 2 mg or more of one or more therapeutic agents. In such embodiments, the receptacle will contain between 1.5 mg and about 5 mg or less, or about 2 mg and about 5 mg or less of total dry powder mass. The receptacles that contain the dry powder mass can be sealed if desired.

The total content of therapeutic agent or agents in the respirable dry powder is at least 20%, at least 25%, at least 35%, at least 50%, at least 65%, or at least 80% by weight (i.e., dry weight relative to the total dry weight of dry powder). The one or more metal cation salt can be present in the respirable dry particles in any desired amount, such as about 3% by weight or more of the respirable particles, 5% by weight of the respirable particles, 10% by weight of the respirable particles, 15% by weight of the respirable particles, or in 20% by weight of the respirable particles. The one or more metal cation salt can independently be selected from the group consisting of a sodium salt, a potassium salt, a magnesium salt, and a calcium salt.

The respirable dry powder is filled or deposited into receptacles using standard filling equipment such as a vacuum dosator, for example, a rotating drum vacuum dosator, e.g., the Omnidose TT (Harro Hofliger, Germany). The volume of the receptacle into which the respirable dry powder is filled can be 400 microliters or less, 330 microliters or less, 250 microliters or less, 150 microliters or less, 70 microliters or less, 40 microliters or less, or 20 microliters or less. In one aspect, the respirable dry powder can be filled into two or more receptacles that are physically attached to each other or in an array, for example, using an interconnected blister piece comprising 30 blisters or more, 60 blisters or more, 90 blisters or more, or 120 blisters or more. Each receptacle or array of receptacles (e.g., an interconnected blister piece) can be filled at a rate of about every 10 seconds or less, about every 8 seconds or less, about every 6 seconds or less, about every 4 seconds or less, about every 2 seconds or less, or about every 1 second or less. Preferably, the relative standard deviation (RSD) is about 3% or less, about 2.5% or less, about 2% or less, or about 1.5% or less. A dry powder inhaler (DPI) that contains the receptacles can be any suitable DPI, such as a multi-dose blister DPI, a single-dose capsule DPI, or other DPI. The angle of repose of the respirable dry powder that is filled into the receptacles can be 50° or less, 40″ or less, or 30° or less. The processable powder may be essentially free of non-respirable carrier particles, such as lactose, that have a VMGD that is greater than 10 micrometers, about 20 micrometers or greater, 30 micrometers or greater, or 40 micrometers or greater.

In other examples, the processable powders can be metered in a multi-dose reservoir dry powder inhaler (DPI), the metering achieved by a dosing cup, disk, or other structure for dosing in the reservoir DPI itself. Unit doses can be metered which are 100 cubic millimeters or less, 75 cubic millimeters or less, 50 cubic millimeters or less, 35 cubic millimeters or less, 20 cubic millimeters or less, 10 cubic millimeters or less, 5 cubic millimeters or less, or 2.5 cubic millimeters or less. In some aspects, the metering mechanism can possess one receptacle to measure a unit dose, and in other aspects, the metering mechanism can possess multiple receptacles to measure a unit dose. Alternatively or in addition, the processable powders can further be characterized as processable in that the mass of the metered dose from a multi-dose reservoir DPI is within 80% to 120% of a target mass 85% or more of the time, or within 85% to 115% of a target mass 90% of the time, or within 90% to 110% of a target mass 90% of the time. Preferably, the mass of the metered dose from a multi-dose reservoir DPI is within 85% to 115% of a target mass 90% or more of the time, or within 90% to 110% of a target mass 90% or more of the time. The processable dry powders can be further be characterized by an angle of repose of 50° or less, 40° or less, 30° or less. Angle of repose is a characteristic that can describe both respirable dry powder as well as the powder's processability.

In addition or alternatively to any of the forgoing processability characteristics, the processable powders can further be filled into a receptacle for use in a DPI at a rate of one receptacle about every 10 seconds or less, about every 8 seconds or less, about every 6 seconds or less, about every 4 seconds or less, about every 2 seconds or less, about every 1 second or less, or about every 0.5 seconds or less; and/or filled into receptacles for use in a DPI at a rate of 300 receptacles every hour, 500 receptacles every hour, 750 receptacles every hour. 1 100 receptacles every hour, 1500 receptacles every hour, 2000 receptacles every hour, 2500 receptacles every hour, or 3000 receptacles every hour. The rate of filling the receptacles can also be 800 receptacles or more every hour, 1600 receptacles or more every hour or 2400 receptacles or more every hour. Preferably at least 70% of the receptacles are filled within 80% to 120% of the target fill weight, at least 80% of the receptacles are filled within 85% to 115% of the target fill weight, or 85% of the receptacles are filled within 90% to 110% of the target fill weight. More preferably, at least 85% of the receptacles are filled within 90% to 110% of the target fill weight.

In addition or alternatively to any of the forgoing processability characteristics, the processable powders can further be filled into a receptacle for use in a DPI at a rate of 300 receptacles or more every hour, 500 receptacles or more every hour, 750 receptacles or more every hour, 1100 receptacles or more every hour, 1500 receptacles or more every hour, 2000 receptacles or more every hour, 2500 receptacles or more every hour, or 3000 receptacles or more every hour. Preferably, at least 70% of the receptacles are filled within 80% to 120% of the target fill weight, at least 80% of the receptacles are filled within 85% to 115% of the target fill weight, or at least 85% of the receptacles are filled within 90% to 110% of the target fill weight. In addition or alternatively, the relative standard deviation of the fill weight is 3% or less, 2.5% or less, 2% or less, or 1.5% or less. Filling equipment that may be used include pilot scale equipment and commercial scale equipment.

In some embodiments, the respirable dry powders are processable and dispersible. Such dry powders can contain a proportionately large mass of one or more therapeutic agents (e.g., 50% or more (w/w) by dry weight) and be administered to a subject to deliver an effective amount of the therapeutic agent to the respiratory tract. For example, a unit dosage form of the dry powder, provided as a small volume receptacle (e.g., capsule or blister) with the dry powder disposed therein or as a reservoir-based DPI metered to dispense a small volume, can be used to deliver an effective amount of the therapeutic agent to the respiratory tract of a subject in need thereof. In one aspect, at least 20 milligrams of one or more therapeutic agent can be delivered to the respiratory tract from a small volume unit dosage form. For example, at least about 25 milligrams, at least about 30 milligrams, at least about 45 milligrams, at least about 60 milligrams, at least about 80 milligrams, at least about 100 milligrams, at least about 130 milligrams, at least about 160 milligrams, or at least about 200 milligrams of one or more therapeutic agent can be delivered to the respiratory tract from a unit dosage form provided as a small volume receptacle (e.g., volume of about 400 microliters or less, about 370 microliters or less, less than 370 microliters, about 300 microliters or less, less than about 300 microliters, preferably, about 370 microliters, or about 300 microliters) with the dry powder disposed therein. In some embodiments, the receptacle is a size 2 or a size 3 capsule.

Features of the dry powders, receptacle and/or inhaler can be adjusted to achieve the desired delivery of an effective amount of the therapeutic agent to the respiratory tract of a subject in need thereof. Such features include 1) the therapeutic agent load in the dry particles or dry powder; 2) the bulk density of the dry powder, 3) the degree to which the receptacle is filled with the dry powder, and 4) the processability and dispersibility of the dry powder. The therapeutic agent load in the dry powder is generally at least about 25%, at least about 35%, at least about 50%, at least about 65%, at least about 80%, or at least about 90% by weight, on a dry basis. The bulk density of the dry powder is generally greater than 0.1 g cc, between about 0.2 g/cc and about 0.9 g cc, and preferably, at least about 0.3 g/ml, at least about 0.4 g/ml, or at least 0.5 g/ml. The bulk density, also referred to as the apparent density, is a measure that indicates how much dry powder can be filled into a fixed volume without the intense compaction experienced when determining the tap density of a dry powder. The receptacle is generally filled with dry powder to be at least 50% full, preferably, at least 60% full, at least 70% full, or at least 90% full. The processability and dispersibility of the dry powder can be altered, as desired, by including appropriate amounts of one or more monovalent and/or divalent metal cation salts, (e.g., a sodium salt, a potassium salt, a magnesium salt, a calcium salt, or a combination thereof, total metal cation salts less than about 75%, equal to or less than about 60%, about 50%, about 40%, about 30%, about 20%, about 10%, about 5%), and optionally, one or more other excipients (e.g., carbohydrates, sugar alcohols, and/or amino acids, total excipients equal to or less than about 70%, about 55%, about 40%, about 30%, about 20%, about 10%, about 5%) in the dry powders or dry particles. If desired, the therapeutic agent load may be at least about 20% by weight, on a dry basis. Although it is preferable that the receptacle is filled at least 50%, the receptacle can be filled to any desired degree, such as at least 10% filled, at least 20% filled, at least 30% filled, or at least 40% filled.

Using ghrelin or ghrelin variants as disclosed herein as a therapeutic may reduce poor outcomes following injury, especially neuropsychological and neurodegenerative disorders including Chronic Traumatic Encephalopathy (CTE) and Post-Traumatic Stress Disorder (PTSD) linked to repetitive brain injuries, an increasing concern for today's military personnel and athletes.

“Post-Traumatic Stress Disorder (PTSD)” is an anxiety disorder that can develop after exposure to a terrifying event or ordeal in which grave physical harm occurred or was threatened to oneself or others. A single or repetitive mBIs may lead the patients to high risks for longer-term neuropsychological and neurodegenerative disorders, such as PTSD, which is associated with a high rate of suicide. PTSD was described in veterans of the American Civil War, and was called “shell shock,” “combat neurosis,” and “operational fatigue.” PTSD symptoms can be grouped into three categories: (1) re-experiencing symptoms; (2) avoidance symptoms; and (3) hyperarousal symptoms. Exemplary re-experience symptoms include flashbacks (e.g., reliving the trauma over and over, including physical symptoms like a racing heart or sweating), bad dreams, and frightening thoughts. Re-experiencing symptoms may cause problems in a person's everyday routine. They can start from the person's own thoughts and feelings. Words, objects, or situations that are reminders of the event can also trigger re-experiencing. Symptoms of avoidance include staying away from places, events, or objects that are reminders of the experience; feeling emotionally numb; feeling strong guilt, depression, or worry; losing interest in activities that were enjoyable in the past; and having trouble remembering the dangerous event. Things that remind a person of the traumatic event can trigger avoidance symptoms. These symptoms may cause a person to change his or her personal routine. For example, after a bad car accident, a person who usually drives may avoid driving or riding in a car. Hyperarousal symptoms include being easily startled, feeling tense or “on edge”, having difficulty sleeping, and/or having angry outbursts. Hyperarousal symptoms are usually constant, instead of being triggered by things that remind one of the traumatic event. They can make the person feel stressed and angry. These symptoms may make it hard to do daily tasks, such as sleeping, eating, or concentrating. Therefore, generally, PTSD symptoms can include nightmares, flashbacks, emotional detachment or numbing of feelings (emotional self-mortification or dissociation), insomnia, avoidance of reminders and extreme distress when exposed to the reminders (“triggers”), loss of appetite, irritability, hypervigilance, memory loss (may appear as difficulty paying attention), excessive startle response, clinical depression, stress, and anxiety. The symptoms may last for a month, for three months, or for longer periods of time. Some embodiments herein, relate to methods of treating, reducing, delaying the onset of, and/or minimizing the severity of PTSD, including PTSD resulting from one or more mild brain injuries. Such methods can include, for example, providing or administering any compound or combination described herein to a subject that has experienced a mild brain injury or concussion. For example, the compounds can be ghrelin or a ghrelin variant, alone or in combination with any of the other compounds and materials described herein. In some cases, the method can apply to or can include the selection of a subject that is susceptible to PTSD, has experienced it previously, or whose injury is of a type of injury or any injury that occurred in connection with an activity with a higher incidence of PTSD (e.g., military combat, injury where there was death of another person as part of the same injury causing event, etc.). In some embodiments, using ghrelin or a ghrelin variant (or any other agents described herein) as a therapeutic for treating PTSD associated with a single mild brain injury or concussion may reduce the severity of PTSD and/or the related symptoms, and/or delay the onset of PTSD and/or the related symptoms. In some embodiments, ghrelin or a ghrelin variant (or any other agents described herein) is used as a therapeutic for preventing the onset of PTSD or delaying PTSD resulted from one or more of mild brain injuries. In some embodiments, using ghrelin or a ghrelin variant (or any other agents described herein) as a therapeutic for treating PTSD linked to repetitive or multiple mild brain injuries may reduce or delay the severity of PTSD and/or the related symptoms, and/or delay the onset of PTSD and/or the related symptoms. In some embodiments, using ghrelin or a ghrelin variant (or any other agents described herein) as a therapeutic in a combination with one or more therapeutic agents disclosed herein for treating PTSD associated with a single mild brain injury or concussion may reduce the severity of PTSD and/or the related symptoms, and/or delay the onset of PTSD and/or the related symptoms. In some embodiments, using ghrelin or a ghrelin variant (or any other agents described herein) as a therapeutic in a combination with one or more therapeutic agents disclosed herein for treating PTSD linked to repetitive or multiple mild brain injuries may reduce the severity of PTSD and/or the related symptoms, and/or delay the onset of PTSD and/or the related symptoms. In some embodiments, using ghrelin per se as a therapeutic for treating PTSD associated with a single mild brain injury or concussion may reduce the severity of PTSD and/or the related symptoms, and/or delay the onset of PTSD and/or the related symptoms. In some embodiments, ghrelin per se is used as a therapeutic for preventing the onset of PTSD or delaying PTSD resulted from one or more of mild brain injuries. In some embodiments, using ghrelin per se as a therapeutic for treating PTSD linked to repetitive or multiple mild brain injuries may reduce the severity of PTSD and/or the related symptoms, and/or delay the onset of PTSD and/or the related symptoms. In some embodiments, using ghrelin per se as a therapeutic in a combination with one or more therapeutic agents disclosed herein for treating PTSD associated with a single mild brain injury or concussion may reduce the severity of PTSD and/or the related symptoms, and/or delay the onset of PTSD and/or the related symptoms. In some embodiments, using ghrelin per se as a therapeutic in a combination with one or more therapeutic agents disclosed herein for treating PTSD linked to repetitive or multiple mild brain injuries may reduce the severity of PTSD and/or the related symptoms, and/or delay the onset of PTSD and/or the related symptoms.

In some embodiments, the present invention provides compositions comprising ghrelin or the ghrelin variant that are applied as nasal drops, eye drops and nasal sprays. For the nasal application, a solution or suspension may be used which is applied as spray, i.e., in the form of a fine dispersion in air or by means of a conventional spray-squeeze bottle or pump. Suitable nontoxic pharmaceutically acceptable carriers for use in a drug delivery system for intranasal administration of ghrelin may include, but not limited to, carriers used for nasal pharmaceutical formulations for other steroids, such as estrogen.

In some embodiments, formulations of the present invention may contain a preservative and/or stabilizer. These include, but not limited to, ethylene diamine tetraacetic acid (EDTA) and its alkali salts (for example dialkali salts such as disodium salt, calcium salt, calcium-sodium salt), lower alkyl p-hydroxybenzoates, chlorhexidine (for example in the form of the acetate or gluconate) and phenyl mercury borate. Other suitable preservatives are: pharmaceutically useful quaternary ammonium compounds, for example cetylpyridinium chloride, tetradecyltrimethyl ammonium bromide, generally known as “cetrimide”, N-Benzyl-N,N-dimethyl-2-{2-[4-(2,4,4-trimethylpentan-2-yl)phenoxy]ethoxy}ethanaminium chloride, generally known as “benzethonium chloride” and myristyl picolinium chloride. Each of these compounds may be used in a concentration of about 0.002 to 0.05%, for example about 0.02% (weight/volume in liquid formulations, otherwise weight/weight). In some embodiments, preservatives among the quaternary ammonium compounds are, but not limited to, alkylbenzyl dimethyl ammonium chloride and mixtures thereof, for example, benzalkonium chloride.

In some embodiments, the present invention provides for a treatment strategy for athletes who have suffered a mBI that may not only reduce the time required for safe return to play but also provide protection from future mBI.

Intranasal (IN) administrations may have fewer side effects than intraperitoneal (IP) administrations due to a shift in pharmaceutical research to nasal sprays, drops and gels: the nasal route of drug administration continues to receive increasing attention from pharmaceutical scientists and clinicians because this route circumvents hepatic first pass elimination associated with oral delivery, is easily accessible and suitable for self-medication. Intranasal administration also particularly suits drugs targeting the brain because certain drug solutions can bypass the blood-brain barrier (BBB) and reach the central nervous system (CNS) directly from the nasal cavity—uptake of these drugs depends on their molecular weight and lipophilicity. The intranasal delivery increases brain levels of the drug while decreasing systemic concentrations and thus should have less harmful side effects.

In some embodiments, the compositions comprising ghrelin or ghrelin variants are delivered by Precisa™ Platform. Precisa™ is Edge Therapeutics' proprietary, programmable, biodegradable polymer-based development platform. It allows creating polymer-based therapeutics capable of delivering therapeutics directly to the site of injury to potentially avoid serious systemic side effects often associated with oral or intravenous delivery. Precisa™ enables high and sustained drug exposure with only a single dose at the initial time of procedural or surgical intervention.

Precise™ can be designed based on specific physical and chemical properties (size, shape, surface area) of the drug to be delivered that allow for one-time administration at or near the targeted injured organ, vessel or cell. In some embodiments, the specific form of Precise™ microparticles containing the compositions comprising ghrelin or ghrelin variants that is small enough to allow easy administration through a brain catheter, yet large enough to prevent macrophages from carrying the microparticles away from the site of injury.

Precisa™ is programmed with a specific blend of polymers in order to obtain the desired release profile of the selected therapeutic. This is accomplished by immersing the specified therapeutic in a matrix of clinically validated, biodegradable and biocompatible polymers. The foundation of Precisa™ is poly (DL-Lactic-co-glycolide), or PLGA, the polymer in dissolvable sutures that has been used since the 1970s. PLGA is biodegradable, has minimal toxicity in humans, even when used intracranially, and is one of the few matrix delivery systems where drug release can be sustained over weeks.

Upon administration, the therapeutic that is on the surface of the polymer immediately releases to provide high initial concentrations of such therapeutic. Subsequently, the therapeutic begins to diffuse through the polymer-based matrix and the polymer breaks down into lactic acid, a compound naturally found in the body, in order to deliver the therapeutic with the desired release profile.

In some embodiments, the compositions comprising ghrelin or ghrelin variants are delivered by Quanterix's proprietary Simoa™ technology. Simoa™ technology is based upon the isolation of individual immunocomplexes on paramagnetic beads using standard ELISA reagents. The main difference between Simoa and conventional immunoassays lies in the ability to trap single molecules in femtoliter-sized wells, allowing for a “digital” readout of each individual bead to determine if it is bound to the target analyte or not.

In some embodiments, the present invention provides a method of prophylactically administering a composition comprising ghrelin or the ghrelin variant to individuals who are involved in activities, such as contact sports or serving in the armed forces, where there is a possibility of the individuals suffering mBI. In some embodiments, the present invention provides a method for acutely treating individuals who have suffered mBI. For acute treatments, nasal administration of the composition comprising the ghrelin variant may reduce the time for uptake and increase the concentration of the ghrelin variant that reaches the blood or brain.

In some embodiments, the ghrelin variant is administered in a single dose. In some embodiments, the ghrelin variant is administered in multi-doses. In some embodiments, the ghrelin variant is administered at a dosage from 10 ng/kg per day to 10 mg/kg per day.

In some embodiments, ghrelin variant compositions may be formulated in an oral administration dosage forms. The pharmaceutical compositions and dosage forms may comprise the compounds disclosed herein or their pharmaceutically acceptable salt or crystal forms thereof as the active component.

The pharmaceutical acceptable carriers can be either solid or liquid. Solid form preparations include powders, tablets, pills, capsules, cachets, suppositories, and dispersible granules. A solid carrier can be one or more substances, which may also act as diluents, flavoring agents, solubilizers, lubricants, suspending agents, binders, preservatives, wetting agents, tablet disintegrating agents, or an encapsulating material.

For oral administration, such excipients include, e.g., pharmaceutical grades of mannitol, lactose, starch, magnesium stearate, sodium saccharine, talcum, cellulose, glucose, gelatin, sucrose, magnesium carbonate, and the like. In powders, the carrier is a finely divided solid, which is a mixture with the finely divided active component. In tablets, the active component is mixed with the carrier having the necessary binding capacity in suitable proportions and compacted in the shape and size desired. The powders and tablets contain from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include a composition comprising an active compound disclosed herein with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops may comprise sterile or nonsterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Other forms suitable for oral administration include liquid form preparations including emulsions, syrups, elixirs, aqueous solutions, aqueous suspensions, toothpaste, gel dentifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well-known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The administration of ghrelin variants are based on suitable dosing regimens that take into account factors well-known in the art including, e.g., type of subject being dosed; age, weight, sex and medical condition of the subject; the route of administration; the renal and hepatic function of the subject; the desired effect; and the particular compound employed. Optimal precision in achieving concentrations of drug within the range that yields efficacy without toxicity requires a regimen based on the kinetics of the drug's availability to target sites. This involves a consideration of the distribution, equilibrium, and elimination of a drug.

In some embodiments, ghrelin and ghrelin variants are administered subcutaneously. In some embodiments, ghrelin and ghrelin variants are administered as a bolus, wherein the administration form may be any suitable parenteral form.

Pharmaceutical compositions for parenteral administration include sterile aqueous and non-aqueous injectable solutions, dispersions, suspensions or emulsions, as well as sterile powders to be reconstituted in sterile injectable solutions or dispersions prior to use. Other suitable administration forms include suppositories, sprays, ointments, creams, gels, inhalants, dermal patches, implants, pills, tablets, lozenges and capsules.

A typical non-limiting dosage is in a concentration equivalent to from 10 ng to 10 mg ghrelin variant per kg bodyweight. The concentrations and amounts herein are given in equivalents of amount ghrelin variant. For example, where the ghrelin variant is a 28 amino acid human ghrelin (SEQ ID NO:1) and/or a 24 amino acid human ghrelin splice variant having a Dpr residue at the third position (SEQ ID NO:3) and/or a 24 amino acid human ghrelin splice variant having Dpr residues at the second and third positions (SEQ ID NO:4) and being optionally octanoylated on the Dpr residue in the third position. In some embodiments, the dosage can be the same for smaller peptides (e.g., RM-131 pentapeptide) or other ghrelin mimetics, antagonists, or agonists described herein. In some embodiments, a ghrelin or ghrelin variant is administered in a concentration equivalent to from about 0.1 μg to about 1 mg ghrelin per kg bodyweight, such as from about 0.5 μg to about 0.5 mg ghrelin per kg bodyweight, such as from about 1.0 μg to about 0.1 mg ghrelin per kg bodyweight, such as from about 1.0 μg to about 50 μg ghrelin per kg bodyweight, such as from about 1.0 μg to about 10 μg ghrelin per kg bodyweight.

In some embodiments, ghrelin variants are administered in a concentration equivalent to from 0.1 μg to 1 mg ghrelin variant per kg bodyweight, such as from 0.5 μg to 0.5 mg ghrelin variant per kg bodyweight, such as from 1.0 μg to 0.1 mg ghrelin variant per kg bodyweight, such as from 1.0 μg to 50 μg ghrelin variant per kg bodyweight, such as from 1.0 μg to 10 μg ghrelin variant per kg bodyweight.

In some embodiments, an intravenous injection of ghrelin variant is employed. The administration route must ensure that the non-degraded, bioactive form of the peptide will be the dominating form in the circulation, which will reach and stimulate the ghrelin variant receptors in order to obtain the maximum effect of ghrelin variant treatment on mBI. In some embodiments, the ghrelin variant is administered within about 30 minutes of the incident that results in mBI. In some embodiments, the ghrelin variant is administered within about 30 minutes to about 2 hours of the incident that results in mBI. In some embodiments, the ghrelin variant is administered within about 30 minutes to about 6 hours of the incident that results in mBI. In some embodiments, the ghrelin variant is administered within about 30 minutes to about 12 hours of the incident that results in mBI.

Ghrelin variant compositions may also be formulated for nasal administration. The solutions or suspensions are applied directly to the nasal cavity by conventional means, for example with a dropper, pipette or spray. The compositions may be provided in a single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Ghrelin variant compositions may be formulated for aerosol administration, particularly to the respiratory tract and including intranasal administration. The compound will generally have a small particle size, for example of the order of 5 microns or less. Such a particle size may be obtained by means known in the art, for example by micronization. The active ingredient is provided in a pressurized pack with a suitable propellant such as a hydrofluoroalkane (HFA) for example hydrofluoroalkane-134a and hydrofluoroalkane-227, carbon dioxide or other suitable gas. The aerosol may conveniently also contain a surfactant such as lecithin. The dose of drug may be controlled by a metered valve.

Alternatively, the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of, e.g., gelatin or blister packs from which the powder may be administered by means of an inhaler. Compositions administered by aerosols may be prepared, for example, as solutions in saline, employing benzyl alcohol or other suitable preservatives, absorption promoters to enhance bioavailability, employing fluorocarbons, and/or employing other solubilizing or dispersing agents.

Ghrelin variant compositions may also be formulated for administration by injection pen in a similar way as for cartridge growth hormone (GH) or Insulin. The cartridge contains compounds disclosed herein in solvents. The pen, which is basically a needle, syringe and vial in one piece, is operated by a turning movement and allows different doses to be administrated. This device offers simplicity, convenience, and enhanced safety features for compounds delivery. It provides a simple device design, few administration steps and one-step dial-back dose knob. Such injection pen can be obtained by means known in art.

Ghrelin variant compositions may be formulated for parenteral administration (e.g., by injection, for example bolus injection or continuous infusion) and may be presented in unit dose form in ampules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water. Aqueous solutions should be suitably buffered if necessary, and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Ghrelin variant compositions may be prepared in solutions, such as water or saline, and optionally mixed with a nontoxic surfactant. Compositions for intravenous or intra-arterial administration may include sterile aqueous solutions that may also contain buffers, liposomes, diluents and other suitable additives. Oils useful in parenteral compositions include petroleum, animal, vegetable, or synthetic oils. Specific examples of oils useful in such compositions include peanut, soybean, sesame, cottonseed, corn, olive, petrolatum, and mineral. Suitable fatty acids for use in parenteral compositions include oleic acid, stearic acid, and isostearic acid. Ethyl oleate and isopropyl myristate are examples of suitable fatty acid esters.

The parenteral compositions typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such compositions will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral compositions can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The pharmaceutical dosage forms suitable for injection or infusion can include sterile aqueous solutions or dispersions comprising the active ingredient that are adapted for administration by encapsulation in liposomes. In all cases, the ultimate dosage form must be sterile, fluid and stable under the conditions of manufacture and storage.

Sterile injectable solutions are prepared by incorporating a ghrelin variant or pharmaceutical acceptable salt thereof in the required amount in the appropriate solvent with various of the other ingredients enumerated above, as required, followed by, e.g., filter sterilization.

Ghrelin variant compounds can also be delivered topically. Regions for topical administration include the skin surface and also mucous membrane tissues of the rectum, nose, mouth, and throat. Compositions for topical administration via the skin and mucous membranes should not give rise to signs of irritation, such as swelling or redness.

Ghrelin variant compounds may include a pharmaceutical acceptable carrier adapted for topical administration. Thus, the composition may take the form of, for example, a suspension, solution, ointment, lotion, cream, foam, aerosol, spray, suppository, implant, inhalant, tablet, capsule, dry powder, syrup, balm or lozenge. Methods for preparing such compositions are well known in the pharmaceutical industry.

Ghrelin variant compounds may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Compositions suitable for topical administration in the mouth include lozenges, pastilles, and mouthwashes.

Ghrelin variant compounds may be administered transdermally, which involves the delivery of a pharmaceutical agent for percutaneous passage of the drug into the systemic circulation of the patient. The skin sites include anatomic regions for transdermally administering the drug and include the forearm, abdomen, chest, back, buttock, and the like. Transdermal delivery is accomplished by exposing a source of the active compound to a patient's skin for an extended period of time. Transdermal patches can add advantage of providing controlled delivery of a compound complex to the body. Such dosage forms can be made by dissolving, dispersing, or otherwise incorporating a ghrelin variant compound in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

Ghrelin variant compounds may be formulated for administration as suppositories. A typical suppository is produced by providing a low melting wax, such as a mixture of fatty acid glycerides or cocoa butter, that is first melted and the active component is dispersed homogeneously therein, for example, by stirring. The molten homogeneous mixture is then poured into convenient sized molds, allowed to cool, and to solidify. The active compound may be formulated into a suppository comprising, for example, about 0.5% to about 50% of a compound disclosed herein, disposed in a polyethylene glycol (PEG) carrier (e.g., PEG 1000 [96%] and PEG 4000 [4%]).

### Combination Therapies, Products and Compositions

Some embodiments relate to products that comprise two or more agents as discussed herein that can be utilized in combination. For example, the at least two agents can be selected from ghrelin, a ghrelin variant, an anti-inflammatory agent, anti-pain medication, acetylsalicylic acid, an antiplatelet agent, a thrombolytic enzyme, an aggregation inhibitor, a glycoprotein IIb/IIIa inhibitor, a glycosaminoglycan, a thrombin inhibitor, an anticoagulant, heparin, coumarin, tPA, GCSF, streptokinase, urokinase, Ancrod, melatonin, a caspase inhibitor, an NMDA receptor agonist or antagonist, an anti-TNF-α compound, an antibody, erythropoietin/EPO, angiotensin II lowering agent, selective androgen receptor modulator, leptin, an agonists of the renin-angiotensin system, an opioid receptor agonist, progesterone, a peroxisome proliferator-activated receptor gamma agonist, P7C3, P2Y purinergic receptor agonist, UCP-2 agonists, glypromate, NNZ-2591 (Neuren), NNZ-2566 (Neuren), cyclosporine A (NeuroVive Pharmaceutical), NTx-265 (Stem Cell Therapeutics), DP-b99 (D-Pharm), apomorphine, Epoetin Alfa (EPO), progesterone, KN 38-7271/BAY 38-7271 (KeyNeurotek/Bayer AG), VAS-203 (Vasopharm), SAR 127963 (Sanofi), BHR100 (BHR Pharma), Oxycyte (Oxygen Biotherapeutics), sulfonamide compounds, Ebselen (2-phenyl-1,2-benzisoselenazol-3(2H)-one), SPI-1005 (Sound Pharmaceuticals), glutathione peroxidase, glutathione peroxidase mimics and inducers, aducanumab (BIIB037, Biogen ldec), and any other compounds described herein, and the like.

UCP-2 agonists include, but are not limited to, β3 agonists, β3-adrenergic receptor (β3-AR), trecadrine, PPAR agonists (e.g., Wy-14643), NPY1 antagonists, NPY4 antagonists, leptin, leptin agonists, and uncoupling protein (“UCP”) activating agents.

Glypromate is a naturally occurring peptide fragment that is found in normal brain tissue. When injected intravenously, glypromate has been shown to act by multiple pathways in protecting brain tissue from injury. NNZ-2591 (cyclo-L-glycyl-L-2-allylproline), a diketopiperazine, has been shown to be neuroprotective after ischemic brain injury and also improves motor function in a rat model of Parkinson's disease. NNZ-2566, a synthetic analogue of neuroprotective tripeptide glypromate, is an IGF-1 like neuropeptide that is a caspase-3 inhibitor. Cyclosporine (cyclosporin, cyclosporine, cyclosporine A, or CsA) is an immunosuppressant drug used in organ transplantation to prevent rejection that suppresses the activity of the immune system by interfering with T cell activation and proliferation. KN 38-7271 is a CB1-2 agonist. VAS-203 is an allosteric NO synthase inhibitor. SAR127963 is a P75 receptor antagonist. BHR100 is a progesterone receptor agonist. Oxycyte is a perfluorocarbon oxygen carrier. KN 38-7271/BAY 38-7271 is a cannabinoid receptor agonist. DP-b99 is a Membrane Active Chelator (MAC) derivative of the known calcium chelator, BAPTA. Apomorphine is a non-selective dopamine agonist which activates both D1-like and D2-like receptors. NTx-265 is a drug comprising human Chorionic Gonadotropin (hCG) and Epoetin Alfa (EPO).

In some embodiments, at least two agents are separate agents that can be administered as part of the therapy for mBI, but not necessarily at the same time or as part of the same composition, although in some embodiments, the at least two agents are part of the same composition and/or are administered concurrently or at the same time. In some embodiments, the at least two agents are bound together. The at least two agents can form, for example, a dimer, a trimer, a tetramer, a pentamer, etc. In some cases, they can be conjugated, fused or otherwise bound together. In some aspects the agents can be bound together in such a manner that upon administration in vivo, the agents separate, for example, thereby releasing the two agents from each other. The agents can be bound together via any suitable manner. A variety of kits are commercially available and when taken in the context of the instant disclosure, one of skill in the art also can utilize various known methodologies for peptide-peptide, peptide-nonpeptide chemical, and peptide to pharmaceutical binding, fusion and conjugation.

In some embodiments, at least one of the at least two agents can be ghrelin. In some embodiments, at least two of the at least two agents are ghrelin molecules. The at least two ghrelin molecules further may include a pharmaceutically acceptable excipient, such as for example, sterile saline. In some embodiments, at least one of the at least two agents is a ghrelin variant, for example, a peptide of between 15 amino acids and 40 amino acids, a peptide of between 4 amino acids and 14 amino acids, a small molecule pharmaceutical, and the like. In some embodiments, the at least two agents can include at least one compound from the categories described herein (including, but not limited to those specific compounds and categories disclosed herein).

Some embodiments relate to methods of treating mild brain injury or concussion by administering the combinations and products described herein. Still other embodiments relate to methods of reducing the onset of or severity of a mild brain injury or concussion, comprising administering a therapeutically effect amount of the therapeutic products and combinations as set forth herein. In some embodiments, the ghrelin variant compounds may be administered in combination with additional pharmacologically-active substances or other pharmacologically-active material and/or may be administered in combination with another therapeutic method, which is administered before, during (including concurrently with) and/or after treatment of an individual with a ghrelin variant compound. The combination may be in the form of kit-in-part systems, wherein the combined active substances may be used for simultaneous, sequential or separate administration. The combination therapies are administered in pharmaceutically effective amounts, i.e., an administration involving a total amount of each active component of the medicament or pharmaceutical composition or method that is sufficient to show a meaningful patient benefit.

In some embodiments, ghrelin or a ghrelin variant can be used together or administered in combination with each other. In some embodiments, ghrelin and/or a ghrelin variant can be administered in combination with a therapeutic agent. In some embodiments, the therapeutic agent is one or more of an anti-inflammatory agent, anti-pain medication, acetylsalicylic acid, an antiplatelet agent, a thrombolytic enzyme, an aggregation inhibitor, a glycoprotein IIb/IIIa inhibitor, a glycosaminoglycan, a thrombin inhibitor, an anticoagulant, heparin, coumarin, tPA, GCSF, streptokinase, urokinase, Ancrod, melatonin, a caspase inhibitor, an NMDA receptor agonist or antagonist (e.g. gacyclidine-OTO-311), amantadine (e.g. ADS-5102), an anti-TNF-α compound, an antibody, erythropoietin/EPO, angiotensin II lowering agent, selective androgen receptor modulator, leptin or leptin variants, an agonists of the renin-angiotensin system, an opioid receptor agonist, progesterone or progesterone mimetics and variants, a peroxisome proliferator-activated receptor gamma agonist, a PPAR gamma/LXR inhibitor, an orphan nuclear receptor family 4A (NR4A) inhibitor or modulator, an ERbeta inhibitor or modulator, an inhibitor of STriatal-Enriched protein tyrosine Phosphatase (STEP), a STEP-derived peptide, P7C3, P2Y purinergic receptor agonist, glypromate, NNZ-2591 (Neuren), NNZ-2566 (Neuren), cyclosporine A (NeuroVive Pharmaceutical), NTx-265 (Stem Cell Therapeutics), DP-b99 (D-Pharm), apomorphine, Epoetin Alfa (EPO), progesterone, KN 38-7271/BAY 38-7271 (KeyNeurotek/Bayer AG), VAS-203 (Vasopharm), SAR 127963 (Sanofi), BHR100 (BHR Pharma), Oxycyte (Oxygen Biotherapeutics), glucagon, GLP-1R agonists, GLP-1, GLP-1 analog, synthetic form of GLP-1, GLP-1 (7-36) amide, Exendin-4 (Ex-4), Ex-4 analog, synthetic form of Ex-4, Lixisenatide, Liraglutide, a molecule in a biological pathway involving GLP-1R signaling pathway, incretin, incretin mimetic, Gastric inhibitory polypeptide (GIP), sulfonamide compounds, Ebselen (2-phenyl-1,2-benzisoselenazol-3(2H)-one), SPI-1005 (Sound Pharmaceuticals), glutathione peroxidase, glutathione peroxidase mimics and inducers, aducanumab (BIIB037, Biogen ldec) or a combination thereof.

The present disclosure provides for a number of GLP-1R agonists or derivatives, as well as incretin and incretin mimetics to be used in a therapeutic combination with ghrelin or ghrelin variants in treating mBI. GLP-1 is an endogenous metabolic hormone that stimulates insulin secretion, which is a naturally-occurring peptide that is released after a meal. GLP-1 is known to suppress glucagon secretion from pancreatic alpha cells and stimulate insulin secretion by pancreatic beta cells. GLP-1 receptor agonists are in development as an add-on treatment for type 2 diabetes. Glucagon-like peptide-1 (GLP-1) receptor signaling pathway in preclinical models of several CNS related neurological disorders such as Alzheimer's disease (AD), Parkinson's disease (PD), stroke, amyotrophic lateral sclerosis (ALS) and Huntington's disease (HD). Studies have shown that a GLP-1 receptor (GLP-1R) agonist, Exendin-4 (Ex-4), which readily crosses the blood-brain barrier, can be used to treat mild brain injury or concussion. The peptide Ex-4 can be obtained from Bachem (Torrance, Calif.). Lixisenatide (intended trade name Lyxumia) is a once-daily injectable GLP-1 receptor agonist. Liraglutide (NN2211) is a long-acting glucagon-like peptide-1 receptor agonist, binding to GLP-1R as does GLP-1. lncretins are a group of metabolic hormones that stimulate a decrease in blood glucose levels. lncretins can increase in the amount of insulin released from pancreatic beta cells. Gastric inhibitory polypeptide (GIP), also known as the glucose-dependent insulinotropic peptide, along with GLP-1, are members of the incretin class. In some embodiments, the therapeutic agent is a GLP-1R agonist. In some embodiments, the therapeutic agent is GLP-1. In some embodiments, the therapeutic agent is a GLP-1 analog, synthetic form of GLP-1, or GLP-1 (7-36) amide. In some embodiments, the therapeutic agent is Exendin-4 (Ex-4), Ex-4 analog, or synthetic form of Ex-4. In some embodiments, the therapeutic agent is Lixisenatide. In some embodiments, the therapeutic agent is Liraglutid. In some embodiments, the therapeutic agent is a molecule in a biological pathway involving GLP-1R signaling pathway. In some embodiments, the therapeutic agent is an incretin or incretin mimetic. In some embodiments, the therapeutic agent is a Gastric inhibitory polypeptide (GIP).

The present disclosure provides for a number of STEP-derived peptides or STEP derivatives to be used in a therapeutic combination with ghrelin or ghrelin variants in treating mBI. The brain-enriched tyrosine phosphatase STEP (also known as STriatal Enriched Phosphatase or PTPN5) is activated following stimulation of NMDARs and is emerging as an important regulator of neuronal survival and death. STEP is expressed specifically in neurons of the striatum, neo-cortex and hippocampus. STEP61 and STEP46, the two STEP isoforms contain a highly conserved substrate-binding domain termed as the kinase interacting motif or KIM domain. Phosphorylation of a critical serine residue within the KIM domain is mediated through dopamine/D1 receptor dependent activation of the Protein Kinase A (PKA) pathway. Dephosphorylation of this residue by Ca2+ dependent phosphatase calcineurin, following glutamate/NMDA receptor stimulation, renders STEP active in terms of its ability to bind to its substrates. Active STEP, in turn can bind to and modulate the activity of its substrate through tyrosine dephosphorylation of a regulatory tyrosine residue. Known substrates of STEP include ERK (extracellular regulated kinase 1/2) and p38 MAPKs, Src family tyrosine kinases and NMDAR subunits, all of which are involved in neuronal survival and death.

STEP is an intracellular protein tyrosine phosphatase (PTP) that is exclusively expressed in the central nervous system. STEP is preferentially expressed in neurons of the basal ganglia, hippocampus, cortex and related structures. The STEP-family of PTPases includes both membrane associated (STEP61) and cytosolic (STEP46) variants that are formed by alternative splicing of a single gene. STEP61 differs from STEP46 by the presence of an additional 172 amino acids at its N-terminus. For the purposes of this disclosure, specific amino acids residues within the STEP protein are frequently referred to using the numbering from the STEP46 variant. The 107 amino acid sequence of the STEP protein discussed herein is highly conserved between animal species with 95% sequence homology between rat (SEQ ID NO. 27) and human (SEQ ID NO. 28).

The present disclosure provides for a number of STEP-derived peptides containing mutations of the STEP protein. Where the described mutation sites are conserved between species, it will be well understood that the various STEP-based peptides described herein may be based on or derived from the STEP proteins from a variety of species and that such peptides may or may not be derived from proteins that arc endogenous to the specific species being treated and/or studied. Accordingly, the peptides disclosed herein should not be construed as being limited to the specific peptides sequences included in the sequence listing.

STEP along with two other PTPs, PTPRR and HePTP belongs to a family of PTPs that contains a highly conserved 16-amino acid substrate-binding domain termed the kinase interacting motif (KIM domain). A regulatory serine residue, ser 49 (ser 221 in STEP61) lies in the middle of the KIM domain and dephosphorylation of this residue renders STEP active in terms of its ability to bind to its substrates. Phosphorylation of ser 49 is mediated by dopamine/D1 receptor dependent activation of the cAMP/PKA pathway while dephosphorylation is mediated by glutamate/NMDA receptor induced activation of the Ca2+ dependent phosphatase, calcineurin.

A second conserved domain carboxy-terminal to the KIM domain is present in STEP, PTP-SL and HePTP. As described in further detail below, the domain, termed the kinase specificity sequence (MS domain) includes two phosphorylation sites, Thr 59 (Thr 231 in STEP61) and Ser 72 (Ser 244 in STEP61), which are important in regulating the stability of the STEP protein.

STEP61 and STEP46 contain the phosphatase domain, putative proteolytic sites (PEST), transmembrane Domain™, polyproline rich domain (PP), kinase interacting motif (KIM), kinase specificity sequence (KIS) and the above-mentioned phosphorylation sites involved in the activation and subsequent degradation of STEP. Additionally, the position of a cysteine residue (Cys 23 in STEP46/Cys 195 in STEP61) that has been shown to be involved in intermolecular dimerization and a threonine residue (Thr 18 in STEP46/Thr 190 in STEP61) that is known to be phosphorylatable by both ERK and p38 MAPKs.

In its active form STEP can modulate synaptic plasticity by regulating the activity of extracellular regulated kinase 1/2 (ERK1/2), a key protein involved in memory formation. Active STEP can also modulate NMDA receptor-dependent long term potentiation by interfering with NMDA receptor trafficking to synaptic membrane, possibly through regulation of the upstream kinase Fyn and tyrosine dephosphorylation of NR2B-NMDA receptor subunits. Several studies also indicate a role of active STEP in neuroprotection through its regulation of p38 MAPK.

Because the STEP protein is known to interfere with NMDAR, a constitutively active peptide based on the STEP protein is a likely candidate for treatment, amelioration and/or prevention of ischemic brain damage. A constitutively active STEP-derived peptide according to an embodiment of the present disclosure. The peptide contains the first 107 amino acids of STEP46 including the KIM and KIS domains. Furthermore, as shown in SEQ ID No 29, the serine residue (Ser 49) which acts as a PKA phosphorylation site has been modified. In some embodiments, Ser 49 has been converted, using standard point mutation techniques, to alanine, which is non-phosphorylatable, resulting in a constitutively active peptide. Modification of the PKA phosphorylation site addresses the problem of inactivation of the STEP-derived peptide due to phosphorylation.

Additional studies show that the active STEP protein is more susceptible to degradation. The study highlights the role of the KIS domain in regulation of the level of active STEP. Two SP/TP sites (i.e., Thr 59 and Ser 72) in the KIS domain are phosphorylated primarily through the basal activity of ERK and p38 MAPKs. Dephosphorylation of these two sites selectively results in ubiquitin-mediated proteasomal degradation of the active form of STEP. These findings imply that ubiquitin-mediated proteasomal degradation of active STEP may also lead to secondary activation of p38 MAPK.

In some embodiments, an active STEP-derived peptide that remains stable and is not susceptible to ubiquitin-mediated proteasomal degradation in vivo is desired. In some embodiments, the STEP-derived peptide is where Ser49 has been converted to alanine, which is non-phosphorylatable and Thr 59 and Ser 72 are converted, to glutamic acid to mimic the phosphorylatable form (SEQ ID No. 30). It will be appreciated that other phophomimics may be used including, for example, Aspartic acid.

Studies show that enzymatic activity of both STEP61 and STEP46 are also regulated by intermolecular dimerization. Dimerization of STEP involves intermolecular disulfide bond formation involving several cysteine residues and oxidative stress leads to increase in dimerization of STEP resulting in reduced activity. One such cysteine residue (Cys 23 in STEP46/Cys 195 in STEP61) that is involved in intermolecular dimerization is present in the STEP derived peptide described herein. It is possible that oxidative stress during an ischemic insult and/or reperfusion can lead to dimerization, at least in part, of the STEP derived peptide, thereby reducing its therapeutic efficacy. Accordingly, the present disclosure further provides a STEP peptide including a mutated Cys 23 residue (cysteine to alanine) (SEQ ID No. 31).

In some embodiments, the phosphorylation site in the STEP-derived peptide are (Thr 18 in STEP46/Thr 190 in STEP61). In vitro studies show that this site is phosphorylatable by both ERK and p38 MAPKs. Based on the functional significance of phosphorylation, the present disclosure further provides a STEP peptide including a nonphosphorylatable or mimic phosphorylatable form of Thr 18 (SEQ ID No. 32, Thr18 mutated to Glutamic Acid and SEQ ID No. 33, Thr18 mutated to Alanine). In some embodiments, SEQ ID No. 34 provides the amino acid sequence of the human STEP Peptide including all the mutations points discussed above.

Constitutively active STEP-derived peptides including a TAT sequence at the N-terminal of the peptide. In some embodiments, the phosphorylation site in the KIM domain has been altered (SEQ ID No. 35). In some embodiments, the phosphorylation sites in both the KIM and MS domains have been altered (SEQ ID No. 36). TAT is an 11 amino acid peptide that renders peptide sequences cell permeable and enables these peptides to cross the blood brain barrier. The ability of the STEP-derived peptide to cross the blood brain barrier enables the peptide to be delivered to a patient's brain via the significantly less invasive mechanism of intravenous injection, for example via the femoral vein, rather than previous treatment mechanisms that require direct surgical access to the brain. Those of skill in the art will be familiar with other suitable delivery mechanisms that could be employed including, for example, known targeted and viral-based delivery systems.

SEQ. ID NO. 27
MEEKVEDDFLDLDAVPETPVFDCVMDIKPETDPASLTVKSMGLQERRGSNV
SLTLDMCTPGCNEEGFGYLVSPREESAHEYLLSASRVLRAEELHEKALDPF
LLQAE
SEQ. ID NO. 28
MEEKIEDDFLDLDPVPETPVFDCVMDIKPEADPTSLTVKSMGLQERRGSNV
SLTLDMCTPGCNEEGFGYLMSPREESAREYLLSASRVLQAEELHEKALDPF
LLQAE
SEQ. ID NO. 29
MEEKIEDDFLDLDPVPETPVFDCVMDIKPEADPTSLTVKSMGLQERRGANV
SLTLDMCTPGCNEEGFGYLMSPREESAREYLLSASRVLQAEELHEKALDPF
LLQAE
SEQ. ID NO. 30
MEEKIEDDFLDLDPVPETPVFDCVMDIKPEADPTSLTVKSMGLQERRGANV
SLTLDMCEPGCNEEGFGYLMEPREESAREYLLSASRVLQAEELHEKALDPF
LLQAE
SEQ. ID NO. 31
MEEKIEDDFLDLDPVPETPVFDAVMDIKPEADPTSLTVKSMGLQERRGSNV
SLTLDMCTPGCNEEGFGYLMSPREESAREYLLSASRVLQAEELHEKALDPF
LLQAE
SEQ. ID NO. 32
MEEKIEDDFLDLDPVPEEPVFDCVMDIKPEADPTSLTVKSMGLQERRGSNV
SLTLDMCTPGCNEEGFGYLMSPREESAREYLLSASRVLQAEELHEKALDPF
LLQAE
SEQ. ID NO. 33
MEEKIEDDFLDLDPVPEAPVFDCVMDIKPEADPTSLTVKSMGLQERRGSNV
SLTLDMCTPGCNEEGFGYLMSPREESAREYLLSASRVLQAEELHEKALDPF
LLQAE
SEQ. ID NO. 34
MEEKIEDDFLDLDPVPEEPVFDAVMDIKPEADPTSLTVKSMGLQERRGANV
SLTLDMCEPGCNEEGFGYLMEPREESAREYLLSASRVLQAEELHEKALDPF
LLQAE
SEQ. ID NO. 35
MALYGRKKRRQRRRGEEKIEDDFLDLDPVPETPVFDCVMDIKPEADPTSLT
VKSMGLQERRGANVSLTLDMCEPGCNEEGFGYLMEPREESAREYLLSASRV
LQAEELHEKALDPFLLQAE
SEQ. ID NO. 36
MALYGRKKRRQRRRGEEKVEDDFLDLDAVPETPVFDCVMDIKPETDPASLT
VKSMGLQERRGANVSLTLDMCEPGCNEEGFGYLVEPREESAHEYLLSASRV
LRAEELHEKALDPFLLQAE

In some embodiments, the therapeutic agent is P7C3 as explained in U.S. Pat. No. 5,082,767, U.S. Pat. Appl. No. 2011/0015217 and 2014/0094480, which disclosures are hereby incorporated by reference in their entirety.

In some embodiments, the therapeutic agent is one or more of the compound having formula: 1-(3,6-Dibromo-9H-carbazol-9-yl)-3-(3-phenylamino)-propan-2-o; R-1-(3,6-Dibromo-9H-carbazol-9-yl)-3-(3-methoxyphenylamino)-propan-2-o; S-1-(3,6-Dibromo-9H-carbazol-9-y)-3-(3-methoxyphenylamino)-propan-2-ol; 1-(3,6-dibromo-9H-carbazol-9-yl)-3-(2-iminopyridin-1(2H)-yl)propan-2-ol; 1-(3,6-dibromo-9H-carbazol-9-yl)-3-(phenylthio)propan-2-o; N-(3-(3,6-dibromo-9H-carbazol-9-y)-2-hydroxypropyl)-N-(3 methoxyphenyl)acetamide; 5-((3,6-dibromo-9H-carbazol-9-yl)methyl)-3-(3-methoxyphenyl)-oxazolidin-2-one; N-(3-(3,6-dibromo-9H-carbazol-9-yl)-2-fluoropropyl)-3-methoxyaniline; 1-(3,6-dibromo-9H-carbazol-9-yl)-3-(3-methoxyphenylamino)-propan-2-one; N-(3-(3,6-dibromo-9H-carbazol-9-yl)-2-methoxypropyl)-3-methoxyaniline; 1-(3,6-Dimethyl-9H-carbazol-9-yl)-3-(3-methoxyphenylamino)propan-2-ol; 1-(3-Bromo-6-methyl-9H-carbazol-9-yl)-3-(3-methoxyphenylamino)-propan-2-ol; 1-(3,6-Dichloro-9H-carbazol-9-yl)-3-(3-methoxyphenylamino)propan-2-o; 1-(5-bromo-2,3-dimethyl-1H-indol-1-yl)-3-(phenylamino)propan-2-o; 1-(3,6-Dibromo-9H-pyrido[3,4-b]indol-9-yl)-3-(phenylamino)propan-2-ol; 1-(3-Azidophenylamino)-3-(3,6-dibromo-9H-carbazol-9-yl)propan-2-ol; 1,3-Bis(3,6-dibromo-9H-carbazol-9-yl)propan-2-o; 1-(9H-Carbazol-9-yl)-3-(3,6-dibromo-9H-carbazol-9-yl)propan-2-ol; 3-(3,6-Dibromo-9H-carbazol-9-yl)-2-hydroxy-N-(3-methoxyphenyl)-propanamide; Ethyl 5-(2-Hydroxy-3-(3-methoxyphenylamino)propyl)-8-methyl-3,4-dihydro-1H-pyrido[4,3-b] indole-2(5H)-carboxylate; 4-(3,6-dibromo-9H-carbazol-9-yl)-1-(phenylamino)butan-2-ol; N-(3-(3,6-dibromo-9H-carbazol-9-yl)propyl)aniline; 1-(3,6-dibromo-9H-carbazol-9-yl)-4-(phenylamino)butan-2-ol; 1-(3,6-dibromo-9H-carbazol-9-yl)-3-(pyridin-2-ylamino)propan-2-ol; 1-(3,6-dibromo-9H-carbazol-9-yl)-3-((3-methoxyphenyl)(methyl)-amino)propan-2-ol; 3-(3,6-dibromo-9H-carbazol-9-yl)-1-(3-methoxyphenylamino)-1-(methylthio)propan-2-one; 3-amino-1-(3-(3,6-dibromo-9H-carbazol-9-yl)-2-hydroxypropyl)pyridinium; 1-(3,6-dibromo-9H-carbazol-9-yl)-3-(pyrimidin-2-ylamino)propan-2-ol; N-(3-(3,6-dibromo-9H-carbazol-9-yl)-2-fluoropropyl)-3-methoxy-N-methylaniline; 1-(3,6-dibromo-9H-carbazol-9-yl)-3-methoxypropan-2-ol; 1-(3,6-dibromo-9H-carbazol-9-y)-4-phenylbutan-2-ol; 1-(3,6-dibromo-9H-carbazol-9-y)-3-(1H-indol-1-yl)propan-2-o; 3-(1-(3-(3,6-dibromo-9H-carbazol-9-yl)-2-hydroxypropyl)-1H-1,2,3-triazol-4-yl)propan-1-o; 1-(3,6-dibromo-9H-carbazol-9-yl)-3-(3-ethoxyphenylamino)propan-2-ol; 1-(3,6-dibromo-9H-carbazol-9-y)-3-(3,5-dimethyl-1H-pyrazol-1-yl)propan-2-o; 1-(3,6-dibromo-9H-carbazol-9-yl)-3-(phenylsulfinyl)propan-2-ol; 1-(3,6-dibromo-9H-carbazol-9-yl)-3-(phenylsulfonyl)propan-2-o; 1-(3-bromo-9H-carbazol-9-yl)-3-(3-methoxyphenylamino)propan-2-o; N-(5-(3-(3-(3,6-dibromo-9H-carbazol-9-yl)-2-hydroxypropylamino)phenoxy)pentyl)-2-(7-(dimethylamino)-2-oxo-2H-chromen-4-yl)acetamide; 1-(3,6-dibromo-9H-carbazol-9-yl)-3-phenoxypropan-2-o; N-(2-(3-(3,6-dibromo-9H-carbazol-9-yl)-2-hydroxypropoxy)ethyl)-acetamide; 1-(3,6-dibromo-9H-carbazol-9-yl)-3-(pyridin-3-ylamino)propan-2-o; 1-(3,6-dibromo-9H-carbazol-9-yl)-3-(pyridin-4-ylamino)propan-2-ol; 1-(2,8-dimethyl-3,4-dihydro-1H-pyrido[4,3-b]indol-5(2H)-yl)-3-(phenylamino)propan-2-ol; N-(3-(3,6-dibromo-9H-carbazol-9-yl)-2,2-difluoropropyl)-3-methoxyaniline; 1-(3,6-dibromo-9H-carbazol-9-yl)-3-phenoxypropan-2-ol; 1-(3,6-dibromo-9H-carbazol-9-yl)-3-(phenylamino)propan-2-o; 1-(3,6-dibromo-9H-carbazol-9-yl)-3-(o-tolylamino)propan-2-ol; 1-(3,6-dibromo-9H-carbazol-9-yl)-3-(m-tolylamino)propan-2-o; 1-(3,6-dibromo-9H-carbazol-9-yl)-3-(2-methoxyphenylamino)propan-2-o; 1-(3,6-dibromo-9H-carbazol-9-yl)-3-(naphthalen-1-ylamino)propan-2-o; 1-(4-bromophenylamino)-3-(3,6-dichloro-9H-carbazol-9-yl)propan-2-ol; 1-(4-bromophenylamino)-3-(3,6-dibromo-9H-carbazol-9-yl)propan-2-ol; 1-(3,6-dibromo-9H-carbazol-9-yl)-3-(4-ethoxyphenylamino)propan-2-o; 1-(4-chlorophenylamino)-3-(3,6-dibromo-9H-carbazol-9-y)propan-2-o; 1-(3,6-dibromo-9H-carbazol-9-yl)-3-(phenethylamino)propan-2-ol; 1-(3,6-dibromo-9H-carbazol-9-y)-3-(2-hydroxyethylamino)propan-2-ol; 1-(3,6-dibromo-9H-carbazol-9-yl)-3-(2,4-dimethoxyphenylamino)propan-2-o; 1-(3,6-dibromo-9H-carbazol-9-yl)-3-(2,3-dimethylphenylamino)propan-2-o; 1-(2-chlorophenylamino)-3-(3,6-dibromo-9H-carbazol-9-yl)propan-2-ol; 1-(tert-butylamino)-3-(3,6-dibromo-9H-carbazol-9-y)propan-2-o; 1-(3,6-dibromo-9H-carbazol-9-yl)-3-(isopropylamino)propan-2-ol; 1-(3,6-dibromo-9H-carbazol-9-y)-3-(4-methoxyphenylamino)propan-2-o; 1-(3,6-dibromo-9H-carbazol-9-yl)-3-(3-methoxyphenylamino)propan-2-o; 1-(3,6-dibromo-9H-carbazol-9-yl)-3-(m-tolylamino)propan-2-ol; 1-(3,6-dibromo-9H-carbazol-9-yl)-3-(3,5-dimethylphenylamino)propan-2-o; 1-(3,6-dibromo-9H-carbazol-9-yl)-3-(3,4-dimethylphenylamino)propan-2-o; 1-(3,6-dibromo-9H-carbazol-9-yl)-3-(3,4-dimethylphenylamino)propan-2-o; 1-(3,6-dibromo-9H-carbazol-9-yl)-3-(2,5-dimethylphenylamino)propan-2-ol; 1-(4-bromophenylamino)-3-(2,3-dimethyl-1H-indol-1-yl)propan-2-ol; 1-(2,3-dimethyl-1H-indol-1-yl)-3-(4-methoxyphenylamino)propan-2-ol; 1-(2,3-dimethyl-1H-indol-1-yl)-3-(4-ethoxyphenylamino)propan-2-o; 1-(2,3-dimethyl-1H-indol-1-yl)-3-(p-tolylamino)propan-2-ol; 1-(2,3-dimethyl-1H-indol-1-yl)-3-(phenylamino)propan-2-o oxalate; 1-(1H-indol-1-yl)-3-(4-methoxyphenylamino)propan-2-ol hydrochloride; 1-(1H-indol-1-yl)-3-(phenylamino)propan-2-ol oxalate; 1-(3,4-dihydro-1H-carbazol-9(2H)-yl)-3-(m-tolylamino)propan-2-ol; 1-(9H-carbazol-9-yl)-3-(phenylamino)propan-2-ol; 1-(3,6-dichloro-9H-carbazol-9-yl)-3-(phenylamino)propan-2-ol; 1-(9H-carbazol-9-yl)-3-(p-tolylamino)propan-2-ol; 1-(3,6-dichloro-9H-carbazol-9-yl)-3-(p-tolylamino)propan-2-ol; 1-(3,6-dibromo-9H-carbazol-9-yl)-3-(p-tolylamino)propa n-2-ol; N-(4-(3-(9H-carbazol-9-yl)-2-hydroxypropoxy)phenyl)acetamide; 1-(9H-carbazol-9-yl)-3-phenoxypropan-2-ol; 1-(9H-carbazol-9-yl)-3-(4-methoxyphenylamino)propan-2-ol; 1-(benzylamino)-3-(9H-carbazol-9-yl)propan-2-ol; methyl 4-(3-(9H-carbazol-9-yl)-2-hydroxypropoxy)benzoate; 1-(9H-carbazol-9-yl)-3-(4-methoxyphenoxy)propan-2-ol; 1-amino-3-(3,6-dibromo-9H-carbazol-9-yl)propan-2-ol; (S)-1-(3,6-dibromo-9H-carbazol-9-yl)-3-phenoxypropan-2-ol; (R)-1-(3,6-dibromo-9H-carbazol-9-yl)-3-phenoxypropan-2-ol; 3,6-dibromo-9-(2-fluoro-3-phenoxypropyl)-9H-carbazole; 1-(3,6-dibromo-9H-carbazol-9-yl)-3-(3-methoxyphenylamino)-2-methylpropa n-2-ol; 1-(2,8-di methyl-3,4-dihydro-1H-pyrido[4,3-b]indol-5(2H)-yl)-3-(3-methoxyphenylamino)propan-2-ol; 1-(4-azidophenylamino)-3-(3,6-dibromo-9H-carbazol-9-yl)propan-2-ol; 1-(3-azido-6-bromo-9H-carbazol-9-yl)-3-(3-methoxyphenylamino)propan-2-ol; 1-(3,6-dibromo-9H-carbazol-9-yl)-3-(4-methoxyphenoxy)propan-2-ol; 1-(3,6-dichloro-9H-carbazol-9-yl)-3-(phenylsulfonyl)propan-2-ol; 3,6-dibromo-9-(2-fluoro-3-(phenylsulfonyl)propyl)-9H-carbazole; (S)-1-(3,6-dibromo-9H-carbazol-9-yl)-3-(phenylsulfonyl) propan-2-ol; (R)-1-(3,6-dibromo-9H-carbazol-9-yl)-3-(phenylsulfonyl)propan-2-ol; 1-(3,6-dicyclopropyl-9H-carbazol-9-yl)-3-(phenylamino) propan-2-ol; 1-(3,6-diiodo-9H-carbazol-9-yl)-3-(phenylamino)propan-2-ol; 1-(3,6-diethynyl-9H-carbazol-9-yl)-3-(3-methoxyphenylamino) propan-2-ol; 9-(2-hydroxy-3-(3-methoxyphenylamino)propyl)-9H-carbazole-3,6-dicarbonitrile; N-(3-(3,6-dibromo-9H-carbazol-9-yl)-2-fluoropropyl)aniline; 3,6-dibromo-9-(2,2-difluoro-3-phenoxypropyl)-9H-carbazole; N-(3-(3,6-dibromo-9H-carbazol-9-yl)-2-fluoropropyl)-4-methoxyaniline; N-(2-bromo-3-(3,6-dibromo-9H-carbazol-9-yl)propyl)-N-(4-methoxyphenyl)-4-nitrobenzenesulfonamide; Ethyl 2-(4-(3-(3,6-dibromo-9H-carbazol-9-yl)-2-fluoropropylamino)phenoxy)acetate; and N-(3-(3,6-dibromo-9H-carbazol-9-yl)-2-fluoropropyl)-4-(2-(2-methoxyethoxy)ethoxy)aniline; or a pharmaceutically acceptable salt thereof.

In some embodiments, the therapeutic agent is a compound having formula 1-(3,6-dibromo-9H-carbazol-9-yl)-3-(phenylamino)propan-2-ol. In some embodiments, the therapeutic agent is a compound having formula R-1-(3,6-Dibromo-9H-carbazol-9-yl)-3-(3-methoxyphenylamino)-propan-2-ol. In some embodiments, the therapeutic agent is a compound having formula R-1-(3,6-Dibromo-9H-carbazol-9-yl)-3-(3-methoxyphenylamino)-propan-2-ol. In some embodiments, the therapeutic agent is a compound having formula S-1-(3,6-Dibromo-9H-carbazol-9-yl)-3-(3-methoxyphenylamino)-propan-2-ol. In some embodiments, the therapeutic agent is a compound having formula S-1-(3,6-Dibromo-9H-carbazol-9-yl)-3-(3-methoxyphenylamino)-propan-2-ol.

In some embodiments, the therapeutic agent is a compound having formula S-1-(3,6-Dibromo-9H-carbazol-9-yl)-3-(3-methoxyphenylamino)-propan-2-ol. In some embodiments, the therapeutic agent is a compound having formula R-1-(3,6-Dibromo-9H-carbazol-9-yl)-3-(3-methoxyphenylamino)-propan-2-ol. In some embodiments, the therapeutic agent is a compound having formula (+) (dextrorotatory) enantiomer of 1-(3,6-Dibromo-9H-carbazol-9-yl)-3-(3-methoxyphenylamino)-propan-2-ol. In some embodiments, the therapeutic agent is a compound having formula (+) (dextrorotatory) enantiomer of 1-(3,6-Dibromo-9H-carbazol-9-yl)-3-(3-methoxyphenylamino)-propan-2-ol.

In some embodiments, the therapeutic agent is a compound having formula (−) (levorotatory) enantiomer of 1-(3,6-Dibromo-9H-carbazol-9-yl)-3-(3-methoxyphenylamino)-propan-2-ol. In some embodiments, the therapeutic agent is a compound having formula (−) (levorotatory) enantiomer of 1-(3,6-Dibromo-9H-carbazol-9-yl)-3-(3-methoxyphenylamino)-propan-2-ol. In some embodiments, the therapeutic agent is a compound having formula (−) (levorotatory) enantiomer of 1-(3,6-Dibromo-9H-carbazol-9-yl)-3-(3-methoxyphenylamino)-propan-2-ol. In some embodiments, the therapeutic agent is a compound having formula(+) (dextrorotatory) enantiomer of 1-(3,6-Dibromo-9H-carbazol-9-yl)-3-(3-methoxyphenylamino)-propan-2-ol.

In some embodiments, the therapeutic agent is a compound having molecular formula C₂₁H₁₈Br₂N₂O; C₂₁H₁₈F₃N₃OS; C₁₆H₁₉N₅O₂S₂; C₁₅H₁₈N₄O₄S; C₁₄H₁₈IN₅O₂S; C₁₄H₁₈IN₅O₂; C₁₁H₁₄HBrN₃O₂S₂; C₂₁H₂₂N₄O₅; or C₂₀H₁₈ClFN₆O.

In some embodiments, ghrelin and/or a ghrelin variant, is administered in combination with anti-inflammatory compounds, such as an NSAID, indomethacin, COX1/COX2 inhibitors, anti-TNF-α compounds, infliximab, etanercept, adalimumab, erythropoietin/EPO, angiotensin II lowering agents, selective androgen receptor modulators, leptin, agonists of the renin-angiotensin system, opioid receptor agonists, progesterone, amantadine (adamantan-1-amine, C₁₀H₁₇N), peroxisome proliferator-activated receptor gamma agonists, or combinations of the same. In some embodiments, ghrelin and/or a ghrelin variant, is administered in combination with purinergic ligand 2-methylthioladenosine 5′ diphosphate (2MeSADP).

P2Y purinergic receptor agonist can include uridine 5′-di- and triphosphate (UDP, UTP) and their analogs, adenosine 5′-diphosphate (ADP) and its analogs, cytidine 5′-di- and triphosphate (CDP, CTP) and their analogs, and dinucleoside polyphosphate compounds. Exemplary though non-limiting P2Y receptor agonists suitable for use in combination with ghrelin or ghrelin variants may include uridine 5′-di′- and triphosphate (UDP, UTP) and their analogs (Formulae Ia and Ib), 5′-adenosine monophosphate (AMP) and its analogs, adenosine 5′-di- and triphosphate (ADP, ATP) and their analogs (Formulae IIa and IIIb), and cytidine 5′-di- and triphosphate (CDP, CTP) and their analogs (Formulae IIa and IIIb). P2Y receptor agonists also include dinucleotide polyphosphate compounds of general Formula (IV). Examples of P2Y receptor agonists that may be useful to be used in a combination with ghrelin or ghrelin variants for treatment and/or protection of brain injury include 2-MeSADP and N-methanocarba-2MeSADP (“MRS2365”) as disclosed in U.S. Pat. No. 8,618,074, which is incorporated by reference herein.

The present disclosure provides for methods of using a pharmaceutical composition comprising ghrelin or ghrelin variants and P2Y receptor agonists for purpose of treating mild brain injury or concussion. P2Y receptor agonists, including analogs, derivatives and pharmaceutically acceptable salts thereof that may find use in the present treatment methods include, but are not limited to, nucleoside mono-, di-, and triphosphates and dinucleoside polyphosphates. Nucleoside monophosphates may include adenosine 5′-monophosphate (AMP) and its derivatives such as 2-thioether-substituted AMP, e.g., 2-hexylthio AMP. Nucleoside di- and triphosphates may include uridine 5′-di- and triphosphate (UDP and UTP) and their analogs of general formulae Ia and Ib; adenosine 5′-di- and triphosphate (ADP and ATP) and their analogs of general formulae IIa and IIb; and cytosine 5′-di- and triphosphate (CDP and CTP) and their analogs of general formulae IIIa and IIIb; and dinucleoside polyphosphates of general formula IV.

UDP and its analogs are depicted by general formula Ia:

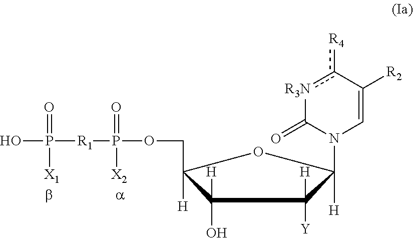

wherein: X₁, and X₂ are each independently either —OH, —O⁻, —SH, or —S⁻; Y is H or OH; R₁ is selected from the group consisting of O, imido, methylene, and dihalomethylene (e.g., dichloromethylene, difluoromethylene); R₂ is selected from the group consisting of H, halogen, alkyl, substituted alkyl, alkoxyl, nitro and azido; R₃ is selected from the group consisting of nothing, H, alkyl, acyl (including arylacyl), and arylalkyl; and R₄ is selected from the group consisting of —OR′, —SR′, NR′, and NR′R″, wherein R′ and R″ are independently selected from the group consisting of H, alkyl, substituted alkyl, aryl, substituted aryl, arylalkyl, alkoxyl, and aryloxyl, and with the proviso that R′ is absent when R⁴ is double bonded from an oxygen or sulfur atom to the carbon at the 4-position of the pyrimidine ring.

Compounds illustrative of the compounds of Formula (Ia) may include, though are not limited to: uridine 5′-diphosphate (UDP); uridine 5′-O-(2-thiodiphosphate)(UDPBS); 5-bromouridine 5′-diphosphate (5-BrUDP); 5-(1-phenylethynyl)-uridine 5′-diphosphate (5-(1-phenylethynyl)UDP); 5-methyluridine 5′-diphosphate (5-methylUDP); 4-hexylthiouridine 5′-diphosphate (4-hexylthioUDP); 4-mercaptouridine 5′-diphosphate (4-mercaptoUDP); 4-methoxyuridine 5′-diphosphate (4-methoxyUDP); 4-(N-morpholino)uridine 5′-diphosphate (4-(N-morpholino)UDP; 4-hexyloxyuridine 5′-diphosphate (4-hexyloxyUDP); N,N-dimethylcytidine 5′-diphosphate (N,N-dimethylCDP); N-hexylcytidine 5′-diphosphate (N-hexylCDP); and N-cyclopentylcytidine 5′-diphosphate (N-cyclopentylCDP).

Certain compounds of Formula Ia (e.g., UDP, dUDP, UDPβS, and 4-mercaptoUDP) are known and may be made in accordance with known procedures or variations thereof, which will be apparent to those skilled in the art. For example, the identification and preparation of certain thiophosphate analogues of nucleoside diphosphates (such as UTPβS) are set forth in U.S. Pat. No. 3,846,402 (Eckstein et al.), and in R. S. Goody and F. Eckstein, J. Am. Chem. Soc. 93: 6252-6257 (1971). Alternatively, UDP, and other analogs thereof are also commercially available from vendors such as Sigma (St. Louis, Mo.) and Pharmacia (Uppsala, Sweden).

UTP and its analogs are depicted by general formula Ib;

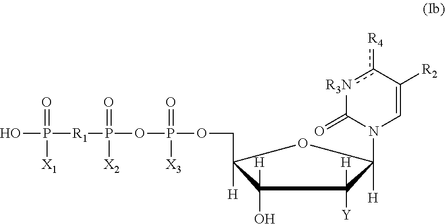

wherein: X₁, X₂ and X₃ are each independently either —OH, —O—, —SH, or —S—, Y is H or OH; R₁, R₂, R₃ and R₄ are defined as above in Formula Ia. Preferably, X₂ and X₃ are O—, R₁ is oxygen or imido, and R₂ is H. Particularly preferred compounds of Formula Ib may include uridine 5′-triphosphate (UTP) and uridine 5′-O-(3-thiotriphosphate) (UTPγS).

ADP and its analogs are depicted by general Formula IIa:

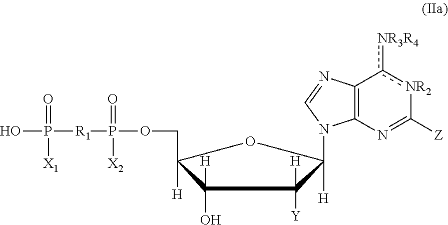

wherein: R₁, X₁, X₂ and Y are defined as in Formula Ia; Z is H, Cl, or SR, wherein R is alkyl (C₁-C₂₀, saturated or unsaturated); R₃ and R₄are H while R₂ is nothing and there is a double bond between N-1 and C-6 (adenine), or R₃ and R₄ are H while R₂ is nothing and Z is SR, or R₃ and R₄ are H while R₂ is O and there is a double bond between N-1 and C-6 (adenine 1-oxide), or R₃, R₄, and R₂ taken together are —CH═CH—, forming a ring from N-6 to N-1 with a double bond between N-6 and C-6 (1,N⁶-ethenoadenine). Particularly preferred compounds of Formula IIa may include 5′-adenosine diphosphate (ADP), 2-methyl-SADP and N-methanocarba-2MeSADP (“MRS2365”).

ATP and its analogs are depicted by general Formula IIb:

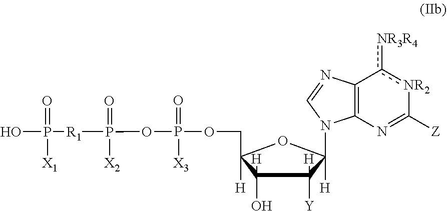

wherein: R₁, X₁, X₂, X₃ and Y are defined as in Formula Ib, and R₂, R₃, R₄ and Z are defined as in Formula IIa.

CDP and its analogs are depicted by general Formula IIIa:

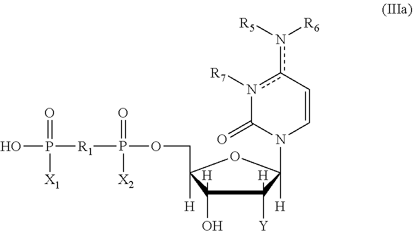

wherein: R₁, X₁, X₂ and Y are defined as in Formula Ia; R₅ and R₆are H while R₇ is nothing and there is a double bond between N-3 and C-4 (cytosine), or R₅, R₆ and R₇taken together are —CH═CH—, forming a ring from N-3 to N-4 with a double bond between N-4 and C-4 (3,N⁴-ethenocytosine), optionally the hydrogen of the 4- or 5-position of the etheno ring is substituted with alkyl, substituted alkyl, aryl; substituted aryl (heteroaryl, etc.), alkoxyl, nitro, halogen, or azido.

CTP and its analogs are depicted by general Formula IIIb:

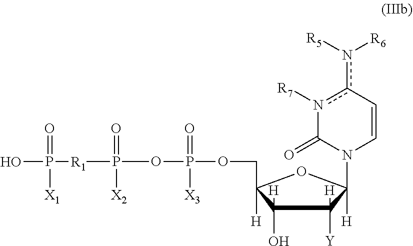

wherein: R₁, X₁, X₂, X₃ and Y are defined as in Formula Ib, and R₅, R₆ and R₇are defined as in Formula IIa. Preferred compounds of Formula IIIb may include cytidine 5′-triphosphate (CTP) and 4-nitrophenyl ethenocytidine 5′-triphosphate.

For simplicity, Formulas I, II, and III, herein illustrate the active compounds in the naturally occurring D-configuration, but it is to be understood that, unless otherwise indicated, the present disclosure also encompasses compounds in the L-configuration, and mixtures of compounds in the D- and L-configurations.

Dinucleoside polyphosphates are depicted by general Formula IV:

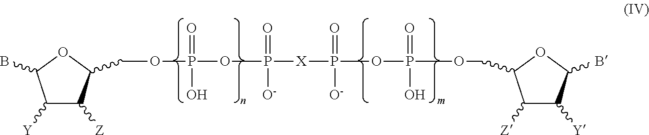

wherein: X is oxygen, methylene, difluoromethylene, imido; n=0, 1 or 2; m=0, 1 or 2; n+m=0, 1, 2, 3 or 4; B and B′ are each independently a purine residue or a pyrimidine residue linked through the 9- or 1-position, respectively; Z═OH or N₃; Z′═OH or N₃; Y═H or OH; and Y′═H or OH. The ribosyl moieties are in the D configuration, as shown, but may be L-, or D- and L-.

A preferred compound of Formula IV includes Formula IVa:

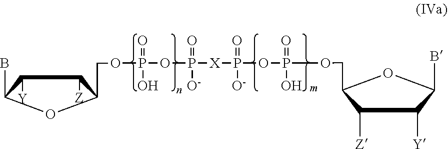

wherein: X═O; n+m=1 or 2; Z, Z′, Y and Y′OH; B and B′ are uracil, thymine, cytosine, guanine, adenine, xanthine, hypoxanthine or as defined in Formulas V and VI; or X═O; n+m=3 or 4; Z, Z′, Y and Y′OH; B=uracil; B′ is uracil, thymine, cytosine, guanine, adenine, xanthine, hypoxanthine or as defined in Formulas V and VI; or X═O; n+m=1 or 2; Z, Y and Z′═OH; Y′═H; B=uracil; B′ is uracil, thymine, cytosine, guanine, adenine, xanthine, hypoxanthine or as defined in Formulas V and VI; or X═O; n+m=0, 1 or 2; Z and Y═OH; Z′═N₃; Y′═H; B=uracil; B′=thymine; or X═O; n+m=0, 1 or 2; Z and Z′═N₃; Y and Y′═H; B and B′=thymine; or X═CH₂, CF₂ or NH; n and m=1; Z, Z′, Y and Y′═OH; B and B′ are uracil, thymine, cytosine, guanine, adenine, xanthine, hypoxanthine or as defined in Formulas V and VI:

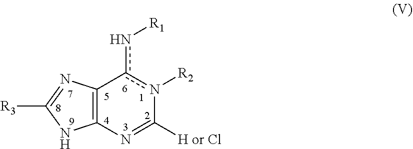

wherein R₁ is hydrogen, C₁₋₈ alkyl, C₃₋₆ cycloalkyl, phenyl, or phenyloxy; wherein at least one hydrogen of said C₁₋₈ alkyl, phenyl, phenyloxy, is optionally substituted with a moiety selected from the group consisting of halogen, hydroxy, C₁₋₄ alkoxy, C₁₋₄ alkyl, C₆₋₁₀ aryl, carboxy, cyano, nitro, sulfonamido, sulfonate, phosphate, sulfonic acid, amino, C₁₋₄ alkylamino, di-C₁₋₄ alkylamino wherein said alkyl groups are optionally linked to form a heterocycle, ω-A(alkyl)CONH(alkyl)-, and ω-A(alkyl)NHCO(alkyl)-, wherein A is amino, mercapto, hydroxy or carboxyl; R₂ is 0 or is absent; or R₁ and R₂ taken together form a 5-membered fused imidazole ring optionally substituted on the 4- or 5-positions of the etheno moiety with C₁₋₄ alkyl, phenyl or phenyloxy, wherein at least one hydrogen of said C₁₋₄ alkyl, phenyl, phenyloxy, is optionally substituted with a moiety selected from the group consisting of halogen, hydroxy, C₁₋₄ alkoxy, C₁₋₄ alkyl, C₆₋₁₀aryl, C₇₋₁₂arylalkyl, carboxy, cyano, nitro, sulfonamido, sulfonate, phosphate, sulfonic acid, amino, C₁₋₄ alkylamino, and di-C₄ alkylamino wherein said dialkyl groups are optionally linked to form a heterocycle; and R₃ is hydrogen, NH₂, C₁₋₈ alkyl, C₃₋₆ cycloalkyl, phenyl; or phenyloxy; wherein at least one hydrogen of said NH₂, C₁₋₈ alkyl, phenyl, or phenyloxy, is optionally substituted with a moiety selected from the group consisting of halogen, hydroxy, C₁₋₄ alkyl, C₆₋₁₀ aryl, C₇₋₁₂ arylalkyl, C₁₋₄ alkoxy, C₇₋₁₂ arylalkyloxy, C₁₋₄ alkylthio, phenylthio, C₇₋₁₂ arylalkylthio, carboxy, cyano, nitro, sulfonamido, sulfonate, phosphate, sulfonic acid, amino, C₁₋₄ alkylamino, phenylamino, C₇₋₁₂ arylalkyamino, di-C₁₋₄ alkyl amino wherein said dialkyl groups are optionally linked to form a heterocycle, ω-A(alkyl)CONH(alkyl)B—, and ω-A(alkyl)NHCO(alkyl)B—, wherein A and B are independently amino, mercapto, hydroxy or carboxyl.

The substituted derivatives of adenine (Formula V) may include adenine 1-oxide; 1,N6-(4- or 5-substituted etheno) adenine; 6-substituted adenine; or 8-substituted aminoadenine, [6-aminohexyl]carbamoylmethyl-ade-nine; and ω-acylated-amino(hydroxy, thiol and carboxy)alkyl(C₂₋₁₀)-adenine, wherein the acyl group is chosen from among, but not limited to, acetyl, trifluororoacetyl, benzoyl, substituted-benzoyl, etc., or the carboxylic moiety is present as its ester or amide derivative, for example, the ethyl or methyl ester or its methyl, ethyl or benzamido derivative.

B and B′, can also be a pyrimidine with the general formula of Formula VI, linked through the 1-position to ribosyl residue:

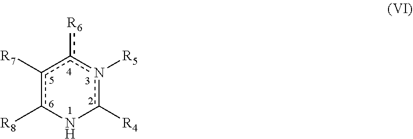

wherein: R⁴ is hydrogen, hydroxy, mercapto, amino, cyano, C₇₋₁₂ arylalkoxy, C₁₋₆ alkylthio, C₁₋₆ alkoxy, C₁₋₆ alkylamino or diC₁₋₄ alkylamino, wherein the alkyl groups are optionally linked to form a heterocycle; R is hydrogen, acetyl, benzoyl, C₁₋₆alkyl, phenyloxy, C₁₋₅alkanoyl, aroyl, or sulphonate; R₆ is hydroxy, mercapto, C₁₋₄ alkoxy, C₇₋₁₂arylalkoxy, C₁.alkylthio, amino, S-phenyl, C₁₋₅ disubstituted amino, triazolyl, C₁₋₆ alkylamino, or di-C₁₋₄ alkylamino wherein said dialkyl groups are optionally linked to form a heterocycle or linked to N₃ to form a substituted ring; or R₅ and R₆ taken together form a 5-membered fused imidazole ring between positions 3 and 4 of the pyrimidine ring and form a 3,N⁴-ethenocytosine derivative, wherein said etheno moiety is optionally substituted on the 4- or 5-positions with C₁₋₄ alkyl; phenyl; or phenyloxy; wherein at least one hydrogen of said C₁₋₄ alkyl; phenyl or phenyloxy is optionally substituted with a moiety selected from the group consisting of halogen, hydroxy, C₁₋₄ alkoxy, C₁₋₄ alkyl, C₆₋₁₀ aryl, C₇₋₁₂ arylalkyl, carboxy, cyano, nitro, sulfonamido, sulfonate, phosphate, sulfonic acid, amino, C₁₋₄ alkylamino, and di C₁₋₄ alkylamino wherein said dialkyl groups are optionally linked to form a heterocycle; R₇ is hydrogen, hydroxy, cyano, nitro, or C₂₋₈ alkenyl; wherein said alkenyl moiety is optionally linked through an oxygen to form a ring, wherein at least one hydrogen of said alkenyl moiety on the carbon adjacent to said oxygen is optionally substituted with C₁₋₆alkyl, phenyl, substituted C₂₋₈ alkynyl, halogen, substituted C₁₋₄ alkyl, CF₃, C₂₋₃ alkenyl, C₂₋₃ alkynyl, allylamino, bromovinyl, ethyl propenoate, or propenoic acid; or R₆ and R₇ together form a 5 or 6-membered saturated or unsaturated ring bonded through N or O at R₆, such ring optionally contains substituents that themselves contain functionalities; provided that when R₈ is amino or substituted amino, R₇ is hydrogen; and R₈ is hydrogen, amino or di-C₁₋₄ alkylamino, C₁₋₄ alkoxy, C₇₋₁₂ arylalkoxy, C₁₋₄ alkylthio, C₇₋₁₂ arylalkylthio, carboxamidomethyl, carboxymethyl, methoxy, methylthio, phenoxy or phenylthio.

In the general structure of Formulae I, II, III, V, and VI above, the dotted lines in the 2- to 6-positions are intended to indicate the presence of single or double bonds in these positions; the relative positions of the double or single bonds being determined by whether the R₄, R₅ and R₆ substituents are capable of keto-enol tautomerism. In the general structures of Formula V and VI above, the acyl groups comprise alkanoyl or aroyl groups. The alkyl groups contain 1 to 8 carbon atoms, particularly 1 to 4 carbon atoms optionally substituted by one or more appropriate substituents, as described below. The aryl groups including the aryl moieties of such groups as aryloxy are preferably phenyl groups optionally substituted by one or more appropriate substituents, as described below. The above-mentioned alkenyl and alkynyl groups contain 2 to 8 carbon atoms, particularly 2 to 6 carbon atoms, e.g., ethenyl or ethynyl, optionally substituted by one or more appropriate substituents as described below.

Appropriate substituents on the above-mentioned alkyl, alkenyl, alkynyl, and aryl groups are selected from, but not limited to, halogen, hydroxy, C₁₋₄alkoxy, C₁₋₄ alkyl, C₆₋₁₂ aryl, C₆₋₁₂ arylalkoxy, carboxy, cyano, nitro, sulfonamido, sulfonate, phosphate, sulfonic, amino and substituted amino wherein the amino is singly or doubly substituted by a C₁₋₄ alkyl, and when doubly substituted, the alkyl groups optionally being linked to form a heterocycle. Dinucleoside polyphosphate compounds useful in this disclosure are P¹, P⁴-di (urdine-5′)-tetraphosphate, dUP₄U, U₂P₃, U₂P₅, dCP₄U, CP₄U, IP51, AP₄A, CP₃U, UP₃A and A²P³.

Some compounds of Formula I, II and III can be made by methods known those skilled in the art; some compounds are commercially available, for example, from Sigma Chemical Co. (St. Louis, Mo. 63178). Compounds of Formulae Ia (UDP and its analogs) can be prepared according to WO 99/09998. Compounds of Formulae Ib, IIb and IIIb (UTP, ATP, CTP and their analogs) can be prepared according to U.S. Pat. No. 5,763,447. Compounds of Formula IV can be made in accordance with known procedures described by Zamecnik, et al., Proc. Natl. Acad. Sci. USA 89, 838-42 (1981); and Ng and Orgel, Nucleic Acids Res. 15:3572-80 (1987), Pendergast et al., U.S. Pat. No. 5,837,861, or variations thereof.

The compounds of the present disclosure also encompass their non-toxic pharmaceutically acceptable salts, such as, but not limited to, an alkali metal salt such as sodium or potassium; an alkaline earth metal salt such as manganese, magnesium or calcium; or an ammonium or tetraalkyl ammonium salt, i.e., NX₄+ (wherein X is C₁₋₄). Pharmaceutically acceptable salts are salts that retain the desired biological activity of the parent compound and do not impart undesired toxicological effects. The present disclosure also encompasses the acylated prodrugs of the compounds disclosed herein. Those skilled in the art recognize various synthetic methodologies that may be employed to prepare non-toxic pharmaceutically acceptable salts and acylated prodrugs of the compounds.

### Sulfonamide Compounds

The present disclosure provides for a number of sulfonamide compounds to be used in a therapeutic combination with ghrelin or ghrelin variants in treating mBI, for example heteroaryl sulfonamide compounds, and other sulfonamide compounds having cyclic moieties. Examples of heteroaryl compounds include oxadiazole and triazole compounds. The compounds can be used in therapeutic applications, including modulation of disorders, diseases or disease symptoms in a subject (e.g., mammal, human, dog, cat, horse). The compounds include useful GHS-R antagonists. Additional compounds are disclosed in U.S. Pat. No. 7,829,589, which is incorporated herein by reference in its entirety for all of its disclosure, including all methods, materials, etc.

The compounds, including stereoisomers thereof, can be created either singly, in small clusters, or in a combinatorial fashion to give structurally diverse libraries of compounds.

In one aspect, the invention features a compound of formula (I)

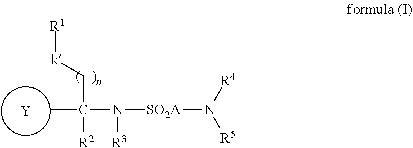

wherein,
R¹ is hydrogen, halo (e.g., fluoro), aryl, heteroaryl, arylalkyl, heteroarylalkyl, cyclyl, cyclylalkyl, heterocyclyl, heterocyclylalkyl, alkyl, alkenyl, alkynyl, or R¹ can be taken together with R² or R³ to form a ring; each of which is optionally substituted with 1-4 R⁶;
k′ is a bond, O, C(O), C(O)O, OC(O), C(O)NR³, NR³C(O), S, SO, SO₂, CR²═R², or C≡C;
n is 0-6, preferably 1-3;
R² is hydrogen, halo (e.g., fluoro), C₁-C₆alkyl, C₂-C₆alkenyl, or C₂-C₆ alkynyl; or R² can be taken together with R¹ to form a ring;
R³ is hydrogen, C₁-C₆alkyl, C₂-C₆alkenyl, or C₂-C₆alkynyl, or R³ can be taken together with R², R⁴, or R⁵ to form a ring; each of which can be optionally substituted with 1-2 R^{6′};

### A is

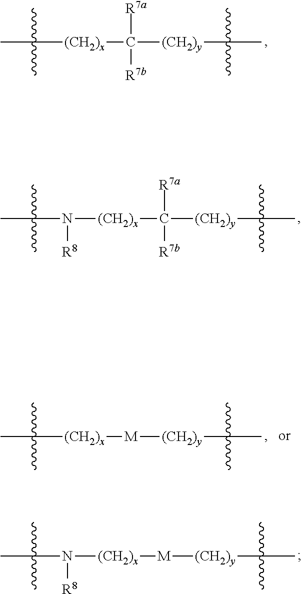

x and y are each independently 0-6;
M is aryl, heteroaryl, cyclyl, or heterocyclyl, each of which is optionally substituted with 1-4 R⁹;
R⁴ and R⁵are each independently hydrogen, alkyl, alkenyl, haloalkyl, cyclyl, or heterocyclyl, or R⁴ and R⁵ can be taken together to form a heterocyclic ring, or R⁴ and R⁵ can be taken together to form an azido moiety, or one or both of R⁴ and R⁵ can independently be joined to one or both or R^{7a} and R^{7b} to form one or more bridges between the nitrogen to which the R⁴ and R⁵ are attached and R^{7a} and R^{7b}, wherein each bridge contains 1 to 5 carbons; or one or both of R⁴ and R⁵ can independently be joined to one or both of R^{7a} and R^{7b} to form to form one or more heterocyclic rings including the nitrogen to which the R⁴ and R⁵ are attached, or one or both of R⁴ and R⁵ can independently be joined to R³ to form a ring, or one or both of R⁴ and R⁵ can independently be joined to R⁸ to form a ring; wherein each R⁴ and R⁵ are optionally independently substituted with 1-5 halo, 1-3 hydroxy, 1-3 alkyl, 1-3 alkoxy, 1-3 amino, 1-3 alkylamino, 1-3 dialklyamino, 1-3 nitrile, or 1-3 haloalkyl;
Y is a monocyclic aryl or monocyclic heteroaryl; each of which is optionally substituted with 1-4 R¹⁰;
each R⁶ and R^{6′} are independently halo, alkyl, alkenyl, alkynyl, cyclyl, heterocyclyl, aryl, heteroaryl, alkoxy, haloalkyl, haloalkyloxy, haloalkylthio, acetyl, cyano, nitro, hydroxy, oxo, C(O)OR², OC(O)R², N(R³)₂, C(O)N(R³)₂, NR³C(O)R², or SR²;
R^{7a} and R^{7b} are each independently hydrogen, alkyl, alkenyl, haloalkyl, cyclyl, cyclylalkyl, or heterocyclyl; or one or both of R^{7a} and R^{7b} can independently be joined to one or both of R⁴ and R⁵ to form one or more bridges between the nitrogen to which the R⁴ and R⁵are attached and R^{7a} and R^{7b}, wherein each bridge contains 1 to 5 carbons; or one or both of R^{7a} and R^{7b} can independently be joined to one or both or R⁴ and R⁵ to form to form one or more heterocyclic rings including the nitrogen to which the R⁴ and R⁵ are attached, or one or both of R^{7a} and R^{7b} can independently be joined with R⁸ to form a ring; wherein each R^{7a} and R^{7b} can be independently optionally substituted with 1-5 halo, 1-3 hydroxy, 1-3 alkyl, 1-3 alkoxy, 1-3 amino, 1-3 alkylamino, 1-3 dialklyamino, 1-3 nitrile, or 1-3 haloalkyl;
R⁸ is hydrogen or C₁-C₆alkyl, or R⁸can be joined with R⁴, R⁵, r^{7a} or R^{7b} to form a ring;
R⁹ is halo, alkyl, cyclyl, heterocyclyl, aryl, heteroaryl, alkoxy, haloalkyl, haloalkyloxy, haloalkylthio, acetyl, cyano, nitro, hydroxy, oxo, C(O)OR², OC(O)R², N(R²)₂, C(O)N(R²)₂, NR²C(O)R²,SR²;
each R¹⁰ is independently alkyl, alkenyl, alkynyl, halo, cyano, carbonyl, aryl, arylalkyl, arylalkenyl, arylalkynyl, cyclyl, cyclylalkyl, alkoxy, alkoxyalkyl, aryloxy, aryloxyalkyl, heterocyclyl, heterocyclylalkyl, heteroaryl, heteroarylalkyl, —OR¹¹, —NR¹¹R^{11′}, —CF₃, —SOR¹², —SO₂R¹², —OC(O)R¹¹, —SO₂NR¹²R^{12′}, —(CH₂)ₘR¹⁴ or R¹⁵; each of which is optionally independently substituted with 1-3 R¹⁶;
R¹¹ and R^{11′} are each independently hydrogen, alkyl, alkenyl, alkynyl, cyclyl, heterocyclyl, aryl or heteroaryl;
R¹² and R^{12′} are each independently hydrogen, alkyl, alkenyl, alkynyl, alkylthioalkyl, alkoxyalkyl, aryl, arylalkyl, heterocyclyl, heteroaryl, heteroarylalkyl, heterocycloalkyl or cyclyl, cyclylalkyl, or R¹² and R^{12′} taken together can be cyclized to form —(CH₂)_{q}X(CH₂)ₛ—; wherein each R¹² and R^{12′} may independently optionally be substituted with 1 to 3 substituents selected from the group consisting of halogen, OR¹¹, alkoxy, heterocycloalkyl, —NR¹C(O)NR¹¹R^{11′}, —C(O)NR¹¹R¹¹, —NR¹¹C(O)R^{11′}, —CN, oxo, —NR¹¹SO₂R^{11′}, —OC(O)R¹¹, —SO₂NR¹¹R^{11′}, —SOR¹³, —S(O)₂R¹³, —COOH and —C(O)OR¹³; each R¹³ is independently alkyl, aryl, arylalkyl, heteroaryl, or heteroarylalkyl, each of which may optionally be substituted with —(CH₂),OH;
each R¹⁴ is independently alkoxy, alkoxycarbonyl, —C(O)NR¹²R^{12′}, —NR¹¹R^{11′}, —C(O)R¹², —NR¹¹C(O)NR¹¹R¹¹′ or —N-heteroaryl;
each R¹⁵ is independently —(CH₂(ₚN(R¹²)C(O)R^{12′}, —(CH₂)ₚCN, —(CH₂)p(N(R¹²)C(O)OR^{12′}, —(CH₂)ₚN(R¹²)C(O)NR¹²R′, —(CH₂)ₚN(R¹²)SO₂R¹², —CH₂)SO₂NR¹²R^{12′}, —(CH₂)ₚC(O)NR¹²R^{12′}, —(CH₂)C(O)OR¹², —(CH²)ₚOC(O)OR¹², —(CH₂)ₚOC(O)R¹², —(CH₂)ₚOC(O)NR¹²R^{12′}, —(CH₂ₚN(R¹²)SO₂NR¹²R^{12′}, —(CH₂)ₚOR¹², —(CH₂)ₚOC(O)N(R¹²)(CH₂)ₘOH, —(CH2)ₚSOR¹², —(CH₂)ₚSO₂R¹², —(CH₂)ₚNR¹¹R¹¹ or —(CH₂)ₚOCH₂C(O)N(R¹²)(CH₂)ₘOH;
each R¹⁶ is independently halo, alkyl, alkenyl, alkynyl, alkoxy, —(CH₂)ₚNR¹¹C(O)NR¹¹R^{11′}, —(CH₂)ₚC(O)NR¹¹R^{11′}, —(CH₂)ₚNR¹¹C(O)R^{11′}, —CN, —(CH₂)ₚNR¹¹SO₂R¹¹, —(CH₂)ₚOC(O)R¹¹, —(CH₂)ₚSO₂NR¹¹R¹¹′, —(CH₂)ₚSOR¹³, —(CH₂)ₚCOOH or —(CH₂)ₚC(O)OR¹³;
X is CR¹¹R^{11′}, O, S, S(O), S(O)₂, or NR¹¹;
m is an integer between 1 and 6;
p is an integer from 0 to 5;
q and s are each independently an integer between 1 and 3; and
w is an integer between 0 and 5.

In some embodiments, formula (I), comprises an enriched preparation of formula (I′) formula (I′)

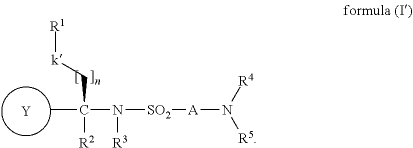

In some embodiments, formula (I), comprises an enriched preparation of formula (I″)

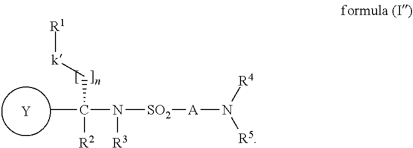

In some embodiments, n is 1; k′ is a bond or O; and R¹ is aryl, heteroaryl, arylalkyl, or heteroarylalkyl.

In some embodiments, n is 1; k′ is O; and R¹ is arylalkyl. For example, R¹ can be phenylmethyl.

In some embodiments, n is 2; k′ is a bond; and R¹ is aryl.

In some embodiments, n is 0 or 1; k′ is a bond; and

R¹ is alkyl, for example unsubstituted or substituted with one R⁶. For example, R¹ can be a branched alkyl such as one of the following.

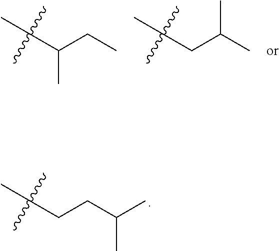

In some embodiments, R² is hydrogen or C₁-C₃alkyl.

In some embodiments n is 0 and k′ is a bond. Exemplary R¹ moieties include methyl, and ethyl. Preferred R¹ moieties include methyl. In some embodiments R¹ is unsubstituted methyl or methyl or ethyl substituted with C(O)N(R³)₂.

In some embodiments n is 0 and k′ is a bond, and R¹ and R² are both methyl.

In some embodiments, n is 0; k′ is a bond; and R¹ is hydrogen.

In some embodiments R³ is hydrogen.

In some embodiments, R¹ and R³ together from a heterocyclic ring such as a pyrrolidine or an azetidine ring. The heterocyclic ring can be unsubstituted or substituted, for example, with 1-2 R⁶.

In some embodiments, R¹ and R² together form a ring.

In some embodiments, A is

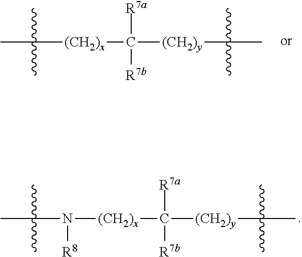

For example, A can be

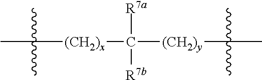

### or A can be

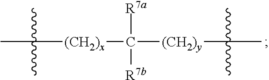

wherein
R^{7a} and R^{7b} are H;
x is 1; and
y is 0 or 1.

In some embodiments, A is CH₂CH₂ or CH₂CH₂CH₂; and each R⁴ and R⁵ is independently alkyl, or R⁴ and R⁵, when taken together, form a heterocyclic ring. In some embodiments, R^{7a} and R^{7b} can each be H.

In some embodiments, at least one of R^{7a} or R^{7b} is taken together with at least one or R⁴ or R⁵ to form a heterocyclic ring including the nitrogen to which the R⁴ and R⁵ are attached.

In some embodiments, R^{7a} and R^{7b}are each independently alkyl; R⁴ and R⁵ are each independently hydrogen or alkyl; and x and y are each independently 0 or 1;

In some embodiments,

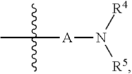

taken together is

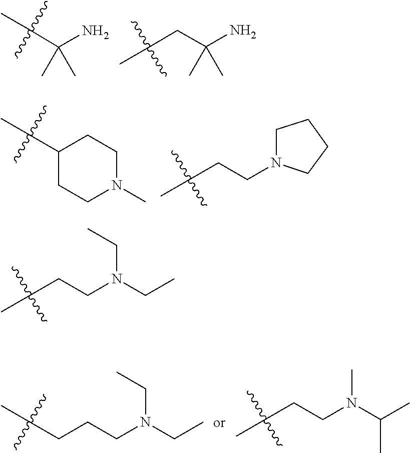

In some embodiments,

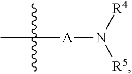

taken together is

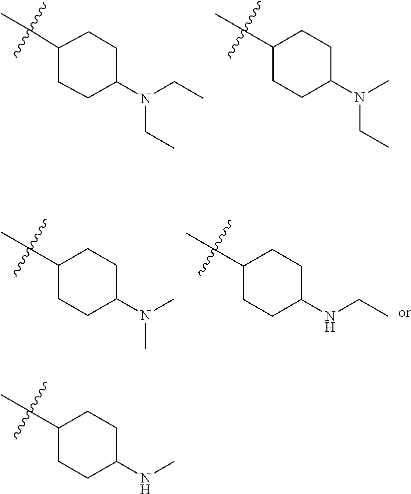

In some embodiments,

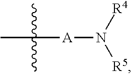

taken together is

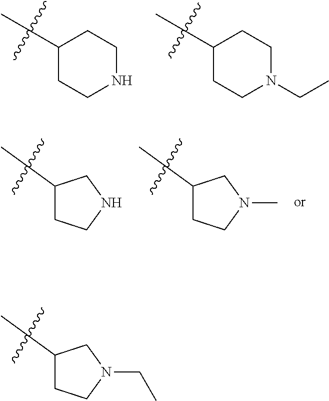

In some embodiments,

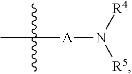

taken together is

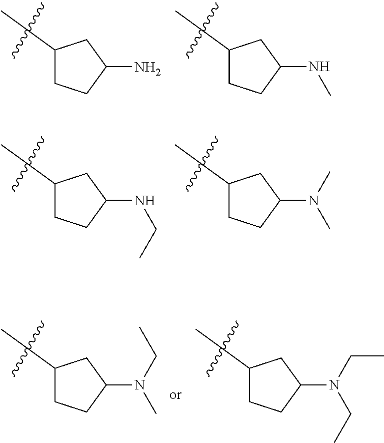

In some embodiments,

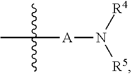

taken together is

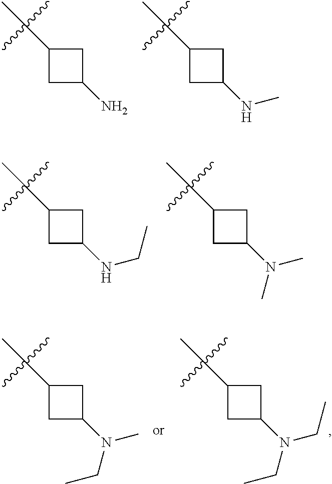

In some embodiments, Y is a monocyclic heteroaromatic moiety, for example a nitrogen containing heteraromatic moiety such as nitrogen containing five membered heteroaromatic moiety.

In some embodiments, Y is a heterocyclic moiety containing at least two heteroatoms, for example, a five membered heterocyclic moiety containing at least two heteroatoms or at least three heteroatoms.

In some embodiments Y is substituted with one R¹⁰. R¹⁰can be positioned, for example, 1,3 relative to the point of attachment of Y to the adjacent chain carbon or 1,2 relative to the point of attachment of Y to the adjacent chain carbon.

In some embodiments, R¹⁰ is aryl or heteroaryl, for example a monocyclic aryl or monocyclic heteroaryl such as phenyl, pyridyl, oxazolyl, thiazolyl, or thiophenyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is a bicyclic heteroaryl, for example indolyl, benzimidazolyl, benzoxazolyl, benzofuranyl, benzothiophenyl, or benzthiazolyl. In some embodiments, R¹⁰ is substituted with 1-3 R¹⁶I. In some embodiments, R¹⁶ is halo, alkyl, or alkoxy, for example chloro, fluoro, methyl, cyano, or methoxy.

In some embodiments, R¹⁰ is arylalkyl or heteroarylalky, for example a monocyclic or bicyclic arylalkyl or monocyclic or bicyclic heteroaryalkyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ includes an unsaturated or partially unsaturated cyclic moiety, for example a cyclyl or heterocyclyl moiety. The cyclic moiety can either be directly attached to Y or attached via a linker such as an alkylenyl linker. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments Y is oxadiazole or triazole.

In another aspect, the invention features a compound of formula (II),

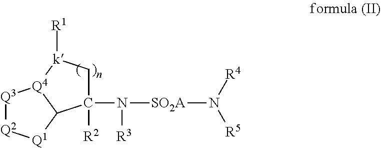

wherein,
Q¹, Q², Q³ and Q⁴ together with the carbon to which they are attached form a heteroaryl moiety, and each Q¹, Q², Q³ and Q⁴ is independently S, O, N, CR², CR¹⁰, NR², or NR¹⁰.

In some embodiments, the compound of formula (II), comprises an enriched preparation of formula (II′)

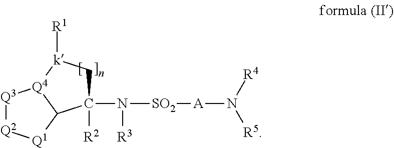

In some embodiments, the compound of formula (II), comprises an enriched preparation of formula (II″)

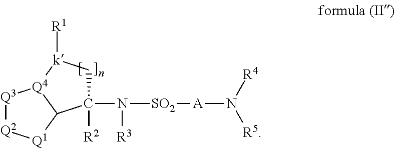

In some embodiments, Q¹ and Q⁴ are each independently S, O, N, or NR¹⁰.

In some embodiments, Q¹ and Q³ are each independently S, O, N, or NR¹⁰.

In some embodiments, Q² is CR² or CR¹⁰.

In some embodiments, Q² is S, O, N, or NR¹⁰.

In some embodiments, at least one of Q² or Q³ is CR² or CR¹⁰.

In some embodiments, at least two of Q¹, Q², Q³, or Q⁴ is S, O, N, or NR¹⁰.

In some embodiments, Q¹, Q², and Q³are each independently S, O, N, or NR¹⁰.

In some embodiments, Q¹ is NR¹⁰.

In some embodiments, one of Q², Q³, or Q⁴ is CR².

In some embodiments, Q² is CR¹⁰.

In some embodiments, Q³ is CR².

In some embodiments, Q¹, Q², Q³ and Q⁴together form

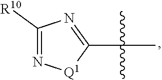

In some embodiments, Q¹ is NR².

In some embodiments, Q¹, Q², Q³ and Q⁴ together form

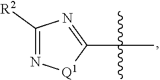

In some embodiments, Q¹ is NR¹⁰.

In another aspect, the invention features a compound of formula (III),

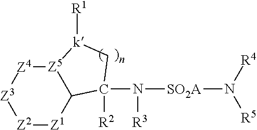

wherein,
Z¹, Z², Z³, Z⁴, and Z⁵ together form an aryl or heteroaryl moiety, and each Z¹, Z², Z³, Z⁴, and Z⁵ is independently N, CR¹⁰, or CR².

In some embodiments, the compound of formula (III), comprises an enriched preparation of formula (III′)

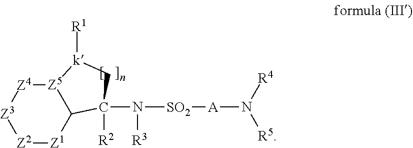

In some embodiments, the compound of formula (III), comprises an enriched preparation of formula (III′)

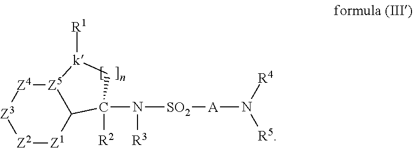

In some embodiments, one of Z¹, Z², Z³, Z⁴, and Z⁵ is N.

In some embodiments, two of Z¹, Z², Z³, Z⁴, and Z⁵ are N.

In some embodiments, three of Z¹, Z², Z³, Z⁴, and Z⁵ is N.

In some embodiments, two of Z¹ and Z² are N.

In some embodiments, two of Z¹ and Z³ are N.

In some embodiments, two of Z¹ and Z⁴ are N.

In some embodiments, two of Z¹, Z³, and Z⁵ are N.

In some embodiments, the compound is a compound of formula (I), wherein Y is substituted with a single substituent R¹⁰. For example, R¹⁰ can be aryl or heteroaryl, optionally substituted with up to three independent R¹⁶.

In some embodiments, R¹⁰ is aryl or heteroaryl, for example a monocyclic aryl or monocyclic heteroaryl such as phenyl, pyridyl, oxazolyl, thiazolyl, or thiophenyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is a bicyclic heteroaryl, for example indolyl, benzimidazolyl, benzoxazolyl, benzofuranyl, benzothiophenyl, or benzthiazolyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is arylalkyl or heteroarylalky, for example a monocyclic or bicyclic arylalkyl or monocyclic or bicyclic heteroaryalkyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ includes an unsaturated or partially unsaturated cyclic moiety, for example a cyclyl or heterocyclyl moiety. The cyclic moiety can either be directly attached to Y or attached via a linker such as an alkylenyl linker. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is R¹⁵.

In some embodiments, Y is substituted with a second R¹⁰, for example an alkyl, halo or alkoxy.

In some embodiments, R¹ is aryl, heteroaryl, arylalkyl, or heteroarylalkyl; k′ is a bond or O; n is 1 or 2; R² and R³are both hydrogen;

### A is

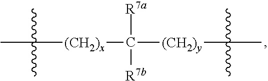

x and y are each independently 0-6;
R⁴ and R⁵ are each independently hydrogen or alkyl;
Y is a monocyclic aryl or monocyclic heteroaryl; each of which is optionally substituted with 1-4 R¹⁰;
each R¹⁰ is independently alkyl, alkenyl, alkynyl, halo, cyano, carbonyl, aryl, arylalkyl, arylalkenyl, arylalkynyl, cyclyl, cyclylalkyl, alkoxy, alkoxyalkyl, aryloxy, aryloxyalkyl, heterocyclyl, heterocyclylalkyl, heteroaryl, heteroarylalkyl, —OR, —NR¹¹R^{11′}, —CF₃, —SOR¹², —SO₂R¹², —OC(O)R¹¹, —SO₂NR¹²R^{12′}, —(CH₂)ₘR¹⁴ or R¹⁵; each of which is optionally independently substituted with 1-3 R¹⁶;
R¹¹ and R^{11′} are each independently hydrogen, alkyl, alkenyl, alkynyl, cyclyl, heterocyclyl, aryl or heteroaryl;
R¹² and R^{12′} are each independently hydrogen, alkyl, alkenyl, alkynyl, alkylthioalkyl, alkoxyalkyl, aryl, arylalkyl, heterocyclyl, heteroaryl, heteroarylalkyl, heterocycloalkyl or cyclyl, cyclylalkyl, or R¹² and R^{12′} taken together can be cyclized to form —(CH₂)_{q}X(CH₂)ₛ—; wherein each R¹² and r^{12′} may independently optionally be substituted with 1 to 3 substituents selected from the group consisting of halogen, OR¹¹, alkoxy, heterocycloalkyl, —NR¹¹C(O)NR¹¹R^{11′}, —C(O)NR¹¹R^{11′}, —NR¹¹C(O)R^{11′}, —CN, oxo, —NR¹¹SO₂R^{11′}, —OC(O)R¹¹, —SO₂NR¹¹R^{11′}, —SOR¹³, —S(O)₂R¹³, —COOH and —C(O)OR¹³; each R¹³ is independently alkyl, aryl, arylalkyl, heteroaryl, or heteroarylalkyl, each of which may optionally be substituted with —(CH₂)_{w}OH;
each R¹⁴ is independently alkoxy, alkoxycarbonyl, —C(O)NR¹²R^{12′}, —NR¹¹R^{11′}, —C(O)R¹², —NR¹¹C(O)NR¹¹R^{11′} or —N-heteroaryl;
each R¹⁵ is independently —(CH₂)ₚN(R¹²)C(O)R^{12′}, —(CH₂)ₚCN, —(CH₂)ₚN(R¹²)C(O)OR¹², —(CH₂)ₚN(R¹²)C(O)NR¹²R′, —(CH₂)ₚN(R¹²)SO₂R¹², —(CH₂)SO₂NR¹²R^{12′}, —(CH₂)ₚC(O)NR¹²R^{12′}, —(CH₂)ₚC(O)OR¹², —(CH²)ₚOC(O)OR¹², —(CH₂)ₚOC(O)R¹², —(CH₂)ₚOC(O)NR¹²R^{12′}, —(CH₂)ₚN(R¹²)SO₂NR¹²R^{12′}, —(CH₂)ₚOR¹², —(CH₂)ₚOC(O)N(R¹²)(CH₂)ₘOH, —(CH₂)ₚSOR¹², —(CH₂)ₚSO₂R¹², —(CH₂)ₚNR¹¹R¹¹ or —(CH₂)ₚOCH₂C(O)N(R¹²)(CH₂)ₘOH;
each R¹⁶ is independently halo, alkyl, alkenyl, alkynyl, alkoxy, —(CH₂)ₚNR¹¹C(O)NR¹¹R^{11′}, —(CH₂)ₚC(O)NR¹¹R^{11′}, —(CH₂)ₚNR¹¹C(O)R^{11′}, —CN, —(CH₂)ₚNR¹¹SO₂R^{11′}, —(CH₂)ₚOC(O)R¹¹, —(CH₂)ₚSO₂NR¹¹R^{11′}, —(CH₂)ₚSOR¹³, —(CH₂)ₚCOOH or —(CH₂)ₚC(O)OR¹³;
X is CR¹¹R^{11′}, O, S, S(O)₂, or NR¹¹;
m is an integer between 1 and 6;
p is an integer from 0 to 5;
q and s are each independently an integer between 1 and 3; and
w is an integer between 0 and 5.

For example, in some embodiments, n is 1; k′ is a bond or O; and R¹ is aryl, heteroaryl, arylalkyl, or heteroarylalkyl. In some embodiments, n is 1; k′ is O; and R¹ is arylalkyl, for example phenylmethyl. In some embodiments, n is 2; k′ is a bond; and r¹ is aryl.

For example, in some embodiments, R^{7a} and R^{7b} are H; x is 1; and y is 0 or 1. In some embodiments, A is CH₂CH₂ or CH₂CH₂CH₂.

In some embodiments, each R⁴ and R⁵ is independently alkyl, for example, methyl or ethyl, preferably ethyl.

In some embodiments, Y is a monocyclic heteroaromatic moiety, for example a nitrogen containing heteraromatic moiety such as nitrogen containing five membered heteraromatic moiety.

In some embodiments, Y is a heterocyclic moiety containing at least two heteroatoms, for example, a five membered heterocyclic moiety containing at least two heteroatoms or at least three heteroatoms.

In some embodiments Y is substituted with one R¹⁰. R¹⁰can be positioned, for example, 1,3 relative to the point of attachment of Y to the adjacent chain carbon or 1,2 relative to the point of attachment of Y to the adjacent chain carbon.

In some embodiments, R¹⁰ is aryl or heteroaryl, for example a monocyclic aryl or monocyclic heteroaryl such as phenyl, pyridyl, oxazolyl, thiazolyl, or thiophenyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is a bicyclic heteroaryl, for example indolyl, benzimidazolyl, benzoxazolyl, benzofuranyl, benzothiophenyl, or benzthiazolyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is arylalkyl or heteroarylalky, for example a monocyclic or bicyclic arylalkyl or monocyclic or bicyclic heteroaryalkyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ includes an unsaturated or partially unsaturated cyclic moiety, for example a cyclyl or heterocyclyl moiety. The cyclic moiety can either be directly attached to Y or attached via a linker such as an alkylenyl linker. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments Y is oxadiazole or triazole.

In some embodiments, Y is

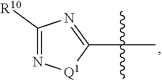

wherein Q1 is O or NR², preferably O or NH. In some embodiments, R¹⁰ is aryl, arylalkyl, heteroaryl, or heteroarylalkyl, for example optionally substituted with one or more R¹⁶. In some embodiments, R¹⁰ is substituted with one R¹⁶, such as halo (e.g., fluoro or chloro) or alkoxy.

In some embodiments, the compounds has a formula (Ia)

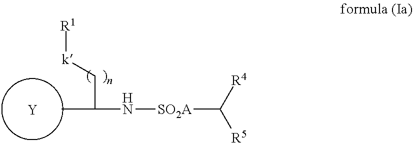

In some embodiments, R¹ is aryl, heteroaryl, arylalkyl, or heteroarylalkyl;

k′ is a bond or O;
n is 1 or 2;
A is CH₂, CH₂CH₂, or CH₂CH₂CH₂;
R⁴ and R⁵ are each independently hydrogen or alkyl;
Y is a monocyclic aryl or monocyclic heteroaryl; each of which is optionally substituted with 1-4 R¹⁰;
each R¹⁰ is independently alkyl, alkenyl, alkynyl, halo, cyano, carbonyl, aryl, arylalkyl, arylalkenyl, arylalkynyl, cyclyl, cyclylalkyl, alkoxy, alkoxyalkyl, aryloxy, aryloxyalkyl, heterocyclyl, heterocyclylalkyl, heteroaryl, heteroarylalkyl, —OR¹¹, —NR¹¹R^{11′}, —CF₃, —SOR¹², —SO₂R¹², —OC(O)R¹¹, —SO₂NR¹²R^{12′}, —(CH₂)ₘR¹⁴ or R¹⁵; each of which is optionally independently substituted with 1-3 R¹⁶;
R¹¹ and R^{11′} are each independently hydrogen, alkyl, alkenyl, alkynyl, cyclyl, heterocyclyl, aryl or heteroaryl;
R¹² and R^{12′} are each independently hydrogen, alkyl, alkenyl, alkynyl, alkylthioalkyl, alkoxyalkyl, aryl, arylalkyl, heterocyclyl, heteroaryl, heteroarylalkyl, heterocycloalkyl or cyclyl, cyclylalkyl, or R¹² and R^{12′} taken together can be cyclized to form —(CH₂)_{q}X(CH₂)ₛ—; wherein each R¹² and R^{12′} may independently optionally be substituted with 1 to 3 substituents selected from the group consisting of halogen, OR¹¹, alkoxy, heterocycloalkyl, —NR¹¹C(O)NR¹¹R^{11′}, —C(O)NR¹¹R^{11′}, —NR¹¹C(O)R^{11′}, —CN, oxo, —NR¹¹SO₂R^{11′}, —OC(O)R¹¹, —SO₂NR¹¹R^{11′}, —SOR¹³, —S(O)₂R¹³, —COOH and —C(O)OR¹³; each R¹³ is independently alkyl, aryl, arylalkyl, heteroaryl, or heteroarylalkyl, each of which may optionally be substituted with —(CH₂)_{w}OH;
each R¹⁴ is independently alkoxy, alkoxycarbonyl, —C(O)NR¹²R^{12′}, —NR¹¹R^{11′}, —C(O)R¹², —NR¹¹C(O)NR¹¹R^{11′} or —N-heteroaryl;
each R¹⁵ is independently —(CH₂)ₚN(R¹²)C(O)R^{12′}, —(CH₂)ₚCN, —(CH₂)ₚN(R¹²)C(O)OR^{12′}, —(CH₂)ₚN(R¹²)C(O)NR¹²R^{12′}, —(CH₂)ₚN(R¹²)SO₂R¹², —(CH₂)ₚSO₂NR¹²R^{12′}, —(CH₂)ₚC(O)NR¹²R^{12′}, —(CH₂)ₚC(O)OR¹², —(CH²)ₚOC(O)OR¹², —(CH₂)ₚOC(O)R¹², —(CH₂)ₚOC(O)NR¹²R^{12′}, —(CH₂)ₚN(R¹²)SO₂NR¹²R^{12′}, —(CH₂)ₚOR¹², —(CH2)ₚOC(O)N(R¹²)(CH2)ₘOH, —(CH2)SOR¹², —(CH₂)ₚSO₂R¹², —(CH₂)ₚNR¹¹R¹¹ or —(CH₂)ₚOCH₂C(O)N(R¹²)(CH₂)ₘOH;
each R¹⁶ is independently halo, alkyl, alkenyl, alkynyl, alkoxy, —(CH₂)ₚNR¹¹C(O)NR¹¹R^{11′}, —(CH₂)ₚC(O)NR¹¹R^{11′}, —(CH₂)ₚNR¹¹C(O)R^{11′}, —CN, —(CH₂)ₚNR¹¹SO₂R^{11′}, —(CH₂)ₚOC(O)R¹¹, —(CH₂)ₚSO₂NR¹¹R^{11′}, —(CH₂)ₚSOR¹³, —(CH₂)ₚCOOH or —(CH₂)ₚC(O)OR¹³;
X is CR¹¹R^{11′}, O, S, S(O)₂, OR NR¹¹;
m is an integer between 1 and 6;
p is an integer from 0 to 5;
q and s are each independently an integer between 1 and 3; and
w is an integer between 0 and 5.

For example in some embodiments, n is 1; k′ is a bond or O; and R¹ is aryl, heteroaryl, arylalkyl, or heteroarylalkyl. In some embodiments, n is 1; k′ is O; and R¹ is arylalkyl, for example phenylmethyl. In some embodiments, n is 2; k′ is a bond; and R¹ is aryl.

In some embodiments, A is CH₂CH₂ or CH₂CH₂CH₂, preferably CH₂CH₂CH₂.

In some embodiments, each R⁴ and R⁵ is independently alkyl, for example, methyl or ethyl, preferably ethyl.

In some embodiments, Y is a monocyclic heteroaromatic moiety, for example a nitrogen containing heteraromatic moiety such as a nitrogen containing five membered heteraromatic moiety.

In some embodiments, Y is a heterocyclic moiety containing at least two heteroatoms, for example, a five membered heterocyclic moiety containing at least two heteroatoms or at least three heteroatoms.

In some embodiments Y is substituted with one R¹⁰. R¹⁰ can be positioned, for example, 1,3 relative to the point of attachment of Y to the adjacent chain carbon or 1,2 relative to the point of attachment of Y to the adjacent chain carbon.

In some embodiments, R¹⁰ is aryl or heteroaryl, for example a monocyclic aryl or monocyclic heteroaryl such as phenyl, pyridyl, oxazolyl, thiazolyl, or thiophenyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is a bicyclic heteroaryl, for example indolyl, benzimidazolyl, benzoxazolyl, benzofuranyl, benzothiophenyl, or benzthiazolyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is arylalkyl or heteroarylalky, for example a monocyclic or bicyclic arylalkyl or monocyclic or bicyclic heteroaryalkyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ includes an unsaturated or partially unsaturated cyclic moiety, for example a cyclyl or heterocyclyl moiety. The cyclic moiety can either be directly attached to Y or attached via a linker such as an alkylenyl linker. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments Y is oxadiazole or triazole.

In some embodiments, Y is

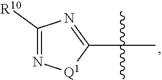

wherein Q1 is O or NR², preferably O or NH. In some embodiments, R¹⁰ is aryl, arylalkyl, heteroaryl, or heteroarylalkyl, for example optionally substituted with one or more R¹⁶. In some embodiments, R¹⁰ is substituted with one R¹⁶, such as halo (e.g., fluoro or chloro) or alkoxy.

In some embodiments, R¹ is hydrogen or alkyl, for example unsubstituted or substituted with one R⁶;

n is 0 or 1;
k′ is a bond; and
R² and R³ each independently hydrogen or C₁-C₆ alkyl;

### A is

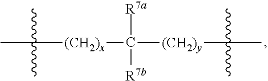

x and y are each independently 0-6;
R⁴ and R⁵ are each independently hydrogen or alkyl;
Y is a monocyclic aryl or monocyclic heteroaryl; each of which is optionally substituted with 1-4 R¹⁰;
each R¹⁰ is independently alkyl, alkenyl, alkynyl, halo, cyano, carbonyl, aryl, arylalkyl, arylalkenyl, arylalkynyl, cyclyl, cyclylalkyl, alkoxy, alkoxyalkyl, aryloxy, aryloxyalkyl, heterocyclyl, heterocyclylalkyl, heteroaryl, heteroarylalkyl, —OR″, —NR¹¹R^{11′}, —CF₃—SOR¹², —SO₂R¹², —OC(O)R¹¹, —SO₂NR¹²R^{12′}, —(CH₂)ₘR¹⁴ or R¹⁵; each of which is optionally independently substituted with 1-3 R¹⁶;
R¹¹ and R^{11′} are each independently hydrogen, alkyl, alkenyl, alkynyl, cyclyl, heterocyclyl, aryl or heteroaryl;
R¹² and R^{12′} are each independently hydrogen, alkyl, alkenyl, alkynyl, alkylthioalkyl, alkoxyalkyl, aryl, arylalkyl, heterocyclyl, heteroaryl, heteroarylalkyl, heterocycloalkyl or cyclyl, cyclylalkyl, or R¹² and R^{12′} taken together can be cyclized to form —(CH₂)_{q}X(CH₂)ₛ—; wherein each R¹² and R^{12′} may independently optionally be substituted with 1 to 3 substituents selected from the group consisting of halogen, OR¹¹, alkoxy, heterocycloalkyl, —NR¹¹C(O)NR¹¹R^{11′}, —C(O)NR¹¹R^{11′}, —NR¹¹C(O)R^{11′}, —CN, oxo, —NR¹¹SO₂R^{11′}, —OC(O)R¹¹, —SO₂NR¹¹R^{11′}, —SOR¹³, —S(O)₂R¹³, —COOH and —C(O)OR¹³;
each R¹³ is independently alkyl, aryl, arylalkyl, heteroaryl, or heteroarylalkyl, each of which may optionally be substituted with —(CH₂)_{w}OH;
each R¹⁴ is independently alkoxy, alkoxycarbonyl, —C(O)NR¹²R^{12′}, —NR¹¹R^{11′}, —C(O)R¹², —NR¹¹C(O)NR¹¹R^{11′} or —N-heteroaryl;
each R¹⁵ is independently —(CH₂)ₚN(R¹²)C(O)R^{12′}, —(CH₂)ₚCN, —(CH₂)ₚN(R¹²)C(O)OR¹², —(CH₂)ₚN(R¹²)C(O)NR¹²R^{12′}, —(CH₂)ₚN(R¹²)SO₂R¹², —(CH₂)SO₂NR¹²R^{12′}, —(CH₂)ₚC(O)NR¹²R^{12′}, —(CH₂)ₚC(O)OR¹², —(CH²)ₚOC(O)OR¹², —(CH₂)ₚOC(O)R¹², —(CH₂)ₚOC(O)NR¹²R^{12′}, —(CH₂)ₚN(R¹²)SO₂NR¹²R^{12′}, —(CH₂)ₚOR¹², —(CH₂)ₚOC(O)N(R¹²)(CH₂)ₘOH, —(CH₂)ₚSOR¹², —(CH₂)ₚSO₂R¹², —(CH₂)ₚNR¹¹R¹¹ or —(CH₂)ₚOCH₂C(O)N(R¹²)(CH₂)ₘOH;
each R¹⁶ is independently halo, alkyl, alkenyl, alkynyl, alkoxy, —(CH₂)ₚNR¹¹C(O)NR¹¹R^{11′}, —(CH₂)ₚC(O)NR¹¹R^{11′}, —(CH₂)ₚNR¹¹C(O)R^{11′}, —CN, —(CH₂)ₚNR¹¹SO₂R^{11′}, —(CH₂)ₚOC(O)R¹¹, —(CH₂)ₚSO₂NR¹¹R^{11′}, —(CH₂)ₚSOR¹³, —(CH₂)ₚCOOH or —(CH₂)ₚC(O)OR¹³;
X is CR¹¹R^{11′}, O, S, S(O), S(O)₂, or NR¹¹;
m is an integer between 1 and 6;
p is an integer from 0 to 5;
q and s are each independently an integer between 1 and 3; and
w is an integer between 0 and 5.

In some embodiments, n is 0 or 1; k′ is a bond; and R¹ is alkyl, for example unsubstituted or substituted with one R⁶.

In some embodiments n is 0 and k′ is a bond. Exemplary R¹ moieties include methyl, and ethyl. Preferred R¹ moieties include methyl. In some embodiments R¹ is unsubstituted methyl or methyl or ethyl substituted with C(O)N(R³)₂.

In some embodiments,

n is 0 or 1;
k′ is a bond; and
R¹ is alkyl, for example unsubstituted or substituted with one R⁶. For example, R¹ can be a branched alkyl such as one of the following

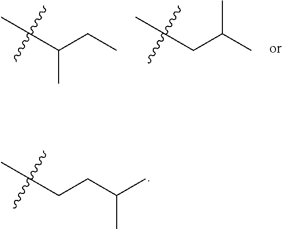

In some embodiments n is 0 and k′ is a bond, and R¹ and R³are both methyl.

In some embodiments, n is 0; k′ is a bond; and R¹ is hydrogen.

In some embodiments, A is CH₂CH₂ or CH₂CH₂CH₂, preferably CH₂CH₂CH₂.

In some embodiments, each R⁴ and R⁵ is independently alkyl, for example, methyl or ethyl, preferably ethyl.

In some embodiments, Y is a monocyclic heteroaromatic moiety, for example a nitrogen containing heteraromatic moiety such as a nitrogen containing five membered heteraromatic moiety.

In some embodiments, Y is a heterocyclic moiety containing at least two heteroatoms, for example, a five membered heterocyclic moiety containing at least two heteroatoms or at least three heteroatoms.

In some embodiments Y is substituted with one R¹⁰. R¹⁰can be positioned, for example, 1,3 relative to the point of attachment of Y to the adjacent chain carbon or 1,2 relative to the point of attachment of Y to the adjacent chain carbon.

In some embodiments, R¹⁰ is aryl or heteroaryl, for example a monocyclic aryl or monocyclic heteroaryl such as phenyl, pyridyl, oxazolyl, thiazolyl, or thiophenyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is a bicyclic heteroaryl, for example indolyl, benzimidazolyl, benzoxazolyl, benzofuranyl, benzothiophenyl, or benzthiazolyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is arylalkyl or heteroarylalky, for example a monocyclic or bicyclic arylalkyl or monocyclic or bicyclic heteroaryalkyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ includes an unsaturated or partially unsaturated cyclic moiety, for example a cyclyl or heterocyclyl moiety. The cyclic moiety can either be directly attached to Y or attached via a linker such as an alkylenyl linker. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments Y is oxadiazole or triazole.

In some embodiments, Y is

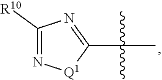

wherein Q1 is O or NR², preferably O or NH. In some embodiments, R¹⁰ is aryl, arylalkyl, heteroaryl, or heteroarylalkyl, for example optionally substituted with one or more R¹⁶. In some embodiments, R¹⁰ is substituted with one R¹⁶, such as halo (e.g., fluoro or chloro) or alkoxy.

In some embodiments, the compounds has a formula (Ib)

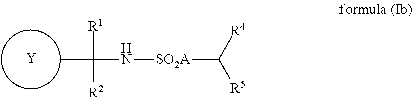

In some embodiments, R¹ is hydrogen or alkyl;

A is CH₂, CH₂CH₂, or CH₂CH₂CH₂;
R² is hydrogen or C₁-C₃ alkyl;
R⁴ and R⁵ are each independently hydrogen or alkyl;
Y is a monocyclic aryl or monocyclic heteroaryl; each of which is optionally substituted with 1-4 R¹⁰;
each R¹⁰ is independently alkyl, alkenyl, alkynyl, halo, cyano, carbonyl, aryl, arylalkyl, arylalkenyl, arylalkynyl, cyclyl, cyclylalkyl, alkoxy, alkoxyalkyl, aryloxy, aryloxyalkyl, heterocyclyl, heterocyclylalkyl, heteroaryl, heteroarylalkyl, —OR¹¹, —NR¹¹R^{11′}, —CF₃, —SOR¹², —SO₂R¹², —OC(O)R¹¹, —SO₂NR¹²R^{12′}, —(CH₂)ₘR¹⁴ or R¹⁵; each of which is optionally independently substituted with 1-3 R¹⁶;
R¹¹ and R^{11′} are each independently hydrogen, alkyl, alkenyl, alkynyl, cyclyl, heterocyclyl, aryl or heteroaryl;
R¹² and R^{12′} are each independently hydrogen, alkyl, alkenyl, alkynyl, alkylthioalkyl, alkoxyalkyl, aryl, arylalkyl, heterocyclyl, heteroaryl, heteroarylalkyl, heterocycloalkyl or cyclyl, cyclylalkyl, or
R¹² and R^{12′} taken together can be cyclized to form —(CH₂)_{q}X(CH₂)ₛ—; wherein each R¹² and R^{12′} may independently optionally be substituted with 1 to 3 substituents selected from the group consisting of halogen, OR¹¹, alkoxy, heterocycloalkyl, —NR¹¹C(O)NR¹¹R^{11′}, —C(O)NR¹¹R^{11′}, —NR¹¹C(O)R^{11′}, —CN, oxo, —NR¹¹SO₂R^{11′}, —OC(O)R¹¹, —SO₂NR¹¹R^{11′}, —SOR¹³, —S(O)₂R¹³, —COOH and —C(O)OR¹³; each R¹³ is independently alkyl, aryl, arylalkyl, heteroaryl, or heteroarylalkyl, each of which may optionally be substituted with —(CH₂),OH;
each R¹⁴ is independently alkoxy, alkoxycarbonyl, —C(O)NR¹²R^{12′}, —NR¹¹R^{11′}, —C(O)R¹², —NR¹¹C(O)NR¹¹R^{11′}, or —N-heteroaryl;
each R¹⁵ is independently —(CH₂)ₚN(R¹²)C(O)R^{12′}, —(CH₂)ₚCN, —(CH₂)ₚN(R¹²)C(O)OR¹², —(CH₂)ₚN(R¹²)C(O)NR¹²R^{12′}, —(CH₂)ₚN(R¹²)SO₂R¹², —(CH₂),SO₂NR¹²R^{12′}, —(CH₂)ₚC(O)NR1R^{12′}, —(CH₂)ₚC(O)OR¹², —(CH²)ₚOC(O)OR¹², —(CH₂)ₚOC(O)R¹², —(CH₂)ₚOC(O)NR¹²R^{12′}, —(CH₂)ₚN(R¹²)SO₂NR¹²R^{12′}, —(CH₂)ₚOR¹², —(CH₂)ₚOC(O)N(R¹²)(CH₂)ₘOH, —(CH₂)ₚSOR¹², —(CH₂)ₚSO₂R¹², —(CH₂)ₚNR¹¹R¹¹ or —(CH₂)ₚOCH₂C(O)N(R¹²)(CH₂)ₘOH;
each R¹⁶ is independently halo, alkyl, alkenyl, alkynyl, alkoxy, —(CH₂)ₚNR¹¹C(O)NR¹¹R^{11′}, —(CH₂)ₚC(O)NR¹¹R^{11′}, —(CH₂)ₚNR¹¹C(O)R^{11′}, —CN, —(CH₂)ₚNR¹¹SO₂R^{11′}, —(CH₂)ₚOC(O)R¹¹, —(CH₂)ₚSO₂NR¹¹R^{11′}, —(CH₂)ₚSOR¹³, —(CH₂)ₚCOOH or —(CH₂)ₚ(C(O)OR¹³;
X is CR¹¹R^{11′}, O, S, S(O), S(O)₂, or NR¹¹;
m is an integer between 1 and 6;
p is an integer from 0 to 5;
q and s are each independently an integer between 1 and 3; and
w is an integer between 0 and 5.

In some embodiments, A is CH₂CH₂ or CH₂CH₂CH₂, preferably CH₂CH₂CH₂.

In some embodiments, each R⁴ and R⁵ is independently alkyl, for example methyl or ethyl, preferably ethyl.

In some embodiments, Y is a monocyclic heteroaromatic moiety, for example a nitrogen containing heteraromatic moiety such as a nitrogen containing five membered heteraromatic moiety.

In some embodiments, Y is a heterocyclic moiety containing at least two heteroatoms, for example, a five membered heterocyclic moiety containing at least two heteroatoms or at least three heteroatoms.

In some embodiments Y is substituted with one R¹⁰. R¹⁰can be positioned, for example, 1,3 relative to the point of attachment of Y to the adjacent chain carbon or 1,2 relative to the point of attachment of Y to the adjacent chain carbon.

In some embodiments, R¹⁰ is aryl or heteroaryl, for example a monocyclic aryl or monocyclic heteroaryl such as phenyl, pyridyl, oxazolyl, thiazolyl, or thiophenyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is a bicyclic heteroaryl, for example indolyl, benzimidazolyl, benzoxazolyl, benzofuranyl, benzothiophenyl, or benzthiazolyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is arylalkyl or heteroarylalky, for example a monocyclic or bicyclic arylalkyl or monocyclic or bicyclic heteroaryalkyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ includes an unsaturated or partially unsaturated cyclic moiety, for example a cyclyl or heterocyclyl moiety. The cyclic moiety can either be directly attached to Y or attached via a linker such as an alkylenyl linker. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments Y is oxadiazole or triazole.

In some embodiments, Y is

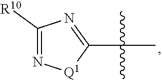

wherein Q1 is O or NR², preferably O or NH. In some embodiments, R¹⁰ is aryl, arylalkyl, heteroaryl, or heteroarylalkyl, for example optionally substituted with one or more R¹⁶. In some embodiments, R¹⁰ is substituted with one R¹⁶, such as halo (e.g., fluoro or chloro) or alkoxy.

In some embodiments, R¹ and R³ together form a heterocyclic ring such as a pyrrolidine or an azetidine ring (The heterocyclic ring can be unsubstituted or substituted, for example, with 1-2 R⁶);

n is 0 or 1;
k′ is a bond;
R² hydrogen or C₁-C₆ alkyl, preferably hydrogen;

### A is

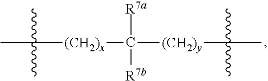

x and y are each independently 0-6;
R⁴ and R⁵ are each independently hydrogen or alkyl;
Y is a monocyclic aryl or monocyclic heteroaryl; each of which is optionally substituted with 1-4 R¹⁰;
each R¹⁰ is independently alkyl, alkenyl, alkynyl, halo, cyano, carbonyl, aryl, arylalkyl, arylalkenyl, arylalkynyl, cyclyl, cyclylalkyl, alkoxy, alkoxyalkyl, aryloxy, aryloxyalkyl, heterocyclyl, heterocyclylalkyl, heteroaryl, heteroarylalkyl, —OR, —NR¹¹R^{11′}, —CF₃, —SOR¹², —SO₂R¹², —OC(O)R¹¹, —SO₂NR¹²R^{12′}, —(CH₂)ₘR¹⁴ or R¹⁵; each of which is optionally independently substituted with 1-3 R¹⁶;
R¹¹ and R^{11′} are each independently hydrogen, alkyl, alkenyl, alkynyl, cyclyl, heterocyclyl, aryl or heteroaryl;
R¹² and R^{12′} are each independently hydrogen, alkyl, alkenyl, alkynyl, alkylthioalkyl, alkoxyalkyl, aryl, arylalkyl, heterocyclyl, heteroaryl, heteroarylalkyl, heterocycloalkyl or cyclyl, cyclylalkyl, or R¹² and R^{12′} taken together can be cyclized to form —(CH₂)_{q}X(CH₂)ₛ—; wherein each R¹² and R^{12′} may independently optionally be substituted with 1 to 3 substituents selected from the group consisting of halogen, OR¹¹, alkoxy, heterocycloalkyl, —NR¹¹C(O)NR¹¹R^{11′}, —C(O)NR¹¹R¹¹, —NR¹¹C(O)R^{11′}, —CN, oxo, —NR¹¹SO₂R^{11′}; —OC(O)R¹¹, —SO₂NR¹¹R^{11′}, —SOR¹³, —S(O)₂R¹³, —COOH and —C(O)OR¹³; each R¹³ is independently alkyl, aryl, arylalkyl, heteroaryl, or heteroarylalkyl, each of which may optionally be substituted with —(CH₂)_{w}OH;
each R¹⁴ is independently alkoxy, alkoxycarbonyl, —C(O)NR¹²R^{12′}, —NR¹¹R^{11′}, —C(O)R¹², —NR¹¹C(O)NR¹¹R^{11′} or —N-heteroaryl;
each R¹⁵ is independently —(CH₂)ₚN(R¹²)C(O)R^{12′}, —(CH₂)ₚCN, —(CH₂)ₚN(R)¹²)C(O)OR^{12′}, —(CH₂)ₚN(R¹²)C(O)NR¹²R^{12′}, —(CH₂)ₚN(R¹²)SO₂R¹², —(CH₂)SO₂NR¹²R^{12′}, —(CH₂)ₚC(O)NR¹²R^{12′}, —(CH₂)ₚC(O)OR¹², —(CH²)ₚOC(O)OR¹², —(CH₂)ₚOC(O)R¹², —(CH₂)ₚOC(O)NR¹²R^{12′}, —(CH₂)ₚN(R¹²)SO₂NR¹²R^{12′}, —(CH₂)ₚOR¹², —(CH₂)ₚOC(O)N(R¹²)(CH₂)ₘOH, —(CH₂)ₚSOR¹², —(CH₂)ₚSO₂R¹², —(CH₂)ₚNR¹¹R¹¹ or —(CH₂)ₚOCH₂C(O)N(R¹²)(CH₂)ₘOH;
each R¹⁶ is independently halo, alkyl, alkenyl, alkynyl, alkoxy, —(CH₂)ₚNR¹²C(O)NR¹¹R^{11′}, —(CH₂)ₚC(O)NR¹¹R^{11′}, —(CH₂)ₚNR¹¹C(O)R^{11′}, —CN, —(CH₂)ₚNR¹¹SO₂R^{11′}, —(CH₂)ₚOC(O)R¹¹, —(CH₂)ₚSO₂NR¹¹R^{11′}, —(CH₂)SOR¹³, —(CH₂)ₚCOOH or —(CH₂)ₚC(O)OR¹³;
X is CR¹¹R^{11′}, O, S, S(O), S(O)₂, or NR¹¹;
m is an integer between 1 and 6;
p is an integer from 0 to 5;
q and s are each independently an integer between 1 and 3; and
w is an integer between 0 and 5;

In some embodiments, A is CH₂CH₂ or CH₂CH₂CH₂, preferably CH₂CH₂CH₂.

In some embodiments, each R⁴ and R⁵ is independently alkyl, for example, methyl or ethyl, preferably ethyl.

In some embodiments, Y is monocyclic heteroaromatic moiety, for example a nitrogen containing heteraromatic moiety such as a nitrogen containing five membered heteraromatic moiety.

In some embodiments, Y is a heterocyclic moiety containing at least two heteroatoms, for example, a five membered heterocyclic moiety containing at least two heteroatoms or at least three heteroatoms.

In some embodiments, R¹⁰ is aryl or heteroaryl, for example a monocyclic aryl or monocyclic heteroaryl such as phenyl, pyridyl, oxazolyl, thiazolyl, or thiophenyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is a bicyclic heteroaryl, for example indolyl, benzimidazolyl, benzoxazolyl, benzofuranyl, benzothiophenyl, or benzthiazolyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is arylalkyl or heteroarylalky, for example a monocyclic or bicyclic arylalkyl or monocyclic or bicyclic heteroaryalkyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ includes an unsaturated or partially unsaturated cyclic moiety, for example a cyclyl or heterocyclyl moiety. The cyclic moiety can either be directly attached to Y or attached via a linker such as an alkylenyl linker. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments Y is oxadiazole or triazole.

In some embodiments, Y is

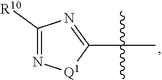

wherein Q1 is O or NR², preferably O or NH. In some embodiments, R¹⁰ is aryl, arylalkyl, heteroaryl, or heteroarylalkyl, for example optionally substituted with one or more R¹⁶. In some embodiments, R¹⁰ is substituted with one R¹⁶, such as halo (e.g., fluoro or chloro) or alkoxy.

In some embodiments, the compounds has a formula (Ic)

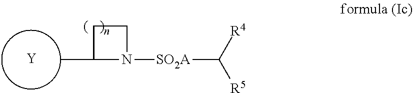

n is 0, 1, 2, 3, or 4; preferably 1 or 2;
A is CH₂, CH₂CH₂, or CH₂CH₂CH₂;
R⁴ and R⁵ are each independently hydrogen or alkyl;
Y is a monocyclic aryl or monocyclic heteroaryl; each of which is optionally substituted with 1-4 R¹⁰;
each R¹⁰ is independently alkyl, alkenyl, alkynyl, halo, cyano, carbonyl, aryl, arylalkyl, arylalkenyl, arylalkynyl, cyclyl, cyclylalkyl, alkoxy, alkoxyalkyl, aryloxy, aryloxyalkyl, heterocyclyl, heterocyclylalkyl, heteroaryl, heteroarylalkyl, —OR, —NR¹¹R^{11′}, —CF₃, —SOR¹², —SO₂R¹², —OC(O)R¹¹, —SO₂NR¹²R^{12′}, —(CH₂)ₘR¹⁴ or R¹⁵; each of which is optionally independently substituted with 1-3 R¹⁶;
R¹¹ and R^{11′} are each independently hydrogen, alkyl, alkenyl, alkynyl, cyclyl, heterocyclyl, aryl or heteroaryl;
R¹² and R^{12′} are each independently hydrogen, alkyl, alkenyl, alkynyl, alkylthioalkyl, alkoxyalkyl, aryl, arylalkyl, heterocyclyl, heteroaryl, heteroarylalkyl, heterocycloalkyl or cyclyl, cyclylalkyl, or R¹² and R^{12′} taken together can be cyclized to form —(CH₂)_{q}X(CH₂)ₛ—; wherein each R¹² and R^{12′} may independently optionally be substituted with 1 to 3 substituents selected from the group consisting of halogen, OR¹¹, alkoxy, heterocycloalkyl, —NR¹¹C(O)NR¹¹R^{11′}, —C(O)NR¹¹R¹¹, —NR¹¹C(O)R^{11′}, —CN, oxo, —NR¹¹SO₂R^{11′}, —OC(O)R¹¹, —SO₂NR¹¹R^{11′}, —SOR¹³, —S(O)₂R¹³, —COOH and —C(O)OR¹³; each R¹³ is independently alkyl, aryl, arylalkyl, heteroaryl, or heteroarylalkyl, each of which may optionally be substituted with —(CH₂),OH;
each R¹⁴ is independently alkoxy, alkoxycarbonyl, —C(O)NR¹²R^{12′}, —NR¹¹R^{11′}, —C(O)R¹², —NR¹¹C(O)NR¹¹R^{11′} or —N-heteroaryl;
each R¹⁵ is independently —(CH₂)ₚN(R¹²)C(O)R^{12′}, —(CH₂)ₚCN, —(CH₂)ₚN(R¹²)C(O)OR^{12′}, —(CH₂)ₚN(R¹²)C(O)NR¹²R^{12′}, —(CH₂)ₚN(R¹²)SO₂R¹², —(CH₂)SO₁₂NR¹²R^{12′}, —(CH₂)ₚC(O)NR³²R^{12′}, —(CH₂ₚC(O)OR¹², —(CH²)ₚOC(O)OR¹², —(CH₂)ₚOC(O)R¹², —(CH₂)ₚOC(O)NR¹²R^{12′}, —(CH₂)ₚN(R¹²)SO₂NR¹²R^{12′}, —(CH₂)ₚOR¹², —(CH₂)ₚOC(O)N(R¹²)(CH₂)ₘOH, —(CH₂)ₚSOR¹², —(CH₂)ₚSO₂R¹², —(CH₂)ₚNR¹¹R¹¹ or —(CH₂)ₚOCH₂C(O)N(R¹²)(CH₂)ₘOH;
each R¹⁶ is independently halo, alkyl, alkenyl, alkynyl, alkoxy, —(CH₂)ₚNR¹¹C(O)NR¹¹R^{11′}, —(CH₂)ₚC(O)NR¹¹R^{11′}, —(CH₂)ₚNR¹¹C(O)R^{11′}, —CN, —(CH₂)ₚNR¹¹SO₂R^{11′}, —(CH₂)ₚOC(O)R¹¹, —(CH₂)ₚSO₂NR¹¹R^{11′}, —(CH₂)ₚSOR¹³, —(CH₂)ₚCOOH or —(CH₂)ₚC(O)OR¹³;
X is CR¹¹R^{11′}, O, S, S(O), S(O)₂, or NR¹¹;
m is an integer between 1 and 6;
p is an integer from 0 to 5;
q and s are each independently an integer between 1 and 3; and
w is an integer between 0 and 5.

In some embodiments, A is CH₂CH₂ or CH₂CH₂CH₂, preferably CH₂CH₂CH₂.

In some embodiments, each R⁴ and R⁵ is independently alkyl, for example, methyl or ethyl, preferably ethyl.

In some embodiments, Y is a monocyclic heteroaromatic moiety, for example a nitrogen containing heteraromatic moiety such as nitrogen containing five membered heteraromatic moiety.

In some embodiments, Y is a heterocyclic moiety containing at least two heteroatoms, for example, a five membered heterocyclic moiety containing at least two heteroatoms or at least three heteroatoms.

In some embodiments Y is substituted with one R¹⁰. R¹⁰can be positioned, for example, 1,3 relative to the point of attachment of Y to the adjacent chain carbon or 1,2 relative to the point of attachment of Y to the adjacent chain carbon.

In some embodiments, R¹⁰ is aryl or heteroaryl, for example a monocyclic aryl or monocyclic heteroaryl such as phenyl, pyridyl, oxazolyl, thiazolyl, or thiophenyl. In some embodiments, R¹⁰ is substituted with 1-3 R¹⁶. In some embodiments, R¹⁶ halo, alkyl, or alkoxy, for example chloro, fluoro, methyl, cyano, or methoxy.

In some embodiments, R¹⁰ is a bicyclic heteroaryl, for example indolyl, benzimidazolyl, benzoxazolyl, benzofuranyl, benzothiophenyl, or benzthiazolyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ is arylalkyl or heteroarylalky, for example a monocyclic or bicyclic arylalkyl or monocyclic or bicyclic heteroaryalkyl. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments, R¹⁰ includes an unsaturated or partially unsaturated cyclic moiety, for example a cyclyl or heterocyclyl moiety. The cyclic moiety can either be directly attached to Y or attached via a linker such as an alkylenyl linker. In some embodiments, R¹⁰ is substituted with 1-3 R¹⁶. In some embodiments, R¹⁶ is halo, alkyl, or alkoxy, for example chloro, fluoro, methyl, cyano, or methoxy.

In some embodiments Y is oxadiazole or triazole.

In some embodiments, Y is

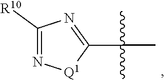

wherein Q1 is O or NR², preferably O or NH. In some embodiments, R¹⁰ aryl, arylalkyl, heteroaryl, or heteroarylalkyl, for example optionally substituted with one or more R¹⁶. In some embodiments, R¹⁰ is substituted with one R¹⁶, such as halo (e.g., fluoro or chloro) or alkoxy.

In another aspect, the invention features a compound of formula (IV)

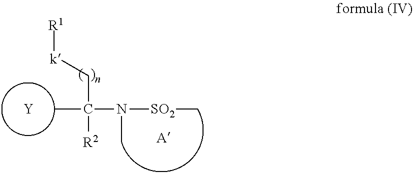

wherein,
R¹ is hydrogen, aryl, heteroaryl, arylalkyl, heteroarylalkyl, cyclyl, cyclylalkyl, heterocyclyl, heterocyclylalkyl, alkyl, alkenyl, alkynyl, or R¹ can be taken together with
R² or R³ to form a ring; each of which is optionally substituted with 1-4 R⁶;
k′ is a bond, O, C(O), C(O)O, OC(O), C(O)NR³, NR³C(O), S, SO, SO₂, CR²═CR², OR C≡C;
n is 0-6, preferably 1-3;
R² is hydrogen, C₁-C₆ alkyl, C₂-C₆ alkenyl, or C₂-C₆ alkynyl;
A′ is heterocyclyl; optionally substituted with 1-3 R⁹;
Y is a monocyclic aryl or monocyclic heteroaryl; each of which is optionally substituted with 1-4 R¹⁰;
each R⁶ is independently halo, alkyl, alkenyl, alkynyl, cyclyl, heterocyclyl, aryl, heteroaryl, alkoxy, haloalkyl, haloalkyloxy, haloalkylthio, acetyl, cyano, nitro, hydroxy, oxo, C(O)OR², OC(O)R², N(R³)₂, C(O)N(R³)₂, NR³C(O)R², or SR²;
R⁹ is halo, alkyl, cyclyl, heterocyclyl, aryl, heteroaryl, alkoxy, haloalkyl, haloalkyloxy, haloalkylthio, acetyl, cyano, nitro, hydroxy, oxo, C(O)OR², OC(O)R², N(R²)₂, C(O)N(R²)₂, NR²C(O)R², SR²;
each R¹⁰ is independently alkyl, alkenyl, alkynyl, halo, cyano, carbonyl, aryl, arylalkyl, arylalkenyl, arylalkynyl, cyclyl, cyclylalkyl, alkoxy, alkoxyalkyl, aryloxy, aryloxyalkyl, heterocyclyl, heterocyclylalkyl, heteroaryl, heteroarylalkyl, —OR, —NR¹¹R^{11′}, —CF₃, —SO₂R¹², —OC(O)R¹¹, —SO₂NR¹²R^{12′}, —(CH₂)ₘR¹⁴ or R¹⁵; each of which is optionally independently substituted with 1-3 R¹⁶;
R¹¹ and R^{11′} are each independently hydrogen, alkyl, alkenyl, alkynyl, cyclyl, heterocyclyl, aryl or heteroaryl;
R¹² and R^{12′} are each independently hydrogen, alkyl, alkenyl, alkynyl, alkylthioalkyl, alkoxyalkyl, aryl, arylalkyl, heterocyclyl, heteroaryl, heteroarylalkyl, heterocycloalkyl or cyclyl, cyclylalkyl, or R¹² and R^{12′} taken together can be cyclized to from —(CH₂)_{q}X(CH₂)ₛ—; wherein each R¹² and R^{12′} may independently optionally be substituted with 1 to 3 substituents selected from the group consisting of halogen, OR¹¹, alkoxy, heterocycloalkyl, —NR¹¹C(O)NR¹¹R^{11′}, —C(O)NR¹¹R^{11′}, —NR¹¹C(O)R^{11′}, —CN, oxo, —NR¹¹SO₂R^{11′}, —OC(O)R¹¹, —SO₂NR¹¹R^{11′}, —SOR¹³, —S(O)₂R¹³, —COOH and —C(O)OR¹³;
each R¹³ is independently alkyl, aryl, arylalkyl, heteroaryl, or heteroarylalkyl, each of which may optionally be substituted with —(CH₂)_{w}OH;
each R¹⁴ is independently alkoxy, alkoxycarbonyl, —C(O)NR¹²R^{12′}, —NR¹¹R^{11′}, —C(O)R¹², —NR¹¹C(O)NR¹¹R¹¹′ or —N-heteroaryl;
each R¹⁵ is independently heterocycloalkyl, heteroaryl, —CN, —(CH₂)ₚN(R¹²)C(O)R^{12′}, —(CH₂)ₚCN, —(CH₂)ₚN(R¹²)C(O)OR^{12′}, —(CH₂)ₚN(R¹²)C(O)NR¹²R^{12′}, —(CH₂)ₚN(R³²)SO₂R¹², —(CH₂)ₚSO₂NR¹²R^{12′}, —(CH₂)ₚC(O)NR¹²R^{12′}, —(CH₂)ₚN(R¹²)SO₂NR¹²R^{12′}, —(CH₂)ₚOR¹², —(CH₂)ₚOC(O)N(R¹²)(CH₂)ₘOH, —(CH₂)SOR¹² or —(CH₂)ₚOCH₂C(O)N(R¹²)(CH₂)ₘOH; each R¹⁶ is independently halo, alkyl, alkenyl, alkynyl, alkoxy, —(CH₂)ₚNR¹¹C(O)NR¹¹R^{11′}, —(CH₂)ₚC(O)NR¹¹R^{11′}, —(CH₂)ₚNR¹¹C(O)R^{11′}, —CN, —(CH₂)ₚNR¹¹SO₂R^{11′}, —(CH₂)ₚOC(O)R¹¹, —(CH₂)ₚSO₂NR¹¹R^{11′}, —(CH₂)SOR¹³, —(CH₂)ₚCOOH or —(CH₂)ₚC(O)OR¹³;
X is CR¹¹R^{11′}, O, S, S(O), S(O)₂, or NR¹¹;
m is an integer between 1 and 6;
p is an integer from 0 and 5.
q and s are each independently an integer between 1 and 3; and
w is an integer between 0 and 5.

In some embodiments, the compound of formula (IV), comprises an enriched preparation of formula (IV′).

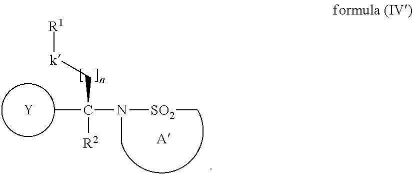

In some embodiments, the compound of formula (IV), comprises an enriched preparation of formula (IV″)

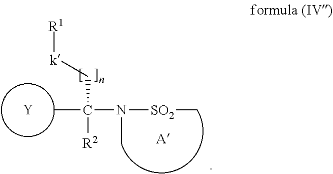

In some embodiments, A′ is a 5 or 6 membered heterocyclyl.

In some embodiments, the 5 or 6 membered heterocyclyl includes at least two nitrogen atoms.

In some embodiments, A′ is

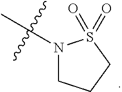 [SMILES: *N1CCCS1(=O)=O]

In some embodiments, A′ is substituted with one R⁹, for example, N(R²)₂.

In some embodiments, n is 1; k′ is a bond or O; and R¹ is aryl, heteroaryl, arylalkyl, or heteroarylalkyl. In some embodiments, n is 1; k′ is O; and R¹ is arylalkyl. For example, R¹ can be phenylmethyl. In some embodiments, n is 2; k′ is a bond; and R¹ is aryl.

In some embodiments, Y is a monocyclic heteroaromatic moiety, for example, a nitrogen containing heteraromatic moiety, such as a nitrogen containing 5 membered heteraromatic moiety.

In some embodiments, Y is a heterocyclic moiety containing at least two heteroatoms, for example, a 5 membered heterocyclic moiety containing at least two heteroatoms or a heterocyclic moiety containing at least 3 heteroatoms.

In some embodiments, Y is substituted with 1 R¹⁰. The R¹⁰can be positioned, for example, 1,3 relative to the point of attachment of Y to the adjacent chain carbon or can be positioned, for example, 1,2 relative to the point of attachment of Y to the adjacent chain carbon.

In some embodiments, R¹⁰ is aryl or heteroaryl, for example a monocyclic aryl or monocyclic heteroaryl such as phenyl, pyridyl, or thiophenyl. In some embodiments, R¹⁰ is substituted with 1-3 R¹⁶. In some embodiments, R¹⁶ is halo, alkyl, or alkoxy, for example chloro, fluoro, methyl, or methoxy.

In some embodiments, R¹⁰ is a bicyclic heteroaryl, for example indolyl, imidazolyl, benzoxazolyl, or benzthiazolyl. In some embodiments, R¹⁰ is substituted with 1-3 R¹⁶. In some embodiments, R¹⁶ is halo, alkyl, or alkoxy, for example chloro, fluoro, methyl, or methoxy.

In some embodiments, Y is oxadiazole or triazole.

In another aspect, the invention features a compound of formula (V),

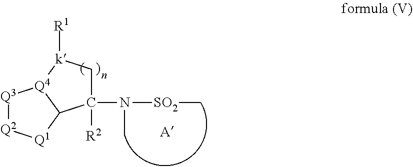

wherein,
Q¹, Q², Q³ and Q⁴ together with the carbon to which they are attached form a heteroaryl moiety, and each Q¹, Q², Q³ and Q⁴ is independently S, O, N, CR², CR¹⁰, NR², or N¹⁰.

In some embodiments, the compound of formula (V), comprises an enriched preparation of formula (V′)

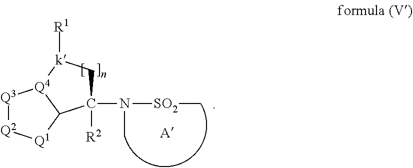

In some embodiments, the compound of formula (V), comprises an enriched preparation of formula (V″)

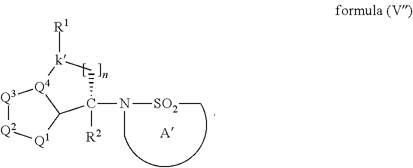

In some embodiments, Q¹ and Q⁴ are each independently S, O, N, or NR¹⁰

In some embodiments, Q¹ and Q³ are each independently S, O, N, or NR¹⁰. In some embodiments, Q² is CR² or CR¹⁰. In some embodiments, Q² is S, O, N, or NR¹⁰. In some embodiments, at least one of Q² or Q³ is CR² or CR¹⁰. In some embodiments, at least two of Q¹, Q², Q³, or Q⁴ is S, O, N, or NR¹⁰. In some embodiments, Q¹, Q², and Q³are each independently S, O, N, or NR¹⁰. In some embodiments, Q¹ is NR¹⁰. In some embodiments, one of Q², Q³, or Q⁴ is CR². In some embodiments, Q² is CR¹⁰. In some embodiments, Q³ is CR².

In some embodiments, Q¹, Q², Q³ and Q⁴ together form

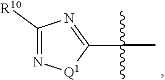

In some embodiments, Q¹ is NR².

In some embodiments, Q¹, Q², Q³ and Q⁴ together form

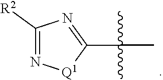

In some embodiments, Q¹ is NR¹.

In another aspect, the invention features a compound of formula (VI),

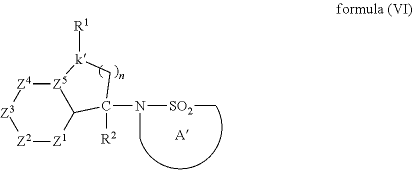

wherein
Z¹, Z², Z³, Z⁴, and Z⁵ together form an aryl or heteroaryl moiety, and each Z¹, Z², Z³, Z⁴, and Z⁵ is independently N, CR¹⁰, or CR².

In some embodiments, the compound of formula (IV), comprises an enriched preparation of a compound of formula (VI′).

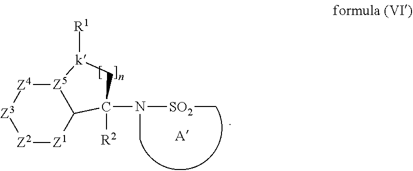

In some embodiments, the compound of formula (VI), comprises an enriched preparation of a compound of formula (VI″).

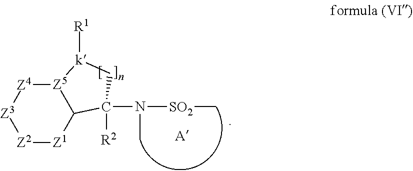

In some embodiments, one of Z¹, Z², Z³, Z⁴, and Z⁵ is N. In some embodiments, two of Z¹, Z², Z³, Z⁴, and Z⁵are N. In some embodiments, three of Z¹, Z², Z³, Z⁴, and Z⁵ is N. In some embodiments, two of Z¹ and Z²are N. In some embodiments, two of Z¹ and Z³are N. In some embodiments, two of Z¹ and Z⁴are N. In some embodiments, two of Z¹, Z³, and Z⁵are N.

In some embodiments, the compound is a compound of formula (IV), wherein Y is substituted with a single substituent R¹⁰. For example, R¹⁰can be aryl or heteroaryl, optionally substituted with up to three independent R¹⁶

In some embodiments, R¹⁰ is aryl or heteroaryl, for example a monocyclic aryl or monocyclic heteroaryl such as phenyl, pyridyl, or thiophenyl. In some embodiments, R¹⁰ is substituted with 1-3 R¹⁶. In some embodiments, R¹⁶ is halo, alkyl, or alkoxy, for example chloro, fluoro, methyl, or methoxy.

In some embodiments, R¹⁰ is a bicyclic heteroaryl, for example indolyl, imidazolyl, benzoxazolyl, or benzthiazolyl. In some embodiments, R¹⁰ is substituted with 1-3 R¹⁶. In some embodiments, R¹⁶ is halo, alkyl, or alkoxy, for example chloro, fluoro, methyl, or methoxy.

In some embodiments, R¹⁰ is R¹⁵. In some embodiments, Y is substituted with a second R¹⁰, for example an alkyl, halo or alkoxy. In another aspect, the invention features a pharmaceutically acceptable salt comprising a compound of any of the formulae described herein.

In some embodiments, the compound is an enantiomerically enriched isomer of a stereoisomer described herein. For example, the compound has an enantiomeric excess of at least about 10%, 15%, 20%, 25%, 30%, 35%, 40%, 45%, 50%, 55%, 60%, 65%, 70%, 75%, 80%, 85%, 90%, 95%, 96%, 97%, 98%, or 99%. Enantiomer, when used herein, refers to either of a pair of chemical compounds whose molecular structures have a mirror-image relationship to each other.

In some embodiments, a preparation of a compound disclosed herein is enriched for an isomer of the compound having a selected stereochemistry, e.g., R or S, corresponding to a selected stereocenter, e.g., the position corresponding to the carbon alpha to the sulfonamide nitrogen in formula (I). Exemplary R/S configurations can be those provided in an example described herein, e.g., those described in the Table below, or the configuration of the majority or minority species in a synthetic scheme described herein. For example, the compound has a purity corresponding to a compound having a selected stereochemistry of a selected stereocenter of at least about 60%, 65%, 70%, 75%, 80%, 85%, 90%, 95%, 96%, 97%, 98%, or 99%.

In some embodiments, a compound described herein includes a preparation of a compound disclosed herein that is enriched for a structure or structures having a selected stereochemistry, e.g., R or S, at a selected stereocenter, e.g., the carbon alpha to the sulfonamide nitrogen of a formula described herein e.g., formula (I), (II), (III), (IV), (V), or (VI). Exemplary R/S configurations can be those provided in an example described herein, e.g., those described in the Table below, or the configuration of the majority or minority species in a synthetic scheme described herein. For example, the compound has a purity corresponding to a compound having a selected stereochemistry of a selected stereocenter of at least about 60%, 65%, 70%, 75%, 80%, 85%, 90%, 95%, 96%, 97%, 98%, or 99%.

An “enriched preparation,” as used herein, is enriched for a selected stereoconfiguration of one, two, three or more selected stereocenters within the subject compound. Exemplary selected stereocenters and exemplary stereoconfigurations thereof can be selected from those provided, herein, e.g., in an example described herein, e.g., those described in the Table below. By enriched is meant at least 60%, e.g., of the molecules of compound in the preparation have a selected stereochemistry of a selected stereocenter. In preferred embodiments it is at least 65%, 70%, 75%, 80%, 85%, 90%, 95%, 96%, 97%, 98%, or 99%. Enriched refers to the level of a subject molecule(s) and does not connote a process limitation unless specified.

In some embodiments, a preparation of a compound disclosed herein, is enriched for isomers (subject isomers) which are diastereromers of the compound described herein. For example, a compound having a selected stereochemistry, e.g., R or S, corresponding to a selected stereocenter, e.g., the position corresponding to the carbon alpha to the sulfonamide nitrogen of a formula described herein e.g., formula (I), (II), (III), (IV), (V), or (VI). Exemplary R/S configurations can be those provided in an example described herein, e.g., those described in the Table below, or the configuration of the majority or minority species in a synthetic scheme described herein. For example, the compound has a purity corresponding to a compound having a selected stereochemistry of a selected stereocenter of at least about 60%, 65%, 70%, 75%, 80%, 85%, 90%, 95%, 96%, 97%, 98%, or 99%. Diastereromer, when used herein, refers to a stereoisomer of a compound having two or more chiral centers that is not a mirror image of another stereoisomer of the same compound.

In one embodiment, the compound has a molecular weight less than [D-Lys-3]-GHRP6 or H(2)N-D-arg-Pro-Lys-Pro-d-Phe-Gln-d-Trp-Phe-d-Trp-Leu-Leu-NH(2) (L 756,867) or within 2, 1.5, 1.4, 1.2, 1.1, 0.8, 0.6, or 0.5 fold that of [D-Lys-3]-GHRP-6 or L 756,867.

In another aspect, the invention features a compound listed in Table 4. Representative compounds of the invention are depicted below in Table 4. Other exemplary compounds are within the scope set forth in the Summary or are described elsewhere herein.

TABLE 4
Exemplary GHS-R Modulating Compounds

| Number | Name | Activity* |
|---|---|---|
| 1 | 3-Diethylamino-propane-1-sulfonic acid [(R)-2-benzyloxy-1-(3- | C |
| | phenyl-[1,2,4]oxadiazol-5-yl)-ethyl]-amide | |
| 2 | 3-Diethylamino-propane-1-sulfonic acid {(R)-2-benzyloxy-1-[3- | A |
| | (2,6-dichloro-phenyl)-[1,2,4]oxadiazol-5-yl]-ethyl}-amide | |
| 3 | 3-Diethylamino-propane-1-sulfonic acid {(R)-1-[5-(2,3-dichloro- | A |
| | phenyl)-2H-[1,2,4]triazol-3-yl]-3-phenyl-propyl}-amide | |
| 4 | 3-Diethylamino-propane-1-sulfonic acid [(R)-3-phenyl-1-(3-o- | A |
| | tolyl-[1,2,4]oxadiazol-5-yl)-propyl]-amide | |
| 5 | 3-Diethylamino-propane-1-sulfonic acid {(R)-1-[5-(4-fluoro- | A |
| | benzyl)-2H-[1,2,4]triazol-3-yl]-3-phenyl-propyl}-amide | |
| 6 | 3-Diethylamino-propane-1-sulfonic acid [(R)-3-phenyl-1-(3- | A |
| | phenyl-[1,2,4]oxadiazol-5-yl)-propyl]-amide | |
| 7 | 3-Diethylamino-propane-1 sulfonic acid [(R)-3-phenyl-1-(5- | A |
| | thiophen-3-yl-2H-[1,2,4]triazol-3-yl)-propyl]-amide | |
| 8 | 3-Diethylamino-propane-1-sulfonic acid {(R)-3-phenyl-1-[5- | A |
| | (2,4,6-trifluoro-phenyl)-2H-[1,2,4]triazol-3-yl]-propyl}-amide | |
| 9 | 3-Diethylamino-propane-1-sulfonic acid {(R)-1-[3-(3-methyl- | B |
| | pyridin-2-yl)-[1,2,4]oxadiazol-5-yl]-3-phenyl-propyl}-amide | |
| 10 | 3-Diethylamino-propane-1-sulfonic acid {(R)-1-[3-(2-chloro-6- | B |
| | methyl-phenyl)-[1,2,4]oxadiazol-5-yl]-3-phenyl-propyl}-amide | |
| 11 | 3-Diethylamino-propane-1-sulfonic acid [(R)-1-(5-benzo[1,3]dioxol- | A |
| | 5-yl-2H-[1,2,4]triazol-3-yl)-3-phenyl-propyl]-amide | |
| 12 | 3-Diethylamino-propane-1-sulfonic acid {(R)-1-[3-(2,6-dichloro- | A |
| | phenyl)-[1,2,4]oxadiazol-5-yl]-3-phenyl-propyl}-amide | |
| 13 | 2-Diethylamino-ethanesulfonic acid [(R)-3-phenyl-1-(3-o-tolyl- | C |
| | [1,2,4]oxadiazol-5-yl)-propyl]-amide | |
| 14 | 2-Diethylamino-ethanesulfonic acid {(R)-1-[3-(3-methyl-pyridin- | B |
| | 2-yl)-[1,2,4]oxadiazol-5-yl]-3-phenyl-propyl}-amide | |
| 15 | 3-Diethylamino-propane-1-sulfonic acid {(R)-1-[5-(3-fluoro- | A |
| | phenyl)-2H-[1,2,4]triazol-3-yl]-3-phenyl-propyl}-amide | |
| 16 | 3-Diethylamino-propane-1-sulfonic acid {(R)-1-[3-(2-methoxy- | C |
| | phenyl)-[1,2,4]oxadiazol-5-yl]-3-phenyl-propyl}-amide | |
| 17 | 2-Diethylamino-ethanesulfonic acid [(R)-3-phenyl-1-(3-phenyl- | B |
| | [1,2,4]oxadiazol-5-yl)-propyl]-amide | |
| 18 | 2-Diethylamino-ethanesulfonic acid {(R)-1-[3-(2,6-dichloro- | B |
| | phenyl)-[1,2,4]oxadiazol-5-yl]-3-phenyl-propyl}-amide | |
| 19 | 2-Diethylamino-ethanesulfonic acid {(R)-1-[3-(2-methoxy- | D |
| | phenyl)-[1,2,4]oxadiazol-5-yl]-3-phenyl-propyl}-amide | |
| 20 | 3-Diethylamino-propane-1-sulfonic acid {(R)-1-[5-(1H-indol-5- | A |
| | yl)-2H-{1,2,4]triazol-3-yl]-3-phenyl-propyl}-amide | |
| 21 | 3-Diethylamino-propane-1-sulfonic acid [(S)-3-phenyl-1-(3-o- | B |
| | tolyl-[1,2,4]oxadiazol-5-yl)-propyl]-amide | |
| 22 | 3-Diethylamino-propane-1-sulfonic acid {(R)-1-[5-(4-chloro- | A |
| | phenyl)-2H-[1,2,4]triazol-3-yl]-3-phenyl-propyl}-amide | |
| 23 | 3-Diethylamino-propane-1-sulfonic acid [(R)-3-phenyl-1-(5- | A |
| | phenyl-2H-[1,2,4]triazol-3-yl)-propyl]-amide | |
| 24 | 3-Diethylamino-propane-1-sulfonic acid {(R)-1-[5-(4-chloro- | A |
| | benzyl)-2H-[1,2,4]triazol-3-yl]-3-phenyl-propyl}-amide | |
| 25 | 3-Diethylamino-propane-1-sulfonic acid [(S)-2-benzyloxy-1-(5- | B |
| | phenyl-2H-[1,2,4]triazol-3-yl)-ethyl]-amide | |
| 26 | 3-Diethylamino-propane-1-sulfonic acid [5-(4-chloro-benzyl)- | B |
| | 2H-[1,2,4]triazol-3-ylmethyl]-amide | |
| 27 | 2-Diethylamino-ethanesulfonic acid [(R)-3-phenyl-1-(3-pyridin- | B |
| | 2-yl-[1,2,4]oxadiazol-5-yl)-propyl]-amide | |
| 28 | 2-Diethylamino-ethanesulfonic acid [(R)-3-phenyl-1-(5-phenyl- | A |
| | 2H-[1,2,4]triazol-3-yl)-propyl]-amide | |
| 29 | 3-Diethylamino-propane-1-sulfonic acid {(R)-1-[5-(2-fluoro- | A |
| | phenyl)-2H-[1,2,4]triazol-3-yl]-3-phenyl-propyl}-amide | |
| 30 | 3-Diethylamino-propane-1-sulfonic acid [(R)-1-(5- | A |
| | benzo[1,3]dioxol-5-ylmethyl-2H-[1,2,4]triazol-3-yl)-3-phenyl- | |
| | propyl]-amide | |
| 31 | 3-Diethylamino-propane-1-sulfonic acid {(R)-1-[3-(2-chloro- | A |
| | pyridin-3-yl)-[1,2,4]oxadiazol-5-yl]-3-phenyl-propyl}-amide | |
| 32 | 3-Diethylamino-propane-1-sulfonic acid {(R)-1-[5-(3,4-dimethoxy- | A |
| | benzyl)-2H-[1,2,4]triazol-3-yl]-3-phenyl-propyl}-amide | |
| 33 | 3-Diethylamino-propane-1-sulfonic acid [(R)-2-benzyloxy-1-(3- | D |
| | o-tolyl-[1,2,4] oxadiazol-5-yl)-ethyl]-amide | |
| 34 | 3-Diethylamino-propane-1-sulfonic acid {(R)-2-benzyloxy-1-[3- | C |
| | (3-methyl-pyridin-2-yl)-[1,2,4]oxadiazol-5-yl]-ethyl}-amide | |
| 35 | 4-Diethylamino-cyclohexanesulfonic acid {(R)-1-[5-(4-fluoro- | B |
| | phenyl)-4H-[1,2,4]triazol-3-yl]-3-phenyl-propyl}-amide | |
| 36 | 2-Diethylamino-ethanesulfonic acid {(R)-1-[3-(2,6-dimethyl- | D |
| | phenyl)-[1,2,4]oxadiazol-5-yl]-3-phenyl-propyl}-amide | |
| 37 | 3-Diethylamino-propane-1-sulfonic acid {(R)-1-[3-(2,6-dimethoxy- | C |
| | phenyl)-[1,2,4]oxadiazol-5-yl]-3-phenyl-propyl}-amide | |
| 38 | 3-Diethylamino-propane-1-sulfonic acid {(R)-1-[5-(1H-indol-3- | A |
| | yl)-2H-[1,2,4]triazol-3-yl]-3-phenyl-propyl}-amide | |
| 39 | 3-Diethylamino-propane-1-sulfonic acid [(R)-3-phenyl-1-(3- | C |
| | pyridin-2-yl-[1,2,4]oxadiazol-5-yl)-propyl]-amide | |
| 40 | 3-Diethylamino-propane-1-sulfonic acid {(R)-1-[5-(4-cyano- | A |
| | phenyl)-2H-[1,2,4]triazol-3-yl]-3-phenyl-propyl}-amide | |
| 41 | 3-Diethylamino-propane-1-sulfonic acid [(R)-3-phenyl-1-(3- | B |
| | pyridin-3-yl-[1,2,4]oxadiazol-5-yl)-propyl]-amide | |
| 42 | 3-Diethylamino-propane-1-sulfonic acid {(R)-1-[5-(4-tert-butyl- | D |
| | phenyl)-2H-[1,2,4]triazol-3-yl]-3-phenyl-propyl}-amide | |
| 43 | 2-Diethylamino-ethanesulfonic acid [(R)-3-phenyl-1-(3-pyridin- | B |
| | 3-yl-[1,2,4]oxadiazol-5-yl)-propyl]-amide | |
| 44 | 3-Diethylamino-propane-1-sulfonic acid [(R)-1-(5-benzothiazol- | A |
| | 6-yl-2H-[1,2,4]triazol-3-yl)-3-phenyl-propyl]-amide | |
| 45 | 3-Diethylamino-propane-1-sulfonic acid [(R)-2-benzyloxy-1-(3- | D |
| | pyridin-3-yl-[1,2,4]oxadiazol-5-yl)-ethyl]-amide | |
| 46 | 2-Diethylamino-ethanesulfonic acid {(R)-1-[3-(2,6-dimethoxy- | B |
| | phenyl)-[1,2,4]oxadiazol-5-yl]-3-phenyl-propyl}-amide | |
| 47 | 3-Diethylamino-propane-1-sulfonic acid {(R)-1-[5-(2,3- | A |
| | dihydro-benzo[1,4]dioxin-6-yl)-2H-[1,2,4]triazol-3-yl]-3- | |
| | phenyl-propyl}-amide | |
| 48 | 3-Diethylamino-propane-1-sulfonic acid {(R)-1-[3-(2-chloro- | B |
| | phenyl)-[1,2,4]oxadiazol-5-yl]-3-phenyl-propyl}-amide | |
| 49 | 2-Diethylamino-ethanesulfonic acid {(R)-2-benzyloxy-1-[3-(2- | D |
| | methoxy-phenyl)-[1,2,4]oxadiazol-5-yl]-ethyl}-amide | |
| 50 | 3-Diethylamino-propane-1-sulfonic acid {(R)-1-[5-(3-cyano- | A |
| | phenyl)-2H-[1,2,4]triazol-3-yl]-3-phenyl-propyl}-amide | |
| 51 | 3-Diethylamino-propane-1-sulfonic acid [(R)-2-benzyloxy-1-(3- | D |
| | pyridin-4-yl-[1,2,4]oxadiazol-5-yl)-ethyl]-amide | |
| 52 | 3-Diethylamino-propane-1-sulfonic acid {(R)-1-[3-(2,6- | B |
| | dimethyl-phenyl)-[1,2,4]oxadiazol-5-yl]-3-phenyl-propyl}-amide | |
| 53 | 3-Diethylamino-propane-1-sulfonic acid {(R)-1-[5-(3-bromo- | A |
| | phenyl)-2H-[1,2,4]triazol-3-yl]-3-phenyl-propyl}-amide | |
| 54 | 2-Diethylamino-ethanesulfonic acid {(R)-1-[3-(2-chloro- | B |
| | pyridin-3-yl)-[1,2,4]oxadiazol-5-yl]-3-phenyl-propyl}-amide | |
| 55 | 3-Diethylamino-propane-1-sulfonic acid [(R)-1-(3-benzo[1,3]dioxol- | A |
| | 5-ylmethyl-[1,2,4]oxadiazol-5-yl)-3-phenyl-propyl]-amide | |
| 56 | 3-Diethylamino-propane-1-sulfonic acid {(R)-1-[5-(3,4- | B |
| | difluoro-benzyl)-2H-[1,2,4]triazol-3-yl]-3-phenyl-propyl}-amide | |
| 57 | 3-Diethylamino-propane-1-sulfonic acid {(R)-1-[3-(4-bromo- | A |
| | benzyl)-[1,2,4]oxadiazol-5-yl]-3-phenyl-propyl}-amide | |
| 58 | 3-Diethylamino-propane-1-sulfonic acid {(R)-1-[5-(2,4-dichloro- | A |
| | phenyl)-2H-[1,2,4]triazol-3-yl]-3-phenyl-propyl}-amide | |
| 59 | 3-Diethylamino-propane-1-sulfonic acid {(R)-1-[3-(4-bromo- | A |
| | phenyl)-[1,2,4]oxadiazol-5-yl]-3-phenyl-propyl}-amide | |
| 60 | 3-Diethylamino-propane-1-sulfonic acid {(R)-1-[3-(2-methoxy- | C |
| | ethyl)-[1,2,4]oxadiazol-5-yl]-3-phenyl-propyl}-amide | |
| 61 | 3-Diethylamino-propane-1-sulfonic acid [(R)-3-phenyl-1-(5-m- | A |
| | tolyl-2H-[1,2,4]triazol-3-yl)-propyl]-amide | |
| 62 | 3-Diethylamino-propane-1-sulfonic acid {(R)-1-[3-(3-fluoro- | A |
| | benzyl)-[1,2,4]oxadiazol-5-yl]-3-phenyl-propyl}-amide | |
| 63 | 3-Diethylamino-propane-1-sulfonic acid {(R)-1-[5-(2,5-difluoro- | B |
| | phenyl)-2H-[1,2,4]triazol-3-yl]-3-phenyl-propyl}-amide | |
| 64 | 3-Diethylamino-propane-1-sulfonic acid {(R)-1-[3-(3-bromo- | A |
| | phenyl)-[1,2,4]oxadiazol-5-yl]-3-phenyl-propyl}-amide | |
| 65 | 3-Diethylamino-propane-1-sulfonic acid {(R)-1-[5-(4-nitro- | A |
| | phenyl)-2H-[1,2,4]triazol-3-yl]-3-phenyl-propyl}-amide | |
| 66 | 3-Diethylamino-propane-1-sulfonic acid {(R)-1-[3-(4-fluoro- | A |
| | phenyl)-[1,2,4]oxadiazol-5-yl]-3-phenyl-propyl}-amide | |
| 67 | 3-Diethylamino-propane-1-sulfonic acid {(R)-1-[3-(4-bromo-2- | B |
| | methyl-phenyl)-[1,2,4]oxadiazol-5-yl]-3-phenyl-propyl}-amide | |
| 68 | 3-Diethylamino-propane-1-sulfonic acid {(R)-1-[3-(2-methyl- | A |
| | benzyl)-[1,2,4]oxadiazol-5-yl]-3-phenyl-propyl}-amide | |
| 69 | 3-Diethylamino-propane-1-sulfonic acid {(R)-1-[3-(4-methoxy- | A |
| | phenyl)-[1,2,4]oxadiazol-5-yl]-3-phenyl-propyl}-amide | |
| 70 | 3-Diethylamino-propane-1-sulfonic acid {(R)-1-[5-(3-chloro- | A |
| | phenyl)-2H-[1,2,4]triazol-3-yl]-3-phenyl-propyl}-amide | |
| 71 | 3-Diethylamino-propane-1-sulfonic acid [(R)-3-phenyl-1-(3-p- | A |
| | tolyl-[1,2,4]oxadiazol-5-yl)-propyl]-amide | |
| 72 | 3-Diethylamino-propane-1-sulfonic acid {(R)-1-[3-(2-fluoro- | A |
| | benzyl)-[1,2,4]oxadiazol-5-yl]-3-phenyl-propyl}-amide | |
| 73 | 3-Diethylamino-propane-1-sulfonic acid {(R)-1-[3-(2,6-dichloro- | A |
| | benzyl)-[1,2,4]oxadiazol-5-yl]-3-phenyl-propyl}-amide | |
| 74 | 3-Diethylamino-propane-1-sulfonic acid {(R)-1-[3-(3-bromo- | A |
| | benzyl)-[1,2,4]oxadiazol-5-yl]-3-phenyl-propyl}-amide | |
| 75 | 3-Diethylamino-propane-1-sulfonic acid {(R)-1-[3-(2-bromo- | A |
| | benzyl)-[1,2,4]oxadiazol-5-yl]-3-phenyl-propyl}-amide | |
| 76 | 2-Diethylamino-ethanesulfonic acid {(R)-1-[3-(3-bromo- | D |
| | phenyl)-[1,2,4]oxadiazol-5-yl]-3-phenyl-propyl}-amide | |
| 77 | 2-Diethylamino-ethanesulfonic acid {(R)-1-[3-(4-methyl- | D |
| | pyridin-3-yl)-[1,2,4]oxadiazol-5-yl]-3-phenyl-propyl}-amide | |
| 78 | 2-Diethylamino-ethanesulfonic acid {(R)-1-[3-(2-methoxy- | C |
| | ethyl)-[1,2,4]oxadiazol-5-yl]-3-phenyl-propyl}-amide | |
| 79 | 2-Diethylamino-ethanesulfonic acid {(R)-1-[3-(4-bromo- | B |
| | phenyl)-[1,2,4]oxadiazol-5-yl]-3-phenyl-propyl}-amide | |
| 80 | 3-Diethylamino-propane-1-sulfonic acid {(R)-1-[5-(3,4-dimethoxy- | B |
| | phenyl)-2H-[1,2,4]triazol-3-yl]-3-phenyl-propyl}-amide | |
| 81 | 2-Diethylamino-ethanesulfonic acid [(R)-1-(3-benzo[1,3]dioxol- | B |
| | 5-ylmethyl-[1,2,4]oxadiazol-5-yl)-3-phenyl-propyl]-amide | |
| 82 | 2-Diethylamino-ethanesulfonic acid {(R)-1-[3-(3-fluoro- | A |
| | benzyl)-[1,2,4]oxadiazol-5-yl]-3-phenyl-propyl}-amide | |
| 83 | 3-Diethylamino-propane-1-sulfonic acid {(R)-1-[3-(4-methyl- | B |
| | pyridin-3-yl)-[1,2,4]oxadiazol-5-yl]-3-phenyl-propyl}-amide | |
| 84 | 2-Diethylamino-ethanesulfonic acid {(R)-1-[3-(4-bromo- | B |
| | benzyl)-[1,2,4]oxadiazol-5-yl]-3-phenyl-propyl}-amide | |
| 85 | 2-Diethylamino-ethanesulfonic acid {(R)-1-[3-(4-bromo-2- | D |
| | methyl-phenyl)-[1,2,4]oxadiazol-5-yl]-3-phenyl-propyl}-amide | |
| 86 | 2-Diethylamino-ethanesulfonic acid [(R)-3-phenyl-1-(3-p-tolyl- | B |
| | [1,2,4]oxadiazol-5-yl)-propyl]-amide | |
| 87 | 2-Diethylamino-ethanesulfonic acid {(R)-1-[3-(2-methyl- | B |
| | benzyl)-[1,2,4]oxadiazol-5-yl]-3-phenyl-propyl}-amide | |
| 88 | 2-Diethylamino-ethanesulfonic acid {(R)-1-[3-(4-methoxy- | D |
| | phenyl)-[1,2,4]oxadiazol-5-yl]-3-phenyl-propyl}-amide | |
| 89 | 2-Diethylamino-ethanesulfonic acid {(R)-1-[3-(4-fluoro- | A |
| | phenyl)-[1,2,4]oxadiazol-5-yl]-3-phenyl-propyl}-amide | |
| 90 | 2-Diethylamino-ethanesulfonic acid {(R)-1-[3-(2,6-dichloro- | A |
| | benzyl)-[1,2,4]oxadiazol-5-yl]-3-phenyl-propyl}-amide | |
| 91 | 2-Diethylamino-ethanesulfonic acid {(R)-1-[3-(2-fluoro- | B |
| | benzyl)-[1,2,4]oxadiazol-5-yl]-3-phenyl-propyl}-amide | |
| 92 | 2-Diethylamino-ethanesulfonic acid {(R)-1-[3-(3-bromo- | A |
| | benzyl)-[1,2,4]oxadiazol-5-yl]-3-phenyl-propyl}-amide | |
| 93 | 2-Diethylamino-ethanesulfonic acid {(R)-1-[3-(2-bromo- | A |
| | benzyl)-[1,2,4]oxadiazol-5-yl]-3-phenyl-propyl}-amide | |
| 94 | 3-Diethylamino-propane-1-sulfonic acid {(R)-1-[5-(4-methoxy- | A |
| | phenyl)-2H-[1,2,4]triazol-3-yl]-3-phenyl-propyl}-amide | |
| 95 | 3-Diethylamino-propane-1-sulfonic acid {(S)-1-[5-(4-methoxy- | A |
| | phenyl)-2H-[1,2,4]triazol-3-yl]-2-phenyl-ethyl}-amide | |
| 96 | 3-Diethylamino-propane-1-sulfonic acid {(R)-1-[5-(2,4-difluoro- | A |
| | phenyl)-2H-[1,2,4]triazol-3-yl]-3-phenyl-propyl}-amide | |
| 97 | 3-Diethylamino-propane-1-sulfonic acid [(R)-1-(3-phenethyl- | A |
| | [1,2,4]oxadiazol-5-yl)-3-phenyl-propyl]-amide | |
| 98 | (R)-4-[3-(4-Bromo-benzyl)-[1,2,4]oxadiazol-5-yl]-4-(3- | B |
| | diethylamino-propane-1-sulfonylamino)-butyramide | |
| 99 | 3-Diethylamino-propane-1-sulfonic acid [(R)-1-(3-phenoxymethyl- | A |
| | [1,2,4]oxadiazol-5-yl)-3-phenyl-propyl]-amide | |
| 100 | 3-Diethylamino-propane-1-sulfonic acid {(R)-1-[3-(2-methoxy- | A |
| | benzyl)-[1,2,4]oxadiazol-5-yl]-3-phenyl-propyl}-amide | |
| 101 | 3-Diethylamino-propane-1-sulfonic acid ((R)-1-{3-[2-(2-chloro- | B |
| | phenyl)-ethyl]-[1,2,4]oxadiazol-5-yl}-3-phenyl-propyl)-amide | |
| 102 | (R)-3-[3-(4-Bromo-benzyl)-[1,2,4]oxadiazol-5-yl]-3-(3- | B |
| | diethylamino-propane-1-sulfonylamino)-propionamide | |
| 103 | 3-Diethylamino-propane-1-sulfonic acid {(R)-1-[3-(5-bromo- | B |
| | pyridin-3-yl)-[1,2,4]oxadiazol-5-yl]-phenyl-propyl}-amide | |
| 104 | 3-Diethylamino-propane-1-sulfonic acid [(R)-3-phenyl-1-(5-o- | A |
| | tolyl-2H-[1,2,4]triazol-3-yl)-propyl]-amide | |
| 105 | 3-Diethylamino-propane-1-sulfonic acid {(R)-1-[5-(4-fluoro- | A |
| | phenyl)-2H-[1,2,4]triazol-3-yl]-3-phenyl-propyl}-amide | |
| 106 | 3-Diethylamino-propane-1-sulfonic acid {(R)-3-phenyl-1-[5-(4- | A |
| | trifluoromethoxy-phenyl)-2H-[1,2,4]triazol-3-yl]-propyl}-amide | |
| 107 | 3-Diethylamino-propane-1-sulfonic acid {(R)-1-[5-(4-methoxy- | B |
| | benzyl)-2H-[1,2,4]triazol-3-yl]-3-phenyl-propyl}-amide | |
| 108 | 3-Diethylamino-propane-1-sulfonic acid [(R)-3-phenyl-1-(5-p- | B |
| | tolyl-2H-[1,2,4]triazol-3-yl)-propyl]-amide | |
| 109 | 3-Diethylamino-propane-1-sulfonic acid {(R)-1-[3-(3-methoxy- | A |
| | benzyl)-[1,2,4]oxadiazol-5-yl]-3-phenyl-propyl}-amide | |
| 110 | 3-Diethylamino-propane-1-sulfonic acid {(R)-1-[5-(4-dimethylamino- | D |
| | phenyl)-2H-[1,2,4]triazol-3-yl]-3-phenyl-propyl}-amide | |
| 111 | 3-Diethylamino-propane-1-sulfonic acid {(R)-1-[3-(4-methoxy- | A |
| | benzyl)-[1,2,4]oxadiazol-5-yl]-3-phenyl-propyl}-amide | |
| 112 | 3-Diethylamino-propane-1-sulfonic acid [(R)-3-phenyl-1-(3- | B |
| | pyridin-2-ylmethyl-[1,2,4]oxadiazol-5-yl)-propyl]-amide | |
| 113 | 3-Diethylamino-propane-1-sulfonic acid ((R)-1-{3-[2-(4-bromo- | A |
| | phenyl)-ethyl]-[1,2,4]oxadiazol-5-yl}-3-phenyl-propyl)-amide | |
| 114 | 3-Diethylamino-propane-1-sulfonic acid {(R)-1-[3-(2-chloro- | A |
| | benzyl)-[1,2,4]oxadiazol-5-yl]-3-phenyl-propy}-amide | |
| 115 | 3-Diethylamino-propane-1-sulfonic acid {(R)-1-[5-(2-fluoro-4- | A |
| | methoxy-phenyl)-2H-[1,2,4]triazol-3-yl]-3-phenyl-propyl}-amide | |
| 116 | 3-Diethylamino-propane-1-sulfonic acid [(R)-1-(3-benzyl- | B |
| | [1,2,4]oxadiazol-5-yl)-3-phenyl-propyl]-amide | |
| 117 | 3-Diethylamino-propane-1-sulfonic acid {(R)-3-phenyl-1-[3-(4- | A |
| | trifluoromethyl-benzyl)-[1,2,4]oxadiazol-5-yl]-propyl}-amide | |
| 118 | 3-Diethylamino-propane-1-sulfonic acid {(R)-1-[5-(1H-indol-4- | A |
| | yl)-2H-[1,2,4]triazol-3-yl]-3-phenyl-propyl}-amide | |
| 119 | 3-Diethylamino-propane-1-sulfonic acid {(R)-1-[5-(4-methoxy- | B |
| | phenyl)-2H-[1,2,4]triazol-3-yl]-2-phenyl-ethyl}-amide | |
| 120 | 3-Diethylamino-propane-1-sulfonic acid {(R)-1-[5-(4-hydroxy- | A |
| | phenyl)-2H-[1,2,4]triazol-3-yl]-3-phenyl-propyl}-amide | |
| 121 | 3-Diethylamino-propane-1-sulfonic acid {(R)-1-[5-(3-hydroxy- | A |
| | phenyl)-2H-[1,2,4]triazol-3-yl]-3-phenyl-propyl}-amide | |
| 122 | 3-Diethylamino-propane-1-sulfonic acid [(R)-3-phenyl-1-(5- | A |
| | thiophen-3-ylmethyl-2H-[1,2,4]triazol-3-yl)-propyl]-amide | |
| 123 | 3-Diethylamino-propane-1-sulfonic acid {(R)-1-[5-(3-methylsulfanyl- | A |
| | phenyl)-2H-[1,2,4]triazol-3-yl]-3-phenyl-propyl}-amide | |
| 124 | 3-Diethylamino-propane-1-sulfonic acid [(R)-1-(5-benzotriazol- | A |
| | 1-ylmethyl-2H-[1,2,4]triazol-3-yl)-3-phenyl-propyl]-amide | |
| 125 | 3-Diethylamino-propane-1-sulfonic acid {(R)-1-[5-(3-cyano-4- | A |
| | methoxy-phenyl)-2H-[1,2,4]triazol-3-yl]-3-phenyl-propyl}-amide | |
| 126 | 3-Diethylamino-propane-1-sulfonic acid {(R)-1-[5-(3-chloro-4- | A |
| | cyano-phenyl)-2H-[1,2,4]triazol-3-yl]-3-phenyl-propyl}-amide | |
| 127 | 3-Diethylamino-propane-1-sulfonic acid {(R)-1-[5-(1H-indol-3- | A |
| | ylmethyl)-2H-[1,2,4]triazol-3-yl]-3phenyl-propyl}-amide | |
| 128 | 3-Diethylamino-propane-1-sulfonic acid {(R)-1-[5-(3-cyano-4- | A |
| | fluoro-phenyl)-2H-[1,2,4]triazol-3-yl]-3-phenyl-propyl}-amide | |
| 129 | 3-Diethylamino-propane-1-sulfonic acid [(R)-1-(5-benzo[b]thiophen- | A |
| | 5-yl-2H-[1,2,4]triazol-3-yl)-3-phenyl-propyl]-amide | |
| 130 | 3-Diethylamino-propane-1-sulfonic acid [(R)-1-(5-benzofuran- | A |
| | 5-yl-2H-[1,2,4]triazol-3-yl)-3-phenyl-propyl]-amide | |
| 131 | 3-Diethylamino-propane-1-sulfonic acid {(R)-1-[5-(1-methyl- | A |
| | 1H-imidazol-4-yl)-2H-[1,2,4]triazol-3-yl]-3-phenyl-propyl}-amide | |
| 132 | 3-Diethylamino-propane-1-sulfonic acid {(R)-1-[5-(3,5-difluoro- | A |
| | benzyl)-2H-[1,2,4]triazol-3-yl]-3-phenyl-propyl}-amide | |
| 133 | 3-Diethylamino-propane-1-sulfonic acid {(R)-1-[5-(4-methyl- | A |
| | 3H-1lambda*4*-thiazol-2-ylmethyl)-2H-[1,2,4]triazol-3-yl]-3- | |
| | phenyl-propyl}-amide | |
| 134 | 3-Diethylamino-propane-1-sulfonic acid {(R)-1-[5-(1H- | B |
| | imidazo[1,2,-a]pyridin-6-yl)-2H-[1,2,4]triazol-3-yl]-3-phenyl- | |
| | propyl}-amide | |
| 135 | 3-Diethylamino-propane-1-sulfonic acid {(R)-1-[5-(3-methyl- | A |
| | 3H-imidazol-4-yl)-2H-[1,2,4]triazol-3-phenyl-propyl}-amide | |
| 136 | 3-Diethylamino-propane-1-sulfonic acid {(R)-1-[5-(3,4-dihydro- | A |
| | 2H-benzo[1,4]oxazin-2-yl)-2H-[1,2,4]triazol-3-yl]-3-phenyl- | |
| | propyl}-amide | |
| 137 | 3-Diethylamino-propane-1-sulfonic acid {(R)-1-[5-(2-methyl- | A |
| | thiazol-4-yl)-2H-[1,2,4]triazol-3-yl]-3-phenyl-propyl}-amide | |
| 138 | 3-Diethylamino-propane-1-sulfonic acid {(R)-1-[5-(6-methoxy- | A |
| | pyridin-3-yl)-2H-[1,2,4]triazol-3-yl]-3-phenyl-propyl}-amide | |
| 139 | 3-Diethylamino-propane-1-sulfonic acid [(R)-1-(5-cyclohexyl- | A |
| | 2H-[1,2,4]triazol-3-yl)-3-phenyl-propyl]-amide | |
| 140 | (R)-4-[3-(4-Bromo-benzyl)-[1,2,4]oxadiazol-5-yl]-4-(3- | B |
| | diethylamino-propane-1-sulfonylamino)-butyramide | |
| 141 | (R)-3-[3-(4-Bromo-benzyl)-[1,2,4]oxadiazol-5-yl]-3-(3- | B |
| | diethylamino-propane-1-sulfonylamino)-propionamide | |
| 142 | (S)-(3-{2-[3-(4-Bromo-benzyl)-[1,2,4]oxadiazol-5-yl]- | B |
| | pyrrolidine-1-sulfonyl}-propyl)-diethyl-amine | |
| 143 | (S)-Diethyl-(3-{2-[3-(4-methoxy-phenyl)-[1,2,4]oxadiazol-5- | B |
| | yl]-pyrrolidine-1-sulfonyl}-propyl)-amine | |
| 144 | (R)-Diethyl-(3-{2-[3-(4-methoxy-phenyl)-[1,2,4]oxadiazol-5- | B |
| | yl]-pyrrolidine-1-sulfonyl}-propyl)-amine | |
| 145 | (R)-(3-{2-[3-(4-Bromo-benzyl)-[1,2,4]oxadiazol-5-yl]- | B |
| | pyrrolidine-1-sulfonyl}-propyl)-diethyl-amine | |
| 146 | (S)-3-Diethylamino-propane-1-sulfonic acid {1-[3-(4-bromo- | B |
| | benzyl)-[1,2,4]oxadiazol-5-yl]-ethyl}-amide | |
| 147 | (R)-3-Diethylamino-propane-1-sulfonic acid {1-[3-(4-bromo- | B |
| | benzyl)-[1,2,4]oxadiazol-5-yl]-ethyl}-amide | |
| 148 | 3-Diethylamino-propane-1-sulfonic acid [3-(4-methoxy- | C |
| | phenyl)-[1,2,4]oxadiazol-5-ylmethyl]-amide | |
| 149 | (S)-(3-{2-[3-(4-Bromo-benzyl)-[1,2,4]oxadiazol-5- | A |
| | yl]-azetidine-1-sulfonyl}-propyl)-diethyl-amine | |
| 150 | (S)-3-Diethylamino-propane-1-sulfonic acid {1-[3-(4-methoxy- | B |
| | phenyl)-[1,2,4]oxadiazol-5-yl]-ethyl}-amide | |
| 151 | 3-Diethylamino-propane-1-sulfonic acid [3-(4-bromo-benzyl)- | B |
| | [1,2,4]oxadiazol-5-ylmethyl]-amide | |
| 152 | (R)-3-Diethylamino-propane-1-sulfonic acid {1-[3-(4-methoxy- | B |
| | phenyl)-[1,2,4]oxadiazol-5-yl]-ethyl}-amide | |
| 153 | (S)-Diethyl-(3-{2-[3-(4-methoxy-phenyl)-[1,2,4]oxadiazol-5- | A |
| | yl]-azetidine-1-sulfonyl}-propyl)-amine | |
| 154 | 3-Diethylamino-propane-1-sulfonic acid {1-[3-(4-bromo- | A |
| | benzyl)-[1,2,4]oxadiazol-5-yl]-1-methyl-ethyl}-amide | |
| 155 | 3-Diethylamino-propane-1-sulfonic acid {1-[3-(4-methoxy- | B |
| | phenyl)-[1,2,4]oxadiazol-5-yl]-1-methyl-ethyl}-amide | |
| 156 | 3-Diethylamino-propane-1-sulfonic acid [5-(4-methoxy- | B |
| | phenyl)-2H-[1,2,4]triazol-3-ylmethyl]-amide | |
| 157 | 3-Diethylamino-propane-1-sulfonic acid [5-(4-chloro-benzyl)- | B |
| | 2H-[1,2,4]triazol-3-ylmethyl]-amide | |
| 158 | 3-Diethylamino-propane-1-sulfonic acid [3-(4-fluoro-phenyl)- | E |
| | [1,2,4]oxadiazol-5-ylmethyl]-amide | |
| 159 | (S)-3-Diethylamino-propane-1-sulfonic acid {1-[5-(4-methoxy- | B |
| | phenyl)-2H-[1,2,4]triazol-3-yl]-3-methyl-butyl}-amide | |
| 160 | (R)-3-Diethylamino-propane-1-sulfonic acid {1-[5-(4-methoxy- | B |
| | phenyl)-2H-[1,2,4]triazol-3-yl]-3-methyl-butyl}-amide | |

*A refers to a compound having antagonist activity with a Ki <100 nM in a cell based assay.
B refers to a compound having antagonist activity with a Ki between 100 nM and 500 nM in a cell based assay.
C refers to a compound having antagonist activity with a Ki between 500 nM and 1000 nM in a cell based assay.
D refers to a compound having antagonist activity with Ki, ≤1000 nM in a cell-based assay.
E refers to other exemplary compounds.

Representative compounds that modulate GHS-R include the compounds of formulas (I), (II, (III), (IV), (V), and (VI) below, where all variables are as described herein.

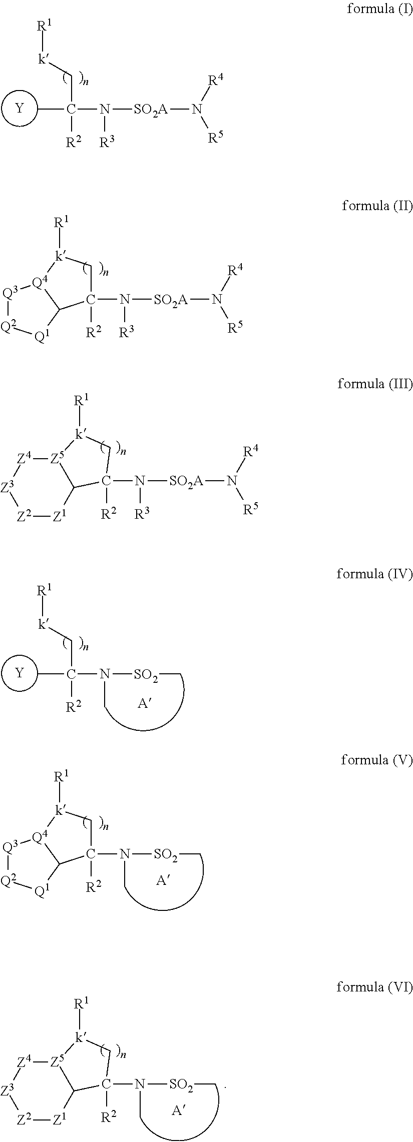

In some preferred embodiments, Y is a 5 membered heteroaromatic moiety substituted with 1 or 2 substituents as described herein. Exemplary Y moieties are reproduced below.

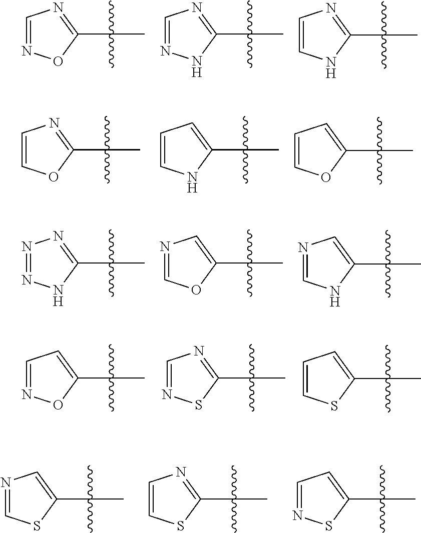

In another aspect, the invention features a compound, including the hydrogens depicted on the nitrogen atoms, can be substituted with R¹⁰. In some preferred embodiments, the heteroaryl moiety includes 1 or 2 R¹⁰substituents. In some preferred embodiments, R¹⁰ is aryl, arylalkyl, or R¹⁵. When two R¹⁰substituents are included, in some embodiments, one R¹⁰ is R¹⁵ and the second R¹⁰ is a different substituent, such as alkyl, alkoxy, halo, etc.

In certain instances, R¹ is an aryl moiety such as a phenyl moiety, for example unsubstituted or substituted aryl moiety. In some instances, R¹ is a heteroaryl moiety such as an indole moiety. In many instances where R¹ is aryl or heteroaryl (or other lipophilic moiety such as alkyl), K is an oxygen or a bond. A and R⁴ and R⁵ can be chosen to vary the compound's type of interaction with GHS-R. For example, in some instances where R⁴ and R⁵ are both hydrogen, the compound is an agonist of GHS-R. In other instances where R⁴ and R⁵ are both independently alkyl, the compound is an antagonist of GHS-R.

Other aspects of this invention relate to a composition having a compound of any of the formulae described herein and a pharmaceutically acceptable carrier; or a compound of any of the formulae described herein, an additional therapeutic compound (e.g., an anti-hypertensive compound or a cholesterol lowering compound), and a pharmaceutically acceptable carrier; or a compound of any of the formulae described herein, an additional therapeutic compound, and a pharmaceutically acceptable carrier.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The compounds described herein can be made using a variety of synthetic techniques. In some embodiments, a Y moiety, or other ring corresponding to a Y moiety, can be synthesized onto an amino acid or amino acid type starting material as depicted in schemes A and Band B′ below.

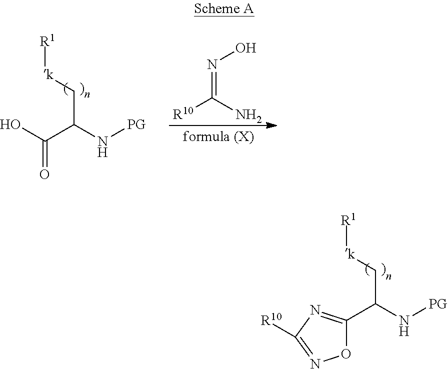

In the schemes provided herein, all variables are defined as herein and PG is a nitrogen protecting group. The nitrogen protected amino acid is reacted with a N-hydroxy imidamide (amidoxime) moiety (which is prepared by reacting a cyano containing moiety with hydroxylamine) to produce an oxadiazole containing moiety. The resulting compound can be further manipulated to form a compound of formula (I) by removing the nitrogen protecting group and reacting the resulting moiety with an activated sulfone, such as a sulfonyl chloride as depicted below.

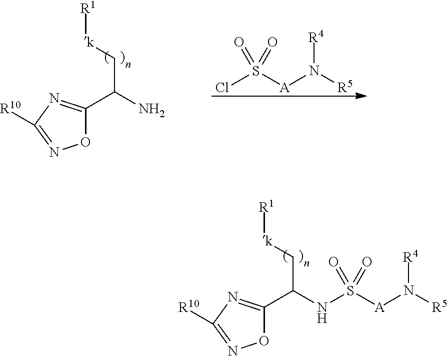

Scheme B below depicts the formation of a triazole containing moiety which can be further reacted in a manner similar to the oxadiazole containing moiety to form a compound of formula (I).

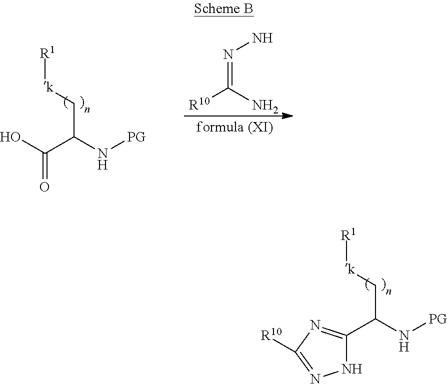

Scheme B′ below depicts an alternative method of forming a triazole containing moiety which can be further reacted in a manner similar to the oxadiazole containing moiety to form a compound of formula (I).

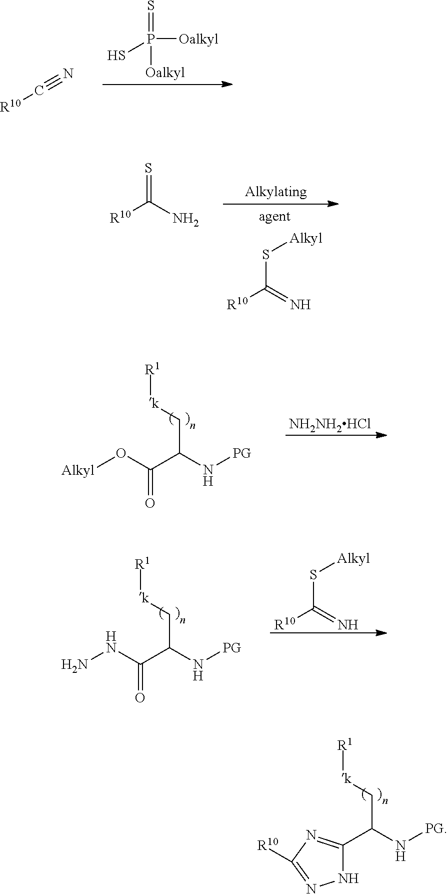

The triazole precursor moiety can be prepared in a variety of manners, for example, by reacting a cyano containing moiety with a hydrazine hydrate (to form the intermediate amidrazone) as depicted in scheme B. Alternatively, the triazole precursor moiety can be prepared as shown in scheme B′ by reacting a nitrile moiety (e.g., an arylnitrile or benzylnitrile) with a dialkyl dithiophosphate moiety such as diethyl dithiophosphate to provide an thioimidate, which is reacted with a acyl hydrazide moiety to provide the triazole precursor. The acyl hydrazide moiety can be prepared, for example, by reacting a carboxylic acid or derivative thereof with hydrazine.

In other embodiments, a compound of formula (I) can be prepared by first reacting an activated sulfone moiety (e.g., a sulfonyl chloride) with an amino acid moiety or protected amino acid, as depicted in Scheme C below.

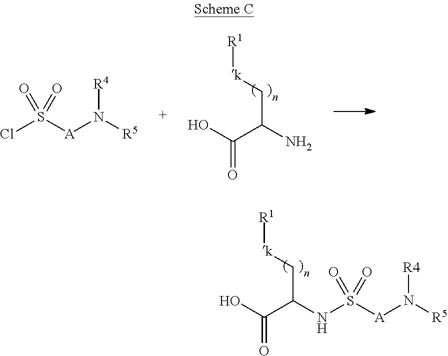

The free carboxyl moiety can then be further manipulated to produce a compound of formula (I). For example, the free carboxyl moiety can be reacted with a compound of formula (X) or (XI) above to form an oxadiazole or triazole containing compound of formula (I) in a manner similar to that described in schemes A, B and B′ above.

As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Additionally, the compounds disclosed herein can be prepared on a solid support or using a solid phase peptide synthesis.

The term “solid support” refers a material to which a compound is attached to facilitate identification, isolation, purification, or chemical reaction selectivity of the compound. Such materials are known in the art and include, for example, beads, pellets, disks, fibers, gels, or particles such as cellulose beads, pore-glass beads, silica gels, polystyrene beads optionally cross-linked with divinylbenzene and optionally grafted with polyethylene glycol, poly-acrylamide beads, latex beads, dimethylacrylamide beads optionally cross-linked with N,N′-bis-acryloyl ethylene diamine, glass particles coated with hydrophobic polymer, and material having a rigid or semi-rigid surface. The solid supports optionally have functional groups such as amino, hydroxy, carboxy, or halo groups, (see, Obrecht, D. and Villalgrodo, J. M., Solid-Supported Combinatorial and Parallel Synthesis of Small-Molecular-Weight Compound Libraries, Pergamon-Elsevier Science Limited (1998), and include those useful in techniques such as the “split and pool” or “parallel” synthesis techniques, solid-phase and solution-phase techniques, and encoding techniques (see, for example, Czarnik, A. W., Curr. Opin. Chem. Bio., (1997) 1, 60).

The term “solid phase peptide” refers to an amino acid, which is chemically bonded to a resin (e.g., a solid support). Resins are generally commercially available (e.g., from SigmaAldrich). Some examples of resins include Rink-resins. Tentagel S RAM, MBHA, and BHA-resins.

The compounds of this invention may contain one or more asymmetric centers and thus occur as racemates and racemic mixtures, single enantiomers and enantiometric mixtures, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention (for example an imidate ester of an amide), which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

The compounds of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undeconoate. Salts derived from appropriate bases include alkali metal (e.g., sodium), alkaline earth metal (e.g., magnesium), ammonium and N-(alkyl)₄ salts. This invention also envisions the quaternization of any basic nitrogen-containing groups of the compounds disclosed herein. Water or oil-soluble or dispersible products may be obtained by such quaternization.

### Glutathione Peroxidase Mimics/Mimetics and Inducers

Glutathione peroxidase is one of the enzymes which are actively involved in the regulation of the concentration of oxygen-derived free radicals formed during various physiological or pathological processes. As used herein, the term “glutathione peroxidase” designates any enzyme having glutathione peroxidase activity. By way of illustration of these enzymes, there may in particular be mentioned as glutathione peroxidase 1 (GPX1), glutathione peroxidase 2 (GPX2), glutathione peroxidase 3 (GPX3), glutathione peroxidase 4 (GPX4), glutathione peroxidase 5 (GPX5), glutathione peroxidase 6 (GPX6), glutathione peroxidase 7 (GPX7), and glutathione peroxidase 8 (GPX8). GPX1 and GPX4 are expressed in most tissues with a clear predominance in the erythrocytes, the liver and the kidneys for GPX1 (Chambers et al; EMBO J 5: 1221-1227 (1986)) and in the testicles for GPX4 [Roveri et al; J. Biol. Chem. 267:6142-6146 (1992)]. GPX3 is produced in the kidneys, the lungs, the heart, the breast, the placenta as well as in the liver (Chu et al. Blood 79: 3233-3238 (1992)) as for GPX2, it has mainly been demonstrated in the gastrointestinal tissues and in the liver [Chu et al. J. Biol. Chem. 268: 2571-257 (1993)]. The DNA sequence encoding glutathione peroxidase may be a cDNA, a genomic DNA (gDNA), or a hybrid construct consisting for example of a cDNA into which one or more introns would be inserted. The nucleic sequence of the cDNA encoding human glutathione peroxidase has been described by [Mullenbach et al., Oxy-Radicals in Molecular Biology and Pathology, 313-326, (1988)]. It may also be synthetic or semisynthetic sequences.

Exemplary proteins that intended to be encompassed by the term “glutathione peroxidase” include those having amino acid sequences disclosed in GenBank with accession number for Homo sapiens (Human)—NP_000572, NP_002074, NP_002075, NP_001034936, NP_001500, NP_874360, NP_056511, NP_001008398, or Mus musculus (Mouse)—NP_032186, NP_109602, NP_032187, NP_001032830, NP-034473, NP_663426, NP_077160, NP_081403. Exemplary nucleic acid molecules that encode glutathione peroxidase are those disclosed in GenBank with accession number for Homo sapiens (Human)—NM_000581, NM_002083, NM_002084, NM_001039847, NM_001509, NM_182701, NM_015696, NM_001008397, or Mus musculus (Mouse)—NM_008160, NM_030677, NM_001083929, NM_001037741, NM_010343, NM_145451, NM_024198, NM_027127.

The present disclosure provides for a number of Glutathione peroxidase mimics to be used in a therapeutic combination with ghrelin or ghrelin variants in treating mBI. Representative non-limiting examples of glutathione peroxidase mimics include: 2-phenyl-1,2-benzoisoselenazol-3(2H)-one (ebselen); 6A,6B-diseleninic acid-6A′,6B′-selenium bridged β-cyclodextrin (6-diSeCD); and 2,2′-diseleno-bis-Beta-cyclodextrin (2-diSeCD) as disclosed in U.S. Pat. No. 7,923,442, which is incorporated herein by reference in its entirety for all of its disclosure, including all methods, materials, etc.

In one embodiment, the glutathione peroxidase mimic/mimetic or its isomer, metabolite, and/or salt thereof is represented by the compound of formula (A):

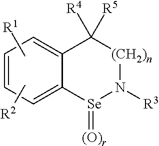

wherein R¹ and R² are independently hydrogen; lower alkyl; OR⁶; —(CH₂)ₘNR⁶R⁷; —(CH₂)_{q}NH₂; —(CH₂)ₘNHSO₂(CH₂)₂NH₂; —NO₂; —CN; —SO₃H; —N⁺(R⁵)₂O⁻; F; Cl; Br; I; —(CH₂)ₘR⁸; —(CH₂)ₘCOR⁸; —S(O)NR⁶R⁷; —SO₂NR⁶R⁷; —CO(CH₂)ₚCOR⁸; R⁹;
R³=hydrogen; lower alkyl; aralkyl; substituted aralkyl; —(CH₂)ₘCOR⁸; —(CH₂)_{q}R⁸; —CO(CH₂)ₚCOR⁸; —(CH₂)ₘSO₂R⁸; —(CH₂)ₘS(O)R⁸;
R⁴=lower alkyl; aralkyl; substituted aralkyl; —(CH₂)ₚCOR⁸; —(CH₂)ₚR⁸; F;
R⁵=lower alkyl; aralkyl; substituted aralkyl;
R⁶=lower alkyl; aralkyl; substituted aralkyl; —(CH₂)ₘCOR⁸; —(CH₂)_{q}R⁸;
R⁷=lower alkyl; aralkyl; substituted aralkyl; —(CH₂)ₘCOR⁸;
R⁸=lower alkyl; aralkyl; substituted aralkyl; aryl; substituted aryl; heteroaryl; substituted heteroaryl; hydroxy; lower alkoxy;
R⁹ is represented by any structure of the following formulae:

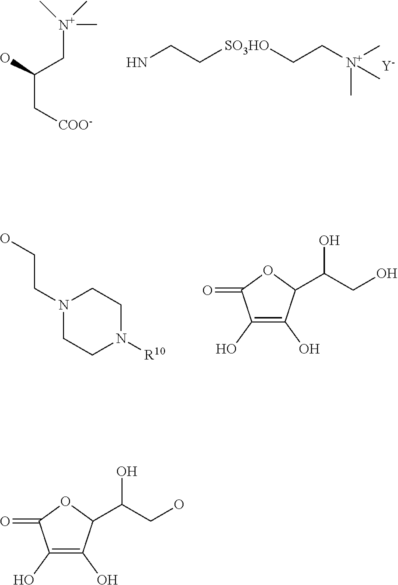

R¹⁰=hydrogen; lower alkyl; aralkyl or substituted aralkyl; aryl or substituted aryl;
Y⁻ represents the anion of a pharmaceutically acceptable acid;
n=0, 1; m=0, 1, 2; p=1, 2, 3; q=2, 3, 4; and
r=O, 1.

In another embodiment, the glutathione peroxidase mimetic or its isomer, metabolite, and/or salt thereof is represented by the compound of formula (B):

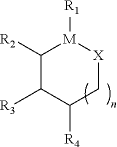

wherein,
   X is O or NH
   M is Se or Te
   n is 0-2
   R₁ is oxygen; and forms an oxo complex with M; or
R₁ is oxygen or NH; and
forms together with the metal, a 4-7 member ring, which optionally is substituted by an oxo or amino group; or
forms together with the metal, a first 4-7 member ring, which is optionally substituted by an oxo or amino group, wherein said first ring is fused with a second 4-7 member ring, wherein said second 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₂, R₃ and R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B}are independently H, alkyl or aryl; or R₂, R₃ or R₄ together with the organometallic ring to which two of the substituents are attached, form a fused 4-7 member ring system wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B}are independently H, alkyl or aryl; wherein R₄ is not an alkyl; and
wherein if R₂, R₃ and R₄ are hydrogen and R₁ forms an oxo complex with M, n is 0 then M is Te; or
if R₂, R₃ and R₄are hydrogen and R₁ is an oxygen that forms together with the metal an unsubstituted, saturated, 5 member ring, n is 0 then M is Te; or
if R₁ is an oxo group, and n is 0, R₂ and R₃ form together with the organometallic ring a fused benzene ring, R₄ is hydrogen, then M is Se; or
if R₄ is an oxo group, and R₂ and R₃ form together with the organometallic ring a fused benzene ring, R₁ is oxygen, n is 0 and forms together with the metal a first 5 membered ring, substituted by an oxo group a to R₁, and said ring is fused to a second benzene ring, then M is Te.

In another embodiment, the glutathione peroxidase mimetic or its isomer, metabolite, and/or salt thereof is represented by the compound of formula (C):

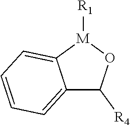

wherein, M, R₁ and R₄are as described above.

In another embodiment, the glutathione peroxidase mimetic or its isomer, metabolite, and/or salt thereof is represented by the compound of formula (D):

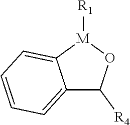

wherein, M, R₂, R₃ and R₄are as described above;

In another embodiment, the glutathione peroxidase mimetic or its isomer, metabolite, and/or salt thereof is represented by the compound of formula (E):

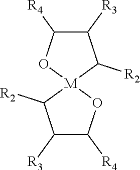

wherein, M, R₂, R₃ and R₄are as described above;

In another embodiment, the glutathione peroxidase mimetic or its isomer, metabolite, and/or salt thereof is represented by the compound of formula (F):

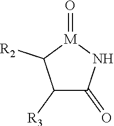

wherein, M, R₂, and R₃are as described above.

In another embodiment, the glutathione peroxidase mimetic or its isomer, metabolite, and/or salt thereof is represented by the compound of formula (G):

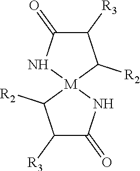

wherein, M, R₂, and R₃are as described above.

In another embodiment, the glutathione peroxidase mimetic or its isomer, metabolite, and/or salt thereof is represented by the compound of formula (H):

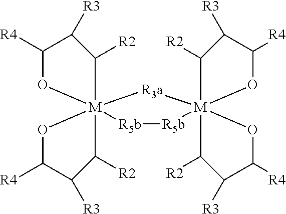

wherein,
M is Se or Te;
R₂, R₃ or R₄are independently hydrogen, alkyl, alkoxy, nitro, aryl, cyano, hydroxy, amino, halogen, oxo, carboxy, thio, thioalkyl, or —NH(C═O)R^{A}, —C(═)NR^{A}R^{B}, —NR^{A}R^{B} or —SO₂R where R^{A} and R^{B}are independently H, alkyl or aryl; or R₂, R₃ or R₄ together with the organometallic ring to which two of the substituents are attached, is a fused 4-7 member ring system, wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B}are independently H, alkyl or aryl; and
R₅ₐ or R_{5b} is one or more oxygen, carbon, or nitrogen atoms and forms a neutral complex with the chalcogen.

In another embodiment, the glutathione peroxidase mimetic or its isomer, metabolite, and/or salt thereof is represented by the compound of formula (I):

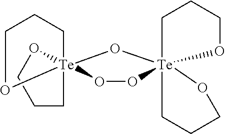

or their combination.

In another embodiment, the glutathione peroxidase mimetic or its isomer, metabolite, and/or salt thereof is represented by the compound of formula (J):

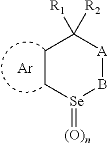

in which: R₁hydrogen; lower alkyl; optionally substituted aryl; optionally substituted lower aralkyl; R₂=hydrogen; lower alkyl; optionally substituted aryl; optionally substituted lower aralkyl; A=CO; (CR₃R₄)ₘ; B═NR₅; O; S; Ar=optionally substituted phenyl or an optionally substituted radical of formula:

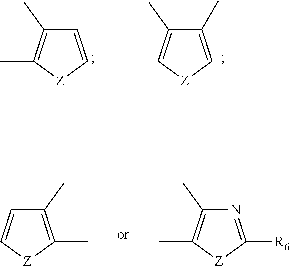

in which: Z═O; S; NR₅; R₃=hydrogen; lower alkyl; optionally substituted aryl; optionally substituted lower aralkyl R₄=hydrogen; lower alkyl; optionally substituted aryl; optionally substituted lower aralkyl; R₅=hydrogen; lower alkyl; optionally substituted aryl; optionally substituted lower aralkyl; optionally substituted heteroaryl; optionally substituted lower heteroaralkyl; CO(lower alkyl); CO(aryl); SO₂ (lower alkyl); SO₂(aryl); R₆=hydrogen; lower alkyl; optionally substituted aryl; optionally substituted lower aralkyl; optionally substituted heteroaryl; optionally substituted lower heteroaralkyl; trifluoromethyl;

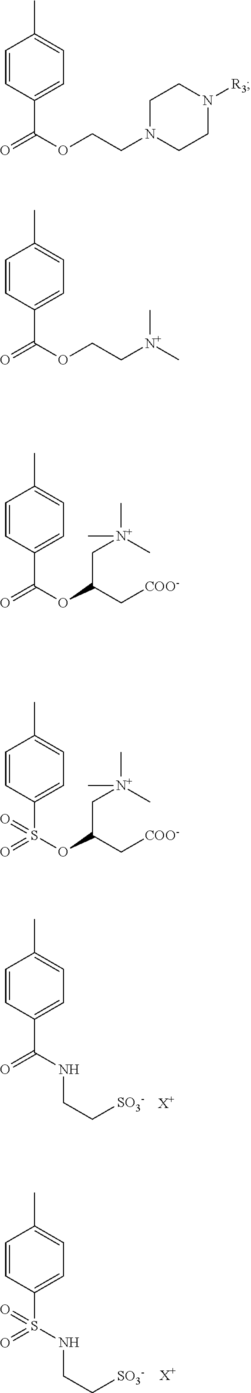

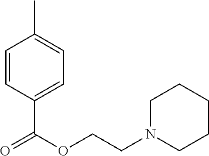 [SMILES: Cc1ccc(C(=O)OCCN2CCCCC2)cc1]

m=0 or 1; n=0 or 1; X⁺ represents the cation of a pharmaceutically acceptable base; and their pharmaceutically acceptable salts of acids or bases.

In other embodiments compounds useful for the purposes herein include 4,4-dimethyl-thieno-[3,2-e]-isoselenazine, 4,4-dimethyl-thieno-[3,2-e]-isoselenazine-1-oxide, 4,4-dimethyl-thieno-[2,3-e]-isoselenazine, and 4,4-dimethyl-thieno-[2,3-e]-isoselenazine-1-oxide.

In another embodiment, the glutathione peroxidase mimetic or its isomer, metabolite, and/or salt thereof is represented by the compound of formula (K):

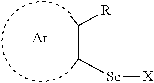

in which: R=hydrogen; —C(R₁R₂)-A-B;
R₁=lower alkyl; optionally substituted aryl; optionally substituted lower aralkyl;
R₂=lower alkyl: optionally substituted aryl: optionally substituted lower aralkyl;
A=CO; (CR₃R₄)ₙ;
B represents NR₅R₆; N³⁰ R₅R₆R₇Y⁻; OR₅; SR₅;
Ar=an optionally substituted phenyl group or an optionally substituted radical of

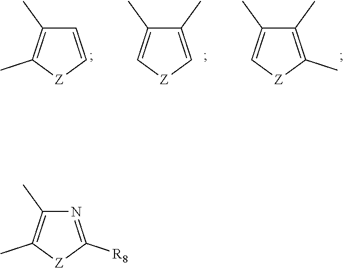

in which Z represents O; S; NR₅; when R═—C(R₁R₂)-A-B or Ar=a radical of formula

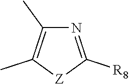

in which Z═O; S; NR₅; when R is hydrogen; X═Ar(R)—Se—; —S-glutathione; —S—N-acetylcysteine; —S-cysteine; —S-penicillamine; —S-albumin; —S-glucose;

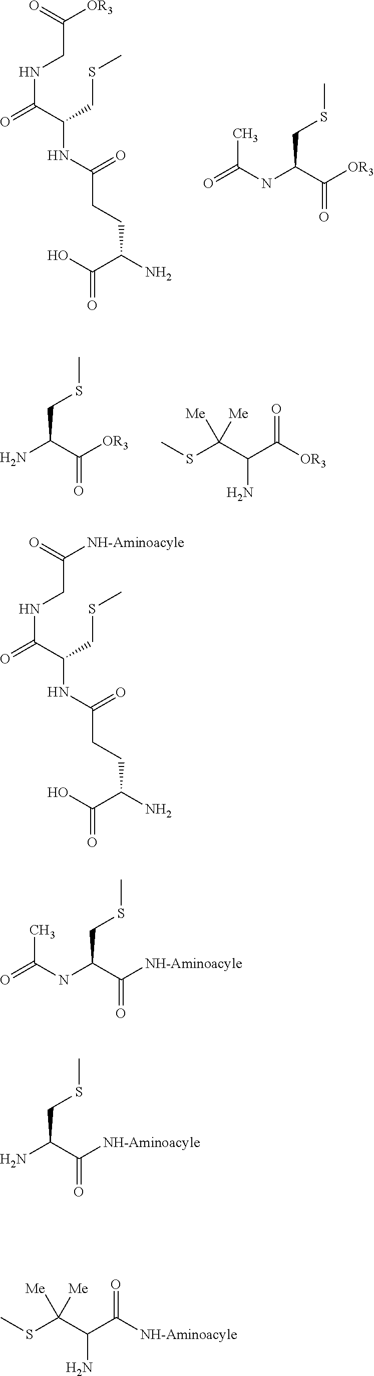

R₃=hydrogen; lower alkyl; optionally substituted aryl, optionally substituted lower aralkyl;
R₄=hydrogen; lower alkyl; optionally substituted aryl: optionally substituted lower aralkyl;
R₅=hydrogen; lower alkyl; optionally substituted aryl: optionally substituted lower aralkyl; optionally substituted heteroaryl; optionally substituted lower heteroaralkyl; CO(lower alkyl); CO(aryl); SO₂(lower alkyl); SO₂ (aryl);
R₆=hydrogen; lower alkyl; optionally substituted aryl; optionally substituted lower aralkyl; optionally substituted heteroaryl; optionally substituted lower heteroaralkyl;
R₇=hydrogen; lower alkyl; optionally substituted aryl: optionally substituted lower aralkyl; optionally substituted heteroaryl; optionally substituted lower heteroaralkyl;
R₈=hydrogen; lower alkyl; optionally substituted aryl; optionally substituted lower aralkyl; optionally substituted heteroaryl; optionally substituted lower heteroaralkyl; trifluoromethyl;

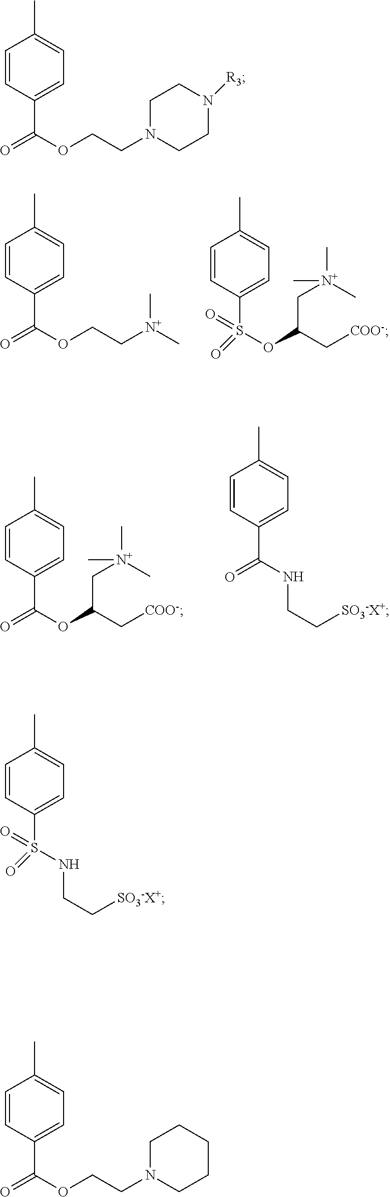

n=0 or 1; X⁺ represents the cation of a pharmaceutically acceptable base;
Y⁻ represents the anion of a pharmaceutically acceptable acid;
and their salts of pharmaceutically acceptable acids or bases. Additional compounds are disclosed in U.S. Patent Application No: 2008/0207679, which is incorporated herein by reference in its entirety for all of its disclosure, including all methods, materials, etc.

The present disclosure provides for a number of glutathione peroxidase inducers to be used in a therapeutic combination with ghrelin or ghrelin variants in treating mBI. Representative non-limiting examples of glutathione peroxidase inducers include: selenium and retinoic acid (Brigelius-Flohe, R., 1999, Free Radicals in Biology and Medicine, 27, 951-965; Chu et al., 1999, Journal of Nutrition 129, 1846-1854).

### Kits

Ghrelin variant compositions may be administered alone or in combination with pharmaceutically acceptable carriers or excipients, in either single or multiple doses. The formulations may conveniently be presented in unit dosage form by methods known to those skilled in the art. The compounds can be provided in a kit. Such a kit typically contains an active compound in dosage forms for administration. The kit comprises an amount of dosage units corresponding to the relevant dosage regimen. In some embodiment, the kit comprises a pharmaceutical composition comprising a ghrelin variant compound or a pharmaceutically acceptable salt thereof and pharmaceutically acceptable carrier, vehicles and/or excipients, said kit having multiple dosage units. The dosage units comprise an amount of a ghrelin variant or a salt thereof equivalent to from about 0.3 μg to about 600 mg ghrelin, such as from about 2.0 μg to about 200 mg ghrelin, such as from about 5.0 μg to about 100 mg ghrelin, such as from about 10 μg to about 50 mg ghrelin, such as from about 10 μg to about 5 mg ghrelin, such as from about 10 μg to about 1.0 mg ghrelin.

The kit contains instructions indicating the use of the dosage form to achieve a desirable affect and the amount of dosage form to be taken over a specified time period. Accordingly, in one embodiment the kit comprises instructions for administering the pharmaceutical composition. In particular said instructions may include instructions referring to administration of said pharmaceutical composition after mBI or concussion, or at the most about 12 hours after the incident causing mBI or concussion, such as at the most about 6 hours after the incident causing mBI or concussion, such as at the most about 3 hours after the incident causing mBI or concussion, such as at the most about 1 hours after the incident causing mBI or concussion, such as at the most about 30 minutes after the incident causing mBI or concussion, such as at the most about 10 minutes after the incident causing mBI or concussion, such as at the most about 5 minutes after the incident causing mBI or concussion.

### EXAMPLES

The following Examples are intended to further illustrate certain embodiments of the disclosure and are not intended to limit its scope.

### Example 1—Ghrelin Variants Reduce Oxidative Burst Following mBI

Ghrelin variant administration reduces oxidative burst in inflammatory cells following mBI. Since no well-accepted animal model exists for concussions, a very small cerebral lesion that closely mimics mBI is used as a model of mBI. C57/B6 mice anesthetized with 5% isoflurane in oxygen (1.7 L/min) are given 0.3 mg/kg buprenorphine subcutaneously for analgesia prior to infliction of the mild brain injury. Anesthesia is assessed by paw pinch reflex. After creating a burr hole through the dura with a dental drill, a lesion using a controlled cortical impactor (CCI) is used to create injury 1 mm lateral and posterior to the bregma (5.0 mm/sec at a depth of 1.0 mm).

Animals are separated into three treatment groups: 1) Sham, 2) mBI, and 3) mBI plus ghrelin variant. A variety of doses can be tested depending upon the particular ghrelin variant. For example, 1 to 50 μg at one or more time points. As one example, treatment with subcutaneous ghrelin variant: 1 dose of 10 μg following mBI and additional dose 10 μg 1 hour after. Brain tissue is harvested at 2-8 hours, preferably about 6 hours post-injury. Reactive oxygen burst (ROS) is measure by neutrophil oxidative burst in leukocytes isolated from brain tissue. Dihydrorhodamine 123 (DHR 123) diffuses across the cell membrane. When it encounters reactive oxygen species (ROS) it fluoresces green. The fluorescence is then measured (AFU), higher intensity means greater oxidative burst and, therefore greater concentration or amount of ROS. Brain cells are isolated (for example by collagenase Dispase/DNAse and a Percoll gradient with resuspension of the pellet using 100 μL of HBSS). Oxidative burst, which is a marker for ROS, can be measured by respiratory burst using flow cytometry (e.g., FL1 channel with 488-nm laser).

Oxidative burst was increased in animals following mBI compared to control. Ghrelin treatment reduced the oxidative burst compared to mBI. Subcutaneous ghrelin reduces oxidative burst in inflammatory cells following mBI. Mild BI plus ghrelin reduced oxidative burst as quantified by Arbitrary Fluorescence Units (AFU). Oxidative burst increased in animals following mBI compared to control. Ghrelin treatment reduced the oxidative burst compared to mBI.

Ghrelin variant administration increases UCP-2 protein in brain tissue. Mild BI or concussion does not cause the massive tissue and cellular damage witnessed in severe or moderate TBI. Rather, concussions cause subtle metabolic changes within the brain, specifically an increase oxidative stress and overproduction of reactive oxygen species (ROS) which in turn can damage neuroconnectivity, lead to neuron damage and encephalopathic changes with recurrent injury.

Ghrelin variant treatment, by increasing uncoupling protein-2 (UCP-2) expression in the brain, has the ability to decrease ROS and the subsequent damage caused to neurons following concussions. This is especially important for chronic concussions where preventing the inflammatory, excitatory milieu of ROS would have significant clinical impact. Ghrelin variant treatment would decrease ROS and therefore prevent the metabolic consequence of concussions and the chronic conditions that are associated thereof.

### Example 2—Using a Binding Assay to Determine the Binding Ability of Ghrelin Variants

The binding ability of ghrelin variants to GHS-R can be determined by a binding assay. Chinese hamster ovary cell line cells, CHO-K1, are prepared to express the human recombinant GHS receptor. The cells can be prepared by any suitable method. One such method can include: The cDNA for human growth hormone secretagogue receptor (hGHS-R1a, or ghrelin receptor) is cloned by Polymerase Chain Reaction (PCR) using human brain RNA as a template (Clontech, Palo Alto, Calif.), gene specific primers flanking the full-length coding sequence of hGHS-R, (S: 5′-ATGTGGAACGCGACGCCCAGCGAAGAG-3′ (SEQ ID NO: 39) and AS: 5′-TCATGTATTAATACTAGATTCTGTCCA-3 (SEQ ID NO: 40)), and Advantage 2 PCR Kit (Clontech). The PCR product is cloned into the pCR2.1 vector using Original TA Cloning Kit (Invitrogen, Carlsbad, Calif.). The full length human GHS-R is subcloned into the mammalian expression vector pcDNA 3.1 (Invitrogen). The plasmid is transfected into the Chinese hamster ovary cell line, CHO-K1 (American Type Culture Collection, Rockville, Md.), by calcium phosphate method (Wigler, M et al., Cell 11, 223, 1977). Single cell clones stably expressing the hGHS-R are obtained by selecting transfected cells grown in cloning rings in RPMI 1640 media supplemented with 10% fetal bovine serum and 1 mM sodium pyruvate containing 0.8 mg/ml G418 (Gibco, Grand Island, N.Y.).

GHS-R binding assay is performed by homogenizing the CHO-K1 cells expressing the human recombinant GHS receptor in 20 ml of ice-cold 50 mM Tris-HCl with a Brinkman Polytron (Westbury, N.Y.). The homogenates are washed twice by centrifugation (39,000 g/10 min), and the final pellets are resuspended in 50 mM Tris-HCl, containing 2.5 mM MgCl₂, and 0.1% BSA. For the GHS-R binding assay, aliquots (0.4 ml) are incubated with 0.05 nM (¹²⁵I)ghrelin variant (“2000 Ci/mmol, Perkin Elmer Life Sciences, Boston, Mass.), with and without 0.05 ml of unlabeled competing test peptides. After a 60 min incubation (4° C.), the bound (¹²⁵I)ghrelin variant is separated from the free by rapid filtration through GF/C filters (Brandel, Gaithersburg, Md.), which have been previously soaked in 0.5% polyethyleneimine/0.1% BSA. The filters then are washed three times with 5 ml aliquots of 50 mM Tris-HCl and 0.1% bovine serum albumin, and the bound radioactivity trapped on the filters is counted by gamma spectrometry (Wallac LKB, Gaithersburg, Md.). Specific binding is defined as the total (¹²⁵I)ghrelin variant bound minus that bound in the presence of 1000 nM ghrelin variant.

### Example 3—Functional Activity of Ghrelin Variants Determined by GHS-R Activity Assays

The functional activity of a ghrelin variant is examined using GHS-R functional activity assays in vitro and in vivo. Ghrelin variant binding to GSH receptor can mediate intracellular iCa²⁺ mobilization in vitro. Ghrelin variant may also be tested for ability to stimulate or suppress release of growth hormone (GH) in vivo.

Cells expressing human GSH receptor can be used. For example, CHO-K1 cells expressing the human GSH receptor are harvested by incubating in a 0.3% EDTA/phosphate buffered saline solution (25° C.), and are washed twice by centrifugation. The washed cells are resuspended in Hank's—buffered saline solution (HBSS) for loading of the fluorescent Ca²⁺ indicator Fura-2AM. Cell suspensions of approximately 10⁶cells/ml are incubated with 2 μM Fura-2AM for 30 min at about 25° C. Unloaded Fura-2AM is removed by centrifugation twice in HBBS, and the final suspensions are transferred to a spectrofluorometer (Hitachi F-2000) equipped with a magnetic stirring mechanism and a temperature-regulated cuvette holder. After equilibration to 37° C., the ghrelin variant is added for measurement of intracellular Ca²⁺ mobilization. The excitation and emission wavelengths can be, for example, 340 and 510 nm, respectively.

Ghrelin variant may be tested for its ability to stimulate or suppress release of growth hormone (GH) in vivo (Deghenghi, R., et al., Life Sciences, 1994, 54, 1321-1328; International Application No. WO 02/08250; each of which is incorporated herein by reference in its entirety). Thus, for example, in order to ascertain a ghrelin variant's ability to stimulate GH release in vivo the compound may be injected subcutaneously in 10-day old rats at a dose of, e.g., 300 mg/kg. The circulating GH may be determined at, e.g., 15 minutes after injection and compared to GH levels in rats injected with a solvent control.

### Example 4—Clinical Study of the Use of Ghrelin Variants for Mild Brain Injury

The use of a ghrelin variant for mild brain injury (concussion) in athletes (e.g., football players) is clinically studied. From 10-200 subjects are selected with one or more of the following exclusion criteria: previous neurologic disorder (seizure, tumor, operative brain injury) or recent concussion (30 days), undocumented concussion mechanism, additional injuries that prevent complete follow-up, no baseline score, outside age range, pregnancy. The study group is randomized, if desired, and then divided into two groups, the treatment group and a placebo group. The treatment group is given one 2 microgram/kg IV infusion with 24 hours of the mild brain injury (e.g., concussion).

Follow up is conducted by analyzing changes from baseline concussion scoring for symptoms, for example, using SCAT-2, SCAT-3, ImPACT methodologies or any of the other methodologies described herein, at Days 1, 3 and 7 following injury. Additional follow up is conducted until the athlete returns to his/her field of play (symptom-free). The primary endpoint is improvement in one or more symptoms on the scoring methodology and/or one or more symptoms or sequelae described herein. Each composite raw score can be analyzed in addition to the total score. An optional secondary endpoint is to analyze or determine the time to return to field of play, the time to individual symptom relief, or the change in total SCAT-2 (or other methodology) score.

In some embodiments, the endpoint or an additional endpoint can be rate of post-concussion syndrome (PCS) diagnosis in the control versus treatment. PCS can be diagnosed using a scorecard or other suitable method, for example, the ICD-10 scorecard. If a subject meets 3 or more of the 8 ICD-10 criteria, for example, and had head trauma with loss of consciousness, then the subject can be categorized as having PCS. PCS can be measured one week post injury, for example. If desired, it can be measured at 1, 2, 3, 4, 6, 8, 12, 16, 20, 24, 36, 52, or more weeks from the initial injury.

### Example 5: Mild Injury and Treatment—Single Injury

Specific Aim 1: Establish a Ghrelin Dosing Regimen and Time Course for Ghrelin Dosing Efficacy.

Rationale: The following series of experiments characterize an effective ghrelin concentration and a subsequent time-course of ghrelin dosing following mild TBI. These experiments should delineate not only optimal dosing of ghrelin following TBI, but as importantly, characterize the time course of ghrelin efficacy following mBI.

Dosing concentration model: Four groups of animals (n=5 per group): 1) Sham (no injury); 2) TBI (controlled cortical impact (CCI) injury mBI); 3) TBI+Drug (CCI mBI+substrate); and 4) Sham+Drug. For the (CCI) model, male 10-12 week old C57BL/6 mice are used. Substrates are dosed at 50 μg/kg via subcutaneous injection. Mice anesthetized with isoflurane and the body temperature maintained with a heating pad and thermostatic rectal probe in a BSL2 hood. The skull skin cut and using a dental drill with a trephine bit a 3 mm craniotomy over the right frontal cortex (1 mm anterior and 1 mm lateral to the bregma) is performed just before the dura. The animal is placed in the stereotactic frame, which restrains its head and keep the animal in place and continuously exposed to isoflurane. A CCI injury device (Leica Microsystems Inc., Buffalo Grove, Ill.) is used for mBI (Washington P. et. al. Journal of Neurotrauma, 2012). This device uses electromagnetic force to produce an impact velocity with speed, and dwell time all being individually manipulated to produce the mBI. The impact is delivered at a velocity of 5.0 m/s, a 3 mm diameter impounder tip, a depth of 1.0 mm, and a dwell time of 200 milliseconds. After the injury, the wound is glued using a tissue adhesive that polymerizes in seconds after contact with tissue. The mice respiratory rate and spontaneous movement, tail and foot pinch are monitored during the entire procedure to determine any discomfort or the need for additional analgesic. The animal's body temperature is maintained by using a water circulating heating pad.

Substrate dosing: The substrate are administered to randomized animals and given at two doses, 10 minutes following injury and at 1 hour following injury in varying concentrations. (50 μg/kg each dose, subcutaneous scruff).

Dosing concentrations: Begin first experiment at 0.1 μg/kg. Then increments increased accordingly, e.g., 0.1 μg/kg, 1 μg/kg, 5 μg/kg, 10 μg/kg, 20 μg/kg, 40 μg/kg, 50 μg/kg, 75 μg/kg, and 100 μg/kg. Following incubation in a dedicated post-surgical quarantine area, mice are sacrificed by inhalation of CO2 and the brains removed at 6 hours post injury for oxidative burst, and 24 hours for MPO.

Measurement of oxidative burst: (References: Chen Y et al. Methods in Molecular Biology, 2012, Banati R B, et al. Respiratory burst in brain macrophages: a flow cytometric study on cultured rat microglia. Neuropathology and Applied Neurobiology, 1991). Reagents used are HBSS 500 mL (GIBCO #14025-092) or PBS, Dihydrorhodamine 123 (DRH) (10 mg; Molecular Probes, Inc, Grand Island, N.Y. Cat # D632)

Oxidative burst in the brain is measured. Brain cells are isolated (incorporating the injury site, ⅛ of a 2 mm slab at injury site), subjected to collagenase Dispase (final 1 mg/ml, stored at −20° C.)+DNase (NEB 4C antibodies box deli fridge, add 50 ul into 20-50 ml at 37° C.) for 20 minutes, clumps are filtered with 70 um filter and washed in PBS. The pellet is resuspended in 200 uL of PBS (approx. 0.1×106 cells/ml). 4 uL of DHR (15 ng/mL) or 4 uL of DMSO is added to 96 ul of the brain cell samples (5 minutes at 37° C.) (protected from light). Respiratory burst using flow cytometry (FL1 channel with 488-nm laser as soon as possible) is measured.

Dosing Time Course: Four groups of animals (n=5 per group): 1) Sham (no injury), 2) TBI (controlled cortical impact (CCI) injury mBI), 3) TBI+Drug (CCI mBI+substrate), and 4) Sham+Drug. For the (CCI) model, male 10-12 week old C57BL/6 mice are used. Ideal dosing strategy are optimized with steps A and B. Time course for dosing are assessed in C. Substrate dosing and time course: The substrate is administered to randomized animals and given at two doses injury at a selected concentration. The second dose at each increment is given 1 hour after the first. Dose times are structured as follows: Time 0 (TBI)+30 minutes; Time 0 (TBI)+60 minutes; Time 0 (TBI)+90 minutes; Time 0 (TBI)+120 minutes; Time 0 (TBI)+240 minutes; Time 0 (TBI)+360 minutes; Time 0 (TBI)+480 minutes; Time 0 (TBI)+720 minutes; Time 0 (TBI)+1,440 minutes; and Time 0 (TBI)+2,880 minutes. Following incubation in a dedicated post-surgical quarantine area, mice are sacrificed by inhalation of CO2 and the brains removed at 6 hours post injury for oxidative burst.

The 30 mM DHR STOCK in DMSO is made by add 10 mg of DHR to 962 uL DMSO (Formula weight 346.38), add 5 uL of 30 mM DHR STOCK to 495 uL DMSO, add 10 uL DHR in DMSO to 267 uL HBSS, and add 4-5 uL to each reaction.

MPO Immunohistochemistry: Samples are cut from paraffin blocks. Water Bath is turned on to 95° C.—check water level. Deparafinization and slides are washed in the following solutions: a) Xylene 2×3 minutes; b) Xylene:ethanol 1:13 minutes; c) 100% ethanol 2×3 minutes; d) 95% ethanol 3 minutes; e) 70% ethanol 3 minutes; f) 50% ethanol 3 minutes; and g) cold dH2O to rinse (keep slides in water—DO NOT LET DRY). Antigen retrieval is performed as follows: a) Sodium citrate buffer-fill container and warm to near boiling in microwave; b) add slides and place into water bath; c) cover container and water bath with lids; and d) incubate for 20 minutes, remove container with slides to cool on counter for 20 minutes, then rinse slides with cold dH2O. The samples are rinsed in TAP water for 5 minutes. The tissues are outlined with PAP pen. Endogenous enzyme activity is blocked by incubating sections with 3% hydrogen peroxide in PBS for 5 minutes making 1:10 dilution of the 30% hydrogen peroxide stock solution in PBS. Sections are washed 5 minutes in PBS. Avidin/Biotin blocking is performed using Vector Avidin/Biotin Blocking Kit (Cat. No. SP-2001), using goat serum (2 drops in 10 ml PBS) for 20 minutes. Primary antibody 150-200ul/section is incubated in 1% BSA for 1 hour or o/n at r/t (MPO, Thermo # RB-373-A use at 1:100:rabbit). Vectastain ABC reagent is prepared in a 15 ml conical, 2 drops of reagent A is added to 5 ml PBS and mixed, 2 drops of Reagent B is added and mixed, and reagents are allowed to site for 30 minutes. The samples are rinsed 3× (5 minutes each) in PBS in slide chamber, and are incubated 30 minutes with biotinlyated secondary antibody (I drop in 10 ml). The slides are washed for 5 minutes in PBS. Vectastain ABC reagent is incubated for 30 minutes, and slides are washed for 5 minutes in PBS. DAB is performed in a 10 ml conical add 5 ml distilled water, 2 drops of Buffer stock solution (DAB kit) and mix, 4 drops of DAB stock solution (DAB kit) and mix, 2 drops of Hydrogen Peroxide solution (DAB kit) and mix apply solution to sections and incubate for 3 minutes. The slides are washed in tap water for 10 minutes, allowed to air-dry fully, and dehydrated by 50% ethanol for 3 minutes, 70% ethanol 3 minutes, 95% ethanol 3 minutes, 100% ethanol 2×3 minutes, Xylene:ethanol 1:1 3 minutes, and Xylene 2×3 minutes. Hematoxylin QS nuclear counterstain is added and incubate for 50 seconds. 10 uL of DPX is added to Mountant for histology over section and place coverslip flat while avoiding bubbles, and the slides are placed in a secure place.

Specific Aim 2: Verify Ghrelin's Effect on Neurocognitive Function Following Mild Brain Injury.

Rationale: Single impact mBI does not cause significant neurocognitive dysfunction compared to severe TBI. However, repeat mBI has been shown to induce neurocognitive defects and induce both amyloid deposition as well as early tauopathy. These experiments provide clarity that ghrelin dosing may lead to decreased cognitive defects, and improved histopathology. Assessment of hippocampal volume further assesses the protective effects of ghrelin. The specific cognitive studies can be done by a Morris-Water Maze test.

Injury and treatment model: Three groups of animals (n=10-15 per group): 1) Sham (no injury), 2) TBI (controlled cortical impact (CCI) injury mBI), and 3) TBI+Drug (CCI mBI+substrate). For the (CCI) model, male 10-12 week old C57BL/6 mice are used. Substrate dosing: The substrate is administered to randomized animals at two doses, 10 minutes following injury and at 1 hour following injury (50 μg/kg each dose, subcutaneous scruff). Repeat injury trial: Injury produced same brain region, same cortex, identical CCI settings: Day 0 CCI perform cognitive testing Day 2; Day 3 CCI perform cognitive testing Day 4; Day 6 CCI perform cognitive testing Day 7; and Day 7 euthanize for brain examination. Brain histopathology is performed on brains and analysis for: β-amyloidosis/Tau deposition; Neurodegeneration (Fluoro-Jade); Hippocampal volume; and Brian Morphology.

Specific Aim 3: Confirm the Mechanism Ghrelin Through UCP-2 Upregulation.

Rationale: Without wishing being bound by theory, it is contemplated that ghrelin's effects are a result of upregulation in UCP-2. By pre-dosing with genepin, a potent UCP-2 inhibitor, it is contemplated that ghrelin does not decrease oxidative burst following mBI. These experiments are designed to test the UCP-2 mechanistic theory of ghrelin. Additionally, a genetic ghrelin receptor (GHS-R1) KO mice may be implemented as an experimental group. It is contemplated that KO mice would not show the decreased oxidative burst patterns following ghrelin treatment in mBI.

Dosing concentration model: Four groups of animals (n=5 per group): 1) Sham (no injury), 2) TBI (controlled cortical impact (CCI) injury mBI), 3) TBI+Drug (CCI mBI+substrate), and 4) TBI+Drug and genepin (genepin to be dosed immediately before TBI at 15 mg/kg SQ). For the (CCI) model, male 10-12 week old C57BL/6 mice are used. Substrate dosing: The substrate are administered to randomized animals and given at two doses, 10 minutes following injury and at 1 hour following injury at an optimized regimen. Following incubation in a dedicated post-surgical quarantine area, mice are sacrificed by inhalation of CO2 and the brains removed at 6 hours post injury for oxidative burst (See Specific Aim 1).

Specific Aim 4: Identify Additional Substrates with Mild TBI Efficacy (Ghrelin Analogs)

Rationale: The following experiments characterize the activity of ghrelin analogs following mild TBI. These experiments identify additional variants that have activity in mild brain injury. Activity is compared to full-length ghrelin to determine the most potent molecule for possible clinical testing.

Injury and treatment model: Four groups of animals (n=5 per group): 1) Sham (no injury), 2) TBI (controlled cortical impact (CCI) injury mBI), 3) TBI+Drug (ghrelin variants disclosed herein) (CCI mBI+substrate), and 4) Sham+Drug. For the (CCI) model, male 10-12 week old C57BL/6 mice are used. Substrate to be dosed at levels consistent with activity demonstrated in previous clinical experiments. Substrate dosing: The substrate is administered to randomized animals at two doses, 10 minutes following injury and at 1 hour following injury (subcutaneous scruff). Following incubation in a dedicated post-surgical quarantine area, mice are sacrificed by inhalation of CO2 and the brains removed at 6 hours post injury for oxidative burst (See Specific Aim 1).

### Example 6: Mild Injury and Treatment—Repeat Injury

Injury and treatment model: Six groups of animals (n=5 per group): 1) Sham (no injury); 2) TBI (controlled cortical impact (CCI) injury mBI)—Single injury; 3) TBI (controlled cortical impact (CCI) injury mBI)—Repeat injury; 4) TBI+Drug (CCI mBI+substrate)—Single injury; 5) TBI+Drug (CCI mBI+substrate)—Repeat injury; and 6) Sham+Drug. For the (CCI) model, male 10-12 week old C57BL/6 mice are used. For single injury groups repeat Example 5, cognitive testing is performed prior to sacrifice. For Repeat injury groups—Injury produced same brain region, same cortex, identical CCI settings as Example 5 and to be produced on following schedule: Day 0 CCI, Day 4 CCI, Day 8 CCI. Same treatment follows as in Example 5 after each injury. On Day 12, cognitive testing is performed and mice are euthanized for brain examination. Cognitive testing is performed per Mouzon, et al, J Neurotrauma, 2012, or another accepted cognitive test such as the Water Maze test. Hippocampal volume is measured via imaging prior to sacrifice. Upon sacrifice, assays are performed as described in Example 5.

### Example 7: Dose Titration

Injury and treatment model: Three groups of animals (n=5 per group): 1) Sham (no injury), 2) TBI (controlled cortical impact (CCI) injury mBI), and 3) TBI+Drug (CCI mBI+substrate). For the (CCI) model, male 10-12 week old C57BL/6 mice are used. Mice anesthetized with isoflurane and the body temperature maintained with a heating pad and thermostatic rectal probe in a BSL2 hood. The skull skin cut and using a dental drill with a trephine bit a 3 mm craniotomy over the right frontal cortex (1 mm anterior and 1 mm lateral to the bregma) is performed just before the dura. The animal is placed in the stereotactic frame, which restrains its head and keep the animal in place and continuously exposed to isoflurane. A CCI injury device (Leica Microsystems Inc., Buffalo Grove, Ill.) is used for mBI (Washington P. et. al. Journal of Neurotrauma, 2012). This device uses electromagnetic force to produce an impact velocity with speed, and dwell time all being individually manipulated to produce the mBI. The impact is delivered at a velocity of 5.0 m/s, a 3 mm diameter impounder tip, a depth of 1.0 mm, and a dwell time of 200 milliseconds. After the injury, the wound is glued using a tissue adhesive that polymerizes in seconds after contact with tissue. The mice respiratory rate and spontaneous movement, tail and foot pinch are monitored during the entire procedure to determine any discomfort or the need for additional analgesic. The animal's body temperature is maintained by using a water circulating heating pad.

Substrate dosing: The substrate is administered to randomized animals and given at two doses, 10 minutes following injury and at 1 hour following injury in varying concentrations.

Dosing concentrations: Begin first experiment at 100 vg/kg. Then increments increased gradually, such as 0.1 μ/kg, 1 μ/kg, 5 μ/kg, 10 μ/kg, 20 μ/kg, 40 μ/kg, 50 μ/kg, 75 μ/kg, and 100 μ/kg. Following incubation in a dedicated post-surgical quarantine area, mice are sacrificed by inhalation of CO2 and the brains removed at 6 hours post injury.

Measurement of oxidative burst: (References: Chen Y et al. Methods in Molecular Biology, 2012, Banati R B, et al. Respiratory burst in brain macrophages: a flow cytometric study on cultured rat microglia. Neuropathology and Applied Neurobiology, 1991). Reagents used are: HBSS 500 mL (GIBCO #14025-092) or PBS, and Dihydrorhodamine 123 (DHR) (10 mg; Molecular Probes, Inc, Grand Island, N.Y. Cat # D632).

Protocol for measurement of oxidative burst in the brain is as follows: 1) Isolation of brain cells (incorporating the injury site, ⅛ of a 2 mm slab at injury site), Collagenase Dispase (final 1 mg/ml, stored at −20° C.)+DNase (NEB 4C antibodies box deli fridge, add 50 uL into 20-50 ml) at 37° C. for 20 minutes, filter clumps with 70 um filter, and wash in PBS; 2) Resuspend pellet in 200 uL of PBS (approx. 0.1×106 cells/ml); 3) Add 4 uL of DHR (15 ng/mL) or 4 uL of DMSO to 96 ul of the brain cell samples (5 minutes at 37° C.) (protected from light); and 4) Measure respiratory burst using flow cytometry (FL1 channel with 488-nm laser as soon as possible).

### Example 8: Mild Injury and Treatment—Repeat Injury+Minimum Efficacious Dose

Injury and treatment model: three groups of animals (n=5 per group): 1) Sham (no injury), 2) TBI (controlled cortical impact (CCI) injury mBI)—Repeat injury, and 3) TBI+Drug (CCI mBI+substrate)—Repeat injury. For the (CCI) model, male 10-12 week old C57BL/6 mice are used. Dose refers to the minimum efficacious dose identified in Example 7. For single injury groups repeat Example 5. Cognitive testing prior to sacrifice is performed. For Repeat injury groups—Injury produced same brain region, same cortex, identical CCI settings as Example 5 and to be produced on following schedule: Day 0 CCI, Day 4 CCI, Day 8 CCI. Treatment to follow Example 5 after each injury. On Day 12, cognitive testing is performed per step three, and the mice are euthanize for brain examination. Cognitive testing per Mouzan, et al, J Neurotrauma, 2012, or another accepted cognitive test such as the Water Maze test is performed. Hippocampal volume is measured via imaging prior to sacrifice. Upon sacrifice, assays are performed described in Example 5. Repeat experiment at next higher dose if no cognitive behavior difference are seen between injured/untreated and injured/treated groups until differences are observed.

### Example 9: Maximum Effective Treatment Window

Injury and treatment model: 17 groups of animals (n=5 per group): 1) Sham (no injury); TBI (CCI injury mBI) groups treated at 2) 10 minutes, 3) 2 hours, 4) 4 hours, 5) 6 hours, 6) 8 hours, 7) 12 hours, 8) 24 hours, 9) 48 hours; TBI+Drug (CCI mBI+substrate) groups treated at 10) 10 minutes, 11) 2 hours, 12) 4 hours, 13) 6 hours, 14) 8 hours, 15) 12 hours, 16) 24 hours, and 17) 48 hours. For the (CCI) model, male 10-12 week old C57BL/6 mice are used. The dose is defined based on results of Example 8. Mice anesthetized with isoflurane and the body temperature maintained with a heating pad and thermostatic rectal probe in a BSL2 hood. The skull skin cut and using a dental drill with a trephine bit a 3 mm craniotomy over the right frontal cortex (1 mm anterior and 1 mm lateral to the bregma) is performed just before the dura. The animal is placed in the stereotactic frame, which restrains its head and keep the animal in place and continuously exposed to isoflurane. A CCI injury device (Leica Microsystems Inc., Buffalo Grove, Ill.) is used for mBI (Washington P. et. al. Journal of Neurotrauma, 2012). This device uses electromagnetic force to produce an impact velocity with speed, and dwell time all being individually manipulated to produce the mBI. The impact is delivered at a velocity of 5.0 m/s, a 3 mm diameter impounder tip, a depth of 1.0 mm, and a dwell time of 200 milliseconds. After the injury, the wound is glued using a tissue adhesive that polymerizes in seconds after contact with tissue. The mice respiratory rate and spontaneous movement, tail and foot pinch is monitored during the entire procedure to determine any discomfort or the need for additional analgesic. The animal's body temperature is maintained by using a water circulating heating pad.

Substrate dosing: The substrate is administered to randomized animals and given at two doses. The first dose is administered at the start time designated for the group and the second dose is given at 1 hour post first dose. The dose is defined based on results of Example 8. Following incubation in a dedicated post-surgical quarantine area, mice are sacrificed by inhalation of CO2 and the brains removed at 6 hours post injury.

Measurement of oxidative burst: References: Chen Y et al. Methods in Molecular Biology, 2012, Banati R B, et al. Respiratory burst in brain macrophages: a flow cytometric study on cultured rat microglia. Neuropathology and Applied Neurobiology, 1991. Reagents used are HBSS 500 mL (GIBCO #14025-092) or PBS, and Dihydrorhodamine 123 (DHR) (10 mg; Molecular Probes, Inc, Grand Island, N.Y. Cat # D632).

Protocol for measurement of oxidative burst in the brain are as follows: 1) isolation of brain cells (incorporating the injury site, ⅛ of a 2 mm slab at injury site) with Collagenase Dispase (final 1 mg/ml, stored at −20° C.)+DNase (NEB 4C antibodies box deli fridge, add 50 uL into 20-50 mL) at 37° C. for 20 minutes, filter clumps with 70 um filter, wash in PBS, resuspend pellet in 200 uL of PBS (approx. 0.1×106 cells/ml), add 4 uL of DHR (15 ng/mL) or 4 uL of DMSO to 96 ul of the brain cell samples (5 minutes at 37° C.) (protected from light), and measure respiratory burst using flow cytometry (FL1 channel with 488-nm laser as soon as possible).

It is to be understood that while the present disclosure has been described in conjunction with the above embodiments, that the foregoing description and examples are intended to illustrate and not limit the scope of the present disclosure. Other aspects, advantages and modifications within the scope of the present disclosure will be apparent to those skilled in the art to which the present disclosure pertains.

The present disclosure is not to be limited in scope by the specific embodiments described which are intended as single illustrations of individual aspects of the present disclosure, and any compositions or methods, which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method of treating mild traumatic brain injury (mTBI) in a subject in need thereof, comprising administering to the subject a therapeutically effective amount of a ghrelin variant, thereby treating the mTBI wherein the ghrelin variant is one or more of RM-131 (or BIM-28131), Dln-101, Growth hormone releasing hexapeptide (GHRP)-6, EP 1572, Ave-Ser(Octyl)-Phe-Leu-aminoethylamide, growth hormone secretagogue receptor GHS-R 1a ligand, LY444711, LY426410, hexarelin/examorelin, growth hormone releasing hexapeptide-1 (GHRP-I), GHRP-2, GHRP-6 (SK&F-110679), ipamorelin, MK-0677, NN703, capromorelin, CP 464709, pralmorelin, macimorelin (acetate), anamorelin, relamorelin, ulimorelin, ipamorelin, tabimorelin, ibutamoren, G7039, G7134, G7203, G-7203, G7502, SM-130686, RC-1291, L-692429, L-692587, L-739943, L-163255, L-163540, L-163833, L-166446, CP-424391, EP-51389, NNC-26-0235, NNC-26-0323, NNC-26-0610, NNC 26-0703, NNC-26-0722, NNC-26-1089, NNC-26-1136, NNC-26-1137, NNC-26-1187, NNC-26-1291, MK-0677, L-692,429, EP 1572, L-252,564, NN703, S-37435, EX-1314, PF-5190457, AMX-213, and a combination thereof.

2-9. (canceled)

10. The method of claim 1, wherein the ghrelin variant comprises a polypeptide comprising the sequence of Gly Ser Xaa Phe Leu Ser Pro Glu His Gin Arg Val Gin Val Arg Pro Pro His Lys Ala Pro His Val Val (SEQ ID No. 3), wherein the third position is a 2,3-diaminopropionic acid (Dpr) and optionally octanoylated.

11. The method of claim 1, wherein the ghrelin variant comprises a polypeptide comprising the sequence of Gly Xaa Xaa Phe Leu Ser Pro Glu His Gin Arg Val Gin Val Arg Pro Pro His Lys Ala Pro His Val Val (SEQ ID No. 4), wherein the second and third position are 2,3-diaminopropionic acid (Dpr) residues, with the Dpr in the third position being optionally octanoylated.

12. (canceled)

13. The method of claim 1, wherein the ghrelin variant comprises a polypeptide comprising the sequence of Inp-D-2Nal-D-Trp-Thr-Lys-NH₂ (SEQ ID No. 6).

14. (canceled)

15. (canceled)

16. The method of claim 1, wherein the mTBI is concussion.

17. The method of claim 1, wherein the ghrelin variant binds to the growth hormone secretagogue receptor GHS-R 1a (GHSR).

18. The method of claim 1, wherein the ghrelin variant has an EC₅₀ potency on the GHSR of less than 500 nM.

19. The method of claim 1, wherein the ghrelin variant has a dissociation constant from the GHSR of less than 500 nM.

20-24. (canceled)

25. The method of claim 1, wherein the ghrelin variant prevents or reduces the rate or incidence of post-concussive syndrome.

26. The method of claim 1, wherein the ghrelin variant is coupled to a protein that extends the serum half-life of the ghrelin variant.

27-29. (canceled)

30. The method of claim 1, wherein the subject is a mammal.

31. The method of claim 30, wherein the subject is a human.

32. The method of claim 1, wherein the ghrelin variant is administered within not more than about 72 hours of the mTBI.

33. The method of claim 32, wherein the ghrelin variant is administered within not more than about 24 hours of the mTBI.

34. (canceled)

35. A method of reducing the incidence of or severity of mild traumatic brain injury (mTBI) in a subject in need thereof, comprising administering to the subject an effective amount of a ghrelin variant, thereby reducing the incidence or severity of the mTBI, wherein the ghrelin variant is one or more of RM-131 (or BIM-28131), Dln-101, Growth hormone (GH) releasing hexapeptide (GHRP)-6, EP 1572, Ape-Ser(Octyl)-Phe-Leu-aminoethylamide, growth hormone secretagague receptor GHS-R 1a ligand, LY444711, LY426410, hexarelin/examorelin, growth hormone releasing hexapeptide-1 (GHRP-I), GHRP-2, GHRP-6 (SK&F-110679), ipamorelin, MK-0677, NN703, capromorelin, CP 464709, pralmorelin, macimorelin (acetate), anamorelin, relamorelin, ulimorelin, ipamorelin, tabimorelin, ibutamoren, G7039, G7134, G7203, G-7203, G7502, SM-130686, RC-1291, L-692429, L-692587, L-739943, L-163255, L-163540, L-163833, L-166446, CP-424391, EP-51389, NNC-26-0235, NNC-26-0323, NNC-26-0610, NNC 26-0703, NNC-26-0722, NNC-26-1089, NNC-26-1136, NNC-26-1137, NNC-26-1187, NNC-26-1291, MK-0677, L-692,429, EP 1572, L-252,564, NN703, S-37435, EX-1314, PF-5190457, AMX-213, and a combination thereof.

36-47. (canceled)

48. The method of claim 1, wherein the ghrelin variant is administered in combination with a therapeutic agent.

49. The method of claim 48, wherein the therapeutic agent is one or more of an anti-inflammatory agent, anti-pain medication, acetylsalicylic acid, an antiplatelet agent, a thrombolytic enzyme, an aggregation inhibitor, a glycoprotein Ilb/IIIa inhibitor, a glycosaminoglycan, a thrombin inhibitor, an anticoagulant, heparin, coumarin, tPA, GCSF, streptokinase, urokinase, Ancrod, melatonin, a caspase inhibitor, an NMDA receptor agonist or antagonist (e.g. OTO-311), an anti-TNF-α compound, an antibody, erythropoietin/EPO, angiotensin II lowering agent, selective androgen receptor modulator, leptin or leptin mimetics and variants, an agonists of the renin-angiotensin system, an opioid receptor agonist, progesterone or progesterone mimetics and variants, a peroxisome proliferator-activated receptor gamma agonist, P2Y purinergic receptor agonists (e.g., 2-MeSADP, MRS2365), amantadine (e.g. ADS-5102), P7C3, or a combination thereof.

50. A therapeutic product comprising ghrelin or a ghrelin variant and an agent selected from an anti-inflammatory agent, anti-pain medication, acetylsalicylic acid, an antiplatelet agent, a thrombolytic enzyme, an aggregation inhibitor, a glycoprotein Ilb/IIIa inhibitor, a glycosaminoglycan, a thrombin inhibitor, an anticoagulant, heparin, coumarin, tPA, GCSF, streptokinase, urokinase, Ancrod, melatonin, a caspase inhibitor, an NMDA receptor agonist or antagonist, an anti-TNF-α compound, an antibody, erythropoietin/EPO, angiotensin II lowering agent, selective androgen receptor modulator, leptin or leptin mimetics and variants, an agonists of the renin-angiotensin system, an opioid receptor agonist, progesterone or progesterone mimetics and variants, a peroxisome proliferator-activated receptor gamma agonist, an NMDA receptor agonist or antagonist (e.g. OTO-311), P2Y purinergic receptor agonists (e.g., 2-MeSADP, MRS2365), P7C3, aducanumab, and amantadine (e.g. ADS-5102).

51-68. (canceled)

69. A method for detecting and treating mild traumatic brain injury or concussion in a subject in need thereof, comprising measuring the amount of biomarkers in a sample of the subject after the occurrence of a mild traumatic brain injury or concussion; comparing the amount of the biomarkers in the sample with a sample from an uninjured subject; and administering to the subject a therapeutically effective amount of a composition comprising ghrelin and/or ghrelin variant.

70. (canceled)
